(19) United States
(12) Patent Application Publication
(10) Pub. No.: US 2025/0099617 A1
(43) Pub. Date: Mar. 27, 2025

(54) AAV TRANSFER PLASMIDS

(71) Applicant: Atsena Therapeutics, Inc., Durham, NC (US)

(72) Inventor: Christopher KOZLOWSKI, Durham, NC (US)

(21) Appl. No.: 18/291,470

(22) PCT Filed: Jul. 27, 2022
(86) PCT No.: PCT/US2022/038520

§ 371 Date: Jan. 23, 2024

Related U.S. Application Data
(60) Provisional application No. 63/226,410, filed on Jul. 28, 2021.

(51) Int. Cl. A61K 48/00; A61P 27/02; C07K 14/47; C12N 15/86

(52) U.S. Cl. CPC A61K 48/0058; A61P 27/02; C07K 14/47; C12N 15/86; C12N 2750/14122; C12N 2750/14143; C12N 2750/14145

(57) ABSTRACT

Provided herein are compositions and methods useful for the production of Adeno-associated virus particles.

### CROSS-REFERENCE

This application is the 371 U.S. National Stage Application of PCT/US2022/038520, filed Jul. 27, 2022, which claims the benefit of U.S. Provisional Application No. 63/226,410, filed Jul. 28, 2021, each of which are incorporated herein by reference in their entirety.

### INCORPORATION BY REFERENCE OF SEQUENCE LISTING

The instant application contains a Sequence Listing which has been submitted electronically in XML format and is hereby incorporated by reference in its entirety. The Sequence Listing is provided as a file entitled 58774-726_831_SL.XML, created Dec. 26, 2023, which is 155,873 bytes in size.

### SUMMARY

In certain aspects, disclosed herein is a nucleic acid stuffer sequence comprising one or more of the following features: no CPG island and no more than four contiguous nucleobases of the same identity; wherein the nucleic acid stuffer sequences does not include an open reading frame greater than 20 amino acids in length. In some embodiments, the nucleic acid stuffer sequence has no CPG island. In some embodiments, the nucleic acid stuffer sequence has no more than four contiguous nucleobases of the same identity. In some embodiments, the nucleic acid stuffer sequence comprises between about 40% and about 50% GC content. In some embodiments, the nucleic acid stuffer sequence does not comprise a restriction enzyme cleavage site. In some embodiments, the nucleic acid stuffer sequence has a length of about 100 nucleobases to about 5000 nucleobases. In some embodiments, the nucleic acid stuffer sequence has a length of about 2200 nucleobases to about 2300 nucleobases. In some embodiments, the nucleic acid stuffer sequence has a length of about 3000 nucleobases to about 3100 nucleobases. In some embodiments, the nucleic acid stuffer sequence has a length of about 300 nucleobases to about 400 nucleobases.

In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 7, 8, or 11. In certain aspects, disclosed herein is a nucleic acid stuffer sequence comprising a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 7. In certain aspects, disclosed herein is a nucleic acid stuffer sequence comprising a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 8. In certain aspects, disclosed herein is a nucleic acid stuffer sequence comprising a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 11. In some embodiments, the nucleic acid stuffer sequence comprises no CPG island. In some embodiments, the nucleic acid stuffer sequence comprises no more than four contiguous nucleobases of the same identity. In some embodiments, the nucleic acid stuffer sequence comprises between about 40% and about 50% GC content. In some embodiments, the nucleic acid stuffer sequence does not comprise a restriction enzyme cleavage site. In some embodiments, the nucleic acid stuffer sequence has a length of about 100 to about 5000 nucleobases. In some embodiments, disclosed herein is an adeno-associated virus (AAV) plasmid comprising the nucleic acid stuffer sequence disclosed herein. In some embodiments, the AAV plasmid further comprises an expression cassette comprising a heterologous sequence positioned between two inverted terminal repeat (ITR) sequences. In some embodiments, the AAV plasmid further comprises a backbone having a length of about 2000 to about 8000 nucleobases. In some embodiments, the AAV plasmid further comprises a backbone having a length of about 5500 to about 6000 nucleobases. In some embodiments, the expression cassette has a length of about 3000 to about 6000 nucleobases. In some embodiments, the expression cassette has a length of about 4000 nucleobases to about 5000 nucleobases. In some embodiments, the heterologous sequence encodes for a therapeutic peptide. In some embodiments, the therapeutic peptide is selected from the group consisting of GUCY2D, MYO7A, RS1, CNBG3, ADAMTS10, ABCA4, and frataxin. In some embodiments, the AAV plasmid further comprises an antibiotic resistance gene. In some embodiments, the antibiotic resistance gene comprises a kanamycin resistance gene. In some embodiments, the AAV plasmid does not comprise an antibiotic resistance gene. In some embodiments, the AAV plasmid does not comprise an ampicillin antibiotic resistance gene. In some embodiments, the ITR is derived from AAV serotype 1, AAV serotype 2, AAV serotype 3, AAV serotype 4, AAV serotype 5, AAV serotype 6, AAV serotype 7, AAV serotype 8, AAV serotype 9, AAV serotype 10, or AAV449.5(e531d). In some embodiments, the AAV plasmid further comprises a promoter. In some embodiments, the AAV plasmid further comprises a splice donor/splice acceptor sequence. In some embodiments, the AAV plasmid further comprises a WPRE sequence. In some embodiments, the nucleic acid stuffer sequence is positioned outside of the expression cassette. In some embodiments, the AAV plasmid further comprises an origin of replication. In some embodiments, the nucleic acid stuffer sequence is positioned 3′ to the origin of replication. In some embodiments, the nucleic acid stuffer sequence is positioned between the origin of replication and an ITR. In some embodiments, the nucleic acid stuffer sequence is located such that the ITR is about 1000 to about 4000 nucleobases away from the origin of replication. In some embodiments, the nucleic acid stuffer sequence comprises a first stuffer sequence and a second stuffer sequence. In some embodiments, the first stuffer sequence has a length of about 3000 to about 3500 nucleobases. In some embodiments, the first stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 8. In some embodiments, the second stuffer sequence has a length of about 100 to about 500 nucleobases. In some embodiments, the second stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 11. In some embodiments, the nucleic acid stuffer sequence is positioned within the expression cassette. In some embodiments, the nucleic acid stuffer sequence has a length of about 2000 to about 3000 nucleobases. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 7. In some embodiments, presence of the nucleic acid stuffer sequence reduces mutation of one or both of ITRs as compared to an AAV plasmid that does not comprise the nucleic acid stuffer sequence. In some embodiments, the AAV plasmid has no more than one origin of replication. In some embodiments, the AAV plasmid does not have a m13 origin of replication. In some embodiments, the AAV plasmid does not comprise a polyG/C sequence. In some embodiments, the AAV plasmid does not comprise a sequence having more than 2, 3, 4, 5, 6, 7, 8, 9, or 10 contiguous guanine bases.

In some embodiments, disclosed herein is a composition comprising the AAV plasmid disclosed herein, and a packaging plasmid comprising a viral replication (rep) gene and/or a viral capsid (cap) gene. In some embodiments, the packaging plasmid comprises the rep gene. In some embodiments, the rep gene encodes for rep78, rep68, rep52, and rep40. In some embodiments, the packaging plasmid comprises the cap gene. In some embodiments, the cap gene encodes for vp1, vp2 and vp3. In some embodiments, the cap gene comprises at least about 70%, 75%, 80%, 85%, 90%, 95%, 96%, 97%, 98%, 99% or 100% sequence identity with SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 31. In some embodiments, the composition comprises a helper plasmid.

In some embodiments, disclosed herein is a composition comprising the AAV plasmid described herein, and a helper plasmid. In some embodiments, the helper plasmid comprises E1a gene, a E1b gene, a E4 gene, a E2a, a e3 gene, a E5 gene, a Fiber gene, or a VA gene, or a combination thereof. In some embodiments, the helper plasmid comprises a mutated Fiber gene. In some embodiments, the helper plasmid comprises a mutated Fiber gene. In some embodiments, the helper plasmid does not comprise a Fiber gene.

In some embodiments, disclosed herein is a cell comprising the AAV plasmid disclosed herein or the composition disclosed herein.

In some embodiments, disclosed herein is an AAV particle comprising a nucleic acid and a capsid, wherein the AAV particle is produced by the AAV plasmid disclosed herein, the composition disclosed herein, or the cell disclosed herein. In some embodiments, described herein is a pharmaceutical composition comprising the AAV particle disclosed herein and a pharmaceutically acceptable, carrier, buffer, diluent, or excipient, or any combination thereof.

In some embodiments, disclosed herein is a method for transducing a cell, the method comprising administering to the cell the AAV vector disclosed herein, the composition disclosed herein, the AAV particle disclosed herein, or the pharmaceutical composition disclosed herein. In some embodiments, the cell is a photoreceptor cell. In some embodiments, the cell is a retinal pigment epithelial (RPE) cell. In some embodiments, the cells is a retinal ganglion cell.

In some embodiments, disclosed herein is a method for treating a disease or condition of the eye in a mammal, the method comprising administering to the mammal the AAV particle disclosed herein or the pharmaceutical composition disclosed herein. In some embodiments, the disease or condition comprises Retinitis pigmentosa, Leber Congenital Amaurosis (e.g., LCA10), Age Related Macular Degeneration (AMD), wet AMD, dry AMD, uveitis, Best disease, Stargardt disease, Usher Syndrome, Geographic Atrophy, Diabetic Retinopathy, Retinoschisis, Achromatopsia, Choroideremia, Bardet Biedl Syndrome, or glycogen storage diseases (ocular manifestation). In some embodiments, the administration is to one or both eyes of the mammal. In some embodiments, the AAV particle is administered intravitreally or subretinally.

In some embodiments, described herein is the nucleic acid stuffer sequence disclosed herein, the AAV plasmid disclosed herein, the composition disclosed herein, the cell disclosed herein, the AAV particle disclosed herein, or the pharmaceutical composition disclosed herein for use in the treatment of a disease or condition of the eye. In some embodiments, disclosed herein is the use of the nucleic acid stuffer sequence disclosed herein, the AAV plasmid disclosed herein, the composition disclosed herein, the cell disclosed herein, the AAV particle disclosed herein, or the pharmaceutical composition disclosed herein in the manufacture of a medicament for use in the treatment of a disease or condition of the eye

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts a representative example of an AAV-backbone plasmid digested with SmaI.

FIG. 1B depicts an AAV backbone plasmid grown in ampicillin resistant cells and digested with SmaI.

FIG. 1C depicts an AAV backbone plasmid grown in kanamycin resistant cells and digested with SmaI.

FIG. 2A depicts a polyG/C sequence in an AAV plasmid backbone.

FIG. 2B depicts an AAV backbone plasmid with the polyG/C sequence removed digested with SmaI.

FIG. 3 depicts the schematic showing different processes which could result in errors in AAV packaging.

FIG. 4A depicts a AAV plasmid backbone with a stuffer sequence located 3′ of the origin.

FIG. 4B depicts the AAV backbone plasmid of FIG. 4A digested with SmaI.

FIG. 5A depicts a AAV plasmid backbone with a stuffer sequence located 5′ of the origin.

FIG. 5B depicts the AAV backbone plasmid of FIG. 5A digested with SmaI.

FIG. 6 depicts an AAV plasmid backbone with a stuffer sequence located 5′ of the origin and a polyG/C sequence digested with SmaI.

FIG. 7 compares ITR stability between AAV backbone plasmids grown in ampicillin-resistant cells or kanamycin-resistant cells.

FIG. 8A depicts a schematic of the pTR-X002-3pSR transfer plasmid used.

FIG. 8B depicts the process of creating the pTR-X002-3pSR transfer plasmid.

FIG. 8C depicts a schematic of a Rep/Cap plasmid.

FIG. 8D depicts a schematic of a helper plasmid.

FIGS. 9A-9C show schematics for six cassettes from exemplary vectors of the disclosure. Exemplary vectors include pTR-X001-3p, which has a length of 4534 bp from the 5′ end of the first ITR to the 3′ end of the second ITR (FIG. 9A), pTR-X001-5p, which has a length of 4528 bp from the 5′ end of the first ITR to the 3′ end of the second ITR (FIG. 9B), and pTR-X002-3p, which has a length of 4549 bp from the 5′ end of the first ITR to the 3′ end of the second ITR (FIG. 9C). pTR-GRK1-hRS1syn was packaged in AAV5 and AAV.SPR. pTR-CBA-hRS1syn vector plasmid (pTR-UF11 backbone) was packaged in AAV5 to serve as a control.

FIG. 10 shows restoration of retinal structure in RS1KO mice treated with rAAV.SPR containing stuffed cassettes. Quantification of schisis cavity scores in RS1KO mice treated with either vehicle or rAAV.SPR vectors containing the following cassettes: X001, X001-3p, X001-5p, or X002-3p. All vectors improved retinoschisis scores at both timepoints, except for X001-5p at 1-month post-injection. Nominal descriptive statistical significance was determined with a two-way ANOVA with a Tukey's post-test on the treated eyes.

FIGS. 11A-11B show restoration of retinal function in RS1KO mice treated with rAAV.SPR containing stuffed cassettes. Average maximum scotopic (left) and photopic (right) b-wave amplitudes in RS1KO mice measured 1- and 2-months after subretinal injection in one eye with either vehicle or rAAV.SPR containing the following cassettes: X001, X001-3p, X001-5p, or X002-3p. Vector was dosed at either 1×10⁸ vg (FIG. 11A) or 5×10⁸ vg (FIG. 11B). Retinal function in eyes treated with all vectors was improved over untreated control eyes. Nominal descriptive statistical significance was determined with two-way ANOVA with Tukey's post-test on each individual data set. *p<0.05, **p<0.01, ***p<0.001, ****p<0.0001.

FIGS. 12A-12B shows RS1 expression in retinas of RS1KO mice treated with rAAV.SPR-X002-3p. Representative retinal cross sections from RS1KO mice treated (top) in one eye only either vehicle or rAAV.SPR containing the X002-3p cassette. Vector was dosed at either 1×10⁸ vg (FIG. 12A) or 5×10⁸ vg (FIG. 12B). Contralateral untreated eyes are shown in the bottom row. All retinas stained with an antibody raised against RS1 (red) and counterstained with DAPI.

### DETAILED DESCRIPTION

### I. Compositions of Matter

Recombinant adeno-associated virus (rAAV) vectors have been used successfully for in vivo gene transfer in numerous pre-clinical animal models of human disease, and have been used successfully for long-term expression of a wide variety of therapeutic. AAV vectors have also generated long-term clinical benefit in humans when targeted to immune-privileged sites, e.g., ocular delivery for Leber's congenital amaurosis. An advantage of this vector is its comparatively low immune profile, eliciting only limited inflammatory responses and, in some cases, even directing immune tolerance to transgene products.

Adeno-associated virus (AAV) is used for ocular gene therapy due to its efficiency, persistence and low immunogenicity. Aspects of the disclosure relate to recombinant adeno-associated virus (rAAV) particles or preparations of such particles for delivery of one or more nucleic acid vectors comprising a protein or polypeptide of interest, into various tissues, organs, and/or cells.

Described herein are improved systems for the manufacturing and packaging of AAV particles. In some embodiments, the systems and methods described herein improve the efficiency of packaging AAV particles, improve the accuracy of replicating AAV particles, or a combination thereof.

### A. Stuffer Sequence

In some embodiments, described herein is a nucleic acid stuffer sequence. As described herein, a “stuffer sequence”, referred to interchangeably as a “nucleic acid stuffer sequence,” may be a nucleic acid sequence that resizes or adjusts the length to near or the normal length of the AAV virus genomic sequence. In some embodiments, the nucleic acid stuffer sequence does not comprise a CpG island, the nucleic acid stuffer sequence does not comprise more than four contiguous nucleobases of the same identity, the nucleic acid stuffer sequence comprises between about 40% and about 50% GC content, the nucleic acid stuffer sequence does not comprise a restriction enzyme cleavage site, or the nucleic acid stuffer sequence does not encode for an open reading frame (ORF) larger than 20 amino acids, or a combination thereof. In some embodiments, the nucleic acid stuffer sequence does not comprise a CpG island, the nucleic acid stuffer sequence does not comprise more than four contiguous nucleobases of the same identify, the nucleic acid stuffer sequence comprises between about 40% and about 50% GC content, the nucleic acid stuffer sequence does not encode for an open reading frame (ORF) larger than 20 amino acids, and the nucleic acid stuffer sequence does not comprise a restriction enzyme cleavage site.

In some embodiments, the nucleic acid stuffer does not comprise a CpG island. In some embodiments, CpG island (CGI) comprises a large number of CpG dinucleotide repeats. In some embodiments, a CpG island comprises a region at least 100 basepairs in length where the GC content exceeds 50% GC. In some embodiments, a CpG island comprises a region at least 100, 200, 300, 400, 500 or more basepairs in length. In some embodiments, a CpG island comprises a region where the GC content is at least 50%, 60%, 70%, 80%, 90% or more than 90%.

In some embodiments, the nucleic acid stuffer sequence does not comprise more than 3, 4, 5, 6, 7, 8, 9, or 10 contiguous nucleobases of the same identity. In some embodiments, the nucleic acid stuffer sequence does not comprise more than 3, 4, 5, 6, 7, 8, 9, or 10 contiguous adenosines. In some embodiments, the nucleic acid stuffer sequence does not comprise more than 3, 4, 5, 6, 7, 8, 9, or 10 contiguous cytosines. In some embodiments, the nucleic acid stuffer sequence does not comprise more than 3, 4, 5, 6, 7, 8, 9, or 10 contiguous guanines. In some embodiments, the nucleic acid stuffer sequence does not comprise more than 3, 4, 5, 6, 7, 8, 9, or 10 contiguous thymines.

In some embodiments, the nucleic acid stuffer sequence comprises between about 30% and about 60% GC content. In some embodiments, the nucleic acid stuffer sequence comprises between about 40% and about 50% GC content. In some embodiments, the nucleic acid stuffer sequence comprises about 45% GC content. In some embodiments, the nucleic acid stuffer sequence comprises between about 0% and 10% GC content, between about 5% and 15% GC content, between about 10% and 20% GC content, between about 15% and 20% GC content, between about 25% and 35% GC content, between about 30% and 40% GC content, between about 35% and 45% GC content, between about 40% and 50% GC content, between about 45% and 55% GC content, between about 50% and 60% GC content, between about 55% and 65% GC content, between about 60% and 70% GC content, between about 65% and 75% GC content, between about 70% and 80% GC content, between about 75% and 85% GC content, between about 80% and 90% GC content, between about 85% and 95% GC content, or between about 05% and 100% GC content.

In some embodiments, the nucleic acid stuffer sequence does not comprise a restriction enzyme cleavage site. In some embodiments, the restriction enzyme cleavage site is a AatII, AbaSI, AccI, Acc65I, AciI, AclI, AcuI, AfeI, AflII, AflIII, AgeI §, AgeI-HF®, AhdI, AleI-v2, AluI, AlwI, AlwNI, ApaI, ApaLI, ApeKI, ApoI §, ApoI-HF, AscI, AseI, AsiSI, AvaI, AvaII, AvrII, BaeGI, BaeI, BamHI §, BamHI-HF®, BanI, BanII, BbsI §, BbsI-HF®, BbvCI, BbvI, BccI, BceAI, BcgI, BciVI, BclI §, BclI-HF, BcoDI, BfaI, BfuAI, BglI, BglII, BlpI, BmgBI, BmrI, BmtI §, BmtI-HF®, BpmI, BpuEI, Bpu10I, BsaAI, BsaBI, BsaHI, BsaI-HF®v2, BsaJI, BsaWI, BsaXI, BseRI, BseYI, BsgI, BsiEI, BsiHKAI, BsiWI §, BsiWI-HF®, BslI, BsmAI, BsmBI-v2, BsmFI, BsmI, BsoBI, BspCNI, BspDI, BspEI, BspHI, Bsp1286I, BspMI, BspQI, BsrBI, BsrDI, BsrFI-v2, BsrGI §, BsrGI-HF®, BsrI, BssHII, BssSI-v2, BstAPI, BstBI, BstEII §, BstEII-HF®, BstNI, BstUI, BstXI, BstYI, BstZ17I-HF®, Bsu36I, BtgI, BtgZI, BtsCI, BtsIMutI, BtsI-v2, Cac8I, ClaI, CspCI, CviAII, CviKI-1, CviQI, DdeI, DpnI, DpnII, DraI, DraIII-HF®, DrdI, EaeI, EagI-HF®, Earl, EciI, Eco53kI, EcoNI, EcoO109I, EcoP15I, EcoRI §, EcoRI-HF®, EcoRV §, EcoRV-HF®, Esp3I, FatI, FauI, Fnu4HI, FokI, FseI, FspEI, FspI, HaeII, HaeIII, HgaI, HhaI, HincII, HindIII §, HindIII-HF®, Hinfl, HinP1I, HpaI, HpaII, HphI, HpyAV, HpyCH4III, HpyCH4IV, HpyCH4V, Hpy188I, Hpy99I, Hpy166II, Hpy188III, I-CeuI, I-SceI, KasI, KpnI §, KpnI-HF®, LpnPI, MboI, MboII, MfeI §, MfeI-HF®, MiuCI, MluI §, MiuI-HF®, MiyI, MmeI, MnlI, MscI, MseI, MsiI, MspA1I, MspI, MspJI, MwoI, NaeI, NarI, Nb.BbvCI, Nb.BsmI, Nb.BsrDI, Nb.BssSI, Nb.BtsI, NciI, NeoI §, NeoI-HF®, NdeI, NgoMIV, NheI-HF®, NlaIII, NiaIV, NmeAIII, NotI §, NotI-HF®, NruI §, NruI-HF®, NsiI §, NsiI-HF®, NspI, Nt.AiwI, Nt.BbvCI, Nt.BsmAI, Nt.BspQI, Nt.BstNBI, Nt.CviPII, PacI, PaeR7I, PaqCI, PciI, PflFI, PflMI, PI-PspI, PI-SceI, PleI, PluTI, PmeI, PmlI, PpuMI, PshAI, PsiI-v2, PspGI, PspOMI, PspXI, PstI §, PstI-HF®, PvuI §, PvuI-HF®, PvuII §, PvuII-HF®, RsaI, RsrII, SacI §, SacI-HF®, SacII, SalI §, SalI-HF®, SapI, Sau3AI, Sau96I, Sbfl §, Sbfl-HF®, ScaI-HF®, ScrFI, SexAI, SfaNI, SfcI, SfiI, SfoI, SgrAI, SmaI, SmlI, SnaBI, SpeI §, SpeI-HF®, SphI §, SphI-HF®, Srfl, SspI §, SspI-HF®, StuI, StyD4I, StyI-HF®, SwaI, TaqI-v2, TfiI, TseI, Tsp45I, TspMI, TspRI, Tth111I, XbaI, XcmI, XhoI, XmaI, XmnI, or a ZraI restriction cleavage site.

In some embodiments, the nucleic acid stuffer does not encode for an open reading frame (ORF) larger than 10, 20, 30, 40, or 50 amino acids. In some embodiments, the nucleic acid stuffer does not encode for an ORF larger than 20 amino acids.

In some embodiments, the nucleic acid stuffer sequence has a length of about 100 to about 5000 nucleobases. In some embodiments, the nucleic acid stuffer sequence has a length of about 100 to about 5000, 100 to about 4000, 100 to about 3000, 100 to about 2000, 100 to about 1000, 100 to about 900, 100 to about 800, 100 to about 700, 100 to about 600, 100 to about 500 or 100 to about 400 nucleobases. In some embodiments, the nucleic acid stuffer sequence has a length of about 100 to about 5000, 500 to about 5000, 1000 to about 5000, 2000 to about 5000, or 3000 to about 5000 nucleobases. In some embodiments, the nucleic acid stuffer sequence has a length of about 1000 to about 5000, 1050 to about 4500, or 2000 to about 3000 nucleobases. In some embodiments, the nucleic acid stuffer sequence has a length of about 3028 nucleobases. In some embodiments, the nucleic acid stuffer sequence has a length of about 2235 nucleobases. In some embodiments, the nucleic acid stuffer sequence has a length of about 381 nucleobases.

In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 7. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 8. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 11. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to 100 nucleotides of SEQ ID NO: 7, 8, or 11.

In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence complementary to SEQ ID NO: 7. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence complementary to SEQ ID NO: 8. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence complementary to SEQ ID NO: 11. In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to a sequence complementary to at least 100 nucleotides of SEQ ID NO: 7, 8 or 11.

In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to bases 1-100, 2-101, 3-102, 4-103, 5-104, 6-105, 7-106, 8-107, 9-108, 10-109, 11-110, 12-111, 13-112, 14-113, 15-114, 16-115, 17-116, 18-117, 19-118, 20-119, 21-120, 22-121, 23-122, 24-123, 25-124, 26-125, 27-126, 28-127, 29-128, 30-129, 31-130, 32-131, 33-132, 34-133, 35-134, 36-135, 37-136, 38-137, 39-138, 40-139, 41-140, 42-141, 43-142, 44-143, 45-144, 46-145, 47-146, 48-147, 49-148, 50-149, 51-150, 52-151, 53-152, 54-153, 55-154, 56-155, 57-156, 58-157, 59-158, 60-159, 61-160, 62-161, 63-162, 64-163, 65-164, 66-165, 67-166, 68-167, 69-168, 70-169, 71-170, 72-171, 73-172, 74-173, 75-174, 76-175, 77-176, 78-177, 79-178, 80-179, 81-180, 82-181, 83-182, 84-183, 85-184, 86-185, 87-186, 88-187, 89-188, 90-189, 91-190, 92-191, 93-192, 94-193, 95-194, 96-195, 97-196, 98-197, 99-198, 100-199, 101-200, 102-201, 103-202, 104-203, 105-204, 106-205, 107-206, 108-207, 109-208, 110-209, 111-210, 112-211, 113-212, 114-213, 115-214, 116-215, 117-216, 118-217, 119-218, 120-219, 121-220, 122-221, 123-222, 124-223, 125-224, 126-225, 127-226, 128-227, 129-228, 130-229, 131-230, 132-231, 133-232, 134-233, 135-234, 136-235, 137-236, 138-237, 139-238, 140-239, 141-240, 142-241, 143-242, 144-243, 145-244, 146-245, 147-246, 148-247, 149-248, 150-249, 151-250, 152-251, 153-252, 154-253, 155-254, 156-255, 157-256, 158-257, 159-258, 160-259, 161-260, 162-261, 163-262, 164-263, 165-264, 166-265, 167-266, 168-267, 169-268, 170-269, 171-270, 172-271, 173-272, 174-273, 175-274, 176-275, 177-276, 178-277, 179-278, 180-279, 181-280, 182-281, 183-282, 184-283, 185-284, 186-285, 187-286, 188-287, 189-288, 190-289, 191-290, 192-291, 193-292, 194-293, 195-294, 196-295, 197-296, 198-297, 199-298, 200-299, 201-300, 202-301, 203-302, 204-303, 205-304, 206-305, 207-306, 208-307, 209-308, 210-309, 211-310, 212-311, 213-312, 214-313, 215-314, 216-315, 217-316, 218-317, 219-318, 220-319, 221-320, 222-321, 223-322, 224-323, 225-324, 226-325, 227-326, 228-327, 229-328, 230-329, 231-330, 232-331, 233-332, 234-333, 235-334, 236-335, 237-336, 238-337, 239-338, 240-339, 241-340, 242-341, 243-342, 244-343, 245-344, 246-345, 247-346, 248-347, 249-348, 250-349, 251-350, 252-351, 253-352, 254-353, 255-354, 256-355, 257-356, 258-357, 259-358, 260-359, 261-360, 262-361, 263-362, 264-363, 265-364, 266-365, 267-366, 268-367, 269-368, 270-369, 271-370, 272-371, 273-372, 274-373, 275-374, 276-375, 277-376, 278-377, 279-378, 280-379, 281-380, 282-381, 283-382, 284-383, 285-384, 286-385, 287-386, 288-387, 289-388, 290-389, 291-390, 292-391, 293-392, 294-393, 295-394, 296-395, 297-396, 298-397, 299-398, 300-399, 301-400, 302-401, 303-402, 304-403, 305-404, 306-405, 307-406, 308-407, 309-408, 310-409, 311-410, 312-411, 313-412, 314-413, 315-414, 316-415, 317-416, 318-417, 319-418, 320-419, 321-420, 322-421, 323-422, 324-423, 325-424, 326-425, 327-426, 328-427, 329-428, 330-429, 331-430, 332-431, 333-432, 334-433, 335-434, 336-435, 337-436, 338-437, 339-438, 340-439, 341-440, 342-441, 343-442, 344-443, 345-444, 346-445, 347-446, 348-447, 349-448, 350-449, 351-450, 352-451, 353-452, 354-453, 355-454, 356-455, 357-456, 358-457, 359-458, 360-459, 361-460, 362-461, 363-462, 364-463, 365-464, 366-465, 367-466, 368-467, 369-468, 370-469, 371-470, 372-471, 373-472, 374-473, 375-474, 376-475, 377-476, 378-477, 379-478, 380-479, 381-480, 382-481, 383-482, 384-483, 385-484, 386-485, 387-486, 388-487, 389-488, 390-489, 391-490, 392-491, 393-492, 394-493, 395-494, 396-495, 397-496, 398-497, 399-498, 400-499, 401-500, 402-501, 403-502, 404-503, 405-504, 406-505, 407-506, 408-507, 409-508, 410-509, 411-510, 412-511, 413-512, 414-513, 415-514, 416-515, 417-516, 418-517, 419-518, 420-519, 421-520, 422-521, 423-522, 424-523, 425-524, 426-525, 427-526, 428-527, 429-528, 430-529, 431-530, 432-531, 433-532, 434-533, 435-534, 436-535, 437-536, 438-537, 439-538, 440-539, 441-540, 442-541, 443-542, 444-543, 445-544, 446-545, 447-546, 448-547, 449-548, 450-549, 451-550, 452-551, 453-552, 454-553, 455-554, 456-555, 457-556, 458-557, 459-558, 460-559, 461-560, 462-561, 463-562, 464-563, 465-564, 466-565, 467-566, 468-567, 469-568, 470-569, 471-570, 472-571, 473-572, 474-573, 475-574, 476-575, 477-576, 478-577, 479-578, 480-579, 481-580, 482-581, 483-582, 484-583, 485-584, 486-585, 487-586, 488-587, 489-588, 490-589, 491-590, 492-591, 493-592, 494-593, 495-594, 496-595, 497-596, 498-597, 499-598, 500-599, 501-600, 502-601, 503-602, 504-603, 505-604, 506-605, 507-606, 508-607, 509-608, 510-609, 511-610, 512-611, 513-612, 514-613, 515-614, 516-615, 517-616, 518-617, 519-618, 520-619, 521-620, 522-621, 523-622, 524-623, 525-624, 526-625, 527-626, 528-627, 529-628, 530-629, 531-630, 532-631, 533-632, 534-633, 535-634, 536-635, 537-636, 538-637, 539-638, 540-639, 541-640, 542-641, 543-642, 544-643, 545-644, 546-645, 547-646, 548-647, 549-648, 550-649, 551-650, 552-651, 553-652, 554-653, 555-654, 556-655, 557-656, 558-657, 559-658, 560-659, 561-660, 562-661, 563-662, 564-663, 565-664, 566-665, 567-666, 568-667, 569-668, 570-669, 571-670, 572-671, 573-672, 574-673, 575-674, 576-675, 577-676, 578-677, 579-678, 580-679, 581-680, 582-681, 583-682, 584-683, 585-684, 586-685, 587-686, 588-687, 589-688, 590-689, 591-690, 592-691, 593-692, 594-693, 595-694, 596-695, 597-696, 598-697, 599-698, 600-699, 601-700, 602-701, 603-702, 604-703, 605-704, 606-705, 607-706, 608-707, 609-708, 610-709, 611-710, 612-711, 613-712, 614-713, 615-714, 616-715, 617-716, 618-717, 619-718, 620-719, 621-720, 622-721, 623-722, 624-723, 625-724, 626-725, 627-726, 628-727, 629-728, 630-729, 631-730, 632-731, 633-732, 634-733, 635-734, 636-735, 637-736, 638-737, 639-738, 640-739, 641-740, 642-741, 643-742, 644-743, 645-744, 646-745, 647-746, 648-747, 649-748, 650-749, 651-750, 652-751, 653-752, 654-753, 655-754, 656-755, 657-756, 658-757, 659-758, 660-759, 661-760, 662-761, 663-762, 664-763, 665-764, 666-765, 667-766, 668-767, 669-768, 670-769, 671-770, 672-771, 673-772, 674-773, 675-774, 676-775, 677-776, 678-777, 679-778, 680-779, 681-780, 682-781, 683-782, 684-783, 685-784, 686-785, 687-786, 688-787, 689-788, 690-789, 691-790, 692-791, 693-792, 694-793, 695-794, 696-795, 697-796, 698-797, 699-798, 700-799, 701-800, 702-801, 703-802, 704-803, 705-804, 706-805, 707-806, 708-807, 709-808, 710-809, 711-810, 712-811, 713-812, 714-813, 715-814, 716-815, 717-816, 718-817, 719-818, 720-819, 721-820, 722-821, 723-822, 724-823, 725-824, 726-825, 727-826, 728-827, 729-828, 730-829, 731-830, 732-831, 733-832, 734-833, 735-834, 736-835, 737-836, 738-837, 739-838, 740-839, 741-840, 742-841, 743-842, 744-843, 745-844, 746-845, 747-846, 748-847, 749-848, 750-849, 751-850, 752-851, 753-852, 754-853, 755-854, 756-855, 757-856, 758-857, 759-858, 760-859, 761-860, 762-861, 763-862, 764-863, 765-864, 766-865, 767-866, 768-867, 769-868, 770-869, 771-870, 772-871, 773-872, 774-873, 775-874, 776-875, 777-876, 778-877, 779-878, 780-879, 781-880, 782-881, 783-882, 784-883, 785-884, 786-885, 787-886, 788-887, 789-888, 790-889, 791-890, 792-891, 793-892, 794-893, 795-894, 796-895, 797-896, 798-897, 799-898, 800-899, 801-900, 802-901, 803-902, 804-903, 805-904, 806-905, 807-906, 808-907, 809-908, 810-909, 811-910, 812-911, 813-912, 814-913, 815-914, 816-915, 817-916, 818-917, 819-918, 820-919, 821-920, 822-921, 823-922, 824-923, 825-924, 826-925, 827-926, 828-927, 829-928, 830-929, 831-930, 832-931, 833-932, 834-933, 835-934, 836-935, 837-936, 838-937, 839-938, 840-939, 841-940, 842-941, 843-942, 844-943, 845-944, 846-945, 847-946, 848-947, 849-948, 850-949, 851-950, 852-951, 853-952, 854-953, 855-954, 856-955, 857-956, 858-957, 859-958, 860-959, 861-960, 862-961, 863-962, 864-963, 865-964, 866-965, 867-966, 868-967, 869-968, 870-969, 871-970, 872-971, 873-972, 874-973, 875-974, 876-975, 877-976, 878-977, 879-978, 880-979, 881-980, 882-981, 883-982, 884-983, 885-984, 886-985, 887-986, 888-987, 889-988, 890-989, 891-990, 892-991, 893-992, 894-993, 895-994, 896-995, 897-996, 898-997, 899-998, 900-999, 901-1000, 902-1001, 903-1002, 904-1003, 905-1004, 906-1005, 907-1006, 908-1007, 909-1008, 910-1009, 911-1010, 912-1011, 913-1012, 914-1013, 915-1014, 916-1015, 917-1016, 918-1017, 919-1018, 920-1019, 921-1020, 922-1021, 923-1022, 924-1023, 925-1024, 926-1025, 927-1026, 928-1027, 929-1028, 930-1029, 931-1030, 932-1031, 933-1032, 934-1033, 935-1034, 936-1035, 937-1036, 938-1037, 939-1038, 940-1039, 941-1040, 942-1041, 943-1042, 944-1043, 945-1044, 946-1045, 947-1046, 948-1047, 949-1048, 950-1049, 951-1050, 952-1051, 953-1052, 954-1053, 955-1054, 956-1055, 957-1056, 958-1057, 959-1058, 960-1059, 961-1060, 962-1061, 963-1062, 964-1063, 965-1064, 966-1065, 967-1066, 968-1067, 969-1068, 970-1069, 971-1070, 972-1071, 973-1072, 974-1073, 975-1074, 976-1075, 977-1076, 978-1077, 979-1078, 980-1079, 981-1080, 982-1081, 983-1082, 984-1083, 985-1084, 986-1085, 987-1086, 988-1087, 989-1088, 990-1089, 991-1090, 992-1091, 993-1092, 994-1093, 995-1094, 996-1095, 997-1096, 998-1097, 999-1098, 1000-1099, 1000-1100, 1001-1101, 1002-1102, 1003-1103, 1004-1104, 1005-1105, 1006-1106, 1007-1107, 1008-1108, 1009-1109, 1010-1110, 1011-1111, 1012-1112, 1013-1113, 1014-1114, 1015-1115, 1016-1116, 1017-1117, 1018-1118, 1019-1119, 1020-1120, 1021-1121, 1022-1122, 1023-1123, 1024-1124, 1025-1125, 1026-1126, 1027-1127, 1028-1128, 1029-1129, 1030-1130, 1031-1131, 1032-1132, 1033-1133, 1034-1134, 1035-1135, 1036-1136, 1037-1137, 1038-1138, 1039-1139, 1040-1140, 1041-1141, 1042-1142, 1043-1143, 1044-1144, 1045-1145, 1046-1146, 1047-1147, 1048-1148, 1049-1149, 1050-1150, 1051-1151, 1052-1152, 1053-1153, 1054-1154, 1055-1155, 1056-1156, 1057-1157, 1058-1158, 1059-1159, 1060-1160, 1061-1161, 1062-1162, 1063-1163, 1064-1164, 1065-1165, 1066-1166, 1067-1167, 1068-1168, 1069-1169, 1070-1170, 1071-1171, 1072-1172, 1073-1173, 1074-1174, 1075-1175, 1076-1176, 1077-1177, 1078-1178, 1079-1179, 1080-1180, 1081-1181, 1082-1182, 1083-1183, 1084-1184, 1085-1185, 1086-1186, 1087-1187, 1088-1188, 1089-1189, 1090-1190, 1091-1191, 1092-1192, 1093-1193, 1094-1194, 1095-1195, 1096-1196, 1097-1197, 1098-1198, 1099-1199, 1100-1200, 1101-1201, 1102-1202, 1103-1203, 1104-1204, 1105-1205, 1106-1206, 1107-1207, 1108-1208, 1109-1209, 1110-1210, 1111-1211, 1112-1212, 1113-1213, 1114-1214, 1115-1215, 1116-1216, 1117-1217, 1118-1218, 1119-1219, 1120-1220, 1121-1221, 1122-1222, 1123-1223, 1124-1224, 1125-1225, 1126-1226, 1127-1227, 1128-1228, 1129-1229, 1130-1230, 1131-1231, 1132-1232, 1133-1233, 1134-1234, 1135-1235, 1136-1236, 1137-1237, 1138-1238, 1139-1239, 1140-1240, 1141-1241, 1142-1242, 1143-1243, 1144-1244, 1145-1245, 1146-1246, 1147-1247, 1148-1248, 1149-1249, 1150-1250, 1151-1251, 1152-1252, 1153-1253, 1154-1254, 1155-1255, 1156-1256, 1157-1257, 1158-1258, 1159-1259, 1160-1260, 1161-1261, 1162-1262, 1163-1263, 1164-1264, 1165-1265, 1166-1266, 1167-1267, 1168-1268, 1169-1269, 1170-1270, 1171-1271, 1172-1272, 1173-1273, 1174-1274, 1175-1275, 1176-1276, 1177-1277, 1178-1278, 1179-1279, 1180-1280, 1181-1281, 1182-1282, 1183-1283, 1184-1284, 1185-1285, 1186-1286, 1187-1287, 1188-1288, 1189-1289, 1190-1290, 1191-1291, 1192-1292, 1193-1293, 1194-1294, 1195-1295, 1196-1296, 1197-1297, 1198-1298, 1199-1299, 1200-1300, 1201-1301, 1202-1302, 1203-1303, 1204-1304, 1205-1305, 1206-1306, 1207-1307, 1208-1308, 1209-1309, 1210-1310, 1211-1311, 1212-1312, 1213-1313, 1214-1314, 1215-1315, 1216-1316, 1217-1317, 1218-1318, 1219-1319, 1220-1320, 1221-1321, 1222-1322, 1223-1323, 1224-1324, 1225-1325, 1226-1326, 1227-1327, 1228-1328, 1229-1329, 1230-1330, 1231-1331, 1232-1332, 1233-1333, 1234-1334, 1235-1335, 1236-1336, 1237-1337, 1238-1338, 1239-1339, 1240-1340, 1241-1341, 1242-1342, 1243-1343, 1244-1344, 1245-1345, 1246-1346, 1247-1347, 1248-1348, 1249-1349, 1250-1350, 1251-1351, 1252-1352, 1253-1353, 1254-1354, 1255-1355, 1256-1356, 1257-1357, 1258-1358, 1259-1359, 1260-1360, 1261-1361, 1262-1362, 1263-1363, 1264-1364, 1265-1365, 1266-1366, 1267-1367, 1268-1368, 1269-1369, 1270-1370, 1271-1371, 1272-1372, 1273-1373, 1274-1374, 1275-1375, 1276-1376, 1277-1377, 1278-1378, 1279-1379, 1280-1380, 1281-1381, 1282-1382, 1283-1383, 1284-1384, 1285-1385, 1286-1386, 1287-1387, 1288-1388, 1289-1389, 1290-1390, 1291-1391, 1292-1392, 1293-1393, 1294-1394, 1295-1395, 1296-1396, 1297-1397, 1298-1398, 1299-1399, 1300-1400, 1301-1401, 1302-1402, 1303-1403, 1304-1404, 1305-1405, 1306-1406, 1307-1407, 1308-1408, 1309-1409, 1310-1410, 1311-1411, 1312-1412, 1313-1413, 1314-1414, 1315-1415, 1316-1416, 1317-1417, 1318-1418, 1319-1419, 1320-1420, 1321-1421, 1322-1422, 1323-1423, 1324-1424, 1325-1425, 1326-1426, 1327-1427, 1328-1428, 1329-1429, 1330-1430, 1331-1431, 1332-1432, 1333-1433, 1334-1434, 1335-1435, 1336-1436, 1337-1437, 1338-1438, 1339-1439, 1340-1440, 1341-1441, 1342-1442, 1343-1443, 1344-1444, 1345-1445, 1346-1446, 1347-1447, 1348-1448, 1349-1449, 1350-1450, 1351-1451, 1352-1452, 1353-1453, 1354-1454, 1355-1455, 1356-1456, 1357-1457, 1358-1458, 1359-1459, 1360-1460, 1361-1461, 1362-1462, 1363-1463, 1364-1464, 1365-1465, 1366-1466, 1367-1467, 1368-1468, 1369-1469, 1370-1470, 1371-1471, 1372-1472, 1373-1473, 1374-1474, 1375-1475, 1376-1476, 1377-1477, 1378-1478, 1379-1479, 1380-1480, 1381-1481, 1382-1482, 1383-1483, 1384-1484, 1385-1485, 1386-1486, 1387-1487, 1388-1488, 1389-1489, 1390-1490, 1391-1491, 1392-1492, 1393-1493, 1394-1494, 1395-1495, 1396-1496, 1397-1497, 1398-1498, 1399-1499, 1400-1500, 1401-1501, 1402-1502, 1403-1503, 1404-1504, 1405-1505, 1406-1506, 1407-1507, 1408-1508, 1409-1509, 1410-1510, 1411-1511, 1412-1512, 1413-1513, 1414-1514, 1415-1515, 1416-1516, 1417-1517, 1418-1518, 1419-1519, 1420-1520, 1421-1521, 1422-1522, 1423-1523, 1424-1524, 1425-1525, 1426-1526, 1427-1527, 1428-1528, 1429-1529, 1430-1530, 1431-1531, 1432-1532, 1433-1533, 1434-1534, 1435-1535, 1436-1536, 1437-1537, 1438-1538, 1439-1539, 1440-1540, 1441-1541, 1442-1542, 1443-1543, 1444-1544, 1445-1545, 1446-1546, 1447-1547, 1448-1548, 1449-1549, 1450-1550, 1451-1551, 1452-1552, 1453-1553, 1454-1554, 1455-1555, 1456-1556, 1457-1557, 1458-1558, 1459-1559, 1460-1560, 1461-1561, 1462-1562, 1463-1563, 1464-1564, 1465-1565, 1466-1566, 1467-1567, 1468-1568, 1469-1569, 1470-1570, 1471-1571, 1472-1572, 1473-1573, 1474-1574, 1475-1575, 1476-1576, 1477-1577, 1478-1578, 1479-1579, 1480-1580, 1481-1581, 1482-1582, 1483-1583, 1484-1584, 1485-1585, 1486-1586, 1487-1587, 1488-1588, 1489-1589, 1490-1590, 1491-1591, 1492-1592, 1493-1593, 1494-1594, 1495-1595, 1496-1596, 1497-1597, 1498-1598, 1499-1599, 1500-1600, 1501-1601, 1502-1602, 1503-1603, 1504-1604, 1505-1605, 1506-1606, 1507-1607, 1508-1608, 1509-1609, 1510-1610, 1511-1611, 1512-1612, 1513-1613, 1514-1614, 1515-1615, 1516-1616, 1517-1617, 1518-1618, 1519-1619, 1520-1620, 1521-1621, 1522-1622, 1523-1623, 1524-1624, 1525-1625, 1526-1626, 1527-1627, 1528-1628, 1529-1629, 1530-1630, 1531-1631, 1532-1632, 1533-1633, 1534-1634, 1535-1635, 1536-1636, 1537-1637, 1538-1638, 1539-1639, 1540-1640, 1541-1641, 1542-1642, 1543-1643, 1544-1644, 1545-1645, 1546-1646, 1547-1647, 1548-1648, 1549-1649, 1550-1650, 1551-1651, 1552-1652, 1553-1653, 1554-1654, 1555-1655, 1556-1656, 1557-1657, 1558-1658, 1559-1659, 1560-1660, 1561-1661, 1562-1662, 1563-1663, 1564-1664, 1565-1665, 1566-1666, 1567-1667, 1568-1668, 1569-1669, 1570-1670, 1571-1671, 1572-1672, 1573-1673, 1574-1674, 1575-1675, 1576-1676, 1577-1677, 1578-1678, 1579-1679, 1580-1680, 1581-1681, 1582-1682, 1583-1683, 1584-1684, 1585-1685, 1586-1686, 1587-1687, 1588-1688, 1589-1689, 1590-1690, 1591-1691, 1592-1692, 1593-1693, 1594-1694, 1595-1695, 1596-1696, 1597-1697, 1598-1698, 1599-1699, 1600-1700, 1601-1701, 1602-1702, 1603-1703, 1604-1704, 1605-1705, 1606-1706, 1607-1707, 1608-1708, 1609-1709, 1610-1710, 1611-1711, 1612-1712, 1613-1713, 1614-1714, 1615-1715, 1616-1716, 1617-1717, 1618-1718, 1619-1719, 1620-1720, 1621-1721, 1622-1722, 1623-1723, 1624-1724, 1625-1725, 1626-1726, 1627-1727, 1628-1728, 1629-1729, 1630-1730, 1631-1731, 1632-1732, 1633-1733, 1634-1734, 1635-1735, 1636-1736, 1637-1737, 1638-1738, 1639-1739, 1640-1740, 1641-1741, 1642-1742, 1643-1743, 1644-1744, 1645-1745, 1646-1746, 1647-1747, 1648-1748, 1649-1749, 1650-1750, 1651-1751, 1652-1752, 1653-1753, 1654-1754, 1655-1755, 1656-1756, 1657-1757, 1658-1758, 1659-1759, 1660-1760, 1661-1761, 1662-1762, 1663-1763, 1664-1764, 1665-1765, 1666-1766, 1667-1767, 1668-1768, 1669-1769, 1670-1770, 1671-1771, 1672-1772, 1673-1773, 1674-1774, 1675-1775, 1676-1776, 1677-1777, 1678-1778, 1679-1779, 1680-1780, 1681-1781, 1682-1782, 1683-1783, 1684-1784, 1685-1785, 1686-1786, 1687-1787, 1688-1788, 1689-1789, 1690-1790, 1691-1791, 1692-1792, 1693-1793, 1694-1794, 1695-1795, 1696-1796, 1697-1797, 1698-1798, 1699-1799, 1700-1800, 1701-1801, 1702-1802, 1703-1803, 1704-1804, 1705-1805, 1706-1806, 1707-1807, 1708-1808, 1709-1809, 1710-1810, 1711-1811, 1712-1812, 1713-1813, 1714-1814, 1715-1815, 1716-1816, 1717-1817, 1718-1818, 1719-1819, 1720-1820, 1721-1821, 1722-1822, 1723-1823, 1724-1824, 1725-1825, 1726-1826, 1727-1827, 1728-1828, 1729-1829, 1730-1830, 1731-1831, 1732-1832, 1733-1833, 1734-1834, 1735-1835, 1736-1836, 1737-1837, 1738-1838, 1739-1839, 1740-1840, 1741-1841, 1742-1842, 1743-1843, 1744-1844, 1745-1845, 1746-1846, 1747-1847, 1748-1848, 1749-1849, 1750-1850, 1751-1851, 1752-1852, 1753-1853, 1754-1854, 1755-1855, 1756-1856, 1757-1857, 1758-1858, 1759-1859, 1760-1860, 1761-1861, 1762-1862, 1763-1863, 1764-1864, 1765-1865, 1766-1866, 1767-1867, 1768-1868, 1769-1869, 1770-1870, 1771-1871, 1772-1872, 1773-1873, 1774-1874, 1775-1875, 1776-1876, 1777-1877, 1778-1878, 1779-1879, 1780-1880, 1781-1881, 1782-1882, 1783-1883, 1784-1884, 1785-1885, 1786-1886, 1787-1887, 1788-1888, 1789-1889, 1790-1890, 1791-1891, 1792-1892, 1793-1893, 1794-1894, 1795-1895, 1796-1896, 1797-1897, 1798-1898, 1799-1899, 1800-1900, 1801-1901, 1802-1902, 1803-1903, 1804-1904, 1805-1905, 1806-1906, 1807-1907, 1808-1908, 1809-1909, 1810-1910, 1811-1911, 1812-1912, 1813-1913, 1814-1914, 1815-1915, 1816-1916, 1817-1917, 1818-1918, 1819-1919, 1820-1920, 1821-1921, 1822-1922, 1823-1923, 1824-1924, 1825-1925, 1826-1926, 1827-1927, 1828-1928, 1829-1929, 1830-1930, 1831-1931, 1832-1932, 1833-1933, 1834-1934, 1835-1935, 1836-1936, 1837-1937, 1838-1938, 1839-1939, 1840-1940, 1841-1941, 1842-1942, 1843-1943, 1844-1944, 1845-1945, 1846-1946, 1847-1947, 1848-1948, 1849-1949, 1850-1950, 1851-1951, 1852-1952, 1853-1953, 1854-1954, 1855-1955, 1856-1956, 1857-1957, 1858-1958, 1859-1959, 1860-1960, 1861-1961, 1862-1962, 1863-1963, 1864-1964, 1865-1965, 1866-1966, 1867-1967, 1868-1968, 1869-1969, 1870-1970, 1871-1971, 1872-1972, 1873-1973, 1874-1974, 1875-1975, 1876-1976, 1877-1977, 1878-1978, 1879-1979, 1880-1980, 1881-1981, 1882-1982, 1883-1983, 1884-1984, 1885-1985, 1886-1986, 1887-1987, 1888-1988, 1889-1989, 1890-1990, 1891-1991, 1892-1992, 1893-1993, 1894-1994, 1895-1995, 1896-1996, 1897-1997, 1898-1998, 1899-1999, 1900-2000, 1901-2001, 1902-2002, 1903-2003, 1904-2004, 1905-2005, 1906-2006, 1907-2007, 1908-2008, 1909-2009, 1910-2010, 1911-2011, 1912-2012, 1913-2013, 1914-2014, 1915-2015, 1916-2016, 1917-2017, 1918-2018, 1919-2019, 1920-2020, 1921-2021, 1922-2022, 1923-2023, 1924-2024, 1925-2025, 1926-2026, 1927-2027, 1928-2028, 1929-2029, 1930-2030, 1931-2031, 1932-2032, 1933-2033, 1934-2034, 1935-2035, 1936-2036, 1937-2037, 1938-2038, 1939-2039, 1940-2040, 1941-2041, 1942-2042, 1943-2043, 1944-2044, 1945-2045, 1946-2046, 1947-2047, 1948-2048, 1949-2049, 1950-2050, 1951-2051, 1952-2052, 1953-2053, 1954-2054, 1955-2055, 1956-2056, 1957-2057, 1958-2058, 1959-2059, 1960-2060, 1961-2061, 1962-2062, 1963-2063, 1964-2064, 1965-2065, 1966-2066, 1967-2067, 1968-2068, 1969-2069, 1970-2070, 1971-2071, 1972-2072, 1973-2073, 1974-2074, 1975-2075, 1976-2076, 1977-2077, 1978-2078, 1979-2079, 1980-2080, 1981-2081, 1982-2082, 1983-2083, 1984-2084, 1985-2085, 1986-2086, 1987-2087, 1988-2088, 1989-2089, 1990-2090, 1991-2091, 1992-2092, 1993-2093, 1994-2094, 1995-2095, 1996-2096, 1997-2097, 1998-2098, 1999-2099, 2000-2100, 2001-2101, 2002-2102, 2003-2103, 2004-2104, 2005-2105, 2006-2106, 2007-2107, 2008-2108, 2009-2109, 2010-2110, 2011-2111, 2012-2112, 2013-2113, 2014-2114, 2015-2115, 2016-2116, 2017-2117, 2018-2118, 2019-2119, 2020-2120, 2021-2121, 2022-2122, 2023-2123, 2024-2124, 2025-2125, 2026-2126, 2027-2127, 2028-2128, 2029-2129, 2030-2130, 2031-2131, 2032-2132, 2033-2133, 2034-2134, 2035-2135, 2036-2136, 2037-2137, 2038-2138, 2039-2139, 2040-2140, 2041-2141, 2042-2142, 2043-2143, 2044-2144, 2045-2145, 2046-2146, 2047-2147, 2048-2148, 2049-2149, 2050-2150, 2051-2151, 2052-2152, 2053-2153, 2054-2154, 2055-2155, 2056-2156, 2057-2157, 2058-2158, 2059-2159, 2060-2160, 2061-2161, 2062-2162, 2063-2163, 2064-2164, 2065-2165, 2066-2166, 2067-2167, 2068-2168, 2069-2169, 2070-2170, 2071-2171, 2072-2172, 2073-2173, 2074-2174, 2075-2175, 2076-2176, 2077-2177, 2078-2178, 2079-2179, 2080-2180, 2081-2181, 2082-2182, 2083-2183, 2084-2184, 2085-2185, 2086-2186, 2087-2187, 2088-2188, 2089-2189, 2090-2190, 2091-2191, 2092-2192, 2093-2193, 2094-2194, 2095-2195, 2096-2196, 2097-2197, 2098-2198, 2099-2199, 2100-2200, 2101-2201, 2102-2202, 2103-2203, 2104-2204, 2105-2205, 2106-2206, 2107-2207, 2108-2208, 2109-2209, 2110-2210, 2111-2211, 2112-2212, 2113-2213, 2114-2214, 2115-2215, 2116-2216, 2117-2217, 2118-2218, 2119-2219, 2120-2220, 2121-2221, 2122-2222, 2123-2223, 2124-2224, 2125-2225, 2126-2226, 2127-2227, 2128-2228, 2129-2229, 2130-2230, 2131-2231, 2132-2232, 2133-2233, 2134-2234, or 2135-2235 of SEQ ID NO: 7 or a sequence complementary to SEQ ID NO: 7.

In some embodiments, the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to bases 1-100, 2-101, 3-102, 4-103, 5-104, 6-105, 7-106, 8-107, 9-108, 10-109, 11-110, 12-111, 13-112, 14-113, 15-114, 16-115, 17-116, 18-117, 19-118, 20-119, 21-120, 22-121, 23-122, 24-123, 25-124, 26-125, 27-126, 28-127, 29-128, 30-129, 31-130, 32-131, 33-132, 34-133, 35-134, 36-135, 37-136, 38-137, 39-138, 40-139, 41-140, 42-141, 43-142, 44-143, 45-144, 46-145, 47-146, 48-147, 49-148, 50-149, 51-150, 52-151, 53-152, 54-153, 55-154, 56-155, 57-156, 58-157, 59-158, 60-159, 61-160, 62-161, 63-162, 64-163, 65-164, 66-165, 67-166, 68-167, 69-168, 70-169, 71-170, 72-171, 73-172, 74-173, 75-174, 76-175, 77-176, 78-177, 79-178, 80-179, 81-180, 82-181, 83-182, 84-183, 85-184, 86-185, 87-186, 88-187, 89-188, 90-189, 91-190, 92-191, 93-192, 94-193, 95-194, 96-195, 97-196, 98-197, 99-198, 100-199, 101-200, 102-201, 103-202, 104-203, 105-204, 106-205, 107-206, 108-207, 109-208, 110-209, 111-210, 112-211, 113-212, 114-213, 115-214, 116-215, 117-216, 118-217, 119-218, 120-219, 121-220, 122-221, 123-222, 124-223, 125-224, 126-225, 127-226, 128-227, 129-228, 130-229, 131-230, 132-231, 133-232, 134-233, 135-234, 136-235, 137-236, 138-237, 139-238, 140-239, 141-240, 142-241, 143-242, 144-243, 145-244, 146-245, 147-246, 148-247, 149-248, 150-249, 151-250, 152-251, 153-252, 154-253, 155-254, 156-255, 157-256, 158-257, 159-258, 160-259, 161-260, 162-261, 163-262, 164-263, 165-264, 166-265, 167-266, 168-267, 169-268, 170-269, 171-270, 172-271, 173-272, 174-273, 175-274, 176-275, 177-276, 178-277, 179-278, 180-279, 181-280, 182-281, 183-282, 184-283, 185-284, 186-285, 187-286, 188-287, 189-288, 190-289, 191-290, 192-291, 193-292, 194-293, 195-294, 196-295, 197-296, 198-297, 199-298, 200-299, 201-300, 202-301, 203-302, 204-303, 205-304, 206-305, 207-306, 208-307, 209-308, 210-309, 211-310, 212-311, 213-312, 214-313, 215-314, 216-315, 217-316, 218-317, 219-318, 220-319, 221-320, 222-321, 223-322, 224-323, 225-324, 226-325, 227-326, 228-327, 229-328, 230-329, 231-330, 232-331, 233-332, 234-333, 235-334, 236-335, 237-336, 238-337, 239-338, 240-339, 241-340, 242-341, 243-342, 244-343, 245-344, 246-345, 247-346, 248-347, 249-348, 250-349, 251-350, 252-351, 253-352, 254-353, 255-354, 256-355, 257-356, 258-357, 259-358, 260-359, 261-360, 262-361, 263-362, 264-363, 265-364, 266-365, 267-366, 268-367, 269-368, 270-369, 271-370, 272-371, 273-372, 274-373, 275-374, 276-375, 277-376, 278-377, 279-378, 280-379, 281-380, 282-381, 283-382, 284-383, 285-384, 286-385, 287-386, 288-387, 289-388, 290-389, 291-390, 292-391, 293-392, 294-393, 295-394, 296-395, 297-396, 298-397, 299-398, 300-399, 301-400, 302-401, 303-402, 304-403, 305-404, 306-405, 307-406, 308-407, 309-408, 310-409, 311-410, 312-411, 313-412, 314-413, 315-414, 316-415, 317-416, 318-417, 319-418, 320-419, 321-420, 322-421, 323-422, 324-423, 325-424, 326-425, 327-426, 328-427, 329-428, 330-429, 331-430, 332-431, 333-432, 334-433, 335-434, 336-435, 337-436, 338-437, 339-438, 340-439, 341-440, 342-441, 343-442, 344-443, 345-444, 346-445, 347-446, 348-447, 349-448, 350-449, 351-450, 352-451, 353-452, 354-453, 355-454, 356-455, 357-456, 358-457, 359-458, 360-459, 361-460, 362-461, 363-462, 364-463, 365-464, 366-465, 367-466, 368-467, 369-468, 370-469, 371-470, 372-471, 373-472, 374-473, 375-474, 376-475, 377-476, 378-477, 379-478, 380-479, 381-480, 382-481, 383-482, 384-483, 385-484, 386-485, 387-486, 388-487, 389-488, 390-489, 391-490, 392-491, 393-492, 394-493, 395-494, 396-495, 397-496, 398-497, 399-498, 400-499, 401-500, 402-501, 403-502, 404-503, 405-504, 406-505, 407-506, 408-507, 409-508, 410-509, 411-510, 412-511, 413-512, 414-513, 415-514, 416-515, 417-516, 418-517, 419-518, 420-519, 421-520, 422-521, 423-522, 424-523, 425-524, 426-525, 427-526, 428-527, 429-528, 430-529, 431-530, 432-531, 433-532, 434-533, 435-534, 436-535, 437-536, 438-537, 439-538, 440-539, 441-540, 442-541, 443-542, 444-543, 445-544, 446-545, 447-546, 448-547, 449-548, 450-549, 451-550, 452-551, 453-552, 454-553, 455-554, 456-555, 457-556, 458-557, 459-558, 460-559, 461-560, 462-561, 463-562, 464-563, 465-564, 466-565, 467-566, 468-567, 469-568, 470-569, 471-570, 472-571, 473-572, 474-573, 475-574, 476-575, 477-576, 478-577, 479-578, 480-579, 481-580, 482-581, 483-582, 484-583, 485-584, 486-585, 487-586, 488-587, 489-588, 490-589, 491-590, 492-591, 493-592, 494-593, 495-594, 496-595, 497-596, 498-597, 499-598, 500-599, 501-600, 502-601, 503-602, 504-603, 505-604, 506-605, 507-606, 508-607, 509-608, 510-609, 511-610, 512-611, 513-612, 514-613, 515-614, 516-615, 517-616, 518-617, 519-618, 520-619, 521-620, 522-621, 523-622, 524-623, 525-624, 526-625, 527-626, 528-627, 529-628, 530-629, 531-630, 532-631, 533-632, 534-633, 535-634, 536-635, 537-636, 538-637, 539-638, 540-639, 541-640, 542-641, 543-642, 544-643, 545-644, 546-645, 547-646, 548-647, 549-648, 550-649, 551-650, 552-651, 553-652, 554-653, 555-654, 556-655, 557-656, 558-657, 559-658, 560-659, 561-660, 562-661, 563-662, 564-663, 565-664, 566-665, 567-666, 568-667, 569-668, 570-669, 571-670, 572-671, 573-672, 574-673, 575-674, 576-675, 577-676, 578-677, 579-678, 580-679, 581-680, 582-681, 583-682, 584-683, 585-684, 586-685, 587-686, 588-687, 589-688, 590-689, 591-690, 592-691, 593-692, 594-693, 595-694, 596-695, 597-696, 598-697, 599-698, 600-699, 601-700, 602-701, 603-702, 604-703, 605-704, 606-705, 607-706, 608-707, 609-708, 610-709, 611-710, 612-711, 613-712, 614-713, 615-714, 616-715, 617-716, 618-717, 619-718, 620-719, 621-720, 622-721, 623-722, 624-723, 625-724, 626-725, 627-726, 628-727, 629-728, 630-729, 631-730, 632-731, 633-732, 634-733, 635-734, 636-735, 637-736, 638-737, 639-738, 640-739, 641-740, 642-741, 643-742, 644-743, 645-744, 646-745, 647-746, 648-747, 649-748, 650-749, 651-750, 652-751, 653-752, 654-753, 655-754, 656-755, 657-756, 658-757, 659-758, 660-759, 661-760, 662-761, 663-762, 664-763, 665-764, 666-765, 667-766, 668-767, 669-768, 670-769, 671-770, 672-771, 673-772, 674-773, 675-774, 676-775, 677-776, 678-777, 679-778, 680-779, 681-780, 682-781, 683-782, 684-783, 685-784, 686-785, 687-786, 688-787, 689-788, 690-789, 691-790, 692-791, 693-792, 694-793, 695-794, 696-795, 697-796, 698-797, 699-798, 700-799, 701-800, 702-801, 703-802, 704-803, 705-804, 706-805, 707-806, 708-807, 709-808, 710-809, 711-810, 712-811, 713-812, 714-813, 715-814, 716-815, 717-816, 718-817, 719-818, 720-819, 721-820, 722-821, 723-822, 724-823, 725-824, 726-825, 727-826, 728-827, 729-828, 730-829, 731-830, 732-831, 733-832, 734-833, 735-834, 736-835, 737-836, 738-837, 739-838, 740-839, 741-840, 742-841, 743-842, 744-843, 745-844, 746-845, 747-846, 748-847, 749-848, 750-849, 751-850, 752-851, 753-852, 754-853, 755-854, 756-855, 757-856, 758-857, 759-858, 760-859, 761-860, 762-861, 763-862, 764-863, 765-864, 766-865, 767-866, 768-867, 769-868, 770-869, 771-870, 772-871, 773-872, 774-873, 775-874, 776-875, 777-876, 778-877, 779-878, 780-879, 781-880, 782-881, 783-882, 784-883, 785-884, 786-885, 787-886, 788-887, 789-888, 790-889, 791-890, 792-891, 793-892, 794-893, 795-894, 796-895, 797-896, 798-897, 799-898, 800-899, 801-900, 802-901, 803-902, 804-903, 805-904, 806-905, 807-906, 808-907, 809-908, 810-909, 811-910, 812-911, 813-912, 814-913, 815-914, 816-915, 817-916, 818-917, 819-918, 820-919, 821-920, 822-921, 823-922, 824-923, 825-924, 826-925, 827-926, 828-927, 829-928, 830-929, 831-930, 832-931, 833-932, 834-933, 835-934, 836-935, 837-936, 838-937, 839-938, 840-939, 841-940, 842-941, 843-942, 844-943, 845-944, 846-945, 847-946, 848-947, 849-948, 850-949, 851-950, 852-951, 853-952, 854-953, 855-954, 856-955, 857-956, 858-957, 859-958, 860-959, 861-960, 862-961, 863-962, 864-963, 865-964, 866-965, 867-966, 868-967, 869-968, 870-969, 871-970, 872-971, 873-972, 874-973, 875-974, 876-975, 877-976, 878-977, 879-978, 880-979, 881-980, 882-981, 883-982, 884-983, 885-984, 886-985, 887-986, 888-987, 889-988, 890-989, 891-990, 892-991, 893-992, 894-993, 895-994, 896-995, 897-996, 898-997, 899-998, 900-999, 901-1000, 902-1001, 903-1002, 904-1003, 905-1004, 906-1005, 907-1006, 908-1007, 909-1008, 910-1009, 911-1010, 912-1011, 913-1012, 914-1013, 915-1014, 916-1015, 917-1016, 918-1017, 919-1018, 920-1019, 921-1020, 922-1021, 923-1022, 924-1023, 925-1024, 926-1025, 927-1026, 928-1027, 929-1028, 930-1029, 931-1030, 932-1031, 933-1032, 934-1033, 935-1034, 936-1035, 937-1036, 938-1037, 939-1038, 940-1039, 941-1040, 942-1041, 943-1042, 944-1043, 945-1044, 946-1045, 947-1046, 948-1047, 949-1048, 950-1049, 951-1050, 952-1051, 953-1052, 954-1053, 955-1054, 956-1055, 957-1056, 958-1057, 959-1058, 960-1059, 961-1060, 962-1061, 963-1062, 964-1063, 965-1064, 966-1065, 967-1066, 968-1067, 969-1068, 970-1069, 971-1070, 972-1071, 973-1072, 974-1073, 975-1074, 976-1075, 977-1076, 978-1077, 979-1078, 980-1079, 981-1080, 982-1081, 983-1082, 984-1083, 985-1084, 986-1085, 987-1086, 988-1087, 989-1088, 990-1089, 991-1090, 992-1091, 993-1092, 994-1093, 995-1094, 996-1095, 997-1096, 998-1097, 999-1098, 1000-1099, 1000-1100, 1001-1101, 1002-1102, 1003-1103, 1004-1104, 1005-1105, 1006-1106, 1007-1107, 1008-1108, 1009-1109, 1010-1110, 1011-1111, 1012-1112, 1013-1113, 1014-1114, 1015-1115, 1016-1116, 1017-1117, 1018-1118, 1019-1119, 1020-1120, 1021-1121, 1022-1122, 1023-1123, 1024-1124, 1025-1125, 1026-1126, 1027-1127, 1028-1128, 1029-1129, 1030-1130, 1031-1131, 1032-1132, 1033-1133, 1034-1134, 1035-1135, 1036-1136, 1037-1137, 1038-1138, 1039-1139, 1040-1140, 1041-1141, 1042-1142, 1043-114, 1044-1144, 1045-1145, 1046-1146, 1047-1147, 1048-1148, 1049-1149, 1050-1150, 1051-1151, 1052-1152, 1053-1153, 1054-1154, 1055-1155, 1056-1156, 1057-1157, 1058-1158, 1059-1159, 1060-1160, 1061-1161, 1062-1162, 1063-1163, 1064-1164, 1065-1165, 1066-1166, 1067-1167, 1068-1168, 1069-1169, 1070-1170, 1071-1171, 1072-1172, 1073-1173, 1074-1174, 1075-1175, 1076-1176, 1077-1177, 1078-1178, 1079-1179, 1080-1180, 1081-1181, 1082-1182, 1083-1183, 1084-1184, 1085-1185, 1086-1186, 1087-1187, 1088-1188, 1089-1189, 1090-1190, 1091-1191, 1092-1192, 1093-1193, 1094-1194, 1095-1195, 1096-1196, 1097-1197, 1098-1198, 1099-1199, 1100-1200, 1101-1201, 1102-1202, 1103-1203, 1104-1204, 1105-1205, 1106-1206, 1107-1207, 1108-1208, 1109-1209, 1110-1210, 1111-1211, 1112-1212, 1113-1213, 1114-1214, 1115-1215, 1116-1216, 1117-1217, 1118-1218, 1119-1219, 1120-1220, 1121-1221, 1122-1222, 1123-1223, 1124-1224, 1125-1225, 1126-1226, 1127-1227, 1128-1228, 1129-1229, 1130-1230, 1131-1231, 1132-1232, 1133-1233, 1134-1234, 1135-1235, 1136-1236, 1137-1237, 1138-1238, 1139-1239, 1140-1240, 1141-1241, 1142-1242, 1143-1243, 1144-1244, 1145-1245, 1146-1246, 1147-1247, 1148-1248, 1149-1249, 1150-1250, 1151-1251, 1152-1252, 1153-1253, 1154-1254, 1155-1255, 1156-1256, 1157-1257, 1158-1258, 1159-1259, 1160-1260, 1161-1261, 1162-1262, 1163-1263, 1164-1264, 1165-1265, 1166-1266, 1167-1267, 1168-1268, 1169-1269, 1170-1270, 1171-1271, 1172-1272, 1173-1273, 1174-1274, 1175-1275, 1176-1276, 1177-1277, 1178-1278, 1179-1279, 1180-1280, 1181-1281, 1182-1282, 1183-1283, 1184-1284, 1185-1285, 1186-1286, 1187-1287, 1188-1288, 1189-1289, 1190-1290, 1191-1291, 1192-1292, 1193-1293, 1194-1294, 1195-1295, 1196-1296, 1197-1297, 1198-1298, 1199-1299, 1200-1300, 1201-1301, 1202-1302, 1203-1303, 1204-1304, 1205-1305, 1206-1306, 1207-1307, 1208-1308, 1209-1309, 1210-1310, 1211-1311, 1212-1312, 1213-1313, 1214-1314, 1215-1315, 1216-1316, 1217-1317, 1218-1318, 1219-1319, 1220-1320, 1221-1321, 1222-1322, 1223-1323, 1224-1324, 1225-1325, 1226-1326, 1227-1327, 1228-1328, 1229-1329, 1230-1330, 1231-1331, 1232-1332, 1233-1333, 1234-1334, 1235-1335, 1236-1336, 1237-1337, 1238-1338, 1239-1339, 1240-1340, 1241-1341, 1242-1342, 1243-1343, 1244-1344, 1245-1345, 1246-1346, 1247-1347, 1248-1348, 1249-1349, 1250-1350, 1251-1351, 1252-1352, 1253-1353, 1254-1354, 1255-1355, 1256-1356, 1257-1357, 1258-1358, 1259-1359, 1260-1360, 1261-1361, 1262-1362, 1263-1363, 1264-1364, 1265-1365, 1266-1366, 1267-1367, 1268-1368, 1269-1369, 1270-1370, 1271-1371, 1272-1372, 1273-1373, 1274-1374, 1275-1375, 1276-1376, 1277-1377, 1278-1378, 1279-1379, 1280-1380, 1281-1381, 1282-1382, 1283-1383, 1284-1384, 1285-1385, 1286-1386, 1287-1387, 1288-1388, 1289-1389, 1290-1390, 1291-1391, 1292-1392, 1293-1393, 1294-1394, 1295-1395, 1296-1396, 1297-1397, 1298-1398, 1299-1399, 1300-1400, 1301-1401, 1302-1402, 1303-1403, 1304-1404, 1305-1405, 1306-1406, 1307-1407, 1308-1408, 1309-1409, 1310-1410, 1311-1411, 1312-1412, 1313-1413, 1314-1414, 1315-1415, 1316-1416, 1317-1417, 1318-1418, 1319-1419, 1320-1420, 1321-1421, 1322-1422, 1323-1423, 1324-1424, 1325-1425, 1326-1426, 1327-1427, 1328-1428, 1329-1429, 1330-1430, 1331-1431, 1332-1432, 1333-1433, 1334-1434, 1335-1435, 1336-1436, 1337-1437, 1338-1438, 1339-1439, 1340-1440, 1341-1441, 1342-1442, 1343-1443, 1344-1444, 1345-1445, 1346-1446, 1347-1447, 1348-1448, 1349-1449, 1350-1450, 1351-1451, 1352-1452, 1353-1453, 1354-1454, 1355-1455, 1356-1456, 1357-1457, 1358-1458, 1359-1459, 1360-1460, 1361-1461, 1362-1462, 1363-1463, 1364-1464, 1365-1465, 1366-1466, 1367-1467, 1368-1468, 1369-1469, 1370-1470, 1371-1471, 1372-1472, 1373-1473, 1374-1474, 1375-1475, 1376-1476, 1377-1477, 1378-1478, 1379-1479, 1380-1480, 1381-1481, 1382-1482, 1383-1483, 1384-1484, 1385-1485, 1386-1486, 1387-1487, 1388-1488, 1389-1489, 1390-1490, 1391-1491, 1392-1492, 1393-1493, 1394-1494, 1395-1495, 1396-1496, 1397-1497, 1398-1498, 1399-1499, 1400-1500, 1401-1501, 1402-1502, 1403-1503, 1404-1504, 1405-1505, 1406-1506, 1407-1507, 1408-1508, 1409-1509, 1410-1510, 1411-1511, 1412-1512, 1413-1513, 1414-1514, 1415-1515, 1416-1516, 1417-1517, 1418-1518, 1419-1519, 1420-1520, 1421-1521, 1422-1522, 1423-1523, 1424-1524, 1425-1525, 1426-1526, 1427-1527, 1428-1528, 1429-1529, 1430-1530, 1431-1531, 1432-1532, 1433-1533, 1434-1534, 1435-1535, 1436-1536, 1437-1537, 1438-1538, 1439-1539, 1440-1540, 1441-1541, 1442-1542, 1443-1543, 1444-1544, 1445-1545, 1446-1546, 1447-1547, 1448-1548, 1449-1549, 1450-1550, 1451-1551, 1452-1552, 1453-1553, 1454-1554, 1455-1555, 1456-1556, 1457-1557, 1458-1558, 1459-1559, 1460-1560, 1461-1561, 1462-1562, 1463-1563, 1464-1564, 1465-1565, 1466-1566, 1467-1567, 1468-1568, 1469-1569, 1470-1570, 1471-1571, 1472-1572, 1473-1573, 1474-1574, 1475-1575, 1476-1576, 1477-1577, 1478-1578, 1479-1579, 1480-1580, 1481-1581, 1482-1582, 1483-1583, 1484-1584, 1485-1585, 1486-1586, 1487-1587, 1488-1588, 1489-1589, 1490-1590, 1491-1591, 1492-1592, 1493-1593, 1494-1594, 1495-1595, 1496-1596, 1497-1597, 1498-1598, 1499-1599, 1500-1600, 1501-1601, 1502-1602, 1503-1603, 1504-1604, 1505-1605, 1506-1606, 1507-1607, 1508-1608, 1509-1609, 1510-1610, 1511-1611, 1512-1612, 1513-1613, 1514-1614, 1515-1615, 1516-1616, 1517-1617, 1518-1618, 1519-1619, 1520-1620, 1521-1621, 1522-1622, 1523-1623, 1524-1624, 1525-1625, 1526-1626, 1527-1627, 1528-1628, 1529-1629, 1530-1630, 1531-1631, 1532-1632, 1533-1633, 1534-1634, 1535-1635, 1536-1636, 1537-1637, 1538-1638, 1539-1639, 1540-1640, 1541-1641, 1542-1642, 1543-1643, 1544-1644, 1545-1645, 1546-1646, 1547-1647, 1548-1648, 1549-1649, 1550-1650, 1551-1651, 1552-1652, 1553-1653, 1554-1654, 1555-1655, 1556-1656, 1557-1657, 1558-1658, 1559-1659, 1560-1660, 1561-1661, 1562-1662, 1563-1663, 1564-1664, 1565-1665, 1566-1666, 1567-1667, 1568-1668, 1569-1669, 1570-1670, 1571-1671, 1572-1672, 1573-1673, 1574-1674, 1575-1675, 1576-1676, 1577-1677, 1578-1678, 1579-1679, 1580-1680, 1581-1681, 1582-1682, 1583-1683, 1584-1684, 1585-1685, 1586-1686, 1587-1687, 1588-1688, 1589-1689, 1590-1690, 1591-1691, 1592-1692, 1593-1693, 1594-1694, 1595-1695, 1596-1696, 1597-1697, 1598-1698, 1599-1699, 1600-1700, 1601-1701, 1602-1702, 1603-1703, 1604-1704, 1605-1705, 1606-1706, 1607-1707, 1608-1708, 1609-1709, 1610-1710, 1611-1711, 1612-1712, 1613-1713, 1614-1714, 1615-1715, 1616-1716, 1617-1717, 1618-1718, 1619-1719, 1620-1720, 1621-1721, 1622-1722, 1623-1723, 1624-1724, 1625-1725, 1626-1726, 1627-1727, 1628-1728, 1629-1729, 1630-1730, 1631-1731, 1632-1732, 1633-1733, 1634-1734, 1635-1735, 1636-1736, 1637-1737, 1638-1738, 1639-1739, 1640-1740, 1641-1741, 1642-1742, 1643-1743, 1644-1744, 1645-1745, 1646-1746, 1647-1747, 1648-1748, 1649-1749, 1650-1750, 1651-1751, 1652-1752, 1653-1753, 1654-1754, 1655-1755, 1656-1756, 1657-1757, 1658-1758, 1659-1759, 1660-1760, 1661-1761, 1662-1762, 1663-1763, 1664-1764, 1665-1765, 1666-1766, 1667-1767, 1668-1768, 1669-1769, 1670-1770, 1671-1771, 1672-1772, 1673-1773, 1674-1774, 1675-1775, 1676-1776, 1677-1777, 1678-1778, 1679-1779, 1680-1780, 1681-1781, 1682-1782, 1683-1783, 1684-1784, 1685-1785, 1686-1786, 1687-1787, 1688-1788, 1689-1789, 1690-1790, 1691-1791, 1692-1792, 1693-1793, 1694-1794, 1695-1795, 1696-1796, 1697-1797, 1698-1798, 1699-1799, 1700-1800, 1701-1801, 1702-1802, 1703-1803, 1704-1804, 1705-1805, 1706-1806, 1707-1807, 1708-1808, 1709-1809, 1710-1810, 1711-1811, 1712-1812, 1713-1813, 1714-1814, 1715-1815, 1716-1816, 1717-1817, 1718-1818, 1719-1819, 1720-1820, 1721-1821, 1722-1822, 1723-1823, 1724-1824, 1725-1825, 1726-1826, 1727-1827, 1728-1828, 1729-1829, 1730-1830, 1731-1831, 1732-1832, 1733-1833, 1734-1834, 1735-1835, 1736-1836, 1737-1837, 1738-1838, 1739-1839, 1740-1840, 1741-1841, 1742-1842, 1743-1843, 1744-1844, 1745-1845, 1746-1846, 1747-1847, 1748-1848, 1749-1849, 1750-1850, 1751-1851, 1752-1852, 1753-1853, 1754-1854, 1755-1855, 1756-1856, 1757-1857, 1758-1858, 1759-1859, 1760-1860, 1761-1861, 1762-1862, 1763-1863, 1764-1864, 1765-1865, 1766-1866, 1767-1867, 1768-1868, 1769-1869, 1770-1870, 1771-1871, 1772-1872, 1773-1873, 1774-1874, 1775-1875, 1776-1876, 1777-1877, 1778-1878, 1779-1879, 1780-1880, 1781-1881, 1782-1882, 1783-1883, 1784-1884, 1785-1885, 1786-1886, 1787-1887, 1788-1888, 1789-1889, 1790-1890, 1791-1891, 1792-1892, 1793-1893, 1794-1894, 1795-1895, 1796-1896, 1797-1897, 1798-1898, 1799-1899, 1800-1900, 1801-1901, 1802-1902, 1803-1903, 1804-1904, 1805-1905, 1806-1906, 1807-1907, 1808-1908, 1809-1909, 1810-1910, 1811-1911, 1812-1912, 1813-1913, 1814-1914, 1815-1915, 1816-1916, 1817-1917, 1818-1918, 1819-1919, 1820-1920, 1821-1921, 1822-1922, 1823-1923, 1824-1924, 1825-1925, 1826-1926, 1827-1927, 1828-1928, 1829-1929, 1830-1930, 1831-1931, 1832-1932, 1833-1933, 1834-1934, 1835-1935, 1836-1936, 1837-1937, 1838-1938, 1839-1939, 1840-1940, 1841-1941, 1842-1942, 1843-1943, 1844-1944, 1845-1945, 1846-1946, 1847-1947, 1848-1948, 1849-1949, 1850-1950, 1851-1951, 1852-1952, 1853-1953, 1854-1954, 1855-1955, 1856-1956, 1857-1957, 1858-1958, 1859-1959, 1860-1960, 1861-1961, 1862-1962, 1863-1963, 1864-1964, 1865-1965, 1866-1966, 1867-1967, 1868-1968, 1869-1969, 1870-1970, 1871-1971, 1872-1972, 1873-1973, 1874-1974, 1875-1975, 1876-1976, 1877-1977, 1878-1978, 1879-1979, 1880-1980, 1881-1981, 1882-1982, 1883-1983, 1884-1984, 1885-1985, 1886-1986, 1887-1987, 1888-1988, 1889-1989, 1890-1990, 1891-1991, 1892-1992, 1893-1993, 1894-1994, 1895-1995, 1896-1996, 1897-1997, 1898-1998, 1899-1999, 1900-2000, 1901-2001, 1902-2002, 1903-2003, 1904-2004, 1905-2005, 1906-2006, 1907-2007, 1908-2008, 1909-2009, 1910-2010, 1911-2011, 1912-2012, 1913-2013, 1914-2014, 1915-2015, 1916-2016, 1917-2017, 1918-2018, 1919-2019, 1920-2020, 1921-2021, 1922-2022, 1923-2023, 1924-2024, 1925-2025, 1926-2026, 1927-2027, 1928-2028, 1929-2029, 1930-2030, 1931-2031, 1932-2032, 1933-2033, 1934-2034, 1935-2035, 1936-2036, 1937-2037, 1938-2038, 1939-2039, 1940-2040, 1941-2041, 1942-2042, 1943-2043, 1944-2044, 1945-2045, 1946-2046, 1947-2047, 1948-2048, 1949-2049, 1950-2050, 1951-2051, 1952-2052, 1953-2053, 1954-2054, 1955-2055, 1956-2056, 1957-2057, 1958-2058, 1959-2059, 1960-2060, 1961-2061, 1962-2062, 1963-2063, 1964-2064, 1965-2065, 1966-2066, 1967-2067, 1968-2068, 1969-2069, 1970-2070, 1971-2071, 1972-2072, 1973-2073, 1974-2074, 1975-2075, 1976-2076, 1977-2077, 1978-2078, 1979-2079, 1980-2080, 1981-2081, 1982-2082, 1983-2083, 1984-2084, 1985-2085, 1986-2086, 1987-2087, 1988-2088, 1989-2089, 1990-2090, 1991-2091, 1992-2092, 1993-2093, 1994-2094, 1995-2095, 1996-2096, 1997-2097, 1998-2098, 1999-2099, 2000-2100, 2001-2101, 2002-2102, 2003-2103, 2004-2104, 2005-2105, 2006-2106, 2007-2107, 2008-2108, 2009-2109, 2010-2110, 2011-2111, 2012-2112, 2013-2113, 2014-2114, 2015-2115, 2016-2116, 2017-2117, 2018-2118, 2019-2119, 2020-2120, 2021-2121, 2022-2122, 2023-2123, 2024-2124, 2025-2125, 2026-2126, 2027-2127, 2028-2128, 2029-2129, 2030-2130, 2031-2131, 2032-2132, 2033-2133, 2034-2134, 2035-2135, 2036-2136, 2037-2137, 2038-2138, 2039-2139, 2040-2140, 2041-2141, 2042-2142, 2043-2143, 2044-2144, 2045-2145, 2046-2146, 2047-2147, 2048-2148, 2049-2149, 2050-2150, 2051-2151, 2052-2152, 2053-2153, 2054-2154, 2055-2155, 2056-2156, 2057-2157, 2058-2158, 2059-2159, 2060-2160, 2061-2161, 2062-2162, 2063-2163, 2064-2164, 2065-2165, 2066-2166, 2067-2167, 2068-2168, 2069-2169, 2070-2170, 2071-2171, 2072-2172, 2073-2173, 2074-2174, 2075-2175, 2076-2176, 2077-2177, 2078-2178, 2079-2179, 2080-2180, 2081-2181, 2082-2182, 2083-2183, 2084-2184, 2085-2185, 2086-2186, 2087-2187, 2088-2188, 2089-2189, 2090-2190, 2091-2191, 2092-2192, 2093-2193, 2094-2194, 2095-2195, 2096-2196, 2097-2197, 2098-2198, 2099-2199, 2100-2200, 2101-2201, 2102-2202, 2103-2203, 2104-2204, 2105-2205, 2106-2206, 2107-2207, 2108-2208, 2109-2209, 2110-2210, 2111-2211, 2112-2212, 2113-2213, 2114-2214, 2115-2215, 2116-2216, 2117-2217, 2118-2218, 2119-2219, 2120-2220, 2121-2221, 2122-2222, 2123-2223, 2124-2224, 2125-2225, 2126-2226, 2127-2227, 2128-2228, 2129-2229, 2130-2230, 2131-2231, 2132-2232, 2133-2233, 2134-2234, 2135-2235, 2136-2236, 2137-2237, 2138-2238, 2139-2239, 2140-2240, 2141-2241, 2142-2242, 2143-2243, 2144-2244, 2145-2245, 2146-2246, 2147-2247, 2148-2248, 2149-2249, 2150-2250, 2151-2251, 2152-2252, 2153-2253, 2154-2254, 2155-2255, 2156-2256, 2157-2257, 2158-2258, 2159-2259, 2160-2260, 2161-2261, 2162-2262, 2163-2263, 2164-2264, 2165-2265, 2166-2266, 2167-2267, 2168-2268, 2169-2269, 2170-2270, 2171-2271, 2172-2272, 2173-2273, 2174-2274, 2175-2275, 2176-2276, 2177-2277, 2178-2278, 2179-2279, 2180-2280, 2181-2281, 2182-2282, 2183-2283, 2184-2284, 2185-2285, 2186-2286, 2187-2287, 2188-2288, 2189-2289, 2190-2290, 2191-2291, 2192-2292, 2193-2293, 2194-2294, 2195-2295, 2196-2296, 2197-2297, 2198-2298, 2199-2299, 2200-2300, 2201-2301, 2202-2302, 2203-2303, 2204-2304, 2205-2305, 2206-2306, 2207-2307, 2208-2308, 2209-2309, 2210-2310, 2211-2311, 2212-2312, 2213-2313, 2214-2314, 2215-2315, 2216-2316, 2217-2317, 2218-2318, 2219-2319, 2220-2320, 2221-2321, 2222-2322, 2223-2323, 2224-2324, 2225-2325, 2226-2326, 2227-2327, 2228-2328, 2229-2329, 2230-2330, 2231-2331, 2232-2332, 2233-2333, 2234-2334, 2235-2335, 2236-2336, 2237-2337, 2238-2338, 2239-2339, 2240-2340, 2241-2341, 2242-2342, 2243-2343, 2244-2344, 2245-2345, 2246-2346, 2247-2347, 2248-2348, 2249-2349, 2250-2350, 2251-2351, 2252-2352, 2253-2353, 2254-2354, 2255-2355, 2256-2356, 2257-2357, 2258-2358, 2259-2359, 2260-2360, 2261-2361, 2262-2362, 2263-2363, 2264-2364, 2265-2365, 2266-2366, 2267-2367, 2268-2368, 2269-2369, 2270-2370, 2271-2371, 2272-2372, 2273-2373, 2274-2374, 2275-2375, 2276-2376, 2277-2377, 2278-2378, 2279-2379, 2280-2380, 2281-2381, 2282-2382, 2283-2383, 2284-2384, 2285-2385, 2286-2386, 2287-2387, 2288-2388, 2289-2389, 2290-2390, 2291-2391, 2292-2392, 2293-2393, 2294-2394, 2295-2395, 2296-2396, 2297-2397, 2298-2398, 2299-2399, 2300-2400, 2301-2401, 2302-2402, 2303-2403, 2304-2404, 2305-2405, 2306-2406, 2307-2407, 2308-2408, 2309-2409, 2310-2410, 2311-2411, 2312-2412, 2313-2413, 2314-2414, 2315-2415, 2316-2416, 2317-2417, 2318-2418, 2319-2419, 2320-2420, 2321-2421, 2322-2422, 2323-2423, 2324-2424, 2325-2425, 2326-2426, 2327-2427, 2328-2428, 2329-2429, 2330-2430, 2331-2431, 2332-2432, 2333-2433, 2334-2434, 2335-2435, 2336-2436, 2337-2437, 2338-2438, 2339-2439, 2340-2440, 2341-2441, 2342-2442, 2343-2443, 2344-2444, 2345-2445, 2346-2446, 2347-2447, 2348-2448, 2349-2449, 2350-2450, 2351-2451, 2352-2452, 2353-2453, 2354-2454, 2355-2455, 2356-2456, 2357-2457, 2358-2458, 2359-2459, 2360-2460, 2361-2461, 2362-2462, 2363-2463, 2364-2464, 2365-2465, 2366-2466, 2367-2467, 2368-2468, 2369-2469, 2370-2470, 2371-2471, 2372-2472, 2373-2473, 2374-2474, 2375-2475, 2376-2476, 2377-2477, 2378-2478, 2379-2479, 2380-2480, 2381-2481, 2382-2482, 2383-2483, 2384-2484, 2385-2485, 2386-2486, 2387-2487, 2388-2488, 2389-2489, 2390-2490, 2391-2491, 2392-2492, 2393-2493, 2394-2494, 2395-2495, 2396-2496, 2397-2497, 2398-2498, 2399-2499, 2400-2500, 2401-2501, 2402-2502, 2403-2503, 2404-2504, 2405-2505, 2406-2506, 2407-2507, 2408-2508, 2409-2509, 2410-2510, 2411-2511, 2412-2512, 2413-2513, 2414-2514, 2415-2515, 2416-2516, 2417-2517, 2418-2518, 2419-2519, 2420-2520, 2421-2521, 2422-2522, 2423-2523, 2424-2524, 2425-2525, 2426-2526, 2427-2527, 2428-2528, 2429-2529, 2430-2530, 2431-2531, 2432-2532, 2433-2533, 2434-2534, 2435-2535, 2436-2536, 2437-2537, 2438-2538, 2439-2539, 2440-2540, 2441-2541, 2442-2542, 2443-2543, 2444-2544, 2445-2545, 2446-2546, 2447-2547, 2448-2548, 2449-2549, 2450-2550, 2451-2551, 2452-2552, 2453-2553, 2454-2554, 2455-2555, 2456-2556, 2457-2557, 2458-2558, 2459-2559, 2460-2560, 2461-2561, 2462-2562, 2463-2563, 2464-2564, 2465-2565, 2466-2566, 2467-2567, 2468-2568, 2469-2569, 2470-2570, 2471-2571, 2472-2572, 2473-2573, 2474-2574, 2475-2575, 2476-2576, 2477-2577, 2478-2578, 2479-2579, 2480-2580, 2481-2581, 2482-2582, 2483-2583, 2484-2584, 2485-2585, 2486-2586, 2487-2587, 2488-2588, 2489-2589, 2490-2590, 2491-2591, 2492-2592, 2493-2593, 2494-2594, 2495-2595, 2496-2596, 2497-2597, 2498-2598, 2499-2599, 2500-2600, 2501-2601, 2502-2602, 2503-2603, 2504-2604, 2505-2605, 2506-2606, 2507-2607, 2508-2608, 2509-2609, 2510-2610, 2511-2611, 2512-2612, 2513-2613, 2514-2614, 2515-2615, 2516-2616, 2517-2617, 2518-2618, 2519-2619, 2520-2620, 2521-2621, 2522-2622, 2523-2623, 2524-2624, 2525-2625, 2526-2626, 2527-2627, 2528-2628, 2529-2629, 2530-2630, 2531-2631, 2532-2632, 2533-2633, 2534-2634, 2535-2635, 2536-2636, 2537-2637, 2538-2638, 2539-2639, 2540-2640, 2541-2641, 2542-2642, 2543-2643, 2544-2644, 2545-2645, 2546-2646, 2547-2647, 2548-2648, 2549-2649, 2550-2650, 2551-2651, 2552-2652, 2553-2653, 2554-2654, 2555-2655, 2556-2656, 2557-2657, 2558-2658, 2559-2659, 2560-2660, 2561-2661, 2562-2662, 2563-2663, 2564-2664, 2565-2665, 2566-2666, 2567-2667, 2568-2668, 2569-2669, 2570-2670, 2571-2671, 2572-2672, 2573-2673, 2574-2674, 2575-2675, 2576-2676, 2577-2677, 2578-2678, 2579-2679, 2580-2680, 2581-2681, 2582-2682, 2583-2683, 2584-2684, 2585-2685, 2586-2686, 2587-2687, 2588-2688, 2589-2689, 2590-2690, 2591-2691, 2592-2692, 2593-2693, 2594-2694, 2595-2695, 2596-2696, 2597-2697, 2598-2698, 2599-2699, 2600-2700, 2601-2701, 2602-2702, 2603-2703, 2604-2704, 2605-2705, 2606-2706, 2607-2707, 2608-2708, 2609-2709, 2610-2710, 2611-2711, 2612-2712, 2613-2713, 2614-2714, 2615-2715, 2616-2716, 2617-2717, 2618-2718, 2619-2719, 2620-2720, 2621-2721, 2622-2722, 2623-2723, 2624-2724, 2625-2725, 2626-2726, 2627-2727, 2628-2728, 2629-2729, 2630-2730, 2631-2731, 2632-2732, 2633-2733, 2634-2734, 2635-2735, 2636-2736, 2637-2737, 2638-2738, 2639-2739, 2640-2740, 2641-2741, 2642-2742, 2643-2743, 2644-2744, 2645-2745, 2646-2746, 2647-2747, 2648-2748, 2649-2749, 2650-2750, 2651-2751, 2652-2752, 2653-2753, 2654-2754, 2655-2755, 2656-2756, 2657-2757, 2658-2758, 2659-2759, 2660-2760, 2661-2761, 2662-2762, 2663-2763, 2664-2764, 2665-2765, 2666-2766, 2667-2767, 2668-2768, 2669-2769, 2670-2770, 2671-2771, 2672-2772, 2673-2773, 2674-2774, 2675-2775, 2676-2776, 2677-2777, 2678-2778, 2679-2779, 2680-2780, 2681-2781, 2682-2782, 2683-2783, 2684-2784, 2685-2785, 2686-2786, 2687-2787, 2688-2788, 2689-2789, 2690-2790, 2691-2791, 2692-2792, 2693-2793, 2694-2794, 2695-2795, 2696-2796, 2697-2797, 2698-2798, 2699-2799, 2700-2800, 2701-2801, 2702-2802, 2703-2803, 2704-2804, 2705-2805, 2706-2806, 2707-2807, 2708-2808, 2709-2809, 2710-2810, 2711-2811, 2712-2812, 2713-2813, 2714-2814, 2715-2815, 2716-2816, 2717-2817, 2718-2818, 2719-2819, 2720-2820, 2721-2821, 2722-2822, 2723-2823, 2724-2824, 2725-2825, 2726-2826, 2727-2827, 2728-2828, 2729-2829, 2730-2830, 2731-2831, 2732-2832, 2733-2833, 2734-2834, 2735-2835, 2736-2836, 2737-2837, 2738-2838, 2739-2839, 2740-2840, 2741-2841, 2742-2842, 2743-2843, 2744-2844, 2745-2845, 2746-2846, 2747-2847, 2748-2848, 2749-2849, 2750-2850, 2751-2851, 2752-2852, 2753-2853, 2754-2854, 2755-2855, 2756-2856, 2757-2857, 2758-2858, 2759-2859, 2760-2860, 2761-2861, 2762-2862, 2763-2863, 2764-2864, 2765-2865, 2766-2866, 2767-2867, 2768-2868, 2769-2869, 2770-2870, 2771-2871, 2772-2872, 2773-2873, 2774-2874, 2775-2875, 2776-2876, 2777-2877, 2778-2878, 2779-2879, 2780-2880, 2781-2881, 2782-2882, 2783-2883, 2784-2884, 2785-2885, 2786-2886, 2787-2887, 2788-2888, 2789-2889, 2790-2890, 2791-2891, 2792-2892, 2793-2893, 2794-2894, 2795-2895, 2796-2896, 2797-2897, 2798-2898, 2799-2899, 2800-2900, 2801-2901, 2802-2902, 2803-2903, 2804-2904, 2805-2905, 2806-2906, 2807-2907, 2808-2908, 2809-2909, 2810-2910, 2811-2911, 2812-2912, 2813-2913, 2814-2914, 2815-2915, 2816-2916, 2817-2917, 2818-2918, 2819-2919, 2820-2920, 2821-2921, 2822-2922, 2823-2923, 2824-2924, 2825-2925, 2826-2926, 2827-2927, 2828-2928, 2829-2929, 2830-2930, 2831-2931, 2832-2932, 2833-2933, 2834-2934, 2835-2935, 2836-2936, 2837-2937, 2838-2938, 2839-2939, 2840-2940, 2841-2941, 2842-2942, 2843-2943, 2844-2944, 2845-2945, 2846-2946, 2847-2947, 2848-2948, 2849-2949, 2850-2950, 2851-2951, 2852-2952, 2853-2953, 2854-2954, 2855-2955, 2856-2956, 2857-2957, 2858-2958, 2859-2959, 2860-2960, 2861-2961, 2862-2962, 2863-2963, 2864-2964, 2865-2965, 2866-2966, 2867-2967, 2868-2968, 2869-2969, 2870-2970, 2871-2971, 2872-2972, 2873-2973, 2874-2974, 2875-2975, 2876-2976, 2877-2977, 2878-2978, 2879-2979, 2880-2980, 2881-2981, 2882-2982, 2883-2983, 2884-2984, 2885-2985, 2886-2986, 2887-2987, 2888-2988, 2889-2989, 2890-2990, 2891-2991, 2892-2992, 2893-2993, 2894-2994, 2895-2995, 2896-2996, 2897-2997, 2898-2998, 2899-2999, 2900-3000, 2901-3001, 2902-3002, 2903-3003, 2904-3004, 2905-3005, 2906-3006, 2907-3007, 2908-3008, 2909-3009, 2910-3010, 2911-3011, 2912-3012, 2913-3013, 2914-3014, 2915-3015, 2916-3016, 2917-3017, 2918-3018, 2919-3019, 2920-3020, 2921-3021, 2922-3022, 2923-3023, 2924-3024, 2925-3025, 2926-3026, 2927-3027, or 2928-3028 of SEQ ID NO: 8 or a sequence complementary to SEQ ID NO: 8.

A nucleic acid stuffer sequence comprising a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to bases 1-100, 2-101, 3-102, 4-103, 5-104, 6-105, 7-106, 8-107, 9-108, 10-109, 11-110, 12-111, 13-112, 14-113, 15-114, 16-115, 17-116, 18-117, 19-118, 20-119, 21-120, 22-121, 23-122, 24-123, 25-124, 26-125, 27-126, 28-127, 29-128, 30-129, 31-130, 32-131, 33-132, 34-133, 35-134, 36-135, 37-136, 38-137, 39-138, 40-139, 41-140, 42-141, 43-142, 44-143, 45-144, 46-145, 47-146, 48-147, 49-148, 50-149, 51-150, 52-151, 53-152, 54-153, 55-154, 56-155, 57-156, 58-157, 59-158, 60-159, 61-160, 62-161, 63-162, 64-163, 65-164, 66-165, 67-166, 68-167, 69-168, 70-169, 71-170, 72-171, 73-172, 74-173, 75-174, 76-175, 77-176, 78-177, 79-178, 80-179, 81-180, 82-181, 83-182, 84-183, 85-184, 86-185, 87-186, 88-187, 89-188, 90-189, 91-190, 92-191, 93-192, 94-193, 95-194, 96-195, 97-196, 98-197, 99-198, 100-199, 101-200, 102-201, 103-202, 104-203, 105-204, 106-205, 107-206, 108-207, 109-208, 110-209, 111-210, 112-211, 113-212, 114-213, 115-214, 116-215, 117-216, 118-217, 119-218, 120-219, 121-220, 122-221, 123-222, 124-223, 125-224, 126-225, 127-226, 128-227, 129-228, 130-229, 131-230, 132-231, 133-232, 134-233, 135-234, 136-235, 137-236, 138-237, 139-238, 140-239, 141-240, 142-241, 143-242, 144-243, 145-244, 146-245, 147-246, 148-247, 149-248, 150-249, 151-250, 152-251, 153-252, 154-253, 155-254, 156-255, 157-256, 158-257, 159-258, 160-259, 161-260, 162-261, 163-262, 164-263, 165-264, 166-265, 167-266, 168-267, 169-268, 170-269, 171-270, 172-271, 173-272, 174-273, 175-274, 176-275, 177-276, 178-277, 179-278, 180-279, 181-280, 182-281, 183-282, 184-283, 185-284, 186-285, 187-286, 188-287, 189-288, 190-289, 191-290, 192-291, 193-292, 194-293, 195-294, 196-295, 197-296, 198-297, 199-298, 200-299, 201-300, 202-301, 203-302, 204-303, 205-304, 206-305, 207-306, 208-307, 209-308, 210-309, 211-310, 212-311, 213-312, 214-313, 215-314, 216-315, 217-316, 218-317, 219-318, 220-319, 221-320, 222-321, 223-322, 224-323, 225-324, 226-325, 227-326, 228-327, 229-328, 230-329, 231-330, 232-331, 233-332, 234-333, 235-334, 236-335, 237-336, 238-337, 239-338, 240-339, 241-340, 242-341, 243-342, 244-343, 245-344, 246-345, 247-346, 248-347, 249-348, 250-349, 251-350, 252-351, 253-352, 254-353, 255-354, 256-355, 257-356, 258-357, 259-358, 260-359, 261-360, 262-361, 263-362, 264-363, 265-364, 266-365, 267-366, 268-367, 269-368, 270-369, 271-370, 272-371, 273-372, 274-373, 275-374, 276-375, 277-376, 278-377, 279-378, 280-379, 281-380, or 282-381 of SEQ ID NO: 11 or a sequence complementary to SEQ ID NO: 11.

### B. Transfer Plasmid

In some embodiments, described herein is an adeno-associated virus (AAV) plasmid comprising the nucleic acid stuffer sequence described herein. In certain embodiments, the AAV plasmid comprises an expression cassette encoding a therapeutic peptide, a regulatory region, or a vector backbone, or a combination thereof. In some embodiments, the rAAV nucleic acid vector comprises a single-stranded (ss) or self-complementary (sc) AAV nucleic acid vectors, such as single-stranded or self-complementary recombinant viral genomes.

In some embodiments, the AAV plasmid comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 32. In some embodiments, the AAV plasmid comprises a sequence identical to SEQ ID NO: 32. In some embodiments, the AAV plasmid comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 33. In some embodiments, the AAV plasmid comprises a sequence identical to SEQ ID NO: 33.

In some embodiments, the AAV plasmid does not comprise a polyG/C sequence. In some embodiments, the AAV plasmid does not comprise a sequence having more than 2, 3, 4, 5, 6, 7, 8, 9, or 10 contiguous guanine bases. In some embodiments, the AAV plasmid does not comprise a sequence having more than 2, 3, 4, 5, 6, 7, 8, 9, or 10 contiguous guanine bases within at least 10, 20, 30, 40, 50, 60, 70, 80, 90, 100, 200, 300, 400, 500, 600, 700 or more nucleobases of a ITR.

In some embodiments, the AAV plasmid comprises an expression cassette, at least one ITR, and a backbone as described herein. In some embodiments, the AAV plasmid comprises, in order, a first ITR, an expression cassette, a second ITR, and a backbone. In some embodiments, the AAV plasmid contains an ITR, a promoter, a splice donor/splice acceptor site, a therapeutic peptide, a polyA sequence, an ITR, a first stuffer sequence, an origin of replication, a selectable marker, and a second stuffer sequence. In some embodiments, the first stuffer sequence, the second stuffer sequence, or both the first stuffer sequence and the second suffer sequence do not comprise a CpG island; the first stuffer sequence, the second stuffer sequence, or both the first stuffer sequence and the second suffer sequence do not comprise more than four contiguous nucleobases of the same identify; the first stuffer sequence, the second stuffer sequence, or both the first stuffer sequence and the second suffer sequence comprise between about 40% and about 50% GC content; the first stuffer sequence, the second stuffer sequence, or both the first stuffer sequence and the second suffer sequence do not encode for an open reading frame (ORF) larger than 20 amino acids; and/or and the first stuffer sequence, the second stuffer sequence, or both the first stuffer sequence and the second suffer sequence do not comprise a restriction enzyme cleavage site.

### Expression Cassette

In some embodiments, the AAV plasmid comprises a heterologous sequence positioned between two inverted terminal repeat (ITR) sequences. In some embodiments, the AAV plasmid comprises an expression cassette comprising a heterologous sequence positioned between two ITR sequences. In some embodiments, the expression cassette is positioned between a L-ITR (left ITR) and a R-ITR (right ITR). In some embodiments, the expression cassette comprises, in 5′ to 3′ order, a L-ITR, the heterologous sequence, and a R-ITR.

In some embodiments, the expression cassette has a length of about 3000 to about 6000 nucleobases. In some embodiments, the expression cassette has a length of about 4000 to about 5000 nucleobases. In some embodiments, the expression cassette has a length of about 4500 to about 5000 nucleobases. In some embodiments, the expression cassette has a length of about 2000 to about 3000 nucleobases. In some embodiments, provided is a vector that is self-complementary, wherein the vector comprises a stuffer sequence such that the expression cassette of the vector has a length of about 2000 to about 3000 nucleobases.

In some embodiments, the expression cassette comprises a stuffer sequence. In some embodiments, the stuffer sequence has a length of about 1000 to about 10000, about 1000 to about 9000, about 1000 to about 8000, about 1000 to about 7000, about 1000 to about 6000, about 1000 to about 5000, about 1000 to about 4000, about 1000 to about 3000 or about 1000 to about 2000 nucleobases. In some embodiments, the stuffer sequence has a length of about 2000 to about 3000 nucleobases. In some embodiments, the stuffer sequence has a length of 2235 nucleobases. In some embodiments, the stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least a 100 contiguous basepair sequence of SEQ ID NO: 7. In some embodiments, the stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 7.

In some embodiments, the expression cassette comprises a therapeutic peptide. In some embodiments, the expression cassette comprises at least one regulatory region. In some embodiments, the stuffer sequence is located between the therapeutic peptide and the regulatory region. In some embodiments, the therapeutic peptide is located between the stuffer sequence and the R-ITR. In some embodiments, the stuffer sequence is located between the therapeutic peptide and the L-ITR.

a. Therapeutic Peptide

In some embodiments, an AAV plasmid disclosed herein comprises a sequence that encodes a diagnostic or therapeutic protein, polypeptide or a biologically active fragment of a molecular marker, a photosensitive opsin, an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, a tumor suppressor, or any combination thereof. In some embodiments, a photosensitive opsin comprises a rhodopsin, a melanopsin, a cone opsin, a channel rhodopsin, or a bacterial, archaea-associated opsin, biologically active fragments of any of these or combinations thereof.

In some embodiments, a AAV plasmid described herein comprises a sequence encoding a therapeutic peptide or a biologically active fragment thereof selected from the group consisting of GUCY2D, RS1, CNBG3, ADAMTS10, ABCA4, or frataxin. In some embodiments, the AAV plasmid comprises a nucleic acid segment that encodes the polypeptide RPE65, Bestrophin (BEST1), REP1, MERTK, SOD2, MYO6A, MFRP, LRAT, KCNJ13, ornithine aminotransferase (OAT), CNTF, GDNF, BDNF, IL6, LIF, XIAP, STATS, nyctalopin (nyx), metabotropic glutamate receptor 6-mGluR6 (Grm6), transient receptor potential melastatin 1 (TRPM1), G protein coupled receptor 179 (GPR179), and G proteins, Gβ5, βP3, Gα0_{1/2}, Gγ13, RGS7, RGS11, REAP, MYO7A, OPN1MW, OPN1LW, CNGA3, CNGB1, Rho, PDE6b, PDE6a, GNAT2, PDE6c, RPGR, RPGR-ORF15, RPGRIP, CLRN1, Ush1c/Harmonin, Mt-ND4, P1-ND4, CYP4V2, any combination or peptide fragment thereof. In some embodiments, the AAV plasmid described herein comprises a sequence encoding a CRISPR-Cas system. In some embodiments, the AAV plasmid described herein comprises a sequence encoding a CRISPR-Cas9 system.

In some embodiments, the sequence encoding a therapeutic peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least a 100 contiguous basepair sequence of SEQ ID NO: 4. In some embodiments, the sequence encodes a therapeutic peptide comprising a sequence about or at least about identical to at least 50 contiguous amino acids of SEQ ID NO: 34, 35, 36, 37, 38, 39, 40, 41, 42, 43, or 44. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4. In some embodiments, the peptide comprises SEQ ID NO. 4. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 34. In some embodiments, the peptide comprises SEQ ID NO. 34. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 35. In some embodiments, the peptide comprises SEQ ID NO. 35. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 36. In some embodiments, the peptide comprises SEQ ID NO. 36. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 37. In some embodiments, the peptide comprises SEQ ID NO. 37. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 38. In some embodiments, the peptide comprises SEQ ID NO. 38. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 39. In some embodiments, the peptide comprises SEQ ID NO. 39. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 40. In some embodiments, the peptide comprises SEQ ID NO. 40. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 41. In some embodiments, the peptide comprises SEQ ID NO. 41. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 42. In some embodiments, the peptide comprises SEQ ID NO. 42. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 43. In some embodiments, the peptide comprises SEQ ID NO. 43. In some embodiments, the peptide comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 44. In some embodiments, the peptide comprises SEQ ID NO. 44.

b. Regulatory Regions

Any of the vector systems of the disclosure may include regulatory elements that are functional in the intended host cell in which the vector is to be expressed. Regulatory elements include, for example, promoters, transcription termination sequences, translation termination sequences, enhancers, and polyadenylation elements.

Any of the vector systems of the disclosure may include a promoter sequence operably linked to a nucleotide sequence encoding a desired polypeptide. Promoters contemplated for use in the disclosure include, but are not limited to, cytomegalovirus (CMV) promoter, SV40 promoter, human myosin 7a gene-derived promoter, Rous sarcoma virus (RSV) promoter, chimeric CMV/chicken 3-actin promoter (CBA) and the truncated form of CBA (smCBA) (see, e.g., Haire et al. 2006 and U.S. Pat. No. 8,298,818, each of which is incorporated herein by reference). Additional photoreceptor-specific, human rhodopsin kinase (hGRK1) promoter, a synapsin promoter, a glial fibrillary acidic protein (GFAP) promoter, rod specific IRBP promoter, VMD2 (vitelliform macular dystrophy/Best disease) promoter, a RPE-specific vitelliform macular dystrophy-2 [VMD2] promoter, EF1-alpha promoter sequences, PGK promoter, Pleiades “PleXXX” promoters, red/green cone opsin promoters PR2.1 and PR1.7 are also contemplated to be useful in the practice of various aspects of the disclosure. Exemplary photoreceptor-cell-specific promoters include, but are not limited to, hGRK1, IRBP, rod opsin, NRL, GNAT2e-IRBP, L/M opsin, and cone arrestin promoters. In some embodiments, the promoter comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2.

In particular embodiments, the promoter is a chimeric CMV-β-actin promoter. In particular embodiments, the promoter is a tissue-specific promoter that shows selective activity in one or a group of tissues but is less active or not active in other tissue. In particular embodiments, the promoter is a photoreceptor-specific promoter. In a further embodiment, the promoter is preferably a cone cell-specific promoter or a rod cell-specific promoter, or any combination thereof. In particular embodiments, the promoter is the promoter for human MYO7A gene. In a further embodiment, the promoter comprises a cone transducin α (TαC) gene-derived promoter. In particular embodiments, the promoter is a human GNAT2-derived promoter. Other promoters contemplated within the scope of the disclosure include, without limitation, a rhodopsin promoter (human or mouse), a cGMP-phosphodiesterase β-subunit promoter, a retinitis pigmentosa-specific promoter, an RPE cell-specific promoter [such as a vitelliform macular dystrophy-2 (VMD2) promoter (Best1) (Esumi et al., 2004)], or any combination thereof.

Promoters can be incorporated into a vector using standard techniques known to those of ordinary skill in the molecular biology and/or virology arts. Multiple copies of promoters, and/or multiple distinct promoters can be used in the vectors of the disclosure. In one such embodiment, a promoter may be positioned about the same distance from the transcription start site as it is from the transcription start site in its natural genetic environment, although some variation in this distance is permitted, of course, without a substantial decrease in promoter activity. In the practice of the disclosure, one or more transcription start site(s) are typically included within the disclosed vectors.

The vectors of the disclosure may further include one or more transcription termination sequences, one or more translation termination sequences, one or more signal peptide sequences, one or more internal ribosome entry sites (IRES), and/or one or more enhancer elements, or any combination thereof. Transcription termination regions can typically be obtained from the 3′-untranslated region of a eukaryotic or viral gene sequence. Transcription termination sequences can be positioned downstream of a coding sequence to provide for efficient termination. In some embodiments, the vectors comprise a self-cleaving peptide. In some embodiments, the self-cleaving peptide allows for the co-expression of more than one. In some embodiments, the self-cleaving peptide comprises a 2A self-cleaving peptide. In some embodiments, the peptide comprises P2A, E2A, F2A, or T2A.

Any of the disclosed polynucleotide vectors may also further include one or more post-transcriptional regulatory sequences or one or more polyadenylation signals, including, for example, but not limited to, a woodchuck hepatitis virus post-transcription regulatory element (WRPE), a polyadenylation signal sequence, or an intron/exon junctions/splicing signals, or any combination thereof. In some embodiments, the expression cassette comprises a woodchuck hepatitis virus posttranscriptional regulatory element (WPRE). In some embodiments, the WPRE comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 5. In some embodiments, the expression cassette comprises a pGH polyA sequence. In some embodiments, the pGH polyA sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6. In some embodiments, the intron/exon junction/splicing signal comprises a SV40SD/SA. In some embodiments, the SV40SD/SA comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 34.

Signal peptide sequences are amino-terminal peptidic sequences that encode information responsible for the location of an operably-linked polypeptide to one or more post-translational cellular destinations, including, for example, specific organelle compartments, or to the sites of protein synthesis and/or activity, and even to the extracellular environment.

Enhancers—cis-acting regulatory elements that increase gene transcription—may also be included in one of the disclosed AAV-based vector systems. A variety of enhancer elements are known to those of ordinary skill in the relevant arts, and include, without limitation, a CaMV 35S enhancer element, a cytomegalovirus (CMV) early promoter enhancer element, an SV40 enhancer element, as well as combinations and/or derivatives thereof. One or more nucleic acid sequences that direct or regulate polyadenylation of the mRNA encoded by a structural gene of interest, may also be optionally included in one or more of the vectors of the disclosure.

### Backbone

In some embodiments, the AAV plasmid comprises a backbone. In some embodiments, the backbone comprises the region of the plasmid outside of the ITRs. In some embodiments, the stuffer sequence is positioned outside of the expression cassette. In some embodiments, the backbone sequence is at least about 4000, about 5000, about 6000, about 7000, about 8000, about 9000, or about 10000 nucleobases. In some embodiments, the backbone sequence is about 4000 to about 10000, about 4000 to about 9000, about 4000 to about 8000, about 4000 to about 7000, about 40000 to about 6000, or about 4000 to about 5000 nucleobases. In some embodiments, the backbone sequence is about 5000 to about 10000, about 5000 to about 9000, about 5000 to about 8000, about 5000 to about 7000, or about 50000 to about 6000 nucleobases. In some embodiments, the backbone sequence is about 1000 to about 8000, about 2000 to about 8000, about 3000 to about 8000, about 4000 to about 8000, about 500o to about 8000 or about 6000 to about 8000 nucleobases. In some embodiments, the backbone sequence is large enough to prevent reverse packaging into an AAV particle.

In some embodiments, the backbone comprises an origin of replication. In some embodiments, the nucleic acid stuffer sequence is positioned 3′ to the origin of replication. In some embodiments, the nucleic acid stuffer sequence is positioned between the origin of replication and an ITR. In some embodiments, the nucleic acid stuffer sequence is located such that the ITR is about 2000 to about 4000 nucleobases away from the origin of replication. In some embodiments, the nucleic acid stuffer sequence is located such that the ITR is about 1000 to about 4000 nucleobases away from the origin of replication. In some embodiments, the nucleic acid stuffer sequence is located such that the ITR is about 3000 to about 3500 nucleobases away from the origin of replication. In some embodiments, the nucleic acid stuffer sequence is located such that the ITR is more than about 500, 1000, 2000, 3000, 4000, or 5000 nucleobases away from the origin of replication. In some embodiments, the nucleic acid stuffer sequence is located such that the ITR is less than about 500, 1000, 2000, 3000, 4000, or 5000 nucleobases away from the origin of replication. In some embodiments, the stuffer sequence is located such that the ITR is about 3124 nucleobases away from the origin of replication.

In some embodiments, the backbone sequence comprises two stuffer sequences. In some embodiments, the first stuffer sequence is located between the R-ITR and the origin or replication. In some embodiments, the first stuffer sequence is located between the R-ITR and an antibiotic resistance gene. In some embodiments, the second stuffer sequence is located between the origin of replication and the L-ITR. In some embodiments, the second stuffer sequence is located between the antibiotic resistance gene and the L-ITR. In some embodiments, the backbone comprises, in a 5′ to 3′ order, the first stuffer sequence, the origin of replication, the antibiotic resistance gene, and the second stuffer sequence. In some embodiments, the backbone comprises, in a 3′ to 5′ order, the first stuffer sequence, the origin of replication, the antibiotic resistance gene, and the second stuffer sequence.

In some embodiments, the first stuffer sequence has a length of about 1000 to about 10000, about 2000 to about 9000, about 3000 to about 8000, about 4000 to about 6000 nucleobases. In some embodiments, the first stuffer sequence has a length of about 3000 to about 3500 nucleobases. In some embodiments, the nucleic acid stuffer sequence has a length of about 3028 nucleobases. In some embodiments, the first stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least a 100 contiguous basepair sequence of SEQ ID NO: 8. In some embodiments, the first stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 8.

In some embodiments, the second stuffer sequence has a length of about 100 to about 1000, about 100 to about 900, about 100 to about 800, about 100 to about 700, about 100 to about 600, about 100 to about 500, about 100 to about 400, about 100 to about 300, or about 100 to about 200 nucleobases. In some embodiments, the second stuffer sequence has a length of about 100 to about 500 nucleobases. In some embodiments, the nucleic acid stuffer sequence has a length of about 381 nucleobases. In some embodiments, the second stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least a 100 contiguous basepair sequence of SEQ ID NO: 11. In some embodiments, the second stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 11.

In some embodiments, the AAV plasmid comprises a selectable marker. In some embodiments, the selectable marker is an antibiotic resistance gene. In some embodiments, the AAV plasmid comprises a kanamycin resistance gene. In some embodiments, the AAV plasmid does not comprise an antibiotic gene. In some embodiments, the AAV plasmid does not comprise an ampicillin antibiotic resistance gene.

In some embodiments, the selectable marker is a non-antibiotic selection system. In some embodiments, the non-antibiotic selection system comprises a RNA-OUT sequence and an R6K sequence.

In some embodiments, the backbone has no more than one origin of replication. In some embodiments, the backbone does not have a M13 origin of replication.

### ITRs

In some embodiments, the plasmids described herein comprise at least one sequence comprising inverted repeats (ITRs). ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV6. In some embodiments, the ITR sequences of the first serotype are derived from AAV3, AAV2 or AAV6. In other embodiments, the ITR sequences of the first serotype are derived from AAV1, AAV5, AAV8, AAV9 or AAV10. In some embodiments, the ITR sequences are the same serotype as the capsid (e.g., AAV3 ITR sequences and AAV3 capsid, etc.).

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. In some embodiments, the nucleic acid vector comprises a pTR-UF-11 plasmid backbone, which is a plasmid that contains AAV2 ITRs. This plasmid is commercially available from the American Type Culture Collection (ATCC MBA-331).

In some embodiments, the ITR sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In some embodiments, the ITR sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 51.

### C. Triple Transfection System

In certain aspects, described herein is a triple plasmid transfection system for the production of an AAV plasmid. In some embodiments, the triple-plasmid transfection system comprises an AAV vector plasmid as described herein, a rep/cap plasmid and a helper plasmid.

In certain aspects, described herein is a double plasmid transfection system for the production of an AAV plasmid. In some embodiments, the double-plasmid transfection system comprises an AAV vector plasmid (also referred to as a transfer plasmid) as described herein and a helper plasmid, wherein the helper plasmid comprises a rep/cap sequence.

For illustration only, either a triple-plasmid system or a double plasmid transfection system could be used to produce the same AAV plasmid. In one instance, a triple transfection system would include pTR-UF11 (CBA-GFP) (SEQ ID NO: 45), helper plasmid pALD-X80 (SEQ ID NO: 47), and rep/cap plasmid pACG2 (SEQ ID NO: 46) transfected into HEK293T cells. In another embodiment, a two plasmid system would consist of pTR-UF11 (CBA-GFP) (SEQ ID NO: 45) and pDG (SEQ ID NO: 48). The plasmid pDG is a helper plasmid which also contains the elements to drive expression of AAV2 rep/cap. The result of these three and two plasmid systems would result in the same AAV2-GFP product.

### Helper Plasmids

In some embodiments, the systems described herein comprise a helper plasmid. In some embodiments, the helper plasmid comprises a E1a gene, a E1b gene, a E4 gene, a E2a, a e3 gene, a E5 gene, a Fiber gene, or a VA gene or a combination thereof. In some embodiments, the helper plasmid comprises a mutated Fiber gene. In some embodiments, the Fiber gene comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% to SEQ ID NO: 12. In some embodiments, the Fiber gene comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% to SEQ ID NO: 13. In some embodiments, the helper plasmid does not comprise a Fiber gene.

In some embodiments, the helper plasmid comprises pDM, pDG, pDP1rs, pDP2rs, pDP3rs, pDP4rs, pDP5rs, pDP6rs, pDG(R484E/R585E), or pDP8.ape plasmids. In some embodiments, the helper plasmid comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% to SEQ ID NO: 47 In some embodiments, the helper plasmid comprises SEQ ID NO: 47.

### Rep/Cap Plasmids

In some embodiments, the systems described herein comprise a rep/cap plasmid comprising a rep gene (e.g., encoding Rep78, Rep68, Rep52 and Rep40) and a cap gene (encoding VP1, VP2, and VP3, including a modified VP3 region as described herein). In some embodiments, a rep/cap plasmid is transfected into a producer cell line such that the rAAV particle can be packaged and subsequently purified. In some embodiments, the rep/cap plasmid comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% to SEQ ID NO: 46 In some embodiments, the rep/cap plasmid comprises SEQ ID NO: 46.

In some embodiments, the rep gene is a rep gene derived from AAV2. In some embodiments the cap gene is derived from AAV2. In some embodiments, the rep gene is a rep gene derived from AAV12. In some embodiments, the Rep gene is Rep2-6. In some embodiments, the Rep gene comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% to SEQ ID NO: 49. In some embodiments, the Rep gene comprises SEQ ID NO: 49.

In some embodiments, the cap gene is derived from AAV12. In some embodiments, the cap gene includes modifications to the gene in order to produce a modified capsid protein described herein. In some embodiments, the rep gene comprises Rep78, Rep68, Rep52 or Rep40. In some embodiments, the cap gene comprises VP1, VP2, VP3 or variants thereof. In some embodiments, the cap gene comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% to SEQ ID NO: 50. In some embodiments, the cap gene comprises SEQ ID NO: 50.

In some embodiments, the disclosure provides improved rAAV particles that have been derived from a number of different serotypes, including but not limited to AAV1, AAV2, AAV3, AAV4, AAV5, AAV6, AAV7, AAV8, AAV9, AAV10, AAV44.9(E531D) and combinations thereof. In some embodiments, the capsid protein sequences are set forth in SEQ ID NO:21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 31. In some embodiments, the capsid protein sequences comprises SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 31. In some embodiments, the capsid protein sequences comprises at least about 70%, 75%, 80%, 85%, 90%, 95%, 96%, 97%, 98%, 99% or 100% sequence identity with SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 31.

In some embodiments, the capsid comprises capsids comprising non-native amino acid substitutions at amino acid residues of a wild-type AAV2 capsid as set forth in SEQ ID NO: 22. In some embodiments, the non-native amino acid substitutions comprise one or more of Y272F, Y444F, T491V, Y500F, Y700F, Y704F Y730F or a combination thereof. In some embodiments, the capsids comprises non-native amino acid substitutions at amino acid residues of a wild-type AAV6 capsid as set forth in SEQ ID NO: 26. In some embodiments, the non-native amino acid substitutions comprise one or more of Y445F, Y705F, Y73IF, T492V, S663V or a combination thereof.

In some embodiments, the capsid comprises AAV2G9, a variant of AAV2.

In some embodiments, the capsid comprises a non-native amino acid substitution at amino acid residue 533 of a wild-type AAV8 capsid as set forth in SEQ ID NO: 28. In some embodiments, the non-native amino acid substitution is E533K, Y733F, or a combination thereof. In some embodiments, the capsid comprises AAV7BP2, a variant of AAV8.

In some embodiments, the capsid comprises non-native amino acid substitutions of a wild-type AAV2 capsid as set forth in SEQ ID NO: 22. In some embodiments, the capsid comprises one or more of:
(a) Y444F;
(b) Y444F+Y500F+Y730F;
(c) Y272F+Y444F+Y500F+Y730F;
(d) Y444F+Y500F+Y730F+T491V; or
(e) Y272F+Y444F+Y500F+Y730F+T491V, or at equivalent amino acid positions corresponding thereto in any one of the wild-type AAV1, AAV3, AAV4, AAV5, AAV7, AAV9, or AAV10 capsid proteins, as set forth, respectively, in SEQ ID NO: 21, SEQ ID NO: 23, SEQ ID NO: 24, SEQ ID NO: 25, SEQ ID NO: 27, SEQ ID NO: 29, or SEQ ID NO: 30.

In some embodiments, the capsid comprises non-native amino acid substitutions of a wild-type AAV6 capsid as set forth in SEQ ID NO: 26. In some embodiments, the capsid comprises one or more of:
(a) Y445F;
(b) Y705F+Y731F;
(c) T492V;
(d) Y705F+Y731F+T492V;
(e) S663V; or
(f) S663V+T492V.

In various embodiments, the rAAV particles comprise one of the following capsids, i.e., capsid variants of AAV2: DGE-DF (also known as ‘V1V4 VR-V’), P2-V2, P2-V3, P2-V1, (also known as ME-B) and ME-B(Y-F+T-V). The DGE-DF capsid variant contains aspartic acid, glycine, glutamic acid, aspartic acid, and phenylalanine at amino acid positions 492, 493, 494, 499, and 500 of wild-type AAV2 VP1. The P2-V2 capsid variant contains alanine, threonine, proline, aspartic acid, phenylalanine, and aspartic acid at positions 263, 490, 492, 499, 500, and 530 of AAV2 VP1. The P2-V3 capsid variant contains asparagine, alanine, phenylalanine, alanine, asparagine, valine, threonine, arginine, aspartic acid, and aspartic acid at positions 263, 264, 444, 451, 454, 455, 459, 527, 530, and 531 of AAV2 VP1. The ME-B(Y-F+T-V) capsid variant contains aspartic acid, glycine, glutamic acid, aspartic acid, and phenylalanine at positions 492, 493, 494, 499, and 500 of AAV2 VP1 (SEQ ID NO: 22), respectively, SAAGADXAXDS (SEQ ID NO: 52) at positions 546-556 of AAV2 VP1, and the following substitutions: Y272F, Y444F, and T491V.

In other embodiments, the rAAV particles comprise a capsid selected from AAV6(3pMut), AAV2(quadYF+T-V), or AAV2(trpYF). In some embodiments, the rAAV particles comprise any of the capsid variants described in International Patent Publication No. WO 2018/156654.

In some embodiments, the AAV particles comprise a capsid comprising a DGE-DF capsid, P2-V2 capsid, P2-V3 capsid, or ME-B(Y-F+T-V) capsid for the enhanced transduction of said rAAV particles in retinal cells. In some embodiments, the AAV particles comprise a capsid selected from AAV2(Y444F), AAV2(Y444F+Y500F+Y730F), AAV2(Y272F+Y444F+Y500F+Y730F), AAV2(Y444F+Y500F+Y730F+T491V) and AAV2(Y272F+Y444F+Y500F+Y730F+T491V), AAV6(Y445F), AAV6(Y705F+Y731F), AAV6(Y705F+Y731F+T492V), AAV6(S663V), AAV6(T492V) or AAV6(S663V+T492V).

In some embodiments, the rAAV polynucleotide or nucleic acid vectors of the present disclosure may be comprised within a virion having a serotype that is selected from the group consisting of AAV serotype 1, AAV serotype 2, AAV serotype 3, AAV serotype 4, AAV serotype 5, AAV serotype 6, AAV serotype 7, AAV serotype 8, AAV serotype 9, or AAV serotype 10, AAV449.5(E531D) or any other serotype as known to one of ordinary skill in the viral arts.

### Production

In some embodiments, described herein is a method of rAAV particle production. In some embodiments, one or more helper plasmids are produced or obtained. In some embodiments, a helper plasmid and a rep/cap plasmid are produced or obtained. In some embodiments, the one or more helper plasmids comprise rep and cap ORFs for the desired AAV serotype and the adenoviral VA, E2A (DBP), and E4 genes. In some embodiments, the rep and cap ORFs for the desired AAV serotype and the adenoviral VA, E2A (DBP), and E4 genes are under the transcriptional control of their native promoters. In some embodiments, the cap ORF comprises one or more modifications to produce a modified capsid protein as described herein. In some embodiments, HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. In some embodiments, the HEK293 cells are incubated for at least about 60 hours to allow for rAAV particle production. In some embodiments, the cells are then incubated for at least 60 hours to allow for rAAV particle production. In some embodiments, the rAAV particles are purified. In some embodiments, the rAAV particles are purified by iodixanol step gradient, CsCl gradient, chromatography, or polyethylene glycol (PEG) precipitation.

### II. Methods

In some aspects, the disclosure provides methods for treating or ameliorating a disease or condition, such as an eye disease, in a human or animal using gene therapy and an AAV-based dual vector system of the disclosure. In particular embodiments, a method of the disclosure comprises administering a vector system of the disclosure that encodes a polypeptide that provides for treatment or amelioration of the disease or condition. In particular embodiments, the vectors of the disclosure are provided in an AAV virus or virion. The vector system can be administered in vivo or ex vivo.

In some embodiments, a rAAV vector construct disclosed herein can be administered via an intravitreal injection, subretinal injection, orally, parenterally, intraocularly, intravenously, intranasally, intra-articularly, intramuscularly, subcutaneously, subILM (vector is placed between the inner limiting membrane and the retina), or a combination thereof. In some embodiments, a rAAV vector construct disclosed herein is administered a single time to a subject. In some embodiments, the rAAV vector construct is administered to the subject in one or more administration periods, for example at least once a day, twice a day, three times per day, once a week, twice a week, once a month, twice a month or at least one a year. In some embodiments, the AAV vector-based therapeutics may be provided successively in one or more daily, weekly, monthly, or less-frequent periods, as may be necessary to achieve treatment, or amelioration of one or more symptoms of the disease or disorder being treated. In some embodiments, a pharmaceutical composition disclosed herein can be administered a single time or multiple times, for example daily, weekly, biweekly, or monthly, hourly, or is administered upon recurrence, relapse or progression of the disease, disorder or condition being treated.

In some embodiments, vector systems are administered to, e.g., hair cells of the ear, by injection into the utricle, which is one of two sac-like otolith organs sensitive to gravity, as described in Lee et al., Hearing Research Vol. 394 (2020) 107882, incorporated by reference herein. Administration to, e.g., hair cells of the ear may be by a round window injection, or during cochlear implant surgery. In particular embodiments, a vector system of the disclosure is administered to the human or animal by intraocular, intravitreal or subretinal injection.

In some embodiments, administration of any of the disclosed vectors, virions, or compositions to a subject in need thereof provides a partial or complete restoration of melanosome migration in retinal pigment epithelium (RPE) cells. In exemplary embodiments, administration of any of the polynucleotide vector systems, virions, or compositions provides a partial or complete restoration of vision loss.

In some embodiments, described herein are methods of use of the described rAAV particles or vectors, virions, expression systems, compositions, and host cells described herein in the preparation of medicaments for diagnosing, preventing, treating or ameliorating at least one or more symptoms of a disease, a dysfunction, a disorder, an abnormal condition, a deficiency, injury, or trauma in an animal, and in particular, in the eye. In some embodiments, the methods comprise direct administration to the vitreous of one or both eyes of a mammal in need thereof, one or more of the described vectors, virions, viral particles, host cells, compositions, or pluralities thereof, in an amount and for a time sufficient to diagnose, prevent, treat, or lessen one or more symptoms of such a disease, dysfunction, disorder, abnormal condition, deficiency, injury, or trauma in one or both eyes of the affected animal.

In some aspects, the present disclosure provides methods of use of the particles, vectors, virions, expression systems, compositions, and host cells described herein in a method for treating or ameliorating the symptoms, or in the preparation of medicaments for, treating or ameliorating the symptoms of various deficiencies in an eye of a mammal, and in particular one or more deficiencies in human photoreceptors or RPE cells. In some embodiments, diseases and disorders of the eye (e.g., caused by one or more genetic deficiencies in a PR or RPE cell) for treatment or amelioration of symptoms comprise Retinitis pigmentosa, Leber Congenital Amaurosis (e.g., LCA10), Age Related Macular Degeneration (AMD), wet AMD, dry AMD, uveitis, Best disease, Stargardt disease, Usher Syndrome, Geographic Atrophy, Diabetic Retinopathy, Retinoschisis, Achromatopsia, Choroideremia, Bardet Biedl Syndrome, Autosomal-dominant cord-rod dystrophy (CORD6), glaucoma including primary open angle glaucoma, Freidreich's ataxia, glycogen storage diseases (ocular manifestation), congenital stationary night blindness, Leber Hereditary Optic neuropathy (LHON), or bietti's crystalline dystrophy. In some embodiments, the methods comprise intravitreal or subretinal administration to one or both eyes of a subject in need thereof, one or more of the described particles vectors, virions, host cells, or compositions, in an amount and for a time sufficient to treat or ameliorate the symptoms of such a deficiency in the affected mammal. In some embodiments, the methods comprise prophylactic treatment of an animals suspected of having such conditions, or administration of such compositions to those animals at risk for developing such conditions either following diagnosis, or prior to the onset of symptoms. In some embodiments, the rAAV particle is not comprised in a chimeric viral/non-viral nanoparticle.

### III. Pharmaceutical Compositions

Pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, e.g., parabens, chlorobutanol, phenol, sorbic acid, thimerosal, and the like. In many cases, it will be preferable to include isotonic agents, e.g., sugars, buffers or sodium chloride. Prolonged absorption of the injectable compositions can be brought about by the inclusion of agents that delay absorption, e.g., aluminum monostearate and gelatin.

The disclosure also provides pharmaceutical compositions comprising a vector system of the disclosure in combination with a pharmaceutically acceptable carrier. Pharmaceutical compositions adapted for topical or parenteral administration, comprising an amount of a compound constitute a preferred embodiment of the disclosure. The dose administered to a patient, particularly a human, in the context of the disclosure should be sufficient to achieve a therapeutic response in the patient over a reasonable timeframe, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

The disclosure also provides kits comprising a vector system of the disclosure in one or more containers. Kits of the disclosure can optionally include pharmaceutically acceptable carriers and/or diluents. In particular embodiments, a kit of the disclosure includes one or more other components, adjuncts, or adjuvants as described herein. In particular embodiments, a kit of the disclosure includes instructions or packaging materials that describe how to administer a vector system contained within the kit to a selected mammalian recipient.

Containers of the disclosed kits may be of any suitable material, e.g., glass, plastic, metal, etc., and of any suitable size, shape, or configuration. In particular embodiments, a vector system of the disclosure is provided in the kit as a solid. In another embodiment, a vector system of the disclosure is provided in the kit as a liquid or solution. In some embodiments, the kits may include one or more ampoules or syringes that contain a vector system of the disclosure in a suitable liquid or solution form.

Further contemplated herein are kits containing a pre-mixture of any of the disclosed dual vectors (front half vector and back half vector). These pre-mixtures may be in a single container and/or a single drug product in a suitable liquid or solution form.

The disclosure also provides for the use of the buffers and compositions disclosed herein in the manufacture of a medicament for treating, preventing or ameliorating the symptoms of a disease, disorder, dysfunction, injury or trauma, including, but not limited to, the treatment, prevention, and/or prophylaxis of a disease, disorder or dysfunction, and/or the amelioration of one or more symptoms of such a disease, disorder or dysfunction.

The amount of AAV compositions and time of administration of such compositions will be within the purview of the skilled artisan having benefit of the present teachings. The administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the AAV vector compositions, either over a relatively short, or over a relatively prolonged period, as may be determined by the medical practitioner overseeing the administration of such compositions.

For example, the number of infectious particles administered to a mammal may be approximately 10⁷, 10¹, 10⁹, 10¹⁰, 10¹¹, 10¹², 10¹³, or even higher, infectious particles/mL, given either as a single dose (or divided into two or more administrations, etc.) as may be required to achieve therapy of the particular disease or disorder being treated. In fact, In some embodiments, it may be desirable to administer two or more different rAAV particle- or vector-based compositions, either alone, or in combination with one or more other diagnostic agents, drugs, bioactives, or such like, to achieve the desired effects of a particular regimen or therapy.

To express a therapeutic agent in accordance with the present disclosure one may prepare a rAAV particle that comprises a therapeutic agent-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (for example, 3′ of) the chosen promoter. The “upstream” promoter stimulates transcription of the DNA and promotes expression of the encoded polypeptide. This is the meaning of “recombinant expression” in this context. In some embodiments, recombinant vector constructs are those that include a capsid-protein modified rAAV vector that contains an RPE cell- or a photoreceptor cell-specific promoter, operably linked to at least one nucleic acid segment encoding one or more diagnostic, and/or therapeutic agents.

When the use of such vectors is contemplated for introduction of one or more exogenous proteins, polypeptides, peptides, ribozymes, and/or antisense oligonucleotides, to a particular cell transfected with the vector, one may employ the rAAV particles disclosed herein to deliver one or more exogenous polynucleotides to a selected host cell, e.g., to one or more selected cells within the mammalian eye. In some embodiments, the cell is a retinal ganglion cell, muller glia, bipolar cell, an astrocyte, an amacrine cell, a trabecular meshwork cell, a photoreceptor cell or a retinal pigment epithelial cell. In some embodiments, the cell is a photoreceptor cell. In some embodiments, the cell is a retinal pigment epithelial (RPE) cell.

In some embodiments, the number of viral particles administered to a subject may be on the order ranging from 10⁶ to 10¹⁴ particles/ml or 103 to 10¹⁵ particles/ml. In one embodiment, viral particles of higher than 10¹³ particles/ml may be administered. In some embodiments, the number of viral particles administered to a subject may be on the order ranging from 10⁶ to 10¹⁴ vector genomes(vgs)/ml or 103 to 10¹⁵ vgs/ml. In one embodiment, viral particles of higher than 10¹³ vgs/ml are administered. The viral particles can be administered as a single dose, or divided into two or more administrations as may be required to achieve therapy of the particular disease or disorder being treated.

In some embodiments, the disclosure provides formulations of one or more viral-based compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particles described herein may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized as described herein.

Formulation of pharmaceutically-acceptable buffer, excipients and carrier solutions is well known to those of skill in the art, as is the development of suitable dosing and treatment regimens for using the particular compositions described herein in a variety of treatment regimens, including e.g., oral, parenteral, intraocular (e.g., subretinal or intravitreal), intravenous, intranasal, intra-articular, intra-utricle, intracochlear and intramuscular administration and formulation.

Typically, these formulations may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The term “excipient” refers to a diluent, adjuvant, carrier, or vehicle with which the rAAV particle is administered. Such pharmaceutical excipients can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Exemplary excipients and vehicles include, but are not limited to, HA, BSS, artificial CSF, PBS, Ringer's lactate solution, TMN200 solution, polysorbate 20, and poloxamer 100.

The amount of rAAV particle compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of viral particles to provide therapeutic benefit to the patient undergoing such treatment. Alternatively, in some circumstances, it may be desirable to provide multiple, or successive administrations of the compositions, either over a relatively short, or a relatively prolonged period of time, as may be determined by the medical practitioner overseeing the administration of such compositions.

Exemplary compositions may include rAAV particles or nucleic acid vectors either alone, or in combination with one or more additional active ingredients, which may be obtained from natural or recombinant sources or chemically synthesized.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “promoter,” as used herein refers to a region or regions of a nucleic acid sequence that regulates transcription.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector. In some embodiments, the vector is an AAV plasmid.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refer to two or more sequences or subsequences that have at least about 70%, at least about 75%, at least about 80%, at least about 85%, at least about 90%, at least about 95, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. In some embodiments, % amino acid sequence identify values can be generated using the sequence comparison program ALIGN 2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The section headings used herein are for organizational purposes only and are not to be construed as limiting the subject matter described.

### V. Examples

The following examples are included for illustrative purposes only and are not intended to limit the scope of the invention.

### Example 1: Loss of ITRs in Ampicillin and Antibiotic Resistant Cells

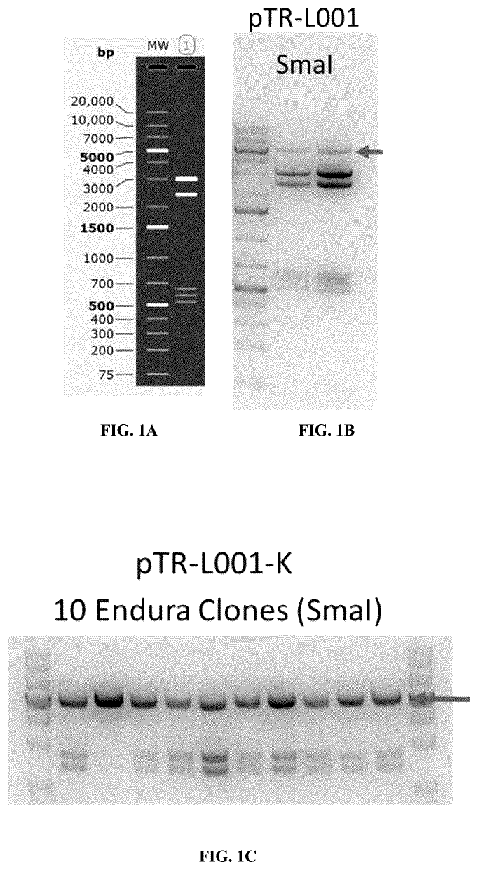

Production of ITR plasmids are vulnerable to replication errors. ITRs are highly unstable in kanamycin resistant compatible cell lines. AAV plasmids containing ITRs were amplified in Kanamycin resistant Endura cells and ampicillin resistant SURE cells. The purified plasmids were linearized with SmaI, resulting in two bands between 2000 and 3000 basepairs and three bands between 500 and 700 basepairs, as indicated by lane 1 of FIG. 1A. Loss of ITRs within the plasmids results in a band at 5000 bp. The plasmids amplified in ampicillin-resistant cells (FIG. 1B) showed a greater low of ITRs as indicated by the band at 5000 bp than the plasmids amplified in kanamycin-resistant cells (FIG. 1C).

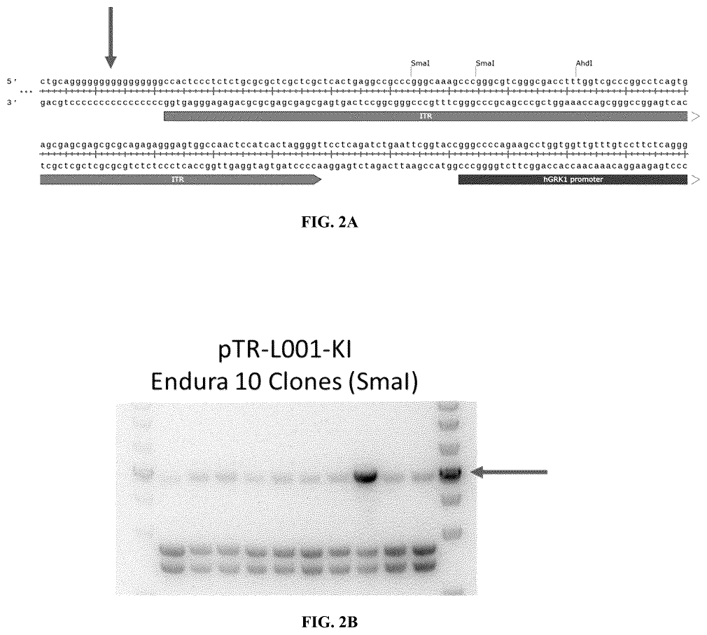

The polyG/C sequence depicted in FIG. 2A (arrow) was removed from the kanamycin-resistant plasmid. Additionally, the polyC run adjacent to the R-ITR was removed. The plasmid was digested with SmaI and run on a gel to identify the stability of the ITRS. As depicted in FIG. 2B, ITR stability was improved by removal of the polyG/C sequence (compare FIG. 2B to FIG. 1B).

### Example 2: Prevention of Reverse Packaging with a Stuffer Sequence

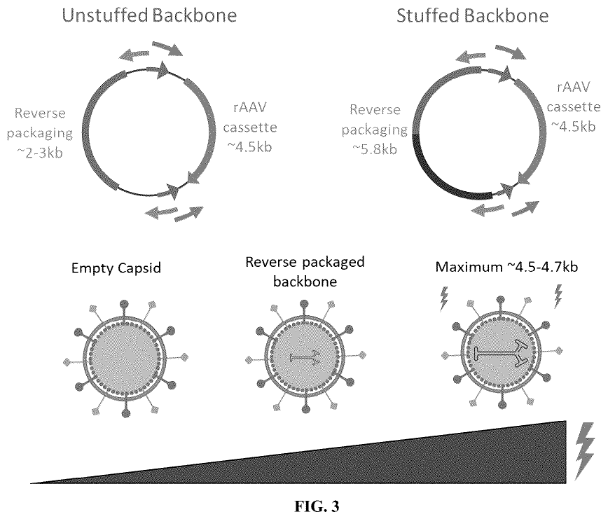

The efficiency of packaging an rAAV cassette is reduced by the production of empty capsids and reverse packaged backbone sequence, as depicted in FIG. 3. A stuffer sequence was designed to increase the size of the backbone to reduce reverse packaging. First a stuffer DNA sequence was randomly generated. Next, all open reading frames larger than 20 amino acids were mutated. Next, CpG islands were mutated until CpG islands were no longer detectable. Next, most restriction enzyme sites were removed. Next, repetitive strings greater than 4 nucleobases were removed.

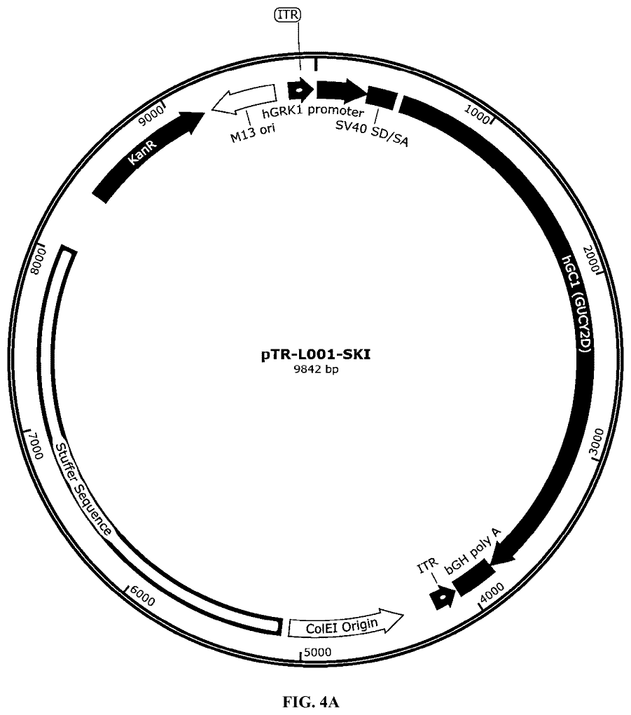

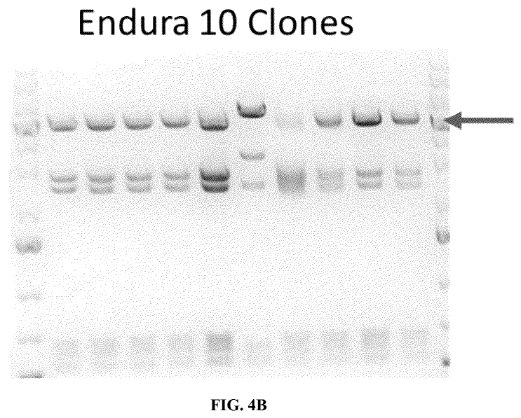

The stuffer sequence was inserted into the backbone at a site located 5′ of the origin, as depicted in FIG. 4A. Plasmids were isolated and digested with SmaI to identify the effect on ITR stability. As depicted in FIG. 4B, greater ITR loss was observed.

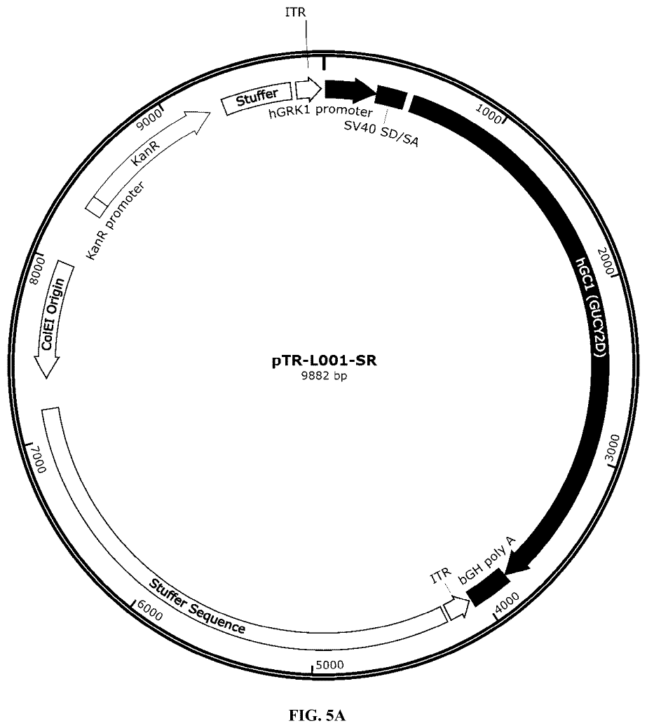

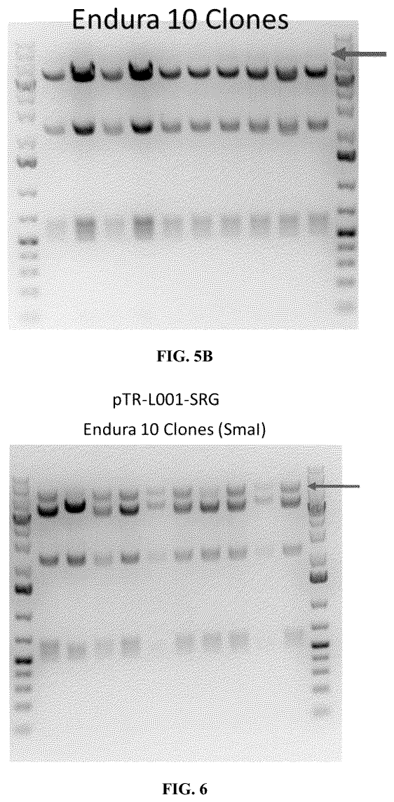

A second plasmid was designed as depicted in FIG. 5A. The stuffer was located 3′ of the origin of replication. The resistance gene was modified to standardized KanR. The M13 origin of replication was removed. The plasmids were isolated and digested with SmaI in order to determine ITR stability. As depicted in FIG. 5B, little to no ITR loss was observed.

The polyG/C sequence was reintroduced to the plasmid shown in FIG. 5B. The plasmids were amplified in kanamycin-resistant cells, isolated, and digested with SmaI. As depicted in FIG. 6, ITR loss was observed. This indicates that both stuffer placement and polyG/C removal are required for enhanced ITR stability.

### Example 3: Stability of Modified Transfer Plasmid Backbone

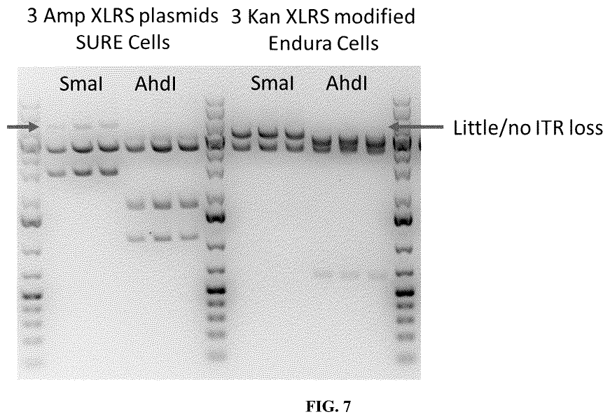

The original transfer plasmid was amplified in SURE cells. The final transfer plasmid (FIG. 5A) was amplified in Endura cells. 10 mg of DNA were produced and isolated. The plasmids were digested with either SmaI or AdhI. The original SmaI plasmid showed greater loss than the modified plasmid, as depicted in FIG. 7.

### Example 4: Use of a Three-Plasmid System for Expression of Human Retinoschisin Transfer Plasmid

This plasmid generates a single-stranded rAAV genome designed to express human retinoschisin (hRS1) in retina. A de novo synthesized RS1 cDNA containing four synonymous substitutions was made to facilitate restriction enzyme molecular cloning. Expression of human RS1 is driven specifically in rod and cone photoreceptors by the hGRK1 promoter which is coupled to the SV40 splice donor/splice acceptor which promotes mRNA transport to the cytoplasm following removal of the SV40 intron. Translation is enhanced by incorporation of a consensus Kozak sequence immediately preceding the hRS1syn start codon and the stability of the transcript is enhanced by inclusion of the WPRE immediately following the hRS1syn open reading frame. The version of WPRE used contains mutations designed to ablate expression of the putative X protein ORF14,15. The expression cassette ends with a bovine growth hormone poly adenylation signal (bGH poly A).

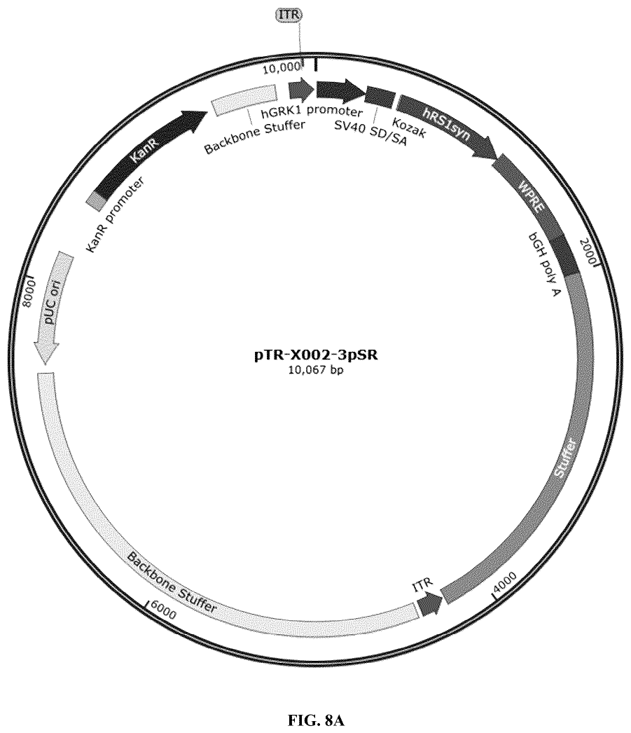

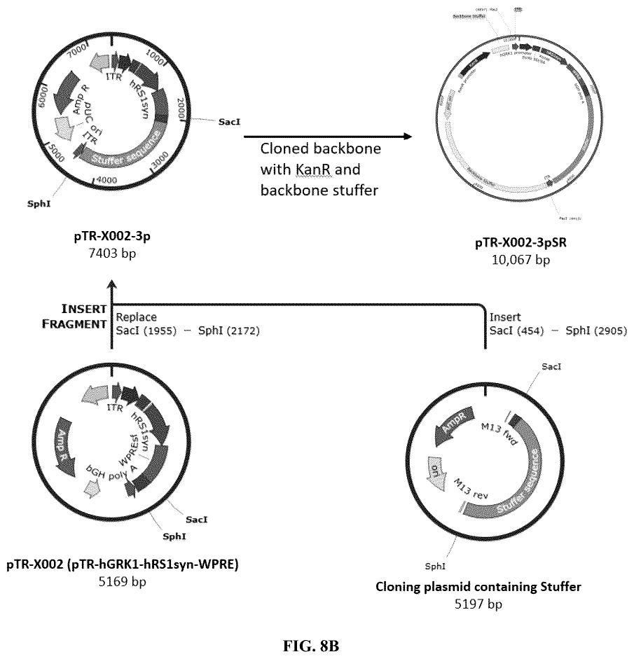

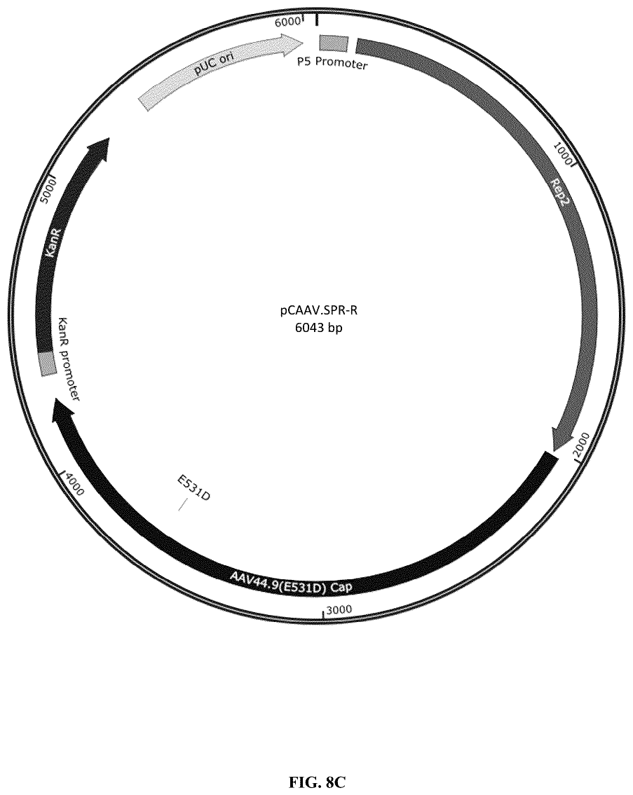

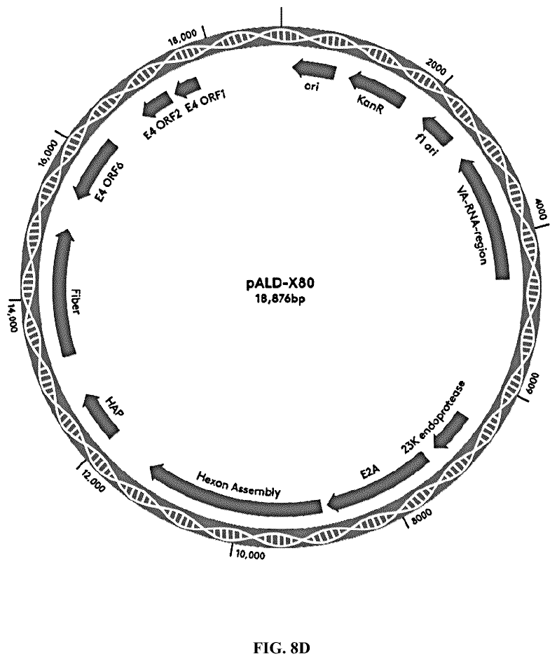

The initial construction of the plasmid began by cloning in the previously described human RS1syn into a previously utilized AAV vector plasmid containing AAV2 ITRs, hGRK1 promoter, SV40 SD/SA and bGH polyA. The WPRE was later cloned into position from pJET-WPRE to create pTR-X002 (pTR-GRK1-RS1syn-WPRE). The small size of the vector (2,311 bp) was not desirable, due to the potential for aberrant packaging; therefore, an inert stuffer sequence was added 3′ to bGH polyA to create a 4,549 bp vector cassette, close to the optimal packaging size of a wild-type AAV genome. The 2,234 bp stuffer sequence was designed in-silico using a random DNA generator (molbiotools.com) and was curated to remove all open reading frames (ORFs) larger than 20 amino acids on both sense/antisense strands, depletion of CpG islands and the removal of repetitive sequences greater than 4 nucleotides. This sequence was de-novo synthesized and cloned using SacI/SphI restriction enzyme cloning to place the stuffer sequence at the 3′ end of the bGH polyA to create pTR-X002-3p (FIG. 8B) This cassette was then packaged into AAV.SPR and determined to have improved efficacy compared to the original unstuffed cassette in an RS1KO mouse model. To create the final version of the plasmid (pTR-X002-3pSR), a Kanamycin resistant backbone (derived from pUC57-KanR) was de novo synthesized to contain additional stuffer sequence (distinct from the internal rAAV cassette stuffer) and cloned in using two PacI restriction sites. This large backbone (5,808 bp ITR-ITR) was designed to prevent reverse packaging by exceeding the rAAV packaging capacity. The entire plasmid was sequence verified by full plasmid next generation sequencing at Massachusetts General Hospital sequencing core (Boston, MA). An annotated map of the pTR-X002-3pSR plasmid is shown in (FIG. 8A) and a diagram demonstrating the cloning strategy shown in FIG. 8B

### Vector Stuffer Sequence

The 2,234 bp stuffer sequence was designed in-silico at Atsena Therapeutics using a random DNA generator (molbiotools.com) and was curated to remove all open reading frames (ORFs) larger than 20 amino acids on both sense/antisense strands, depletion of CpG islands and the removal of repetitive sequences greater than 4 nucleotides. This stuffer sequence and the backbone stuffer sequence are distinct, being derived from different runs of the random DNA generator.

AAV-SPR Rep/Cap Plasmid: pC44.9(E531D)-R

The progenitor AAV2 rep-AAV.SPR plasmid, pCAAV.SPR was constructed. pCAAV.SPR) was constructed from pACG2, where AAV2 VP1 coding sequence was replaced by AAV.SPR coding sequence. Both rep and cap genes are under the control of the endogenous AAV promoter elements. The AAV.SPR cap sequence coding for VP1/VP2/VP3 was created by de novo synthesis (Genscript NJ).

The original pCAAV.SPR backbone contained an ampicillin selectable marker (AmpR) and unneeded legacy sequences associated with the construction of pACG2. The plasmid was modified using restriction enzyme cloning and ligation-independent cloning methods to remove un-needed sequences and replacing the origin of replication and AmpR with a Kanamycin resistance (KanR) gene and origin of replication from pUC57-KanR (Genscript, NJ). This final pCAAV.SPR-R plasmid (FIG. 8C) has been fully validated by next generation sequencing at Massachusetts General Hospital sequencing core and verified to package correctly in a small scale model. An annotated map of the pCAAV.SPR-R plasmid is shown in FIG. 8C.

Helper Plasmid: pALD-X80

Helper plasmid DNA provided E2a, E4, and VA RNA helper genes from Adenovirus Type 5 to the cell to support vector production without the need for wild type viral co-infection. An annotated map of the pALD-X80 plasmid is shown in FIG. 8D.

### Example 5: Evaluation of hRS1-Containing rAAV Vectors with Stuffer Sequences

This study was conducted to evaluate optimized hRS1-containing AAV.SPR vectors with genome sizes conducive to efficient packaging. The goal was to identify a construct that was at least as effective as rAAV.SPR-X001.

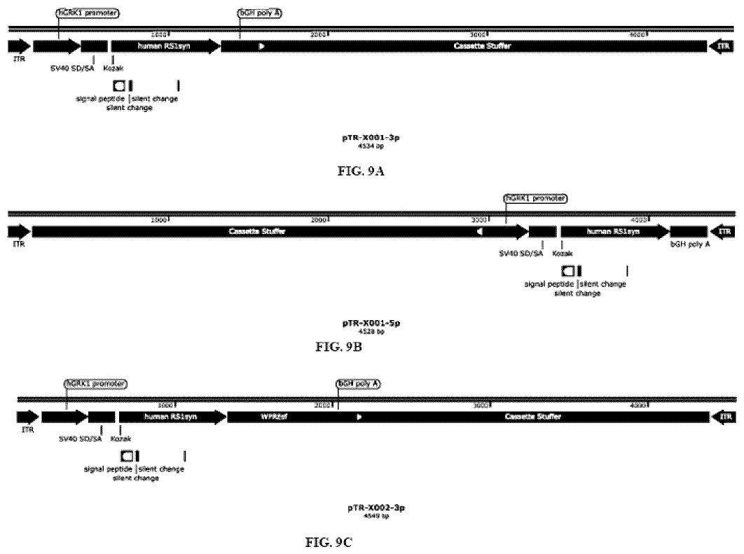

Due to the small packaging size of pTR-X001 (1723 bp), and the possibility for heterologous genome packaging, several new constructs were designed with cassette sizes approaching the natural carrying capacity of AAV (˜4.7 Kb ITR to ITR cassette). This was accomplished by addition of an inert stuffer sequence inserted within the vector cassette either 5′ to (X001-5p), or 3′ to (X001-3p) hGRK1-hRSlsyn-bGH polyA (pTR-X001-5p and pTR-X001-3p, respectively). The stuffer DNA was de novo synthesized (Genscript, NJ). Additionally, a version was created incorporating the woodchuck hepatitis virus post-transcriptional regulatory element (WPRE) positioned between hRSlsyn and bGH polyA (pTR-X002-3p). The mutant version of WPRE used has previously been incorporated in AAV vectors used in other ocular gene therapy clinical trials. All constructs contained the same hGRK1 promoter, SV40 SD/SA and bGH polyA signal sequence. rAAV.SPR vectors were produced by packaging these expression cassettes, and the vector genomes ranged in size from 4534-4549 nucleotides. The multiple constructs evaluated are represented in FIGS. 9A-9C.

A 3-month nonclinical study using two vector doses was performed to test restoration of retinal structure/function in RS1KO mice. The ‘unstuffed’ vector pTR-X001 (rAAV.SPR-X001), which rescued retinal structure/function in the earlier described RS1KO mouse studies, was included as the comparator control.

A summary of the cassette selection study design is presented in Table 1. RS1KO mice were subretinally injected in one eye with either vehicle (Group 1), rAAV.SPR-X001 (Groups 2 and 3), rAAV.SPR-X001-3p (Groups 4 and 5), rAAV.SPR-X001-5p (Groups 6 and 7), or rAAV.SPR-X002-3p (Groups 8 and 9) vectors. Vectors were delivered at either 1.0×10¹¹ vg/mL; 1.0×10⁸ vg/eye (Groups 2,4,6,8) or 5.0×10¹¹ vg/mL; 5.0×10⁸ vg/eye (Groups 3,5,7,9). The contralateral eyes remained un-injected. Retinal structure and function were assessed via OCT and ERG, respectively, at approximately 1-, and 2-months post-injection. Animals were euthanized at approximately 3 months post-injection. Following euthanasia, retinas were cryosectioned and evaluated for RS1 expression via immunohistochemistry.

TABLE 1
Study Design

| | Number of | | Vector Dose | |
|---|---|---|---|---|
| Group | Animals | Test Material | vg/mL | vg/eye |
| 1 | 10 | Vehicle (BSS + | N/A | N/A |
| | | 0.014% Tween-20) | | |
| 2 | 10 | rAAV.SPR -X001 | 1.0 × 10¹¹ | 1.0 × 10⁸ |
| 3 | 10 | rAAV.SPR-X001 | 5.0 × 10¹¹ | 5.0 × 10⁸ |
| 4 | 10 | rAAV.SPR-X001-3p | 1.0 × 10¹¹ | 1.0 × 10⁸ |
| 5 | 10 | rAAV.SPR-X001-3p | 5.0 × 10¹¹ | 5.0 × 10⁸ |
| 6 | 10 | rAAV.SPR-X001-5p | 1.0 × 10¹¹ | 1.0 × 10⁸ |
| 7 | 10 | rAAV.SPR-X001-5p | 5.0 × 10¹¹ | 5.0 × 10⁸ |
| 8 | 10 | rAAV.SPR-X002-3p | 1.0 × 10¹¹ | 1.0 × 10⁸ |
| 9 | 10 | rAAV.SPR-X002-3p | 5.0 × 10¹¹ | 5.0 × 10⁸ |

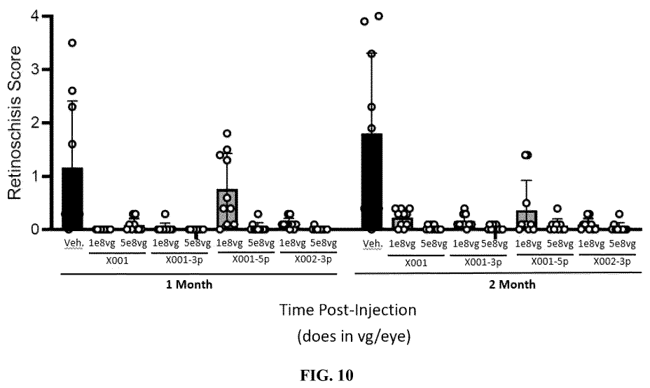

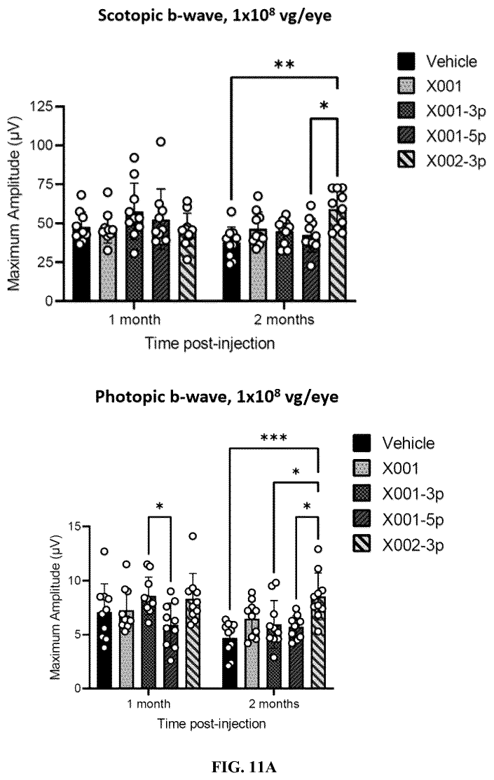

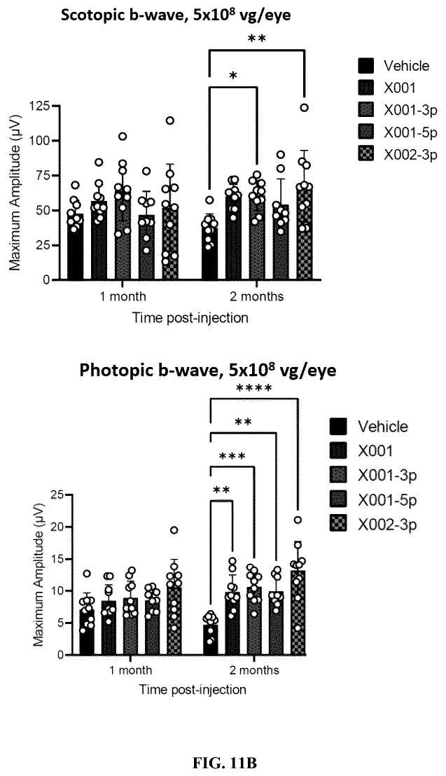

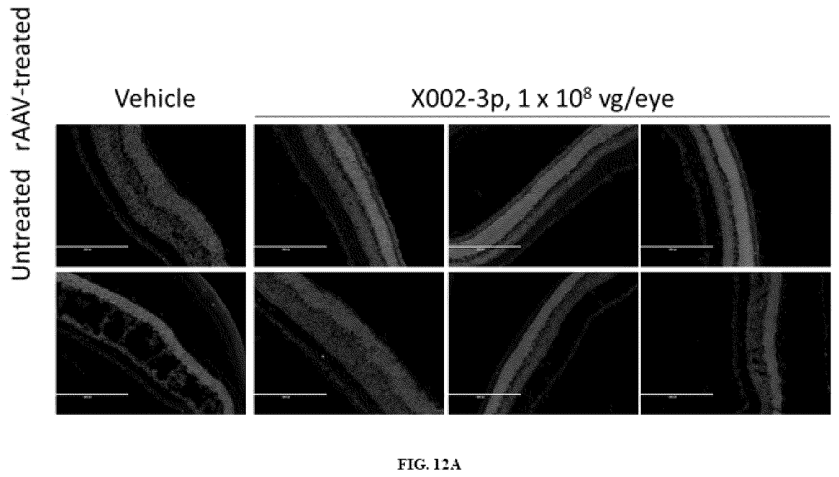

OCT analysis revealed resolution of schisis cavities in vector-treated eyes (FIG. 10). With the exception of eyes treated with the low dose of rAAV.SPR-X001-5p, and evaluated at 1-month post-injection, there was a statically significant difference in the retinoschisis score between vehicle and vector-treated eyes at both timepoints. At a concentration of 1×10¹¹ vg/mL (1.0×10⁸ vg/eye), rAAV.SPR-X002-3p led to significant improvements in rod- and cone-mediated function relative to eyes injected with vehicle alone (FIG. 11A). At the higher concentration of 5×10¹¹ vg/mL (5.0×10⁸ vg/eye), all three stuffer constructs conferred significant improvements in retinal function relative to vehicle-injected controls (FIG. 11B). No significant differences in ERG amplitudes between eyes injected with rAAV.SPR carrying stuffed vs. unstuffed constructs were observed at this higher dose.

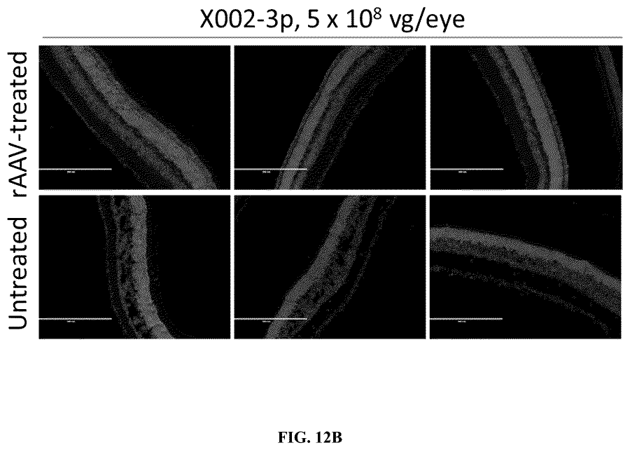

Immunohistochemical analysis revealed the presence of RS1 expression in photoreceptor inner segments of retinas from RS1KO mouse eyes treated with rAAV.SPR-X002-3p. RS1 expression was absent from contralateral untreated eyes and those treated with vehicle alone (FIGS. 12A-12B).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### SEQUENCES

| # | | ANNOTATION |
|---|---|---|
| 1 | ttggccactccctctctgcgcgctcgctcgctcactgaggccgcccgggcaaagcccgg | ITR |
| | gcgtcgggcgacctttggtcgcccggcctcagtgagcgagcgagcgcgcagagagggag | |
| | tggccaactccatcactaggggttcct | |
| 2 | gggccccagaagcctggtggttgtttgtccttctcaggggaaaagtgaggcggcccctt | hGRK1 |
| | ggaggaaggggccgggcagaatgatctaatcggattccaagcagctcaggggattgtct | promoter |
| | ttttctagcaccttcttgccactcctaagcgtcctccgtgaccccggctgggatttagc | |
| | ctggtgctgtgtcagccccggtctcccaggggcttcccagtggtccccaggaaccctcg | |
| | acagggcccggtctctctcgtccagcaagggcagggacgggccacaggccaagggc | |
| 3 | tctagaggatccggtactcgaggaactgaaaaaccagaaagttaactggtaagtttagt | SV40SD/SA |
| | ctttttgtcttttatttcaggtcccggatccggtggtggtgcaaatcaaagaactgctc | |
| | ctcagtggatgttgcctttacttctaggcctgtacggaagtgttac | |
| 4 | atgtcacgcaagatagaaggctttttgttattacttctctttggctatgaagccacatt | RS1 |
| | gggattatcgtctaccgaggatgaaggcgaggacccatggtatcaaaaagcctgcaagt | |
| | gcgattgccaaggaggacccaatgctctgtggtctgcaggtgccacctccttggactgt | |
| | ataccagaatgcccatatcacaagcctctgggtttcgagtcaggggaggtcacaccgga | |
| | ccagatcacctgctctaacccggagcagtatgtgggctggtattcttcgtggactgcaa | |
| | acaaggcccggctcaacagtcaaggctttgggtgtgcctggctctccaagttccaggac | |
| | agtagccagtggttacagatagatctgaaggagatcaaagtgatttcaggcatcctcac | |
| | ccaggggcgctgtgacatcgatgagtggatgaccaagtacagcgtgcagtacaggaccg | |
| | atgagcgcctgaactggatttactacaaggaccagactggaaacaaccgggtcttctat | |
| | ggcaactcggaccgcacctccacggttcagaacctgctgcggccccccatcatctcccg | |
| | cttcatccgcctcatcccgctgggctggcacgtccgcattgccatccggatggagctgc | |
| | tggagtgcgtcagcaagtgtgcctgag | |
| 5 | tcgacctctggattacaaaatttgtgaaagattgactggtattcttaactatgttgctc | WPRE |
| | cttttacgctatgtggatacgctgctttaatgcctttgtatcatgctattgcttcccgt | |
| | atggctttcattttctcctccttgtataaatcctggttgctgtctctttatgaggagtt | |
| | gtggcccgttgtcaggcaacgtggcgtggtgtgcactgtgtttgctgacgcaaccccca | |
| | ctggttggggcattgccaccacctgtcagctcctttccgggactttcgctttccccctc | |
| | cctattgccacggcggaactcatcgccgcctgccttgcccgctgctggacaggggctcg | |
| | gctgttgggcactgacaattccgtggtgttgtcggggaaatcatcgtcctttccttggc | |
| | tgctcgcctgtgttgccacctggattctgcgcgggacgtccttctgctacgtcccttcg | |
| | gccctcaatccagcggaccttccttcccgcggcctgctgccggctctgcggcctcttcc | |
| | gcgtcttcgccttcgccctcagacgagtcggatctccctttgggccgcctccccgcg | |
| 6 | tcgactagagctcgctgatcagcctcgactgtgccttctagttgccagccatctgttgt | pGH polyA |
| | ttgcccctcccccgtgccttccttgaccctggaaggtgccactcccactgtcctttcct | |
| | aataaaatgaggaaattgcatcgcattgtctgagtaggtgtcattctattctggggggt | |
| | ggggggggcaggacagcaagggggaggattgggaagacaatagcaggcatc | |
| 7 | cagtggtccctgcctggagtttgaagggctgtcaaagacattgtggtcacttactcaag | Stuffer |
| | tggtaaattccttacagactggactgcagttaagggtgataacgctactactaggacaa | (within ITRs) |
| | gggtatccttgatcctgtggatctgtagtgacctgagactgtcaaggtatggtgagtat | |
| | gacccttgaccctttccaacggttgtaaccgtgggcgctgtcaaatctgattaaccact | |
| | actaggatagaatttggcagagctactgaaatggttacctggtcaagagactggcccac | |
| | agcataagggcgagcaagacgatgctcagattaactaagtgaccgaagtatcctagatg | |
| | gtccactaccagcaaatgtgtcagacctccctcagttggtagtacaatgtatccttcta | |
| | gtcgcgacttattatatcgacgagcttatgtgtaagagaccagattctttgtaacctga | |
| | cacttgagttcaaggtgtgtagtaggagagtgcaaaccctgaagcaacagtagccgaac | |
| | cgggtcgaggacctagcgttgattgactttgtctggtgtatcctgtaaatttctccaat | |
| | cgagatcgaggtaaagtggacctcacaggactttatcaaacaacagagttaatacctca | |
| | aatctctcgctgatatatgtgacaaattcattgcgtcacttatcgcagctatgttcggt | |
| | cagcatattctggcgaacacccacgctgcagttcgacaaggatagtatctggcgctctc | |
| | gcccaattcaagcacgcgaattactctattaacttgttgtctcttgtgtatatcataat | |
| | caattcctcctagttagtgattgaagacgaccgtgcacttggctgccttgatgcagacc | |
| | acttcttgtggccggcacaccctataatagagatgtgataaataacttagctggactgt | |
| | agttatttcgtcctagggattctgacgggtgcaaatcactaagtgtctttggtattggg | |
| | tctgtaacaatgttgagaactgtggtgtatgtacgaacgatgtgatctgtagactacag | |
| | cacagggtattgctgtttcgcaccagtgtgacacttgtgtggctgtcctctgtgctata | |
| | gcgccgtatatcttatgcaggttgtatagcccaacgactgtttaggtacgtccgtataa | |
| | gaataggagtgctacactctacattgaatctatccagagagagttctgtagaattaggt | |
| | gacgaacaaatctacgcgataggaaggagagacctggttgtcccaagggccgtgggtat | |
| | gatgtttagtacaactaggaggctctaggtgtattggttgttggtcacgtagtgtccct | |
| | acttggtcaggactaaagtttgtcagtgaactctctactattattcatcgaacctgtgg | |
| | tcgtcggcggtctgatatatgctcggtagattgcccgacaggacgtgaaggagcctaac | |
| | tattaaagcactctaactagcctatcgtaacaacttatattgggtatttgggtacgacg | |
| | gtgtactggaaatctatctaattcttctgatcgtatatcgggtcgtatattgatttatg | |
| | acggaccgaccactcacgcggtccgatacgcggtagcgctatccaggaccctcttatga | |
| | attggtatcaatagattgtggaacttgttgcgtacactacagaatgtctttgcaaagaa | |
| | tgccaaattacaagctatagtcctcccactatactgcttcgtgactccaactagggtag | |
| | ttatagttgttcaagactggtactctgttaccaattagtagcgatattgtgtgggatac | |
| | tggtgtctgtcatacgagaactgggagtagcgctgtaaattctaaatagtcacactgtt | |
| | ccagctggacgaccgctgtgtactctttgtagaaactcactgcggcttccacttatgag | |
| | atttggttataggtcacaaagtggcagtccagttgcaggtctcttgttgtaatatgact | |
| | ggattgaattgactaggaagacgtagtctgttcttgttaggtccgacgtggtcccaatt | |
| | tgcttatccaatatgaggtcctagttagcagtcgctccgaggtcgaaatagataccctt | |
| | gtggtttcgtagattgtctgattcgcaacaatatcctgctaatcaatctacggaaacac | |
| | cgcttcagaggaagattgcgacggacagaagagtcaggcgcgccttgcatgc | |
| 8 | catgaccgagtaacgactcagtcgtcccacgaggcataagccagctgtaataagcgtac | Backbone |
| | aagcgcacacacttgagtgggacaccttagggattcccaataattcggcaatctactgt | stuffer (1) |
| | gatacgacacttcagaaacgatgacctgaccgacaagctgaactctacttcacctacta | |
| | ctcaacactacaaccagtcctctgttgacagtataacttgttggtactattatgactga | |
| | agggtaggtttgattggatttgtttgcttcgtctggtcagcaggaggatctctccttta | |
| | tacctgcagataaccgacgggtgcctattgtatcgaagtcattactacatccacctggt | |
| | ccacttgataaatcaatccaaccgatacctacctgactaaggtcccttcacgaggacct | |
| | ttgtcacccagtaatcagatttagaagtcgtattcttacacaacctcacaactgattct | |
| | gaacgcaccgtcgaagtcaacaccacagtatctgaggaagtaagaacagtttgtacttt | |
| | gagcggcgcccactgtgaatacgagagactttaggtcactttcaactatttagtgggtc | |
| | accttcggaactcagacgaggacaccaccacagtagaggtggatagtccaaacacagtt | |
| | gttgtacagacccagtggattgaataggatcttgatcaggtgtgtccagacgctccaga | |
| | ctatagttaaataggagtaagaccgctaagcgcagcaggagttcccaatcgttacaact | |
| | agatcctgtggtgatatatctagtgtccgacttacatgcggcctctgtattagttcata | |
| | ttcattggtctgcacgcctcaggagagatattcgacctgaggataccaggacactttgg | |
| | atcagtatgggtcggaccagttgaacactgataaagtactgaccgagtgggttgtgacg | |
| | gcccagagattcagtcctactttctcaactggagaggaaccacctgttctgagtaaccc | |
| | acaatcgtgtccagagactggacaacactgaatgcaggttcctttctgttcacccagat | |
| | aagggagaccacactaagtggtgtattcgcacgacgatactggtgtcatttatttccct | |
| | ggccagtgtgtacctgcgtgactgcttccgaaggagtgttacaaggagaaaggagagta | |
| | taactacaaggaatgtagacactgcctgcagcccatgaattgcaatataatcggacttg | |
| | ctcggagtccctctactacttaggaagaggattgatcaccacaggtcctctggtcagtt | |
| | taggatgcataatgatccatgccgagacttggaattgatatatactggtatcaccgtat | |
| | aggttgctggttgtagtgtctttagcccacccagtgtaatagcatgtatagttaatctg | |
| | cgtaacgatccctacgaacgacctagaggtcagtcacaccacggcgcgaagaagactct | |
| | tacactaatagtaagctattgtatctagttaagtgaccaacccatttggttcacatcgt | |
| | agttgactttccgggtccttggacccgaaattctatcacatcagtccctaatgcgatga | |
| | gtgcgtctttctggttcacgtggtctgtatcatcaattcctaaatcttagtgactaatt | |
| | attatttcgagacattgtacacctggatggtactgtttctgtcccacgccctacaacgg | |
| | acaaccaacaacgcaccttgttgaacaacaatgatccactacacaagctccgaggtcgt | |
| | tctcgtcttgatttgaaacatgggtgcaatgtgtattagaggtttcaatccatatatcc | |
| | taatacgttgatattgttctttagtaggtggctcctaaagctcaaagttgcaagcaagc | |
| | taacaataccatttcacgacaaggaggataaccacgtgtgttattctgactccatgtaa | |
| | tctgctaatcccaccttcttcagtcgagctttattatattcgcgtaccggagacccagg | |
| | cagaggtgaggacgaatatgttgcaggagtaaagaagctattgaaggatttcttaggac | |
| | gcgctgctggtgttctaaagtagtatagaagctaagtcaagccggagcatactccgata | |
| | tttggagcctgataccagttactgggtgtttagcagaagcgttacgtgacttacacgat | |
| | ataatgactatacttgatctgaaggcatcaagtatcaacgaaagtcctttctgattgac | |
| | aaacagactctcactttacagggataggattggaaccaccaatctgaggtattcaccag | |
| | tatccagccactgctctctcaagtccaacgtaatccaaggagactccggcaattcctta | |
| | attggcgttagctactgtgttgcgagcatttattactatctggcaaggagtgacaaatt | |
| | agcaggattgattgaggtttgtgatccaccagaaggaaggactcaccgttaggtatggt | |
| | gaagatacccagcttgtgctgactgaggatagcaagacagatcacttatcactcgaaca | |
| | gatcggacccaccgcacacctcaatatctctttaggaccaattcaagaacaacttagac | |
| | gctgtacacttaaacagcacagctgtgggtaagagaagtaatagagcgcgatatcagca | |
| | gcagcctcttgtagatcggcttcaagatatctatgtgtggaaataggtcactagagtga | |
| | gcggtactttcgctcttacaggtttgaaaggtcttagcagcgctatttagcgagtcctg | |
| | gagaccacgacaacgagcgctgtggtcctcacaaataggaccaacaataacaatacagg | |
| | atttatcaagagatagaagtcggtataccgattctgctgtttcgtagtcggctacagat | |
| | caatagattattgtgttcagattagttgaggaagttgaatgggtcaaggaccttaccta | |
| | ataccaggtcaccggacttggtaattttccctcttggggttggtgagtagtactgtcta | |
| | ccaactggtgactggtgatcccttacttgtttggaccct | |
| 9 | tgagcaaaaggccagcaaaaggccaggaaccgtaaaaaggccgcgttgctggcgttttt | Origin of |
| | ccataggctccgcccccctgacgagcatcacaaaaatcgacgctcaagtcagaggtggc | replication |
| | gaaacccgacaggactataaagataccaggcgtttccccctggaagctccctcgtgcgc | (pUC) |
| | tctcctgttccgaccctgccgcttaccggatacctgtccgcctttctcccttcgggaag | |
| | cgtggcgctttctcatagctcacgctgtaggtatctcagttcggtgtaggtcgttcgct | |
| | ccaagctgggctgtgtgcacgaaccccccgttcagcccgaccgctgcgccttatccggt | |
| | aactatcgtcttgagtccaacccggtaagacacgacttatcgccactggcagcagccac | |
| | tggtaacaggattagcagagcgaggtatgtaggcggtgctacagagttcttgaagtggt | |
| | ggcctaactacggctacactagaagaacagtatttggtatctgcgctctgctgaagcca | |
| | gttaccttcggaaaaagagttggtagctcttgatccggcaaacaaaccaccgctggtag | |
| | cggtggtttttttgtttgcaagcagcagattacgcgcagaaaaaaaggatctcaagaag | |
| | atcctttgatcttttctacggg | |
| 10 | atgagccatattcaacgggaaacgtcgaggccgcgattaaattccaacatggatgctga | KanR |
| | tttatatgggtataaatgggctcgcgataatgtcgggcaatcaggtgcgacaatctatc | |
| | gcttgtatgggaagcccgatgcgccagagttgtttctgaaacatggcaaaggtagcgtt | |
| | gccaatgatgttacagatgagatggtcagactaaactggctgacggaatttatgcctct | |
| | tccgaccatcaagcattttatccgtactcctgatgatgcatggttactcaccactgcga | |
| | tccccggaaaaacagcattccaggtattagaagaatatcctgattcaggtgaaaatatt | |
| | gttgatgcgctggcagtgttcctgcgccggttgcattcgattcctgtttgtaattgtcc | |
| | ttttaacagcgatcgcgtatttcgtctcgctcaggcgcaatcacgaatgaataacggtt | |
| | tggttgatgcgagtgattttgatgacgagcgtaatggctggcctgttgaacaagtctgg | |
| | aaagaaatgcataaacttttgccattctcaccggattcagtcgtcactcatggtgattt | |
| | ctcacttgataaccttatttttgacgaggggaaattaataggttgtattgatgttggac | |
| | gagtcggaatcgcagaccgataccaggatcttgccatcctatggaactgcctcggtgag | |
| | ttttctccttcattacagaaacggctttttcaaaaatatggtattgataatcctgatat | |
| | gaataaattgcagtttcatttgatgctcgatgagtttttctaa | |
| 11 | ccctgttggttcaagtctccctaatagtttgaggggaaggagttggtgtcaagggtctg | Backbone |
| | accaccatattacgggactagagacaggtggaggtctggacaccctattggtggatcat | stuffer (2) |
| | tcattatggatacggtcctggacaagtggactgatcccaattaggagactggtccagtg | |
| | tcccaccttgagaggtactgatactcacttacctcaaacactaggttcttaagagggtg | |
| | atggagagagattaggaccaaaccagagactttggtcctaactccagaggtagtgagta | |
| | caaaacaccactaggtgaggttgggaccagtctattttctccaaactgagaaggtgtca | |
| | gtggaggcacacttaccctctcactgg | |
| 12 | atgtcagtttcctcctgttcctgtccatccgcacccactatcttcatgttgttgcagat | Fiber |
| | gaagcgcgcaagaccgtctgaagataccttcaaccccgtgtatccatatgacacggaaa | |
| | ccggtcctccaactgtgccttttcttactcctccctttgtatcccccaatgggtttcaa | |
| | gagagtccccctggggtactctctttgcgcctatccgaacctctagttacctccaatgg | |
| | catgcttgcgctcaaaatgggcaacggcctctctctggacgaggccggcaaccttacct | |
| | cccaaaatgtaaccactgtgagcccacctctcaaaaaaaccaagtcaaacataaacctg | |
| | gaaatatctgcacccctcacagttacctcagaagccctaactgtggctgccgccgcacc | |
| | tctaatggtcgcgggcaacacactcaccatgcaatcacaggccccgctaaccgtgcacg | |
| | actccaaacttagcattgccacccaaggacccctcacagtgtcagaaggaaagctagcc | |
| | ctgcaaacatcaggccccctcaccaccaccgatagcagtacccttactatcactgcctc | |
| | accccctctaactactgccactggtagcttgggcattgacttgaaagagcccatttata | |
| | cacaaaatggaaaactaggactaaagtacggggctcctttgcatgtaacagacgaccta | |
| | aacactttgaccgtagcaactggtccaggtgtgactattaataatacttccttgcaaac | |
| | taaagttactggagccttgggttttgattcacaaggcaatatgcaacttaatgtagcag | |
| | gaggactaaggattgattctcaaaacagacgccttatacttgatgttagttatccgttt | |
| | gatgctcaaaaccaactaaatctaagactaggacagggccctctttttataaactcagc | |
| | ccacaacttggatattaactacaacaaaggcctttacttgtttacagcttcaaacaatt | |
| | ccaaaaagcttgaggttaacctaagcactgccaaggggttgatgtttgacgctacagcc | |
| | atagccattaatgcaggagatgggcttgaatttggttcacctaatgcaccaaacacaaa | |
| | tcccctcaaaacaaaaattggccatggcctagaatttgattcaaacaaggctatggttc | |
| | ctaaactaggaactggccttagttttgacagcacaggtgccattacagtaggaaacaaa | |
| | aataatgataagctaactttgtggaccacaccagctccatctcctaactgtagactaaa | |
| | tgcagagaaagatgctaaactcactttggtcttaacaaaatgtggcagtcaaatacttg | |
| | ctacagtttcagttttggctgttaaaggcagtttggctccaatatctggaacagttcaa | |
| | agtgctcatcttattataagatttgacgaaaatggagtgctactaaacaattccttcct | |
| | ggacccagaatattggaactttagaaatggagatcttactgaaggcacagcctatacaa | |
| | acgctgttggatttatgcctaacctatcagcttatccaaaatctcacggtaaaactgcc | |
| | aaaagtaacattgtcagtcaagtttacttaaacggagacaaaactaaacctgtaacact | |
| | aaccattacactaaacggtacacaggaaacaggagacacaactccaagtgcatactcta | |
| | tgtcattttcatgggactggtctggccacaactacattaatgaaatatttgccacatcc | |
| | tcttacactttttcatacattgcccaagaataa | |
| 13 | agctcagtttcctcctgttcctgtccatccgcacccactatcttcatgttgttgcagta | Mutated |
| | aaagcgcgcaagaccgtctgaagataccttcaaccccgtgtatccatatgacacggaaa | Fiber |
| | ccggtcctccaactgtgccttttcttactcctccctttgtatcccccaatgggtttcaa | |
| | gagagtccccctggggtactctctttgcgcctatccgaacctctagttacctccaatgg | |
| | cattcttgcgctcaaaataggcaacggcctctctctggacgaggccggcaaccttacct | |
| | cccaaaatgtaaccactgtgagcccacctctcaaaaaaaccaagtcaaacataaacctg | |
| | gaaatatctgcacccctcacagttacctcagaagccctaactgtggctgccgccgcacc | |
| | tctaatcgtcgcgggcaacacactcaccatacaatcacaggccccgctaaccgtgcacg | |
| | actccaaacttagcattgccacccaaggacccctcacagtgtcagaaggaaagctagcc | |
| | ctgcaaacatcaggccccctcaccaccaccgatagcagtacccttactatcactgcctc | |
| | accccctctaactactgccactggtagcttgggcattgacttgaaagagcccatttata | |
| | cacaaaatggaaaactaggactaaagtacggggctcctttgcatgtaacagacgaccta | |
| | aacactttgaccgtagcaactggtccaggtgtgactattaataatacttccttgcaaac | |
| | taaagttactggagccttgggttttgattcacaaggcaatctgcaacttaatgtagcag | |
| | gaggactaaggattgattctcaaaacagacgccttatacttgatgttagttatccgttt | |
| | gatgctcaaaaccaactaaatctaagactaggacagggccctctttttataaactcagc | |
| | ccacaacttggatattaactacaacaaaggcctttacttgtttacagcttcaaacaatt | |
| | ccaaaaagcttgaggttaacctaagcactgccaaggggttgatatttgacgctacagcc | |
| | atagccattaatgcaggagatgggcttgaatttggttcacctaatgcaccaaacacaaa | |
| | tcccctcaaaacaaaaattggccatggcctagaatttgattcaaacaaggctattgttc | |
| | ctaaactaggaactggccttagttttgacagcacaggtgccattacagtaggaaacaaa | |
| | aataatgataagctaactttgtggaccacaccagctccatctcctaactgtagactaaa | |
| | tgcagagaaagatgctaaactcactttggtcttaacaaaatgtggcagtcaaatacttg | |
| | ctacagtttcagttttggctgttaaaggcagtttggctccaatatctggaacagttcaa | |
| | agtgctcatcttattataagatttgacgaaaatggagtgctactaaacaattccttcct | |
| | ggacccagaatattggaactttagaaatggagatcttactgaaggcacagcctatacaa | |
| | acgctgttggattttaacctaacctatcagcttatccaaaatctcacggtaaaactgcc | |
| | aaaagtaacattgtcagtcaagtttacttaaacggagacaaaactaaacctgtaacact | |
| | aaccattacactaaacggtacacaggaaacaggagacacaactccaagtgcatactcta | |
| | tgtcattttcatgggactggtctggccgctagcacaactacattaatgaaatatttgcc | |
| | acatcctcttacactttttcatacattgcccaagaataa | |
| 21 | AADGYLPDWLEDNLSEGIREWWDLKPGAPKPKANQQKQDDGRGLVLPGYKYLGPFNGLD | AAV1 capsid |
| | KGEPVNAADAAALEHDKAYDQQLKAGDNPYLRYNHADAEFERLQEDTSFGGNLGRAVFQ | |
| | AKKRVLEPLGLVEEGAKTAPGKKRPVEQSPQEPDSSSGIGKTGQQPAKKRLNFGQTGDS | |
| | ESVPDPQPLGEPPATPAAVGTTMASGGGAPMADNNEGADGVGNASGNWHCDSTWLGDRV | |
| | ITTSTRTWALPTYNNHLYKQISSASTGASNDNHYFGYSTPWGYFDFNRFHCHFSPRDWQ | |
| | LINNNWGFRPKRLNFKLFNIQVKEVTTNDGVTTIANNLTSTVQVFSDSEYQLPYVLGSA | |
| | HQGCLPPFPADVFMIPQYGYLTLNNGSQAVGRSSFYCEYFPSQMLRTGNNFTFSYTFEE | |
| | VPFHSSYAHSQSLDRLMNPLIDQYLYYLNRTQNQSGSAQNKDLLFSRGSPAGMSVQPKN | |
| | WLPGPCYRQQRVSKTKTNNNSNFTWTGASKYNLNGRESIINPGTAMASHKDDEDKFFPM | |
| | SGVMIFGKESAGASNTALDNVMITDEEEIKATNPVATERFGTVAVNFQSSTDPATGDVH | |
| | AMGALPGMVWQDRDVYLQGPIWAKIPHTDGHFHPSPLMGGFGLKNPPPQILIKNTPVPA | |
| | NPPAEFSATKFASFITQYSTGQVSVEIEWEQKENSKRWNPEVQYTSNYAKSANVDFTVD | |
| | NNGLYTEPRPIGTRYLTRPL | |
| 22 | AADGYLPDWLEDTLSEGIRQWWKLKPGPPPPKPAERHKDDSRGLVLPGYKYLGPFNGLD | AAV2 capsid |
| | KGEPVNEADAAALEHDKAYDRQLDSGDNPYLKYNHADAEFERLKEDTSFGGNLGRAVFQ | |
| | AKKRVLEPLGLVEEPVKTAPGKKRPVEHSPVEPDSSSGTGKAGQQPARKRLNFGQTGDA | |
| | DSVPDPQPLGQPPAAPSGLGNTMATGSGAPMADNNEGADGVGNSSGNWHCDSTWMGDRV | |
| | ITTSTRTWALPTYNNHLYKQISSQSGASNDNHYFGYSTPWGYFDFNRFHCHFSPRDWQL | |
| | INNNWGFRPKRLNFKLFNIQVKEVTQNDGTTTIANNLTSTVQVFTDSEYQLPYVLGSAH | |
| | QGCLPPFPADVFMVPQYGYLTLNNGSQAVGRSSFYCEYFPSQMLRTGNNFTFSYTFEDV | |
| | PFHSSYAHSQSLDRLMNPLIDQYLYYLSRTNTPSGTTTQSRLQFSQAGASDIRDQSRNW | |
| | LPGPCYRQQRVSKTSANNNSEYSWTGATKYHLNGRDSLVNPGPAMASHKDDEEKFFPQS | |
| | GVLIFGKQGSEKTNVDIEKVMITDEEEIRTTNPVATEQYGSVSTNLQRNRQAATADVNT | |
| | QGVLPGMVWQDRDVYLQGPIWAKIPHTDGHFHPSPLMGGFGLKHPPPQILIKNTPVPAN | |
| | PSTTFSAAKFASFITQYSTGQVSVEIEWEQKENSKRWNPEIQYTSNYNKSVNVDFTVDT | |
| | NGVYSEPRPIGTRYLTRNL | |
| 23 | AADGYLPDWLEDNLSEGIREWWALKPGVPQPKANQQHQDNRRGLVLPGYKYLGPGNGLD | AAV3 capsid |
| | KGEPVNEADAAALEHDKAYDQQLKAGDNPYLKYNHADAEFERLQEDTSFGGNLGRAVFQ | |
| | AKKRILEPLGLVEEAAKTAPGKKGAVDQSPQEPDSSSGVGKSGKQPARKRLNFGQTGDS | |
| | ESVPDPQPLGEPPAAPTSLGNTMASGGGAPMADNNEGADGVGNSSGNWHCDSQWLGDRV | |
| | ITTSTRTWALPTYNNHLYKQISSQSGASNDNHYFGYSTPWGYFDFNRFHCHFSPRDWQL | |
| | INNNWGFRPKKLSFKLFNIQVRGVTQNDGTTTIANNLTSTVQVFTDSEYQLPYVLGSAH | |
| | QGCLPPFPADVFMVPQYGYLTLNNGSQAVGRSSFYCEYFPSQMLRTGNNFQFSYTFEDV | |
| | PFHSSYAHSQSLDRLMNPLIDQYLYYLNRTQGTTSGTTNQSRLLFSQAGPQSMSLQARN | |
| | WLPGPCYRQQRLSKTANNNNSNFPWTAASKYHLNGRDSLVNPGPAMASHKDDEEKFFPM | |
| | HGNLIFGKEGTTASNAELDNVMITDEEEIRTTNPVATEQYGTVANNLQSNTAPTTGTVN | |
| | HQGALPGMVWQDRDVYLQGPIWAKIPHTDGHFHPSPLMGGFGLKHPPPQIMIKNTPVPA | |
| | NPPTTFSPAKFASFITQYSTGQVSVEIEWEQKENSKRWNPEIQYTSNYNKSVNVDFTVD | |
| | TNGVYSEPRPIGTRYLTRNL | |
| 24 | MTDGYLPDWLEDNLSEGVREWWALQPGAPKPKANQQHQDNARGLVLPGYKYLGPGNGLD | AAV4 capsid |
| | KGEPVNAADAAALEHDKAYDQQLKAGDNPYLKYNHADAEFQRLQGDTSFGGNLGRAVFQ | |
| | AKKRVLEPLGLVEQAGETAPGKKRPLIESPQQPDSSTGIGKKGKQPAKKKLVFEDETGA | |
| | GDGPPEGSTSGAMSDDSMRAAAGGAAVEGGQGADGVGNASGDWHCDSTWSEGHVTTTST | |
| | RTWVLPTYNNHLYKRLGESLQSNTYNGFSTPWGYFDFNRFHCHFSPRDWQLINNNWGMR | |
| | PKAMRVKIFNIQVKEVTTSNGETTVANNLTSTVQIFADSSYELPYVMDAGQEGSLPPFP | |
| | NDVFMVPQYGYCGLVTGNTSQQQTDRNAFYCEYFPSQMLRTGNNFEITYSFEKVPFHSM | |
| | YAHSQSLDRLMNPLIDQYLWGLQSTTTGTTLNAGTATTNFTKLRPTNFSNFKKNWLPGP | |
| | SIKQQGFSKTANNYKIPATGSDSLIKYETHSTLDGRWSALTPGPPMATAGPADSKFSNS | |
| | QLIFAGPKQNGNTATVPGTLIFTSEEELAATNATDTDMWGNLPGGDQSSNLPTVDRLTA | |
| | LGAVPGMVWQNRDIYYQGPIWAKIPHTDGHFHPSPLIGGFGLKHPPPQIFIKNTPVPAN | |
| | PATTFSSTPVNSFITQYSTGQVSVQIDWEQKERSKRWNPEVQFTSNYGQQNSLLWAPDA | |
| | AGKYTEPRAIGTRYLTHHL | |
| 25 | SFVDHPPDWLEEVGEGLREFLGLEAGPPKPKPNQQHQDQARGLVLPGYNYLGPGNGLDR | AAV5 capsid |
| | GEPVNRADEVAREHDISYNEQLEAGDNPYLKYNHADAEFEKLADDTSFGGNLGKAVFQA | |
| | KKRVLEPFGLVEEGAKTAPTGKRIDDHFPKRKKARTEEDSKPSTSSDAEAGPSGSQQLQ | |
| | IPAQPASSLGDTMSAGGGGPLGDNNQGADGVGNASGDWHCDSTWMGDRVVTKSTRTWVL | |
| | PSYNNHQYREIKSGSVDGSNANAYFGYSTPWGYFDFNRFHSHWSPRDWQLINNYWGFRP | |
| | RSLRVKIFNIQVKEVTVQDSTTTIANNLTSTVQVFTDDDYQLPYVVGNGTEGCLPAFPP | |
| | QVFTLPQYGYATLNRDNTENPTERSSFFCEYFPSKMLRTGNNFEFTYNFEEVPFHSSFA | |
| | PSQNLFKLANPLVDQYLYRFVSTNNTGGVQFNKNLAGRYANTYKNWFPGPMGRTQGWNL | |
| | GSGNRASVSAFATTNRMELEGASYQVPPQPNGMTNNLQGSNTYALENTMIFNSQPANPG | |
| | TTATYLEGNMLITSESETQPVNRVAYNVGGQMATNNQSTTAPATGTYNLQEIVPGSVWM | |
| | ERDVYLQGPIWAKIPETGAHFHPSPAMGGFGLKHPPPMMLIKNTPVPGNITSFSDVPVS | |
| | SFITQYSTGQVTVEMEWEKKENSKRWNPEIQYTNNYNDPQFVDFAPDSTGEYRTTRPIG | |
| | TRYLTRPL | |
| 26 | AADGYLPDWLEDNLSEGIREWWDLKPGAPKPKANQQKQDDGRGLVLPGYKYLGPFNGLD | AAV6 capsid |
| | KGEPVNAADAAALEHDKAYDQQLKAGDNPYLRYNHADAEFERLQEDTSFGGNLGRAVFQ | |
| | AKKRVLEPFGLVEEGAKTAPGKKRPVEQSPQEPDSSSGIGKTGQQPAKKRLNFGQTGDS | |
| | ESVPDPQPLGEPPATPAAVGTTMASGGGAPMADNNEGADGVGNASGNWHCDSTWLGDRV | |
| | ITTSTRTWALPTYNNHLYKQISSASTGASNDNHYFGYSTPWGYFDFNRFHCHFSPRDWQ | |
| | LINNNWGFRPKRLNFKLFNIQVKEVTTNDGVTTIANNLTSTVQVFSDSEYQLPYVLGSA | |
| | HQGCLPPFPADVFMIPQYGYLTLNNGSQAVGRSSFYCEYFPSQMLRTGNNFTFSYTFED | |
| | VPFHSSYAHSQSLDRLMNPLIDQYLYYLNRTQNQSGSAQNKDLLFSRGSPAGMSVQPKN | |
| | WLPGPCYRQQRVSKTKTNNNSNFTWTGASKYNLNGRESIINPGTAMASHKDDKDKFFPM | |
| | SGVMIFGKESAGASNTALDNVMITDEEEIKATNPVATERFGTVAVNLQSSTDPATGDVH | |
| | VMGALPGMVWQDRDVYLQGPIWAKIPHTDGHFHPSPLMGGFGLKHPPPQILIKNTPVPA | |
| | NPPAEFSATKFASFITQYSTGQVSVEIEWEQKENSKRWNPEVQYTSNYAKSANVDFTVD | |
| | NNGLYTEPRPIGTRYLTRPL | |
| 27 | AADGYLPDWLEDNLSEGIREWWDLKPGAPKPKANQQKQDNGRGLVLPGYKYLGPFNGLD | AAV7 capsid |
| | KGEPVNAADAAALEHDKAYDQQLKAGDNPYLRYNHADAEFERLQEDTSFGGNLGRAVFQ | |
| | AKKRVLEPLGLVEEGAKTAPAKKRPVEPSPQRSPDSSTGIGKKGQQPARKRLNFGQTGD | |
| | SESVPDPQPLGEPPAAPSSVGGTVAAGGGAPMADNNEGADGVGNASGNWHCDSTWLGDR | |
| | VITTSTRTWALPTYNNHLYKQISSETAGSTNDNTYFGYSTPWGYFDFNRFHCHFSPRDW | |
| | QLINNNWGFRPKKLRFKLFNIQVKEVTTNDGVTTIANNLTSTIQVFSDSEYQLPYVLGS | |
| | AHQGCLPPFPADVFMIPQYGYLTLNNGSQSVGRSSFYCEYFPSQMLRTGNNFEFSYSFE | |
| | DVPFHSSYAHSQSLDRLMNPLIDQYLYYLARTQSNPGGTAGNRELQFYQGGPSTMAEQA | |
| | KNWLPGPCFRQQRVSKTLDNNNSNFAWTGATKYHLNGRNSLVNPGVAMATHKDDEDRFF | |
| | PSSGVLIFGKTGATNKTTLENVLMTNEEEIRPTNPVATEEYGIVSSNLQANTAAQTQVV | |
| | NNQGALPGMVWQNRDVYLQGPIWAKIPHTDGNFHPSPLMGGFGLKHPPPQILIKNTPVP | |
| | ANPPEVFTPAKFASFITQYSTGQVSVEIEWEQKENSKRWNPEIQYTSNFEKQTGVDFAV | |
| | DSQGVYSEPRPIGTRYLTRNL | |
| 28 | AADGYLPDWLEDNLSEGIREWWALKPGAPKPKANQQKQDDGRGLVLPGYKYLGPFNGLD | AAV8 capsid |
| | KGEPVNAADAAALEHDKAYDQQLQAGDNPYLRYNHADAEFERLQEDTSFGGNLGRAVFQ | |
| | AKKRVLEPLGLVEEGAKTAPGKKRPVEPSPQRSPDSSTGIGKKGQQPARKRLNFGQTGD | |
| | SESVPDPQPLGEPPAAPSGVGNTMAAGGGAPMADNNEGADGVGSSSGNWHCDSTWLGDR | |
| | VITTSTRTWALPTYNNHLYKQISNGTSGGATNDNTYFGYSTPWGYFDFNRFHCHFSPRD | |
| | WQLINNNWGFRPKRLSFKLFNIQVKEVTQNEGTKTIANNLTSTIQVFTDSEYQLPYVLG | |
| | SAHQGCLPPFPADVFMIPQYGYLTLNNGSQAVGRSSFYCEYFPSQMLRTGNNFQFTYTF | |
| | EDVPFHSSYAHSQSLDRLMNPLIDQYLYYLSRTQTTGGTANTQTLGFSQGGPNTMANQA | |
| | KNWLPGPCYRQQRVSTTTGNNNSNFAWTAGTKYHLNGRNSLANPGIAMATHKDDEERFF | |
| | PSNGILIFGKQNAARDNADYSDVMLTSEEEIKTTNPVATEEYGIVADNLQQNTAPQIGT | |
| | VNSQGALPGMVWQNRDVYLQGPIWAKIPHTDGNFHPSPLMGGFGLKHPPPQILIKNTPV | |
| | PADPPTTFNQSKLNSFITQYSTGQVSVEIEWEQKENSKRWNPEIQYTSNYYKSTSVDFA | |
| | VNTEGVYSEPRPIGTRYLTRNL | |
| 29 | AADGYLPDWLEDNLSEGIREWWALKPGAPQPKANQQHQDNARGLVLPGYKYLGPGNGLD | AAV9 capsid |
| | KGEPVNAADAAALEHDKAYDQQLKAGDNPYLKYNHADAEFERLKEDTSFGGNLGRAVFQ | |
| | AKKRLLEPLGLVEEAAKTAPGKKRPVEQSPQEPDSSAGIGKSGAQPAKKRLNFGQTGDT | |
| | ESVPDPQPIGEPPAAPSGVGLTMASGGGAPVADNNEGADGVGSSSGNWHCDSQWLGDRV | |
| | ITTSTRTWALPTYNNHLYKQISNSTSGGSSNDNAYFGYSTPWGYFDFNRFHCHFSPRDW | |
| | QLINNNWGFRPKRLNFKLFNIQVKEVTDNNGVKTIANNLTSTVQVFTDSDYQLPYVLGS | |
| | AHEGCLPPFPADVFMIPQYGYLTLNDGSQAVGRSSFYCEYFPSQMLRTGNNFQFSYEFE | |
| | NVPFHSSYAHSQSLDRLMNPLIDQYLYYLSKTINGSGQNQQTLKFSVAGPSNMAVQGRN | |
| | YIPGPSYRQQRVSTTVTNNNSEFAWPGASSWALNGRNSLMNPGPAMASHKEGEDRFFPL | |
| | SGSLIFGKQGTGRDNVDADKVMITNEEEIKTTNPVATESYGQVATNHQSQAQAQTGWVQ | |
| | NQGILPGMVWQDRDVYLQGPIWAKIPHTDGNFHPSPLMGGFGMKHPPPQILIKNTPVPA | |
| | DPPTAFNKDKLNSFITQYSTGQVSVEIEWEQKENSKRWNPEIQYTSNYYKSNNVEFAVN | |
| | TEGVYSEPRPIGTRYLTRNL | |
| 30 | MAADGYLPDWLEDNLSEGIREWWDLKPGAPKPKANQQKQDDGRGLVLPGYKYLGPFNGL | AAV10 |
| | DKGEPVNAADAAALEHDKAYDQQLKAGDNPYLRYNHADAEFQERLQEDTSFGGNLGRAV | capsid |
| | FQAKKRVLEPLGLVEEGAKTAPGKKRPVEPSPQRSPDSSTGIGKKGQQPAKKRLNFGQT | |
| | GDSESVPDPQPIGEPPAGPSGLGSGTMAAGGGAPMADNNEGADGVGSSSGNWHCDSTWL | |
| | GDRVITTSTRTWALPTYNNHLYKQISNGTSGGSTNDNTYFGYSTPWGYFDFNRFHCHFS | |
| | PRDWQRLINNNWGFRPKRLNFKLFNIQVKEVTQNEGTKTIANNLTSTIQVFTDSEYQLP | |
| | YVLGSAHQGCLPPFPADVFMIPQYGYLTLNNGSQAVGRSSFYCLEYFPSQMLRTGNNFE | |
| | FSYQFEDVPFHSSYAHSQSLDRLMNPLIDQYLYYLSRTQSTGGTAGTQQLLFSQAGPNN | |
| | MSAQAKNWLPGPCYRQQRVSTTLSQNNNSNFAWTGATKYHLNGRDSLVNPGVAMATHKD | |
| | DEERFFPSSGVLMFGKQGAGKDNVDYSSVMLTSEEEIKTTNPVATEQYGVVADNLQQQN | |
| | AAPIVGAVNSQGALPGMVWQNRDVYLQGPIWAKIPHTDGNFHPSPLMGGFGLKHPPPQI | |
| | LIKNTPVPADPPTTFSQAKLASFITQYSTGQVSVEIEWELQKENSKRWNPEIQYTSNYY | |
| | KSTNVDFAVNTDGTYSEPRPIGTRYLTRNL | |
| 31 | MAADGYLPDWLEDNLSEGIREWWDLKPGAPKPKANQQKQDDGRGLVLPGYKYLGPFNGL | AAV44.9 |
| | DKGEPVNAADAAALEHDKAYDQQLKAGDNPYLRYNHADAEFQERLQEDTSFGGNLGRAV | (E531D) |
| | FQAKKRVLEPLGLVEEGAKTAPGKKRPVEQSPQEPDSSSGIGKTGQQPAKKRLNFGQTG | |
| | DTESVPDPQPLGEPPAAPSGLGPNTMASGGGAPMADNNEGADGVGNSSGNWHCDSTWLG | |
| | DRVITTSTRTWALPTYNNHLYKQISNGTSGGSTNDNTYFGYSTPWGYFDFNRFHCHFSP | |
| | RDWQRLINNNWGFRPKRLNFKLFNIQVKEVTTNEGTKTIANNLTSTVQVFTDSEYQLPY | |
| | VLGSAHQGCLPPFPADVFMVPQYGYLTLNNGSQALGRSSFYCLEYFPSQMLRTGNNFQF | |
| | SYTFEDVPFHSSYAHSQSLDRLMNPLIDQYLYYLVRTQTTGTGGTQTLAFSQAGPSSMA | |
| | SQARNWVPGPSYRQQRVSTTTNQNNNSNFAWTGAAKFKLNGRDSLMNPGVAMASHKDDD | |
| | DRFFPSSGVLIFGKQGAGNDGVDYSQVLITDEEEIKATNPVATEEYGAVAINNQAANTQ | |
| | AQTGLVHNQGVIPGMVWQNRDVYLQGPIWAKIPHTDGNFHPSPLMGGFGLKHPPPQILI | |
| | KNTPVPADPPLTFNQAKLNSFITQYSTGQVSVEIEWELQKENSKRWNPEIQYTSNYYKS | |
| | TNVDFAVNTEGVYSEPRPIGTRYLTRNL | |
| 32 | ggcctcttcgcttaattaacgccaggctgcaggttggccactccctctctgcgcgctcg | Full length |
| | ctcgctcactgaggccgcccgggcaaagcccgggcgtcgggcgacctttggtcgcccgg | sequence |
| | cctcagtgagcgagcgagcgcgcagagagggagtggccaactccatcactaggggttcc | |
| | tcagatctgaattcggtaccgggccccagaagcctggtggttgtttgtccttctcaggg | |
| | gaaaagtgaggcggccccttggaggaaggggccgggcagaatgatctaatcggattcca | |
| | agcagctcaggggattgtctttttctagcaccttcttgccactcctaagcgtcctccgt | |
| | gaccccggctgggatttagcctggtgctgtgtcagccccggtctcccaggggcttccca | |
| | gtggtccccaggaaccctcgacagggcccggtctctctcgtccagcaagggcagggacg | |
| | ggccacaggccaagggctctagaggatccggtactcgaggaactgaaaaaccagaaagt | |
| | taactggtaagtttagtctttttgtcttttatttcaggtcccggatccggtggtggtgc | |
| | aaatcaaagaactgctcctcagtggatgttgcctttacttctaggcctgtacggaagtg | |
| | ttacttctgctctaaaagctgcggaattgtacccgcggccgccaccatgtcacgcaaga | |
| | tagaaggctttttgttattacttctctttggctatgaagccacattgggattatcgtct | |
| | accgaggatgaaggcgaggacccatggtatcaaaaagcctgcaagtgcgattgccaagg | |
| | aggacccaatgctctgtggtctgcaggtgccacctccttggactgtataccagaatgcc | |
| | catatcacaagcctctgggtttcgagtcaggggaggtcacaccggaccagatcacctgc | |
| | tctaacccggagcagtatgtgggctggtattcttcgtggactgcaaacaaggcccggct | |
| | caacagtcaaggctttgggtgtgcctggctctccaagttccaggacagtagccagtggt | |
| | tacagatagatctgaaggagatcaaagtgatttcaggcatcctcacccaggggcgctgt | |
| | gacatcgatgagtggatgaccaagtacagcgtgcagtacaggaccgatgagcgcctgaa | |
| | ctggatttactacaaggaccagactggaaacaaccgggtcttctatggcaactcggacc | |
| | gcacctccacggttcagaacctgctgcggccccccatcatctcccgcttcatccgcctc | |
| | atcccgctgggctggcacgtccgcattgccatccggatggagctgctggagtgcgtcag | |
| | caagtgtgcctgagtcgacctctggattacaaaatttgtgaaagattgactggtattct | |
| | taactatgttgctccttttacgctatgtggatacgctgctttaatgcctttgtatcatg | |
| | ctattgcttcccgtatggctttcattttctcctccttgtataaatcctggttgctgtct | |
| | ctttatgaggagttgtggcccgttgtcaggcaacgtggcgtggtgtgcactgtgtttgc | |
| | tgacgcaacccccactggttggggcattgccaccacctgtcagctcctttccgggactt | |
| | tcgctttccccctccctattgccacggcggaactcatcgccgcctgccttgcccgctgc | |
| | tggacaggggctcggctgttgggcactgacaattccgtggtgttgtcggggaaatcatc | |
| | gtcctttccttggctgctcgcctgtgttgccacctggattctgcgcgggacgtccttct | |
| | gctacgtcccttcggccctcaatccagcggaccttccttcccgcggcctgctgccggct | |
| | ctgcggcctcttccgcgtcttcgccttcgccctcagacgagtcggatctccctttgggc | |
| | cgcctccccgcgtcgactagagctcgctgatcagcctcgactgtgccttctagttgcca | |
| | gccatctgttgtttgcccctcccccgtgccttccttgaccctggaaggtgccactccca | |
| | ctgtcctttcctaataaaatgaggaaattgcatcgcattgtctgagtaggtgtcattct | |
| | attctggggggtggggggggcaggacagcaagggggaggattgggaagacaatagcagg | |
| | catccagtggtccctgcctggagtttgaagggctgtcaaagacattgtggtcacttact | |
| | caagtggtaaattccttacagactggactgcagttaagggtgataacgctactactagg | |
| | acaagggtatccttgatcctgtggatctgtagtgacctgagactgtcaaggtatggtga | |
| | gtatgacccttgaccctttccaacggttgtaaccgtgggcgctgtcaaatctgattaac | |
| | cactactaggatagaatttggcagagctactgaaatggttacctggtcaagagactggc | |
| | ccacagcataagggcgagcaagacgatgctcagattaactaagtgaccgaagtatccta | |
| | gatggtccactaccagcaaatgtgtcagacctccctcagttggtagtacaatgtatcct | |
| | tctagtcgcgacttattatatcgacgagcttatgtgtaagagaccagattctttgtaac | |
| | ctgacacttgagttcaaggtgtgtagtaggagagtgcaaaccctgaagcaacagtagcc | |
| | gaaccgggtcgaggacctagcgttgattgactttgtctggtgtatcctgtaaatttctc | |
| | caatcgagatcgaggtaaagtggacctcacaggactttatcaaacaacagagttaatac | |
| | ctcaaatctctcgctgatatatgtgacaaattcattgcgtcacttatcgcagctatgtt | |
| | cggtcagcatattctggcgaacacccacgctgcagttcgacaaggatagtatctggcgc | |
| | tctcgcccaattcaagcacgcgaattactctattaacttgttgtctcttgtgtatatca | |
| | taatcaattcctcctagttagtgattgaagacgaccgtgcacttggctgccttgatgca | |
| | gaccacttcttgtggccggcacaccctataatagagatgtgataaataacttagctgga | |
| | ctgtagttatttcgtcctagggattctgacgggtgcaaatcactaagtgtctttggtat | |
| | tgggtctgtaacaatgttgagaactgtggtgtatgtacgaacgatgtgatctgtagact | |
| | acagcacagggtattgctgtttcgcaccagtgtgacacttgtgtggctgtcctctgtgc | |
| | tatagcgccgtatatcttatgcaggttgtatagcccaacgactgtttaggtacgtccgt | |
| | ataagaataggagtgctacactctacattgaatctatccagagagagttctgtagaatt | |
| | aggtgacgaacaaatctacgcgataggaaggagagacctggttgtcccaagggccgtgg | |
| | gtatgatgtttagtacaactaggaggctctaggtgtattggttgttggtcacgtagtgt | |
| | ccctacttggtcaggactaaagtttgtcagtgaactctctactattattcatcgaacct | |
| | gtggtcgtcggcggtctgatatatgctcggtagattgcccgacaggacgtgaaggagcc | |
| | taactattaaagcactctaactagcctatcgtaacaacttatattgggtatttgggtac | |
| | gacggtgtactggaaatctatctaattcttctgatcgtatatcgggtcgtatattgatt | |
| | tatgacggaccgaccactcacgcggtccgatacgcggtagcgctatccaggaccctctt | |
| | atgaattggtatcaatagattgtggaacttgttgcgtacactacagaatgtctttgcaa | |
| | agaatgccaaattacaagctatagtcctcccactatactgcttcgtgactccaactagg | |
| | gtagttatagttgttcaagactggtactctgttaccaattagtagcgatattgtgtggg | |
| | atactggtgtctgtcatacgagaactgggagtagcgctgtaaattctaaatagtcacac | |
| | tgttccagctggacgaccgctgtgtactctttgtagaaactcactgcggcttccactta | |
| | tgagatttggttataggtcacaaagtggcagtccagttgcaggtctcttgttgtaatat | |
| | gactggattgaattgactaggaagacgtagtctgttcttgttaggtccgacgtggtccc | |
| | aatttgcttatccaatatgaggtcctagttagcagtcgctccgaggtcgaaatagatac | |
| | ccttgtggtttcgtagattgtctgattcgcaacaatatcctgctaatcaatctacggaa | |
| | acaccgcttcagaggaagattgcgacggacagaagagtcaggcgcgccttgcatgctgg | |
| | ggagagatctgaggaacccctagtgatggagttggccactccctctctgcgcgctcgct | |
| | cgctcactgaggccgggcgaccaaaggtcgcccgacgcccgggctttgcccgggcggcc | |
| | tcagtgagcgagcgagcgcgcagagagggagtggccaacctgcagcctgcttaattaat | |
| | catatgcatgaccgagtaacgactcagtcgtcccacgaggcataagccagctgtaataa | |
| | gcgtacaagcgcacacacttgagtgggacaccttagggattcccaataattcggcaatc | |
| | tactgtgatacgacacttcagaaacgatgacctgaccgacaagctgaactctacttcac | |
| | ctactactcaacactacaaccagtcctctgttgacagtataacttgttggtactattat | |
| | gactgaagggtaggtttgattggatttgtttgcttcgtctggtcagcaggaggatctct | |
| | cctttatacctgcagataaccgacgggtgcctattgtatcgaagtcattactacatcca | |
| | cctggtccacttgataaatcaatccaaccgatacctacctgactaaggtcccttcacga | |
| | ggacctttgtcacccagtaatcagatttagaagtcgtattcttacacaacctcacaact | |
| | gattctgaacgcaccgtcgaagtcaacaccacagtatctgaggaagtaagaacagtttg | |
| | tactttgagcggcgcccactgtgaatacgagagactttaggtcactttcaactatttag | |
| | tgggtcaccttcggaactcagacgaggacaccaccacagtagaggtggatagtccaaac | |
| | acagttgttgtacagacccagtggattgaataggatcttgatcaggtgtgtccagacgc | |
| | tccagactatagttaaataggagtaagaccgctaagcgcagcaggagttcccaatcgtt | |
| | acaactagatcctgtggtgatatatctagtgtccgacttacatgcggcctctgtattag | |
| | ttcatattcattggtctgcacgcctcaggagagatattcgacctgaggataccaggaca | |
| | ctttggatcagtatgggtcggaccagttgaacactgataaagtactgaccgagtgggtt | |
| | gtgacggcccagagattcagtcctactttctcaactggagaggaaccacctgttctgag | |
| | taacccacaatcgtgtccagagactggacaacactgaatgcaggttcctttctgttcac | |
| | ccagataagggagaccacactaagtggtgtattcgcacgacgatactggtgtcatttat | |
| | ttccctggccagtgtgtacctgcgtgactgcttccgaaggagtgttacaaggagaaagg | |
| | agagtataactacaaggaatgtagacactgcctgcagcccatgaattgcaatataatcg | |
| | gacttgctcggagtccctctactacttaggaagaggattgatcaccacaggtcctctgg | |
| | tcagtttaggatgcataatgatccatgccgagacttggaattgatatatactggtatca | |
| | ccgtataggttgctggttgtagtgtctttagcccacccagtgtaatagcatgtatagtt | |
| | aatctgcgtaacgatccctacgaacgacctagaggtcagtcacaccacggcgcgaagaa | |
| | gactcttacactaatagtaagctattgtatctagttaagtgaccaacccatttggttca | |
| | catcgtagttgactttccgggtccttggacccgaaattctatcacatcagtccctaatg | |
| | cgatgagtgcgtctttctggttcacgtggtctgtatcatcaattcctaaatcttagtga | |
| | ctaattattatttcgagacattgtacacctggatggtactgtttctgtcccacgcccta | |
| | caacggacaaccaacaacgcaccttgttgaacaacaatgatccactacacaagctccga | |
| | ggtcgttctcgtcttgatttgaaacatgggtgcaatgtgtattagaggtttcaatccat | |
| | atatcctaatacgttgatattgttctttagtaggtggctcctaaagctcaaagttgcaa | |
| | gcaagctaacaataccatttcacgacaaggaggataaccacgtgtgttattctgactcc | |
| | atgtaatctgctaatcccaccttcttcagtcgagctttattatattcgcgtaccggaga | |
| | cccaggcagaggtgaggacgaatatgttgcaggagtaaagaagctattgaaggatttct | |
| | taggacgcgctgctggtgttctaaagtagtatagaagctaagtcaagccggagcatact | |
| | ccgatatttggagcctgataccagttactgggtgtttagcagaagcgttacgtgactta | |
| | cacgatataatgactatacttgatctgaaggcatcaagtatcaacgaaagtcctttctg | |
| | attgacaaacagactctcactttacagggataggattggaaccaccaatctgaggtatt | |
| | caccagtatccagccactgctctctcaagtccaacgtaatccaaggagactccggcaat | |
| | tccttaattggcgttagctactgtgttgcgagcatttattactatctggcaaggagtga | |
| | caaattagcaggattgattgaggtttgtgatccaccagaaggaaggactcaccgttagg | |
| | tatggtgaagatacccagcttgtgctgactgaggatagcaagacagatcacttatcact | |
| | cgaacagatcggacccaccgcacacctcaatatctctttaggaccaattcaagaacaac | |
| | ttagacgctgtacacttaaacagcacagctgtgggtaagagaagtaatagagcgcgata | |
| | tcagcagcagcctcttgtagatcggcttcaagatatctatgtgtggaaataggtcacta | |
| | gagtgagcggtactttcgctcttacaggtttgaaaggtcttagcagcgctatttagcga | |
| | gtcctggagaccacgacaacgagcgctgtggtcctcacaaataggaccaacaataacaa | |
| | tacaggatttatcaagagatagaagtcggtataccgattctgctgtttcgtagtcggct | |
| | acagatcaatagattattgtgttcagattagttgaggaagttgaatgggtcaaggacct | |
| | tacctaataccaggtcaccggacttggtaattttccctcttggggttggtgagtagtac | |
| | tgtctaccaactggtgactggtgatcccttacttgtttggaccctggtaatacggttat | |
| | ccacagaatcaggggataacgcaggaaagaacatgtgagcaaaaggccagcaaaaggcc | |
| | aggaaccgtaaaaaggccgcgttgctggcgtttttccataggctccgcccccctgacga | |
| | gcatcacaaaaatcgacgctcaagtcagaggtggcgaaacccgacaggactataaagat | |
| | accaggcgtttccccctggaagctccctcgtgcgctctcctgttccgaccctgccgctt | |
| | accggatacctgtccgcctttctcccttcgggaagcgtggcgctttctcatagctcacg | |
| | ctgtaggtatctcagttcggtgtaggtcgttcgctccaagctgggctgtgtgcacgaac | |
| | cccccgttcagcccgaccgctgcgccttatccggtaactatcgtcttgagtccaacccg | |
| | gtaagacacgacttatcgccactggcagcagccactggtaacaggattagcagagcgag | |
| | gtatgtaggcggtgctacagagttcttgaagtggtggcctaactacggctacactagaa | |
| | gaacagtatttggtatctgcgctctgctgaagccagttaccttcggaaaaagagttggt | |
| | agctcttgatccggcaaacaaaccaccgctggtagcggtggtttttttgtttgcaagca | |
| | gcagattacgcgcagaaaaaaaggatctcaagaagatcctttgatcttttctacggggt | |
| | ctgacgctcagtggaacgaaaactcacgttaagggattttggtcatgagattatcaaaa | |
| | aggatcttcacctagatccttttcacgtagaaacccagtccgcagaaacggtgctgacc | |
| | cctgatgaatgtcacctactgggttatctggacaagggaaaacccaagcgcaaagagaa | |
| | agcaggtagcttggagtggggttacatggtgataggtagactgggaggttttatggaca | |
| | ggaaccgaacctcaattcccaggtggggtgtcctctggtaaggttgggaagtcctggaa | |
| | agtaaactggatggctttctcgccgctgttacattgcacaagataaaaatatatcatca | |
| | tgaacaataaaactgtctgcttacataaacagtaatacaaggggtgttatgagccatat | |
| | tcaacgggaaacgtcgaggccgcgattaaattccaacatggatgctgatttatatgggt | |
| | ataaatgggctcgcgataatgtcgggcaatcaggtgcgacaatctatcgcttgtatggg | |
| | aagcccgatgcgccagagttgtttctgaaacatggcaaaggtagcgttgccaatgatgt | |
| | tacagatgagatggtcagactaaactggctgacggaatttatgcctcttccgaccatca | |
| | agcattttatccgtactcctgatgatgcatggttactcaccactgcgatccccggaaaa | |
| | acagcattccaggtattagaagaatatcctgattcaggtgaaaatattgttgatgcgct | |
| | ggcagtgttcctgcgccggttgcattcgattcctgtttgtaattgtccttttaacagcg | |
| | atcgcgtatttcgtctcgctcaggcgcaatcacgaatgaataacggtttggttgatgcg | |
| | agtgattttgatgacgagcgtaatggctggcctgttgaacaagtctggaaagaaatgca | |
| | taaacttttgccattctcaccggattcagtcgtcactcatggtgatttctcacttgata | |
| | accttatttttgacgaggggaaattaataggttgtattgatgttggacgagtcggaatc | |
| | gcagaccgataccaggatcttgccatcctatggaactgcctcggtgagttttctccttc | |
| | attacagaaacggctttttcaaaaatatggtattgataatcctgatatgaataaattgc | |
| | agtttcatttgatgctcgatgagtttttctaaattttgttaaaatttttgttaaatcag | |
| | ctcattttttaaccaataggccctgttggttcaagtctccctaatagtttgaggggaag | |
| | gagttggtgtcaagggtctgaccaccatattacgggactagagacaggtggaggtctgg | |
| | acaccctattggtggatcattcattatggatacggtcctggacaagtggactgatccca | |
| | attaggagactggtccagtgtcccaccttgagaggtactgatactcacttacctcaaac | |
| | actaggttcttaagagggtgatggagagagattaggaccaaaccagagactttggtcct | |
| | aactccagaggtagtgagtacaaaacaccactaggtgaggttgggaccagtctattttc | |
| | tccaaactgagaaggtgtcagtggaggcacacttaccctctcactggctattcgccatt | |
| | caggctgcgcaactgttgggaagggcgatcggtgcg | |
| 33 | cctcttcgcttaattaacgccaggctgcaggttggccactccctctctgcgcgctcgct | Full length |
| | cgctcactgaggccgcccgggcaaagcccgggcgtcgggcgacctttggtcgcccggcc | |
| | tcagtgagcgagcgagcgcgcagagagggagtggccaactccatcactaggggttcctc | |
| | agatctgaattcggtaccgggccccagaagcctggtggttgtttgtccttctcagggga | |
| | aaagtgaggcggccccttggaggaaggggccgggcagaatgatctaatcggattccaag | |
| | cagctcaggggattgtctttttctagcaccttcttgccactcctaagcgtcctccgtga | |
| | ccccggctgggatttagcctggtgctgtgtcagccccggtctcccaggggcttcccagt | |
| | ggtccccaggaaccctcgacagggcccggtctctctcgtccagcaagggcagggacggg | |
| | ccacaggccaagggctctagaggatccggtactcgaggaactgaaaaaccagaaagtta | |
| | actggtaagtttagtctttttgtcttttatttcaggtcccggatccggtggtggtgcaa | |
| | atcaaagaactgctcctcagtggatgttgcctttacttctaggcctgtacggaagtgtt | |
| | acttctgctctaaaagctgcggaattgtacccgcggccgccaccatgtcacgcaagata | |
| | gaaggctttttgttattacttctctttggctatgaagccacattgggattatcgtctac | |
| | cgaggatgaaggcgaggacccatggtatcaaaaagcctgcaagtgcgattgccaaggag | |
| | gacccaatgctctgtggtctgcaggtgccacctccttggactgtataccagaatgccca | |
| | tatcacaagcctctgggtttcgagtcaggggaggtcacaccggaccagatcacctgctc | |
| | taacccggagcagtatgtgggctggtattcttcgtggactgcaaacaaggcccggctca | |
| | acagtcaaggctttgggtgtgcctggctctccaagttccaggacagtagccagtggtta | |
| | cagatagatctgaaggagatcaaagtgatttcaggcatcctcacccaggggcgctgtga | |
| | catcgatgagtggatgaccaagtacagcgtgcagtacaggaccgatgagcgcctgaact | |
| | ggatttactacaaggaccagactggaaacaaccgggtcttctatggcaactcggaccgc | |
| | acctccacggttcagaacctgctgcggccccccatcatctcccgcttcatccgcctcat | |
| | cccgctgggctggcacgtccgcattgccatccggatggagctgctggagtgcgtcagca | |
| | agtgtgcctgagtcgactagagctcgctgatcagcctcgactgtgccttctagttgcca | |
| | gccatctgttgtttgcccctcccccgtgccttccttgaccctggaaggtgccactccca | |
| | ctgtcctttcctaataaaatgaggaaattgcatcgcattgtctgagtaggtgtcattct | |
| | attctggggggtggggggggcaggacagcaagggggaggattgggaagacaatagcagg | |
| | catccgattcgagtgagcagaccacatgatgattggtattctccctgaagaaagaatgt | |
| | cagcaaggagtatctgaagtccgaccaagcctagaatccgcccggttcttatttgctga | |
| | agtaatactgttgtcaagtcagctacagggagtggtccctgcctggagtttgaagggct | |
| | gtcaaagacattgtggtcacttactcaagtggtaaattccttacagactggactgcagt | |
| | taagggtgataacgctactactaggacaagggtatccttgatcctgtggatctgtagtg | |
| | acctgagactgtcaaggtatggtgagtatgacccttgaccctttccaacggttgtaacc | |
| | gtgggcgctgtcaaatctgattaaccactactaggatagaatttggcagagctactgaa | |
| | atggttacctggtcaagagactggcccacagcataagggcgagcaagacgatgctcaga | |
| | ttaactaagtgaccgaagtatcctagatggtccactaccagcaaatgtgtcagacctcc | |
| | ctcagttggtagtacaatgtatccttctagtcgcgacttattatatcgacgagcttatg | |
| | tgtaagagaccagattctttgtaacctgacacttgagttcaaggtgtgtagtaggagag | |
| | tgcaaaccctgaagcaacagtagccgaaccgggtcgaggacctagcgttgattgacttt | |
| | gtctggtgtatcctgtaaatttctccaatcgagatcgaggtaaagtggacctcacagga | |
| | ctttatcaaacaacagagttaatacctcaaatctctcgctgatatatgtgacaaattca | |
| | ttgcgtcacttatcgcagctatgttcggtcagcatattctggcgaacacccacgctgca | |
| | gttcgacaaggatagtatctggcgctctcgcccaattcaagcacgcgaattactctatt | |
| | aacttgttgtctcttgtgtatatcataatcaattcctcctagttagtgattgaagacga | |
| | ccgtgcacttggctgccttgatgcagaccacttcttgtggccggcacaccctataatag | |
| | agatgtgataaataacttagctggactgtagttatttcgtcctagggattctgacgggt | |
| | gcaaatcactaagtgtctttggtattgggtctgtaacaatgttgagaactgtggtgtat | |
| | gtacgaacgatgtgatctgtagactacagcacagggtattgctgtttcgcaccagtgtg | |
| | acacttgtgtggctgtcctctgtgctatagcgccgtatatcttatgcaggttgtatagc | |
| | ccaacgactgtttaggtacgtccgtataagaataggagtgctacactctacattgaatc | |
| | tatccagagagagttctgtagaattaggtgacgaacaaatctacgcgataggaaggaga | |
| | gacctggttgtcccaagggccgtgggtatgatgtttagtacaactaggaggctctaggt | |
| | gtattggttgttggtcacgtagtgtccctacttggtcaggactaaagtttgtcagtgaa | |
| | ctctctactattattcatcgaacctgtggtcgtcggcggtctgatatatgctcggtaga | |
| | ttgcccgacaggacgtgaaggagcctaactattaaagcactctaactagcctatcgtaa | |
| | caacttatattgggtatttgggtacgacggtgtactggaaatctatctaattcttctga | |
| | tcgtatatcgggtcgtatattgatttatgacggaccgaccactcacgcggtccgatacg | |
| | cggtagcgctatccaggaccctcttatgaattggtatcaatagattgtggaacttgttg | |
| | cgtacactacagaatgtctttgcaaagaatgccaaattacaagctatagtcctcccact | |
| | atactgcttcgtgactccaactagggtagttatagttgttcaagactggtactctgtta | |
| | ccaattagtagcgatattgtgtgggatactggtgtctgtcatacgagaactgggagtag | |
| | cgctgtaaattctaaatagtcacactgttccagctggacgaccgctgtgtactctttgt | |
| | agaaactcactgcggcttccacttatgagatttggttataggtcacaaagtggcagtcc | |
| | agttgcaggtctcttgttgtaatatgactggattgaattgactaggaagacgtagtctg | |
| | ttcttgttaggtccgacgtggtcccaatttgcttatccaatatgaggtcctagttagca | |
| | gtcgctccgaggtcgaaatagatacccttgtggtttcgtagattgtctgattcgcaaca | |
| | atatcctgctaatcaatctacggaaacaccgcttcagaggaagattgcgacggacagaa | |
| | gagtcagtttcggctgcttgtggtttggcctaatataaagattacttacggcttaggta | |
| | tactgcacatcctccggccttgtttggtgcttgagattggtgcgcatatatgtaaggtg | |
| | ctatcgtatccgcctccagtacttgatcttgtcctgcggctttagggctgcttatccct | |
| | ccagcggtccaacctctgaagggtcaggccatacttcacctcctttgtctaggattctg | |
| | ttccgaatttccggtctgggtcatgtactctcaccatcgagtgtagggagacgattctc | |
| | gtaacagccgtagccgaatcactttcacttgagtctatgggttgtgcctgagttctagc | |
| | ttagtatttaacaacggcgtcggtccaaagacggtacacacttaaccatagtgaacgaa | |
| | tgaaacagtaatcgctccaggttggcgcgccttgcatgctggggagagatctgaggaac | |
| | ccctagtgatggagttggccactccctctctgcgcgctcgctcgctcactgaggccggg | |
| | cgaccaaaggtcgcccgacgcccgggctttgcccgggcggcctcagtgagcgagcgagc | |
| | gcgcagagagggagtggccaacctgcagcctgcttaattaatcatatgcatgaccgagt | |
| | aacgactcagtcgtcccacgaggcataagccagctgtaataagcgtacaagcgcacaca | |
| | cttgagtgggacaccttagggattcccaataattcggcaatctactgtgatacgacact | |
| | tcagaaacgatgacctgaccgacaagctgaactctacttcacctactactcaacactac | |
| | aaccagtcctctgttgacagtataacttgttggtactattatgactgaagggtaggttt | |
| | gattggatttgtttgcttcgtctggtcagcaggaggatctctcctttatacctgcagat | |
| | aaccgacgggtgcctattgtatcgaagtcattactacatccacctggtccacttgataa | |
| | atcaatccaaccgatacctacctgactaaggtcccttcacgaggacctttgtcacccag | |
| | taatcagatttagaagtcgtattcttacacaacctcacaactgattctgaacgcaccgt | |
| | cgaagtcaacaccacagtatctgaggaagtaagaacagtttgtactttgagcggcgccc | |
| | actgtgaatacgagagactttaggtcactttcaactatttagtgggtcaccttcggaac | |
| | tcagacgaggacaccaccacagtagaggtggatagtccaaacacagttgttgtacagac | |
| | ccagtggattgaataggatcttgatcaggtgtgtccagacgctccagactatagttaaa | |
| | taggagtaagaccgctaagcgcagcaggagttcccaatcgttacaactagatcctgtgg | |
| | tgatatatctagtgtccgacttacatgcggcctctgtattagttcatattcattggtct | |
| | gcacgcctcaggagagatattcgacctgaggataccaggacactttggatcagtatggg | |
| | tcggaccagttgaacactgataaagtactgaccgagtgggttgtgacggcccagagatt | |
| | cagtcctactttctcaactggagaggaaccacctgttctgagtaacccacaatcgtgtc | |
| | cagagactggacaacactgaatgcaggttcctttctgttcacccagataagggagacca | |
| | cactaagtggtgtattcgcacgacgatactggtgtcatttatttccctggccagtgtgt | |
| | acctgcgtgactgcttccgaaggagtgttacaaggagaaaggagagtataactacaagg | |
| | aatgtagacactgcctgcagcccatgaattgcaatataatcggacttgctcggagtccc | |
| | tctactacttaggaagaggattgatcaccacaggtcctctggtcagtttaggatgcata | |
| | atgatccatgccgagacttggaattgatatatactggtatcaccgtataggttgctggt | |
| | tgtagtgtctttagcccacccagtgtaatagcatgtatagttaatctgcgtaacgatcc | |
| | ctacgaacgacctagaggtcagtcacaccacggcgcgaagaagactcttacactaatag | |
| | taagctattgtatctagttaagtgaccaacccatttggttcacatcgtagttgactttc | |
| | cgggtccttggacccgaaattctatcacatcagtccctaatgcgatgagtgcgtctttc | |
| | tggttcacgtggtctgtatcatcaattcctaaatcttagtgactaattattatttcgag | |
| | acattgtacacctggatggtactgtttctgtcccacgccctacaacggacaaccaacaa | |
| | cgcaccttgttgaacaacaatgatccactacacaagctccgaggtcgttctcgtcttga | |
| | tttgaaacatgggtgcaatgtgtattagaggtttcaatccatatatcctaatacgttga | |
| | tattgttctttagtaggtggctcctaaagctcaaagttgcaagcaagctaacaatacca | |
| | tttcacgacaaggaggataaccacgtgtgttattctgactccatgtaatctgctaatcc | |
| | caccttcttcagtcgagctttattatattcgcgtaccggagacccaggcagaggtgagg | |
| | acgaatatgttgcaggagtaaagaagctattgaaggatttcttaggacgcgctgctggt | |
| | gttctaaagtagtatagaagctaagtcaagccggagcatactccgatatttggagcctg | |
| | ataccagttactgggtgtttagcagaagcgttacgtgacttacacgatataatgactat | |
| | acttgatctgaaggcatcaagtatcaacgaaagtcctttctgattgacaaacagactct | |
| | cactttacagggataggattggaaccaccaatctgaggtattcaccagtatccagccac | |
| | tgctctctcaagtccaacgtaatccaaggagactccggcaattccttaattggcgttag | |
| | ctactgtgttgcgagcatttattactatctggcaaggagtgacaaattagcaggattga | |
| | ttgaggtttgtgatccaccagaaggaaggactcaccgttaggtatggtgaagataccca | |
| | gcttgtgctgactgaggatagcaagacagatcacttatcactcgaacagatcggaccca | |
| | ccgcacacctcaatatctctttaggaccaattcaagaacaacttagacgctgtacactt | |
| | aaacagcacagctgtgggtaagagaagtaatagagcgcgatatcagcagcagcctcttg | |
| | tagatcggcttcaagatatctatgtgtggaaataggtcactagagtgagcggtactttc | |
| | gctcttacaggtttgaaaggtcttagcagcgctatttagcgagtcctggagaccacgac | |
| | aacgagcgctgtggtcctcacaaataggaccaacaataacaatacaggatttatcaaga | |
| | gatagaagtcggtataccgattctgctgtttcgtagtcggctacagatcaatagattat | |
| | tgtgttcagattagttgaggaagttgaatgggtcaaggaccttacctaataccaggtca | |
| | ccggacttggtaattttccctcttggggttggtgagtagtactgtctaccaactggtga | |
| | ctggtgatcccttacttgtttggaccctggtaatacggttatccacagaatcaggggat | |
| | aacgcaggaaagaacatgtgagcaaaaggccagcaaaaggccaggaaccgtaaaaaggc | |
| | cgcgttgctggcgtttttccataggctccgcccccctgacgagcatcacaaaaatcgac | |
| | gctcaagtcagaggtggcgaaacccgacaggactataaagataccaggcgtttccccct | |
| | ggaagctccctcgtgcgctctcctgttccgaccctgccgcttaccggatacctgtccgc | |
| | ctttctcccttcgggaagcgtggcgctttctcatagctcacgctgtaggtatctcagtt | |
| | cggtgtaggtcgttcgctccaagctgggctgtgtgcacgaaccccccgttcagcccgac | |
| | cgctgcgccttatccggtaactatcgtcttgagtccaacccggtaagacacgacttatc | |
| | gccactggcagcagccactggtaacaggattagcagagcgaggtatgtaggcggtgcta | |
| | cagagttcttgaagtggtggcctaactacggctacactagaagaacagtatttggtatc | |
| | tgcgctctgctgaagccagttaccttcggaaaaagagttggtagctcttgatccggcaa | |
| | acaaaccaccgctggtagcggtggtttttttgtttgcaagcagcagattacgcgcagaa | |
| | aaaaaggatctcaagaagatcctttgatcttttctacggggtctgacgctcagtggaac | |
| | gaaaactcacgttaagggattttggtcatgagattatcaaaaaggatcttcacctagat | |
| | ccttttcacgtagaaacccagtccgcagaaacggtgctgacccctgatgaatgtcacct | |
| | actgggttatctggacaagggaaaacccaagcgcaaagagaaagcaggtagcttggagt | |
| | ggggttacatggtgataggtagactgggaggttttatggacaggaaccgaacctcaatt | |
| | cccaggtggggtgtcctctggtaaggttgggaagtcctggaaagtaaactggatggctt | |
| | tctcgccgctgttacattgcacaagataaaaatatatcatcatgaacaataaaactgtc | |
| | tgcttacataaacagtaatacaaggggtgttatgagccatattcaacgggaaacgtcga | |
| | ggccgcgattaaattccaacatggatgctgatttatatgggtataaatgggctcgcgat | |
| | aatgtcgggcaatcaggtgcgacaatctatcgcttgtatgggaagcccgatgcgccaga | |
| | gttgtttctgaaacatggcaaaggtagcgttgccaatgatgttacagatgagatggtca | |
| | gactaaactggctgacggaatttatgcctcttccgaccatcaagcattttatccgtact | |
| | cctgatgatgcatggttactcaccactgcgatccccggaaaaacagcattccaggtatt | |
| | agaagaatatcctgattcaggtgaaaatattgttgatgcgctggcagtgttcctgcgcc | |
| | ggttgcattcgattcctgtttgtaattgtccttttaacagcgatcgcgtatttcgtctc | |
| | gctcaggcgcaatcacgaatgaataacggtttggttgatgcgagtgattttgatgacga | |
| | gcgtaatggctggcctgttgaacaagtctggaaagaaatgcataaacttttgccattct | |
| | caccggattcagtcgtcactcatggtgatttctcacttgataaccttatttttgacgag | |
| | gggaaattaataggttgtattgatgttggacgagtcggaatcgcagaccgataccagga | |
| | tcttgccatcctatggaactgcctcggtgagttttctccttcattacagaaacggcttt | |
| | ttcaaaaatatggtattgataatcctgatatgaataaattgcagtttcatttgatgctc | |
| | gatgagtttttctaaattttgttaaaatttttgttaaatcagctcattttttaaccaat | |
| | aggccctgttggttcaagtctccctaatagtttgaggggaaggagttggtgtcaagggt | |
| | ctgaccaccatattacgggactagagacaggtggaggtctggacaccctattggtggat | |
| | cattcattatggatacggtcctggacaagtggactgatcccaattaggagactggtcca | |
| | gtgtcccaccttgagaggtactgatactcacttacctcaaacactaggttcttaagagg | |
| | gtgatggagagagattaggaccaaaccagagactttggtcctaactccagaggtagtga | |
| | gtacaaaacaccactaggtgaggttgggaccagtctattttctccaaactgagaaggtg | |
| | tcagtggaggcacacttaccctctcactggctattcgccattcaggctgcgcaactgtt | |
| | gggaagggcgatcggtgcggg | |
| 34 | MVILQQGDHVWMDLRLGQEFDVPIGAVVKLCDSGQVQVVDDEDNEHWISPQNATHIKPM | Human |
| | HPTSVHGVEDMIRLGDLNEAGILRNLLIRYRDHLIYTYTGSILVAVNPYQLLSIYSPEH | Myo7a - |
| | IRQYTNKKIGEMPPHIFAIADNCYFNMKRNSRDQCCIISGESGAGKTESTKLILQFLAA | GenBank: |
| | ISGQHSWIEQQVLEATPILEAFGNAKTIRNDNSSRFGKYIDIHFNKRGAIEGAKIEQYL | EAW75022.1 |
| | LEKSRVCRQALDERNYHVFYCMLEGMSEDQKKKLGLGQASDYNYLAMGNCITCEGRVDS | |
| | QEYANIRSAMKVLMFTDTENWEISKLLAAILHLGNLQYEARTFENLDACEVLFSPSLAT | |
| | AASLLEVNPPDLMSCLTSRTLITRGETVSTPLSREQALDVRDAFVKGIYGRLFVWIVDK | |
| | INAAIYKPPSQDVKNSRRSIGLLDIFGFENFAVNSFEQLCINFANEHLQQFFVRHVFKL | |
| | EQEEYDLESIDWLHIEFTDNQDALDMIANKPMNIISLIDEESKFPKGTDTTMLHKLNSQ | |
| | HKLNANYIPPKNNHETQFGINHFAGIVYYETQGFLEKNRDTLHGDIIQLVHSSRNKFIK | |
| | QIFQADVAMGAETRKRSPTLSSQFKRSLELLMRTLGACQPFFVRCIKPNEFKKPMLFDR | |
| | HLCVRQLRYSGMMETIRIRRAGYPIRYSFVEFVERYRVLLPGVKPAYKQGDLRGTCQRM | |
| | AEAVLGTHDDWQIGKTKIFLKDHHDMLLEVERDKAITDRVILLQKVIRGFKDRSNFLKL | |
| | KNAATLIQRHWRGHNCRKNYGLMRLGFLRLQALHRSRKLHQQYRLARQRIIQFQARCRA | |
| | YLVRKAFRHRLWAVLTVQAYARGMIARRLHQRLRAEYLWRLEAEKMRLAEEEKLRKEMS | |
| | AKKAKEEAERKHQERLAQLAREDAERELKEKEAARRKKELLEQMERARHEPVNHSDMVD | |
| | KMFGFLGTSGGLPGQEGQAPSGFEDLERGRREMVEEDLDAALPLPDEDEEDLSEYKFAK | |
| | FAATYFQGTTTHSYTRRPLKQPLLYHDDEGDQLAALAVWITILRFMGDLPEPKYHTAMS | |
| | DGSEKIPVMTKIYETLGKKTYKRELQALQGEGEAQLPEGQKKSSVRHKLVHLTLKKKSK | |
| | LTEEVTKRLHDGESTVQGNSMLEDRPTSNLEKLHFIIGNGILRPALRDEIYCQISKQLT | |
| | HNPSKSSYARGWILVSLCVGCFAPSEKFVKYLRNFIHGGPPGYAPYCEERLRRTFVNGT | |
| | RTQPPSWLELQATKSKKPIMLPVTFMDGTTKTLLTDSATTAKELCNALADKISLKDRFG | |
| | FSLYIALFDKVSSLGSGSDHVMDAISQCEQYAKEQGAQERNAPWRLFFRKEVFTPWHSP | |
| | SEDNVATNLIYQQVVRGVKFGEYRCEKEDDLAELASQQYFVDYGSEMILERLLNLVPTY | |
| | IPDREITPLKTLEKWAQLAIAAHKKGIYAQRRTDAQKVKEDVVSYARFKWPLLFSRFYE | |
| | AYKFSGPSLPKNDVIVAVNWTGVYFVDEQEQVLLELSFPEIMAVSSSRGAKTTAPSFTL | |
| | ATIKGDEYTFTSSNAEDIRDLVVTFLEGLRKRSKYVVALQDNPNPAGEESGFLSFAKGD | |
| | LIILDHDTGEQVMNSGWANGINERTKQRGDFPTDCVYVMPTVTMPPREIVALVTMTPDQ | |
| | RQDVVRLLQLRTAEPEVRAKPYTLEEFSYDYFRPPPKHTLSRVMVSKARGKDRLWSHTR | |
| | EPLKQALLKKLLGSEELSQEACLAFIDIPVLKYMGDYPSKRTRSVNELTDQIFEGPLKA | |
| | EPLKDEAYVQILKQLTDNHIRYSEERGWELLWLCTGLFPPSNILLPHVQRFLQSRKHCP | |
| | LAIDCLQRLOKALRNGSRKYPPHLVEVEAIQHKTTQIFHKVYFPDDTDEAFEVESSTKA | |
| | KDFCQNIATRLLLKSSEGFSLFVKIADKVISVPENDFFFDFVRHLTDWIKKARPIKDGI | |
| | VPSLTYQVFFMKKLWTTTVPGKDPMADSIFHYYQELPKYLRGYHKCTREEVLQLGALIY | |
| | RVKFEEDKSYFPSIPKLLRELVPQDLIRQVSPDDWKRSIVAYFNKHAGKSKEEAKLAFL | |
| | KLIFKWPTFGSAFFEQTTEPNFPEILLIAINKYGVSLIDPKTKDILTTHPFTKISNWSS | |
| | GNTYFHITIGNLVRGSKLLCETSLGYKMDDLLTSYISQMLTAMSKQRGSRSGK | |
| 35 | MVILQQGDHVWMDLRLGQEFDVPIGAVVKLCDSGQVQVVDDEDNEHWISPQNATHIKPM | human |
| | HPTSVHGVEDMIRLGDLNEAGILRNLLIRYRDHLIYTYTGSILVAVNPYQLLSIYSPEH | ABCA4 - |
| | IRQYTNKKIGEMPPHIFAIADNCYFNMKRNSRDQCCIISGESGAGKTESTKLILQFLAA | NCBI |
| | ISGQHSWIEQQVLEATPILEAFGNAKTIRNDNSSRFGKYIDIHFNKRGAIEGAKIEQYL | Reference |
| | LEKSRVCRQALDERNYHVFYCMLEGMSEDQKKKLGLGQASDYNYLAMGNCITCEGRVDS | Sequence: |
| | QEYANIRSAMKVLMFTDTENWEISKLLAAILHLGNLQYEARTFENLDACEVLFSPSLAT | NP_000251.3 |
| | AASLLEVNPPDLMSCLTSRTLITRGETVSTPLSREQALDVRDAFVKGIYGRLFVWIVDK | |
| | INAAIYKPPSQDVKNSRRSIGLLDIFGFENFAVNSFEQLCINFANEHLQQFFVRHVFKL | |
| | EQEEYDLESIDWLHIEFTDNQDALDMIANKPMNIISLIDEESKFPKGTDTTMLHKLNSQ | |
| | HKLNANYIPPKNNHETQFGINHFAGIVYYETQGFLEKNRDTLHGDIIQLVHSSRNKFIK | |
| | QIFQADVAMGAETRKRSPTLSSQFKRSLELLMRTLGACQPFFVRCIKPNEFKKPMLFDR | |
| | HLCVRQLRYSGMMETIRIRRAGYPIRYSFVEFVERYRVLLPGVKPAYKQGDLRGTCQRM | |
| | AEAVLGTHDDWQIGKTKIFLKDHHDMLLEVERDKAITDRVILLQKVIRGFKDRSNFLKL | |
| | KNAATLIQRHWRGHNCRKNYGLMRLGFLRLQALHRSRKLHQQYRLARQRIIQFQARCRA | |
| | YLVRKAFRHRLWAVLTVQAYARGMIARRLHQRLRAEYLWRLEAEKMRLAEEEKLRKEMS | |
| | AKKAKEEAERKHQERLAQLAREDAERELKEKEAARRKKELLEQMERARHEPVNHSDMVD | |
| | KMFGFLGTSGGLPGQEGQAPSGFEDLERGRREMVEEDLDAALPLPDEDEEDLSEYKFAK | |
| | FAATYFQGTTTHSYTRRPLKQPLLYHDDEGDQLAALAVWITILRFMGDLPEPKYHTAMS | |
| | DGSEKIPVMTKIYETLGKKTYKRELQALQGEGEAQLPEGQKKSSVRHKLVHLTLKKKSK | |
| | LTEEVTKRLHDGESTVQGNSMLEDRPTSNLEKLHFIIGNGILRPALRDEIYCQISKQLT | |
| | HNPSKSSYARGWILVSLCVGCFAPSEKFVKYLRNFIHGGPPGYAPYCEERLRRTFVNGT | |
| | RTQPPSWLELQATKSKKPIMLPVTFMDGTTKTLLTDSATTAKELCNALADKISLKDRFG | |
| | FSLYIALFDKVSSLGSGSDHVMDAISQCEQYAKEQGAQERNAPWRLFFRKEVFTPWHSP | |
| | SEDNVATNLIYQQVVRGVKFGEYRCEKEDDLAELASQQYFVDYGSEMILERLLNLVPTY | |
| | IPDREITPLKTLEKWAQLAIAAHKKGIYAQRRTDAQKVKEDVVSYARFKWPLLFSRFYE | |
| | AYKFSGPSLPKNDVIVAVNWTGVYFVDEQEQVLLELSFPEIMAVSSSRECRVWLSLGCS | |
| | DLGCAAPHSGWAGLTPAGPCSPCWSCRGAKTTAPSFTLATIKGDEYTFTSSNAEDIRDL | |
| | VVTFLEGLRKRSKYVVALQDNPNPAGEESGFLSFAKGDLIILDHDTGEQVMNSGWANGI | |
| | NERTKQRGDFPTDSVYVMPTVTMPPREIVALVTMTPDQRQDVVRLLQLRTAEPEVRAKP | |
| | YTLEEFSYDYFRPPPKHTLSRVMVSKARGKDRLWSHTREPLKQALLKKLLGSEELSQEA | |
| | CLAFIAVLKYMGDYPSKRTRSVNELTDQIFEGPLKAEPLKDEAYVQILKQLTDNHIRYS | |
| | EERGWELLWLCTGLFPPSNILLPHVQRFLQSRKHCPLAIDCLQRLQKALRNGSRKYPPH | |
| | LVEVEAIQHKTTQIFHKVYFPDDTDEAFEVESSTKAKDFCQNIATRLLLKSSEGFSLFV | |
| | KIADKVLSVPENDFFFDFVRHLTDWIKKARPIKDGIVPSLTYQVFFMKKLWTTTVPGKD | |
| | PMADSIFHYYQELPKYLRGYHKCTREEVLQLGALIYRVKFEEDKSYFPSIPKLLRELVP | |
| | QDLIRQVSPDDWKRSIVAYFNKHAGKSKEEAKLAFLKLIFKWPTFGSAFFEVKQTTEPN | |
| | FPEILLIAINKYGVSLIDPKTKDILTTHPFTKISNWSSGNTYFHITIGNLVRGSKLLCE | |
| | TSLGYKMDDLLTSYISQMLTAMSKQRGSRSGK | |
| 36 | MTDKSIVILSLMVFHSSFINGKTCRRQLVEEWHPQPSSYVVNWTLTENICLDFYRDCWF | Human EYS - |
| | LGVNTKIDTSGNQAVPQICPLQIQLGDILVISSEPSLQFPEINLMNVSETSFVGCVQNT | NCBI |
| | TTEDQLLFGCRLKGMHTVNSKWLSVGTHYFITVMASGPSPCPLGLRLNVTVKQQFCQES | Reference |
| | LSSEFCSGHGKCLSEAWSKTYSCHCQPPFSGKYCQELDACSFKPCKNNGSCINKRENWD | Sequence: |
| | EQAYECVCHPPFTGKNCSEIIGQCQPHVCFHGNCSNITSNSFICECDEQFSGPFCEVSA | NM_001142800.2 |
| | KPCVSLLFWKRGICPNSSSAYTYECPKGSSSQNGETDVSEFSLVPCQNGTDCIKISNDV | |
| | MCICSPIFTDLLCKSIQTSCESFPLRNNATCKKCEKDYPCSCISGFTEKNCEKAIDHCK | |
| | LLSINCLNEEWCFNIIGRFKYVCIPGCTKNPCWFLKNVYLIHQHLCYCGVTFHGICQDK | |
| | GPAQFEYVWQLGFAGSEGEKCQGVIDAYFFLAANCTEDATYVNDPEDNNSSCWFPHEGT | |
| | KEICANGCSCLSEEDSQEYRYLCFLRWAGNMYLENTTDDQENECQHEAVCKDEINRPRC | |
| | SCSLSYIGRLCVVNVDYCLGNHSISVHGLCLALSHNCNCSGLQRYERNICEIDTEDCKS | |
| | ASCKNGTTSTHLRGYFFRKCVPGFKGTQCEIDIDECASHPCKNGATCIDQPGNYFCQCV | |
| | PPFKVVDGFSCLCNPGYVGIRCEQDIDDCILNACEHNSTCKDLHLSYQCVCLSDWEGNF | |
| | CEQESNECKMNPCKNNSTCTDLYKSYRCECTSGWTGQNCSEEINECDSDPCMNGGLCHE | |
| | STIPGQFVCLCPPLYTGQFCHQRYNLCDLLHNPCRNNSTCLALVDANQHCICREEFEGK | |
| | NCEIDVKDCLFLSCQDYGDCEDMVNNFRCICRPGFSGSLCEIEINECSSEPCKNNGTCV | |
| | DLTNRFFCNCEPEYHGPFCELDVNKCKISPCLDEENCVYRTDGYNCLCAPGYTGINCEI | |
| | NLDECLSEPCLHDGVCIDGINHYTCDCKSGFFGTHCETNANDCLSNPCLHGRCTELINE | |
| | YPCSCDADGTSTQCKIKINDCTSIPCMNEGFCQKSAHGFTCICPRGYTGAYCEKSIDNC | |
| | AEPELNSVICLNGGICVDGPGHTFDCRCLPGFSGQFCEININECSSSPCLHGADCEDHI | |
| | NGYVCKCQPGWSGHHCENELECIPNSCVHELCMENEPGSTCLCTPGFMTCSIGLLCGDE | |
| | IRRITCLTPIFQRTDPISTQTYTIPPSETLVSSFPSIKATRIPAIMDTYPVDQGPKQTG | |
| | IVKHDILPTTGLATLRISTPLESYLLQELIVTRELSAKHSLLSSADVSSSRFLNFGIRD | |
| | PAQIVQDKTSVSHMPIRTSAATLGFFFPDRRARTPFIMSSLMSDFIFPTQSLLFENCQT | |
| | VALSATPTTSVIRSIPGADIELNRQSLLSRGFLLIAASISATPVVSRGAQEDIEEYSAD | |
| | SLISRREHWRLLSPSMSPIFPAKVIISKQVTILNSSALHRFSTKAFNPSEYQAITEASS | |
| | NQRLTNIKSQAADSLRELSQTCATCSMTEIKSSREFSDQVLHSKQSHFYETFWMNSAIL | |
| | ASWYALMGAQTITSGHSFSSATEITPSVAFTEVPSLFPSKKSAKRTILSSSLEESITLS | |
| | SNLDVNLCLDKTCLSIVPSQTISSDLMNSDLTSKMTTDELSVSENILKLLKIRQYGITM | |
| | GPTEVLNQESLLDMEKSKGSHTLFKLHPSDSSLDFELNLQIYPDVTLKTYSEITHANDF | |
| | KNNLPPLTGSVPDFSEVTTNVAFYTVSATPALSIQTSSSMSVIRPDWPYFTDYMTSLKK | |
| | EVKTSSEWSKWELQPSVQYQEFPTASRHLPFTRSLTLSSLESILAPQRLMISDFSCVRY | |
| | YGDSYLEFQNVALNPQNNISLEFQTFSSYGLLLYVKQDSNLVDGFFIQLFIENGTLKYH | |
| | FYCPGEAKFKSINTTVRVDNGQKYTLLIRQELDPCNAELTILGRNTQICESINHVLGKP | |
| | LPKSGSVFIGGFPDLHGKIQMPVPVKNFTGCIEVIEINNWRSFIPSKAVKNYHINNCRS | |
| | QGFMLSPTASFVDASDVTQGVDTMWTSVSPSVAAPSVCQQDVCHNGGTCHAIFLSSGIV | |
| | SFQCDCPLHFTGRFCEKDAGLFFPSFNGNSYLELPFLKFVLEKEHNRTVTIYLTIKTNS | |
| | LNGTILYSNGNNCGKQFLHLFLVEGRPSVKYGCGNSQNILTVSANYSINTNAFTPITIR | |
| | YTTPVGSPGVVCMIEMTADGKPPVQKKDTEISHASQAYFESMFLGHIPANVQIHKKAGP | |
| | VYGFRGCILDLQVNNKEFFIIDEARHGKNIENCHVPWCAHHLCRNNGTCISDNENLFCE | |
| | CPRLYSGKLCQFASCENNPCGNGATCVPKSGTDIVCLCPYGRSGPLCTDAINITQPRFS | |
| | GTDAFGYTSFLAYSRISDISFHYEFHLKFQLANNHSALQNNLIFFTGQKGHGLNGDDFL | |
| | AVGLLNGSVVYSYNLGSGIASIRSEPLNLSLGVHTVHLGKFFQEGWLKVDDHKNKSIIA | |
| | PGRLVGLNVFSQFYVGGYSEYTPDLLPNGADFKNGFQGCIFTLQVRTEKDGHFRGLGNP | |
| | EGHPNAGRSVGQCHASPCSLMKCGNGGTCIESGTSVYCNCTTGWKGSFCTETVSTCDPE | |
| | HDPPHHCSRGATCISLPHGYTCFCPLGTTGIYCEQALSISDPSFRSNELSWMSFASFHV | |
| | RKKTHIQLQFQPLAADGILFYAAQHLKAQSGDFLCISLVNSSVQLRYNLGDRTIILETL | |
| | QKVTINGSTWHIIKAGRVGAEGYLDLDGINVTEKASTKMSSLDTNTDFYIGGVSSLNLV | |
| | NPMAIENEPVGFQGCIRQVIINNQELQLTEFGAKGGSNVGDCDGTACGYNTCRNGGECT | |
| | VNGTTFSCRCLPDWAGNTCNQSVSCLNNLCLHQSLCIPDQSFSYSCLCTLGWVGRYCEN | |
| | KTSFSTAKFMGNSYIKYIDPNYRMRNLQFTTISLNFSTTKTEGLIVWMGIAQNEENDFL | |
| | AIGLHNQTLKIAVNLGERISVPMSYNNGTFCCNKWHHVVVIQNQTLIKAYINNSLILSE | |
| | DIDPHKNFVALNYDGICYLGGFEYGRKVNIVTQEIFKTNFVGKIKDVVFFQEPKNIELI | |
| | KLEGYNVYDGDEQNEVT | |
| 37 | MWTLGRRAVAGLLASPSPAQAQTLTRVPRPAELAPLCGRRGLRTDIDATCTPRRASSNQ | Human FXN - |
| | RGLNQIWNVKKQSVYLMNLRKSGTLGHPGSLDETTYERLAEETLDSLAEFFEDLADKPY | NCBI |
| | TFEDYDVSFGSGVLTVKLGGDLGTYVINKQTPNKQIWLSSPSSGPKRYDWTGKNWVYSH | Reference |
| | DGVSLHELLAAELTKALKTKLDLSSLAYSGKDA | Sequence: |
| | | NP_000135.2 |
| 38 | MSRKIEGFLLLLLFGYEATLGLSSTEDEGEDPWYQKACKCDCQGGPNALWSAGATSLDC | Human RS1 - |
| | IPECPYHKPLGFESGEVTPDQITCSNPEQYVGWYSSWTANKARLNSQGFGCAWLSKFQD | Genbank |
| | SSQWLQIDLKEIKVISGILTQGRCDIDEWMTKYSVQYRTDERLNWIYYKDQTGNNRVFY | AF014459.1 |
| | GNSDRTSTVQNLLRPPIISRFIRLIPLGWHVRIAIRMELLECVSKCA | |
| 39 | MFKSLTKVNKVKPIGENNENEQSSRRNEEGSHPSNQSQQTTAQEENKGEEKSLKTKSTP | Human |
| | VTSEEPHTNIQDKLSKKNSSGDLTTNPDPQNAAEPTGTVPEQKEMDPGKEGPNSPQNKP | CNGB3 - |
| | PAAPVINEYADAQLHNLVKRMRQRTALYKKKLVEGDLSSPEASPQTAKPTAVPPVKESD | NCBI |
| | DKPTEHYYRLLWFKVKKMPLTEYLKRIKLPNSIDSYTDRLYLLWLLLVTLAYNWNCCFI | Reference |
| | PLRLVFPYQTADNIHYWLIADIICDIIYLYDMLFIQPRLQFVRGGDIIVDSNELRKHYR | Sequence: |
| | TSTKFQLDVASIIPFDICYLFFGFNPMFRANRMLKYTSFFEFNHHLESIMDKAYIYRVI | NM_019098.5 |
| | RTTGYLLFILHINACVYYWASNYEGIGTTRWVYDGEGNEYLRCYYWAVRTLITIGGLPE | |
| | PQTLFEIVFQLLNFFSGVFVFSSLIGQMRDVIGAATANQNYFRACMDDTIAYMNNYSIP | |
| | KLVQKRVRTWYEYTWDSQRMLDESDLLKTLPTTVQLALAIDVNFSIISKVDLFKGCDTQ | |
| | MIYDMLLRLKSVLYLPGDFVCKKGEIGKEMYIIKHGEVQVLGGPDGTKVLVTLKAGSVF | |
| | GEISLLAAGGGNRRTANVVAHGFANLLTLDKKTLQEILVHYPDSERILMKKARVLLKQK | |
| | AKTAEATPPRKDLALLFPPKEETPKLFKTLLGGTGKASLARLLKLKREQAAQKKENSEG | |
| | GEEEGKENEDKQKENEDKQKENEDKGKENEDKDKGREPEEKPLDRPECTASPIAVEEEP | |
| | HSVRRTVLPRGTSRQSLIISMAPSAEGGEEVLTIEVKEKAKQ | |
| 40 | MAKINTQYSHPSRTHLKVKTSDRDLNRAENGLSRAHSSSEETSSVLQPGIAMETRGLAD | Human |
| | SGQGSFTGQGIARLSRLIFLLRRWAARHVHHQDQGPDSFPDRFRGAELKEVSSQESNAQ | CNGA3 - |
| | ANVGSQEPADRGRSAWPLAKCNTNTSNNTEEEKKTKKKDAIVVDPSSNLYYRWLTAIAL | NCBI |
| | PVFYNWYLLICRACFDELQSEYLMLWLVLDYSADVLYVLDVLVRARTGFLEQGLMVSDT | Reference |
| | NRLWQHYKTTTQFKLDVLSLVPTDLAYLKVGTNYPEVRFNRLLKFSRLFEFFDRTETRT | Sequence: |
| | NYPNMFRIGNLVLYILIIIHWNACIYFAISKFIGFGTDSWVYPNISIPEHGRLSRKYIY | NM_001298.3 |
| | SLYWSTLTLTTIGETPPPVKDEEYLFVVVDFLVGVLIFATIVGNVGSMISNMNASRAEF | |
| | QAKIDSIKQYMQFRKVTKDLETRVIRWFDYLWANKKTVDEKEVLKSLPDKLKAEIAINV | |
| | HLDTLKKVRIFQDCEAGLLVELVLKLRPTVFSPGDYICKKGDIGKEMYIINEGKLAVVA | |
| | DDGVTQFVVLSDGSYFGEISILNIKGSKSGNRRTANIRSIGYSDLFCLSKDDLMEALTE | |
| | YPEAKKALEEKGRQILMKDNLIDEELARAGADPKDLEEKVEQLGSSLDTLQTRFARLLA | |
| | EYNATQMKMKQRLSQLESQVKGGGDKPLADGEVPGDATKTEDKQQ | |
| 41 | MAQQWSLQRLAGRHPQDSYEDSTQSSIFTYTNSNSTRGPFEGPNYHIAPRWVYHLTSVW | Human |
| | MIFVVIASVFTNGLVLAATMKFKKLRHPLNWILVNLAVADLAETVIASTISVVNQVYGY | OPN1MW - |
| | FVLGHPMCVLEGYTVSLCGITGLWSLAIISWERWMVVCKPFGNVRFDAKLAIVGIAFSW | NCBI |
| | IWAAVWTAPPIFGWSRYWPHGLKTSCGPDVFSGSSYPGVQSYMIVLMVTCCITPLSIIV | Reference |
| | LCYLQVWLAIRAVAKQQKESESTQKAEKEVTRMVVVMVLAFCFCWGPYAFFACFAAANP | Sequence: |
| | GYPFHPLMAALPAFFAKSATIYNPVIYVFMNRQFRNCILQLFGKKVDDGSELSSASKTE | NM_000513.2 |
| | VSSVSSVSPA | |
| 42 | MAQQWSLQRLAGRHPQDSYEDSTQSSIFTYTNSNSTRGPFEGPNYHIAPRWVYHLTSVW | Human |
| | MIFVVTASVFTNGLVLAATMKFKKLRHPLNWILVNLAVADLAETVIASTISIVNQVSGY | OPNILW - |
| | FVLGHPMCVLEGYTVSLCGITGLWSLAIISWERWMVVCKPFGNVRFDAKLAIVGIAFSW | NCBI |
| | IWAAVWTAPPIFGWSRYWPHGLKTSCGPDVFSGSSYPGVQSYMIVLMVTCCIIPLAIIM | Reference |
| | LCYLQVWLAIRAVAKQQKESESTQKAEKEVTRMVVVMIFAYCVCWGPYTFFACFAAANP | Sequence: |
| | GYAFHPLMAALPAYFAKSATIYNPVIYVFMNRQFRNCILQLFGKKVDDGSELSSASKTE | NM_020061.6 |
| | VSSVSSVSPA | |
| 43 | MREPEELMPDSGAVFTFGKSKFAENNPGKFWFKNDVPVHLSCGDEHSAVVTGNNKLYMF | Human |
| | GSNNWGQLGLGSKSAISKPTCVKALKPEKVKLAACGRNHTLVSTEGGNVYATGGNNEGQ | RPGR - |
| | LGLGDTEERNTFHVISFFTSEHKIKQLSAGSNTSAALTEDGRLFMWGDNSEGQIGLKNV | NCBI |
| | SNVCVPQQVTIGKPVSWISCGYYHSAFVTTDGELYVFGEPENGKLGLPNQLLGNHRTPQ | Reference |
| | LVSEIPEKVIQVACGGEHTVVLTENAVYTFGLGQFGQLGLGTFLFETSEPKVIENIRDQ | Sequence: |
| | TISYISCGENHTALITDIGLMYTFGDGRHGKLGLGLENFTNHFIPTLCSNFLRFIVKLV | NM_000328.3 |
| | ACGGCHMVVFAAPHRGVAKEIEFDEINDTCLSVATFLPYSSLTSGNVLQRTLSARMRRR | |
| | ERERSPDSFSMRRTLPPIEGTLGLSACFLPNSVFPRCSERNLQESVLSEQDLMQPEEPD | |
| | YLLDEMTKEAEIDNSSTVESLGETTDILNMTHIMSLNSNEKSLKLSPVQKQKKQQTIGE | |
| | LTQDTALTENDDSDEYEEMSEMKEGKACKQHVSQGIFMTQPATTIEAFSDEEVEIPEEK | |
| | EGAEDSKGNGIEEQEVEANEENVKVHGGRKEKTEILSDDLTDKAEDHEFSKTEELKLED | |
| | VDEEINAENVESKKKTVGDDESVPTGYHSKTEGAERTNDDSSAETIEKKEKANLEERAI | |
| | CEYNENPKGYMLDDADSSSLEILENSETTPSKDMKKTKKIFLFKRVPSINQKIVKNNNE | |
| | PLPEIKSIGDQIILKSDNKDADQNHMSQNHQNIPPTNTERRSKSCTIL | |
| 44 | MREPEELMPDSGAVFTFGKSKFAENNPGKFWFKNDVPVHLSCGDEHSAVVTGNNKLYMF | Human |
| | GSNNWGQLGLGSKSAISKPTCVKALKPEKVKLAACGRNHTLVSTEGGNVYATGGNNEGQ | RPGR- |
| | LGLGDTEERNTFHVISFFTSEHKIKQLSAGSNTSAALTEDGRLFMWGDNSEGQIGLKNV | ORF15 - |
| | SNVCVPQQVTIGKPVSWISCGYYHSAFVTTDGELYVFGEPENGKLGLPNQLLGNHRTPQ | NCBI |
| | LVSEIPEKVIQVACGGEHTVVLTENAVYTFGLGQFGQLGLGTFLFETSEPKVIENIRDQ | Reference |
| | TISYISCGENHTALITDIGLMYTFGDGRHGKLGLGLENFTNHFIPTLCSNFLRFIVKLV | Sequence: |
| | ACGGCHMVVFAAPHRGVAKEIEFDEINDTCLSVATFLPYSSLTSGNVLQRTLSARMRRR | NP_001030025.1 |
| | ERERSPDSFSMRRTLPPIEGTLGLSACFLPNSVFPRCSERNLQESVLSEQDLMQPEEPD | |
| | YLLDEMTKEAEIDNSSTVESLGETTDILNMTHIMSLNSNEKSLKLSPVQKQKKQQTIGE | |
| | LTQDTALTENDDSDEYEEMSEMKEGKACKQHVSQGIFMTQPATTIEAFSDEEVEIPEEK | |
| | EGAEDSKGNGIEEQEVEANEENVKVHGGRKEKTEILSDDLTDKAEVSEGKAKSVGEAED | |
| | GPEGRGDGTCEEGSSGAEHWQDEEREKGEKDKGRGEMERPGEGEKELAEKEEWKKRDGE | |
| | EQEQKEREQGHQKERNQEMEEGGEEEHGEGEEEEGDREEEEEKEGEGKEEGEGEEVEGE | |
| | REKEEGERKKEERAGKEEKGEEEGDQGEGEEEETEGRGEEKEEGGEVEGGEVEEGKGER | |
| | EEEEEEGEGEEEEGEGEEEEGEGEEEEGEGKGEEEGEEGEGEEEGEEGEGEGEEEEGEG | |
| | EGEEEGEGEGEEEEGEGEGEEEGEGEGEEEEGEGKGEEEGEEGEGEGEEEEGEGEGEDG | |
| | EGEGEEEEGEWEGEEEEGEGEGEEEGEGEGEEGEGEGEEEEGEGEGEEEEGEEEGEEEG | |
| | EGEEEGEGEGEEEEEGEVEGEVEGEEGEGEGEEEEGEEEGEEREKEGEGEENRRNREEE | |
| | EEEEGKYQETGEEENERQDGEEYKKVSKIKGSVKYGKHKTYQKKSVTNTQGNGKEQRSK | |
| | MPVQSKRLLKNGPSGSKKFWNNVLPHYLELK | |
| 45 | ggggggggggggggggggttggccactccctctctgcgcgctcgctcgctcactgaggc | Transfer |
| | cgggcgaccaaaggtcgcccgacgcccgggctttgcccgggggcctcagtgagcgagcg | plasmid - |
| | agcgcgcagagagggagtggccaactccatcactaggggttcctcagatctgaattcgg | pTR-UF11 |
| | tacctagttattaatagtaatcaattacggggtcattagttcatagcccatatatggag | |
| | ttccgcgttacataacttacggtaaatggcccgcctggctgaccgcccaacgacccccg | |
| | cccattgacgtcaataatgacgtatgttcccatagtaacgccaatagggactttccatt | |
| | gacgtcaatgggtggagtatttacggtaaactgcccacttggcagtacatcaagtgtat | |
| | catatgccaagtacgccccctattgacgtcaatgacggtaaatggcccgcctggcatta | |
| | tgcccagtacatgaccttatgggactttcctacttggcagtacatctacgtattagtca | |
| | tcgctattaccatggtcgaggtgagccccacgttctgcttcactctccccatctccccc | |
| | ccctccccacccccaattttgtatttatttattttttaattattttgtgcagcgatggg | |
| | ggcggggggggggggggggcgcgcgccaggcggggggggggggcgaggggggggggggc | |
| | gaggcggagaggtgcggcggcagccaatcagagcggcgcgctccgaaagtttcctttta | |
| | tggcgaggcggcggcggcggcggccctataaaaagcgaagcgcgcggcgggcgggagtc | |
| | gctgcgcgctgccttcgccccgtgccccgctccgccgccgcctcgcgccgcccgccccg | |
| | gctctgactgaccgcgttactcccacaggtgagcggggggacggcccttctcctccggg | |
| | ctgtaattagcgcttggtttaatgacggcttgtttcttttctgtggctgcgtgaaagcc | |
| | ttgaggggctccgggagggccctttgtgcggggggagcggctcggggggtgcgtgcgtg | |
| | tgtgtgtgcgtggggagcgccgcgtgcggctccgcgctgcccggcggctgtgagcgctg | |
| | cgggcgcggcgcggggctttgtgcgctccgcagtgtgcgcgaggggagcgcggccgggg | |
| | gcggtgccccgcggtgcggggggggctgcgaggggaacaaaggctgcgtgcggggtgtg | |
| | tgcgtgggggggtgagcagggggtgtgggcgcgtcggtcgggctgcaaccccccctgca | |
| | cccccctccccgagttgctgagcacggcccggcttcgggtgcggggctccgtacggggc | |
| | gtggcgcggggctcgccgtgccgggcggggggtggcggcaggtgggggtgccgggcggg | |
| | gggggccgcctcgggccggggagggctcggggggggggggcggcccccggagcgccggc | |
| | ggctgtcgaggcgcggcgagccgcagccattgccttttatggtaatcgtgcgagagggc | |
| | gcagggacttcctttgtcccaaatctgtgcggagccgaaatctgggaggcgccgccgca | |
| | ccccctctagcgggcgcggggcgaagcggtgcggcgccggcaggaaggaaatgggcggg | |
| | gagggccttcgtgcgtcgccgcgccgccgtccccttctccctctccagcctcggggctg | |
| | tccgcggggggacggctgccttcgggggggacggggcagggcggggttcggcttctggc | |
| | gtgtgaccggcggctctagagcctctgctaaccatgttcatgccttcttctttttccta | |
| | cagctcctgggcaacgtgctggttattgtgctgtctcatcattttggcaaagaattcct | |
| | cgaagatctaggcctgcaggcggccgccgccaccatgagcaagggcgaggaactgttca | |
| | ctggcgtggtcccaattctcgtggaactggatggcgatgtgaatgggcacaaattttct | |
| | gtcagcggagagggtgaaggtgatgccacatacggaaagctcaccctgaaattcatctg | |
| | caccactggaaagctccctgtgccatggccaacactggtcactaccctgacctatggcg | |
| | tgcagtgcttttccagatacccagaccatatgaagcagcatgactttttcaagagcgcc | |
| | atgcccgagggctatgtgcaggagagaaccatctttttcaaagatgacgggaactacaa | |
| | gacccgcgctgaagtcaagttcgaaggtgacaccctggtgaatagaatcgagctgaagg | |
| | gcattgactttaaggaggatggaaacattctcggccacaagctggaatacaactataac | |
| | tcccacaatgtgtacatcatggccgacaagcaaaagaatggcatcaaggtcaacttcaa | |
| | gatcagacacaacattgaggatggatccgtgcagctggccgaccattatcaacagaaca | |
| | ctccaatcggcgacggccctgtgctcctcccagacaaccattacctgtccacccagtct | |
| | gccctgtctaaagatcccaacgaaaagagagaccacatggtcctgctggagtttgtgac | |
| | cgctgctgggatcacacatggcatggacgagctgtacaagtgagcggccgcgcggatcc | |
| | agacatgataagatacattgatgagtttggacaaaccacaactagaatgcagtgaaaaa | |
| | aatgctttatttgtgaaatttgtgatgctattgctttatttgtaaccattataagctgc | |
| | aataaacaagttaacaacaacaattgcattcattttatgtttcaggttcagggggaggt | |
| | gtgggaggttttttagtcgacctcgagcagtgtggttttgcaagaggaagcaaaaagcc | |
| | tctccacccaggcctggaatgtttccacccaagtcgaaggcagtgtggttttgcaagag | |
| | gaagcaaaaagcctctccacccaggcctggaatgtttccacccaatgtcgagcaacccc | |
| | gcccagcgtcttgtcattggcgaattcgaacacgcagatgcagtcggggggcgcggtcc | |
| | caggtccacttcgcatattaaggtgacgcgtgtggcctcgaacaccgagcgaccctgca | |
| | gccaatatgggatcggccattgaacaagatggattgcacgcaggttctccggccgcttg | |
| | ggtggagaggctattcggctatgactgggcacaacagacaatcggctgctctgatgccg | |
| | ccgtgttccggctgtcagcgcaggggcgcccggttctttttgtcaagaccgacctgtcc | |
| | ggtgccctgaatgaactgcaggacgaggcagcgcggctatcgtggctggccacgacggg | |
| | cgttccttgcgcagctgtgctcgacgttgtcactgaagcgggaagggactggctgctat | |
| | tgggcgaagtgccggggcaggatctcctgtcatctcaccttgctcctgccgagaaagta | |
| | tccatcatggctgatgcaatgcggcggctgcatacgcttgatccggctacctgcccatt | |
| | cgaccaccaagcgaaacatcgcatcgagcgagcacgtactcggatggaagccggtcttg | |
| | tcgatcaggatgatctggacgaagagcatcaggggctcgcgccagccgaactgttcgcc | |
| | aggctcaaggcgcgcatgcccgacggcgaggatctcgtcgtgacccatggcgatgcctg | |
| | cttgccgaatatcatggtggaaaatggccgcttttctggattcatcgactgtggccggc | |
| | tgggtgtggcggaccgctatcaggacatagcgttggctacccgtgatattgctgaagag | |
| | cttggcggcgaatgggctgaccgcttcctcgtgctttacggtatcgccgctcccgattc | |
| | gcagcgcatcgccttctatcgccttcttgacgagttcttctgaggggatccgtcgacta | |
| | gagctcgctgatcagcctcgactgtgccttctagttgccagccatctgttgtttgcccc | |
| | tcccccgtgccttccttgaccctggaaggtgccactcccactgtcctttcctaataaaa | |
| | tgaggaaattgcatcgcattgtctgagtaggtgtcattctattctggggggtggggggg | |
| | gcaggacagcaagggggaggattgggaagacaatagcaggcatgctggggagagatctg | |
| | aggaacccctagtgatggagttggccactccctctctgcgcgctcgctcgctcactgag | |
| | gccgcccgggcaaagcccgggcgtcgggcgacctttggtcgcccggcctcagtgagcga | |
| | gcgagcgcgcagagagggagtggccaacccccccccccccccccctgcagccctgcatt | |
| | aatgaatcggccaacgcgcggggagaggcggtttgcgtattgggcgctcttccgcttcc | |
| | tcgctcactgactcgctgcgctcggtcgttcggctgcggcgagcggtatcagctcactc | |
| | aaaggcggtaatacggttatccacagaatcaggggataacgcaggaaagaacatgtgag | |
| | caaaaggccagcaaaaggccaggaaccgtaaaaaggccgcgttgctggcgtttttccat | |
| | aggctccgcccccctgacgagcatcacaaaaatcgacgctcaagtcagaggtggcgaaa | |
| | cccgacaggactataaagataccaggcgtttccccctggaagctccctcgtgcgctctc | |
| | ctgttccgaccctgccgcttaccggatacctgtccgcctttctcccttcgggaagcgtg | |
| | gcgctttctcaatgctcacgctgtaggtatctcagttcggtgtaggtcgttcgctccaa | |
| | gctgggctgtgtgcacgaaccccccgttcagcccgaccgctgcgccttatccggtaact | |
| | atcgtcttgagtccaacccggtaagacacgacttatcgccactggcagcagccactggt | |
| | aacaggattagcagagcgaggtatgtaggcggtgctacagagttcttgaagtggtggcc | |
| | taactacggctacactagaaggacagtatttggtatctgcgctctgctgaagccagtta | |
| | ccttcggaaaaagagttggtagctcttgatccggcaaacaaaccaccgctggtagcggt | |
| | ggtttttttgtttgcaagcagcagattacgcgcagaaaaaaaggatctcaagaagatcc | |
| | tttgatcttttctacggggtctgacgctcagtggaacgaaaactcacgttaagggattt | |
| | tggtcatgagattatcaaaaaggatcttcacctagatccttttaaattaaaaatgaagt | |
| | tttaaatcaatctaaagtatatatgagtaaacttggtctgacagttaccaatgcttaat | |
| | cagtgaggcacctatctcagcgatctgtctatttcgttcatccatagttgcctgactcc | |
| | ccgtcgtgtagataactacgatacgggagggcttaccatctggccccagtgctgcaatg | |
| | ataccgcgagacccacgctcaccggctccagatttatcagcaataaaccagccagccgg | |
| | aagggccgagcgcagaagtggtcctgcaactttatccgcctccatccagtctattaatt | |
| | gttgccgggaagctagagtaagtagttcgccagttaatagtttgcgcaacgttgttgcc | |
| | attgctacaggcatcgtggtgtcacgctcgtcgtttggtatggcttcattcagctccgg | |
| | ttcccaacgatcaaggcgagttacatgatcccccatgttgtgcaaaaaagcggttagct | |
| | ccttcggtcctccgatcgttgtcagaagtaagttggccgcagtgttatcactcatggtt | |
| | atggcagcactgcataattctcttactgtcatgccatccgtaagatgcttttctgtgac | |
| | tggtgagtactcaaccaagtcattctgagaatagtgtatgcggcgaccgagttgctctt | |
| | gcccggcgtcaatacgggataataccgcgccacatagcagaactttaaaagtgctcatc | |
| | attggaaaacgttcttcggggcgaaaactctcaaggatcttaccgctgttgagatccag | |
| | ttcgatgtaacccactcgtgcacccaactgatcttcagcatcttttactttcaccagcg | |
| | tttctgggtgagcaaaaacaggaaggcaaaatgccgcaaaaaagggaataagggcgaca | |
| | cggaaatgttgaatactcatactcttcctttttcaatattattgaagcatttatcaggg | |
| | ttattgtctcatgagcggatacatatttgaatgtatttagaaaaataaacaaatagggg | |
| | ttccgcgcacatttccccgaaaagtgccacctgacgtctaagaaaccattattatcatg | |
| | acattaacctataaaaataggcgtatcacgaggccctttcgtctcgcgcgtttcggtga | |
| | tgacggtgaaaacctctgacacatgcagctcccggagacggtcacagcttgtctgtaag | |
| | cggatgccgggagcagacaagcccgtcagggcgcgtcagcgggtgttggcgggtgtcgg | |
| | ggctggcttaactatgcggcatcagagcagattgtactgagagtgcaccatatgcggtg | |
| | tgaaataccgcacagatgcgtaaggagaaaataccgcatcaggaaattgtaaacgttaa | |
| | tattttgttaaaattcgcgttaaatttttgttaaatcagctcattttttaaccaatagg | |
| | ccgaaatcggcaaaatcccttataaatcaaaagaatagaccgagatagggttgagtgtt | |
| | gttccagtttggaacaagagtccactattaaagaacgtggactccaacgtcaaagggcg | |
| | aaaaaccgtctatcagggcgatggcccactacgtgaaccatcaccctaatcaagttttt | |
| | tggggtcgaggtgccgtaaagcactaaatcggaaccctaaagggagcccccgatttaga | |
| | gcttgacggggaaagccggcgaacgtggcgagaaaggaagggaagaaagcgaaaggagc | |
| | gggcgctagggcgctggcaagtgtagcggtcacgctgcgcgtaaccaccacacccgccg | |
| | cgcttaatgcgccgctacagggcgcgtcgcgccattcgccattcaggctacgcaactgt | |
| | tgggaagggcgatcggtgcgggcctcttcgctattacgccaggctgca | |
| 46 | aattcccatcatcaataatataccttattttggattgaagccaatatgataatgagggg | Rep/Cap |
| | gtggagtttgtgacgtggcgcggggcgtgggaacggggcgggtgacgtagtagtctcta | plasmid - |
| | gagtcctgtattagaggtcacgtgagtgttttgcgacattttgcgacaccatgtggtca | pACG2 |
| | cgctgggtatttaagcccgagtgagcacgcagggtctccattttgaagcgggaggtttg | |
| | aacgcgcagccaccacggcggggttttacgagattgtgattaaggtccccagcgacctt | |
| | gacgggcatctgcccggcatttctgacagctttgtgaactgggtggccgagaaggaatg | |
| | ggagttgccgccagattctgacatggatctgaatctgattgagcaggcacccctgaccg | |
| | tggccgagaagctgcagcgcgactttctgacggaatggcgccgtgtgagtaaggccccg | |
| | gaggcccttttctttgtgcaatttgagaagggagagagctacttccacatgcacgtgct | |
| | cgtggaaaccaccggggtgaaatccatggttttgggacgtttcctgagtcagattcgcg | |
| | aaaaactgattcagagaatttaccggggatcgagccgactttgccaaactggttcgcgg | |
| | tcacaaagaccagaaatggcgccggagggggaacaaggtggtggatgagtgctacatcc | |
| | ccaattacttgctccccaaaacccagcctgagctccagtgggcgtggactaatatggaa | |
| | cagtatttaagcgcctgtttgaatctcacggagcgtaaacggttggtggcgcagcatct | |
| | gacgcacgtgtcgcagacgcaggagcagaacaaagagaatcagaatcccaattctgatg | |
| | cgccggtgatcagatcaaaaacttcagccaggtacatggagctggtcgggtggctcgtg | |
| | gacaaggggattacctcggagaagcagtggatccaggaggaccaggcctcatacatctc | |
| | cttcaatgcggcctccaactcgcggtcccaaatcaaggctgccttggacaatgcgggaa | |
| | agattatgagcctgactaaaaccgcccccgactacctggtgggccagcagcccgtggag | |
| | gacatttccagcaatcggatttataaaattttggaactaaacgggtacgatccccaata | |
| | tgcggcttccgtctttctgggatgggccacgaaaaagttcggcaagaggaacaccatct | |
| | ggctgtttgggcctgcaactaccgggaagaccaacatcgcggaggccatagcccacact | |
| | gtgcccttctacgggtgcgtaaactggaccaatgagaactttcccttcaacgactgtgt | |
| | cgacaagatggtgatctggtgggaggaggggaagatgaccgccaaggtcgtggagtcgg | |
| | ccaaagccattctcggaggaagcaaggtgcgcgtggaccagaaatgcaagtcctcggcc | |
| | cagatagacccgactcccgtgatcgtcacctccaacaccaacatgtgcgccgtgattga | |
| | cgggaactcaacgaccttcgaacaccagcagccgttgcaagaccggatgttcaaatttg | |
| | aactcacccgccgtctggatcatgactttgggaaggtcaccaagcaggaagtcaaagac | |
| | tttttccggtgggcaaaggatcacgtggttgaggtggagcatgaattctacgtcaaaaa | |
| | gggtggagccaagaaaagacccgcccccagtgacgcagatataagtgagcccaaacggg | |
| | tgcgcgagtcagttgcgcagccatcgacgtcagacgcggaagcttcgatcaactacgca | |
| | gacaggtaccaaaacaaatgttctcgtcacgtgggcatgaatctgatgctgtttccctg | |
| | cagacaatgcgagagaatgaatcagaattcaaatatctgcttcactcacggacagaaag | |
| | actgtttagagtgctttcccgtgtcagaatctcaacccgtttctgtcgtcaaaaaggcg | |
| | tatcagaaactgtgctacattcatcatatcatgggaaaggtgccagacgcttgcactgc | |
| | ctgcgatctggtcaatgtggatttggatgactgcatctttgaacaataaatgatttaaa | |
| | tcaggtatggctgccgatggttatcttccagattggctcgaggacactctctctgaagg | |
| | aataagacagtggtggaagctcaaacctggcccaccaccaccaaagcccgcagagcggc | |
| | ataaggacgacagcaggggtcttgtgcttcctgggtacaagtacctcggacccttcaac | |
| | ggactcgacaagggagagccggtcaacgaggcagacgccgcggccctcgagcacgacaa | |
| | agcctacgaccggcagctcgacagcggagacaacccgtacctcaagtacaaccacgccg | |
| | acgcggagtttcaggagcgccttaaagaagatacgtcttttgggggcaacctcggacga | |
| | gcagtcttccaggcgaaaaagagggttcttgaacctctgggcctggttgaggaacctgt | |
| | taagacggctccgggaaaaaagaggccggtagagcactctcctgtggagccagactcct | |
| | cctcgggaaccggaaaggcgggccagcagcctgcaagaaaaagattgaattttggtcag | |
| | actggagacgcagactcagtacctgacccccagcctctcggacagccaccagcagcccc | |
| | ctctggtctgggaactaatacgatggctacaggcagtggcgcaccaatggcagacaata | |
| | acgagggcgccgacggagtgggtaattcctcgggaaattggcattgcgattccacatgg | |
| | atgggcgacagagtcatcaccaccagcacccgaacctgggccctgcccacctacaacaa | |
| | ccacctctacaaacaaatttccagccaatcaggagcctcgaacgacaatcactactttg | |
| | gctacagcaccccttgggggtattttgacttcaacagattccactgccacttttcacca | |
| | cgtgactggcaaagactcatcaacaacaactggggattccgacccaagagactcaactt | |
| | caagctctttaacattcaagtcaaagaggtcacgcagaatgacggtacgacgacgattg | |
| | ccaataaccttaccagcacggttcaggtgtttactgactcggagtaccagctcccgtac | |
| | gtcctcggctcggcgcatcaaggatgcctcccgccgttcccagcagacgtcttcatggt | |
| | gccacagtatggatacctcaccctgaacaacgggagtcaggcagtaggacgctcttcat | |
| | tttactgcctggagtactttccttctcagatgctgcgtaccggaaacaactttaccttc | |
| | agctacacttttgaggacgttcctttccacagcagctacgctcacagccagagtctgga | |
| | ccgtctcatgaatcctctcatcgaccagtacctgtattacttgagcagaacaaacactc | |
| | caagtggaaccaccacgcagtcaaggcttcagttttctcaggccggagcgagtgacatt | |
| | cgggaccagtctaggaactggcttcctggaccctgttaccgccagcagcgagtatcaaa | |
| | gacatctgcggataacaacaacagtgaatactcgtggactggagctaccaagtaccacc | |
| | tcaatggcagagactctctggtgaatccgggcccggccatggcaagccacaaggacgat | |
| | gaagaaaagttttttcctcagagcggggttctcatctttgggaagcaaggctcagagaa | |
| | aacaaatgtggacattgaaaaggtcatgattacagacgaagaggaaatcaggacaacca | |
| | atcccgtggctacggagcagtatggttctgtatctaccaacctccagagaggcaacaga | |
| | caagcagctaccgcagatgtcaacacacaaggcgttcttccaggcatggtctggcagga | |
| | cagagatgtgtaccttcaggggcccatctgggcaaagattccacacacggacggacatt | |
| | ttcacccctctcccctcatgggggattcggacttaaacaccctcctccacagattctca | |
| | tcaagaacaccccggtacctgcgaatccttcgaccaccttcagtgcggcaaagtttgct | |
| | tccttcatcacacagtactccacgggacaggtcagcgtggagatcgagtgggagctgca | |
| | gaaggaaaacagcaaacgctggaatcccgaaattcagtacacttccaactacaacaagt | |
| | ctgttaatgtggactttactgtggacactaatggcgtgtattcagagcctcgccccatt | |
| | ggcaccagatacctgactcgtaatctgtaattgcttgttaatcaataaaccgtttaatt | |
| | cgtttcagttgaactttggtctctgcgtatttctttcttatctagtttccatgctctag | |
| | actactacgtcacccgccccgttcccacgccccgcgccacgtcacaaactccaccccct | |
| | cattatcatattggcttcaatccaaaataaggtatattattgatgatgcatcgctggcg | |
| | taatagcgaagaggcccgcaccgatcgcccttcccaacagttgcgcagcctgaatggcg | |
| | aatggaattccagacgattgagcgtcaaaatgtaggtatttccatgagcgtttttcctg | |
| | ttgcaatggctggcggtaatattgttctggatattaccagcaaggccgatagtttgagt | |
| | tcttctactcaggcaagtgatgttattactaatcaaagaagtattgcgacaacggttaa | |
| | tttgcgtgatggacagactcttttactcggtggcctcactgattataaaaacacttctc | |
| | aggattctggcgtaccgttcctgtctaaaatccctttaatcggcctcctgtttagctcc | |
| | cgctctgattctaacgaggaaagcacgttatacgtgctcgtcaaagcaaccatagtacg | |
| | cgccctgtagcggcgcattaagcgcggcgggtgtggtggttacgcgcagcgtgaccgct | |
| | acacttgccagcgccctagcgcccgctcctttcgctttcttcccttcctttctcgccac | |
| | gttcgccggctttccccgtcaagctctaaatcgggggctccctttagggttccgattta | |
| | gtgctttacggcacctcgaccccaaaaaacttgattagggtgatggttcacgtagtggg | |
| | ccatcgccctgatagacggtttttcgccctttgacgttggagtccacgttctttaatag | |
| | tggactcttgttccaaactggaacaacactcaaccctatctcggtctattcttttgatt | |
| | tataagggattttgccgatttcggcctattggttaaaaaatgagctgatttaacaaaaa | |
| | tttaacgcgaattttaacaaaatattaacgtttacaatttaaatatttgcttatacaat | |
| | cttcctgtttttggggcttttctgattatcaaccggggtacatatgattgacatgctag | |
| | ttttacgattaccgttcatcgattctcttgtttgctccagactctcaggcaatgacctg | |
| | atagcctttgtagagacctctcaaaaatagctaccctctccggcatgaatttatcagct | |
| | agaacggttgaatatcatattgatggtgatttgactgtctccggcctttctcacccgtt | |
| | tgaatctttacctacacattactcaggcattgcatttaaaatatatgagggttctaaaa | |
| | atttttatccttgcgttgaaataaaggcttctcccgcaaaagtattacagggtcataat | |
| | gtttttggtacaaccgatttagctttatgctctgaggctttattgcttaattttgctaa | |
| | ttctttgccttgcctgtatgatttattggatgttggaattcctgatgcggtattttctc | |
| | cttacgcatctgtgcggtatttcacaccgcatatggtgcactctcagtacaatctgctc | |
| | tgatgccgcatagttaagccagccccgacacccgccaacacccgctgacgcgccctgac | |
| | gggcttgtctgctcccggcatccgcttacagacaagctgtgaccgtctccgggagctgc | |
| | atgtgtcagaggttttcaccgtcatcaccgaaacgcgcgagacgaaagggcctcgtgat | |
| | acgcctatttttataggttaatgtcatgataataatggtttcttagacgtcaggtggca | |
| | cttttcggggaaatgtgcgcggaacccctatttgtttatttttctaaatacattcaaat | |
| | atgtatccgctcatgagacaataaccctgataaatgcttcaataatattgaaaaaggaa | |
| | gagtatgagtattcaacatttccgtgtcgcccttattcccttttttgcggcattttgcc | |
| | ttcctgtttttgctcacccagaaacgctggtgaaagtaaaagatgctgaagatcagttg | |
| | ggtgcacgagtgggttacatcgaactggatctcaacagcggtaagatccttgagagttt | |
| | tcgccccgaagaacgttttccaatgatgagcacttttaaagttctgctatgtggcgcgg | |
| | tattatcccgtattgacgccgggcaagagcaactcggtcgccgcatacactattctcag | |
| | aatgacttggttgagtactcaccagtcacagaaaagcatcttacggatggcatgacagt | |
| | aagagaattatgcagtgctgccataaccatgagtgataacactgcggccaacttacttc | |
| | tgacaacgatcggaggaccgaaggagctaaccgcttttttgcacaacatgggggatcat | |
| | gtaactcgccttgatcgttgggaaccggagctgaatgaagccataccaaacgacgagcg | |
| | tgacaccacgatgcctgtagcaatggcaacaacgttgcgcaaactattaactggcgaac | |
| | tacttactctagcttcccggcaacaattaatagactggatggaggcggataaagttgca | |
| | ggaccacttctgcgctcggcccttccggctggctggtttattgctgataaatctggagc | |
| | cggtgagcgtgggtctcgcggtatcattgcagcactggggccagatggtaagccctccc | |
| | gtatcgtagttatctacacgacggggagtcaggcaactatggatgaacgaaatagacag | |
| | atcgctgagataggtgcctcactgattaagcattggtaactgtcagaccaagtttactc | |
| | atatatactttagattgatttaaaacttcatttttaatttaaaaggatctaggtgaaga | |
| | tcctttttgataatctcatgaccaaaatcccttaacgtgagttttcgttccactgagcg | |
| | tcagaccccgtagaaaagatcaaaggatcttcttgagatcctttttttctgcgcgtaat | |
| | ctgctgcttgcaaacaaaaaaaccaccgctaccagcggtggtttgtttgccggatcaag | |
| | agctaccaactctttttccgaaggtaactggcttcagcagagcgcagataccaaatact | |
| | gtccttctagtgtagccgtagttaggccaccacttcaagaactctgtagcaccgcctac | |
| | atacctcgctctgctaatcctgttaccagtggctgctgccagtggcgataagtcgtgtc | |
| | ttaccgggttggactcaagacgatagttaccggataaggcgcagcggtcgggctgaacg | |
| | gggggttcgtgcacacagcccagcttggagcgaacgacctacaccgaactgagatacct | |
| | acagcgtgagctatgagaaagcgccacgcttcccgaagggagaaaggcggacaggtatc | |
| | cggtaagcggcagggtcggaacaggagagcgcacgagggagcttccagggggaaacgcc | |
| | tggtatctttatagtcctgtcgggtttcgccacctctgacttgagcgtcgatttttgtg | |
| | atgctcgtcaggggggggagcctatggaaaaacgccagcaacgcggcctttttacggtt | |
| | cctggccttttgctggccttttgctcacatgttctttcctgcgttatcccctgattctg | |
| | tggataaccgtattaccgcctttgagtgagctgataccgctcgccgcagccgaacgacc | |
| | gagcgcagcgagtcagtgagcgaggaagcggaagagcgcccaatacgcaaaccgcctct | |
| | ccccgcgcgttggccgattcattaatgcag | |
| 47 | tcttccgcttcctcgctcactgactcgctgcgctcggtcgttcggctgcggcgagcggt | Helper |
| | atcagctcactcaaaggcggtaatacggttatccacagaatcaggggataacgcaggaa | plasmid - |
| | agaacatgtgagcaaaaggccagcaaaaggccaggaaccgtaaaaaggccgcgttgctg | pALD-X80 |
| | gcgtttttccataggctccgcccccctgacgagcatcacaaaaatcgacgctcaagtca | |
| | gaggtggcgaaacccgacaggactataaagataccaggcgtttccccctggaagctccc | |
| | tcgtgcgctctcctgttccgaccctgccgcttaccggatacctgtccgcctttctccct | |
| | tcgggaagcgtggcgctttctcatagctcacgctgtaggtatctcagttcggtgtaggt | |
| | cgttcgctccaagctgggctgtgtgcacgaaccccccgttcagcccgaccgctgcgcct | |
| | tatccggtaactatcgtcttgagtccaacccggtaagacacgacttatcgccactggca | |
| | gcagccactggtaacaggattagcagagcgaggtatgtaggcggtgctacagagttctt | |
| | gaagtggtggcctaactacggctacactagaagaacagtatttggtatctgcgctctgc | |
| | tgaagccagttaccttcggaaaaagagttggtagctcttgatccggcaaacaaaccacc | |
| | gctggtagcggtggtttttttgtttgcaagcagcagattacgcgcagaaaaaaaggatc | |
| | tcaagaagatcctttgatcttttctacggggtctgacgctcagtggaacgaaaactcac | |
| | gttaagggattttggtcatgagattatcaaaaaggatcttcacctagatccttttaaat | |
| | taaaaatgaagttttaaatcaatctaaagtatatatgagtaaacttggtctgacagtta | |
| | ttagaaaaactcatcgagcatcaaatgaaactgcaatttattcatatcaggattatcaa | |
| | taccatatttttgaaaaagccgtttctgtaatgaaggagaaaactcaccgaggcagttc | |
| | cataggatggcaagatcctggtatcggtctgcgattccgactcgtccaacatcaataca | |
| | acctattaatttcccctcgtcaaaaataaggttatcaagtgagaaatcaccatgagtga | |
| | cgactgaatccggtgagaatggcaaaagtttatgcatttctttccagacttgttcaaca | |
| | ggccagccattacgctcgtcatcaaaatcactcgcatcaaccaaaccgttattcattcg | |
| | tgattgcgcctgagcgagacgaaatacgcgatcgctgttaaaaggacaattacaaacag | |
| | gaatcgaatgcaaccggcgcaggaacactgccagcgcatcaacaatattttcacctgaa | |
| | tcaggatattcttctaatacctggaatgctgtttttccggggatcgcagtggtgagtaa | |
| | ccatgcatcatcaggagtacggataaaatgcttgatggtcggaagaggcataaattccg | |
| | tcagccagtttagtctgaccatctcatctgtaacatcattggcaacgctacctttgcca | |
| | tgtttcagaaacaactctggcgcatcgggcttcccatacaagcgatagattgtcgcacc | |
| | tgattgcccgacattatcgcgagcccatttatacccatataaatcagcatccatgttgg | |
| | aatttaatcgcggcctcgacgtttcccgttgaatatggctcatactcttcctttttcaa | |
| | tattattgaagcatttatcagggttattgtctcatgagcggatacatatttgaatgtat | |
| | ttagaaaaataaacaaataggggttccgcgcacatttccccgaaaagtgccacctgacg | |
| | tctaagaaaccattattatcatgacattaacctataaaaataggcgtatcacgaggccc | |
| | tttcgtctcgcgcgtttcggtgatgacggtgaaaacctctgacacatgcagctcccgga | |
| | gacggtcacagcttgtctgtaagcggatgccgggagcagacaagcccgtcagggcgcgt | |
| | cagcgggtgttgggggtgtcggggctggcttaactatgcggcatcagagcagattgtac | |
| | tgagagtgcaccataaaattgtaaacgttaatattttgttaaaattcgcgttaaatttt | |
| | tgttaaatcagctcattttttaaccaataggccgaaatcggcaaaatcccttataaatc | |
| | aaaagaatagcccgagatagggttgagtgttgttccagtttggaacaagagtccactat | |
| | taaagaacgtggactccaacgtcaaagggcgaaaaaccgtctatcagggcgatggccca | |
| | ctacgtgaaccatcacccaaatcaagttttttggggtcgaggtgccgtaaagcactaaa | |
| | tcggaaccctaaagggagcccccgatttagagcttgacggggaaagccggcgaacgtgg | |
| | cgagaaaggaagggaagaaagcgaaaggagcgggcgctagggcgctggcaagtgtagcg | |
| | gtcacgctgcgcgtaaccaccacacccgccgcgcttaatgcgccgctacagggcgcgta | |
| | ctatggttgctttgacgtatgcggtgtgaaataccgcacagatgcgtaaggagaaaata | |
| | ccgcatcaggcgccattcgccattcaggctgcgcaactgttgggaagggcgatcggtgc | |
| | gggcctcttcgctattacgccagctggcgaaagggggatgtgctgcaaggcgattaagt | |
| | tgggtaacgccagggttttcccagtcacgacgttgtaaaacgacggccagtgccaagct | |
| | taaggtgcacggcccacgtggccactagtacttctcgacagaagcaccatgtccttggg | |
| | tccggcctgctgaatgcgcaggcggtcggccatgccccaggcttcgttttgacatcggc | |
| | gcaggtctttgtagtagtcttgcatgagcctttctaccggcacttcttcttctccttcc | |
| | tcttgtcctgcatctcttgcatctatcgctgcggcggcggcggagtttggccgtaggtg | |
| | gcgccctcttcctcccatgcgtgtgaccccgaagcccctcatcggctgaagcagggcta | |
| | ggtcggcgacaacgcgctcggctaatatggcctgctgcacctgcgtgagggtagactgg | |
| | aagtcatccatgtccacaaagcggtggtatgcgcccgtgttgatggtgtaagtgcagtt | |
| | ggccataacggaccagttaacggtctggtgacccggctgcgagagctcggtgtacctga | |
| | gacgcgagtaagccctcgagtcaaatacgtagtcgttgcaagtccgcaccaggtactgg | |
| | tatcccaccaaaaagtgcggcggcggctggcggtagaggggccagcgtagggtggccgg | |
| | ggctccgggggcgagatcttccaacataaggcgatgatatccgtagatgtacctggaca | |
| | tccaggtgatgccggcggcggtggtggaggcgcgcggaaagtcgcggacgcggttccag | |
| | atgttgcgcagcggcaaaaagtgctccatggtcgggacgctctggccggtcaggcgcgc | |
| | gcaatcgttgacgctctagcgtgcaaaaggagagcctgtaagcgggcactcttccgtgg | |
| | tctggtggataaattcgcaagggtatcatggcggacgaccggggttcgagccccgtatc | |
| | cggccgtccgccgtgatccatgcggttaccgcccgcgtgtcgaacccaggtgtgcgacg | |
| | tcagacaacgggggagtgctccttttggcttccttccaggcgcggcggctgctgcgcta | |
| | gcttttttggccactggccgcgcgcagcgtaagcggttaggctggaaagcgaaagcatt | |
| | aagtggctcgctccctgtagccggagggttattttccaagggttgagtcgcgggacccc | |
| | cggttcgagtctcggaccggccggactgcggcgaacgggggtttgcctccccgtcatgc | |
| | aagaccccgcttgcaaattcctccggaaacagggacgagccccttttttgcttttccca | |
| | gatgcatccggtgctgcggcagatgcgcccccctcctcagcagcggcaagagcaagagc | |
| | agcggcagacatgcagggcaccctcccctcctcctaccgcgtcaggaggggcgacatcc | |
| | gcggttgacgcggcagcagatggtgattacgaacccccgcggcgccgggcccggcacta | |
| | cctggacttggaggagggcgagggcctggcgcggctaggagcgccctctcctgagggca | |
| | cccaagggtgcagctgaagcgtgatacgcgtgaggcgtacgtgccgcggcagaacctgt | |
| | ttcgcgaccgcgagggagaggagcccgaggagatgcgggatcgaaagttccacgcaggg | |
| | cgcgagctgcggcatggcctgaatcgcgagcggttgctgcgcgaggaggactttgagcc | |
| | cgacgcgcgaaccgggattagtcccgcgcgcgcacacgtggcggccgccgacctggtaa | |
| | ccgcatacgagcagacggtgaaccaggagattaactttcaaaaaagctttaacaaccac | |
| | gtgcgtacgcttgtggcgcgcgaggaggtggctataggactgatgcatctgtgggactt | |
| | tgtaagcgcgctggagcaaaacccaaatagcaagccgctcatggcgcagctgttcctta | |
| | tagtgcagcacagcagggacaacgaggcattcagggatgcgctgctaaacatagtagag | |
| | cccgagggccgctggctgctcgatttgataaacatcctgcagagcatagtggtgcagga | |
| | gcgcagcttgagcctggctgacaaggtggccgccatcaactattccatgcttagcctgg | |
| | gcaagttttacgcccgcaagatataccataccccttacgttcccatagacaaggaggta | |
| | aagatcgaggggttctacatgcgcatggcgctgaaggtgcttaccttgagcgacgacct | |
| | gggcgtttatcgcaacgagcgcatccacaaggccgtgagcgtgagccggcggcgcgagc | |
| | tcagcgaccgcgagctgatgcacagcctgcaaagggccctggctggcacgggcagcggc | |
| | gatagagaggccgagtcctactttgacggggcgctgacctgcgctgggccccaagccga | |
| | cgcgccctggaggcagctggggccggacctgggctggcggtggcacccgcgcgcgctgg | |
| | caacgtcggcggcgtggaggaatatgacgaggacgatgagtacgagccagaggacggcg | |
| | agtactaagcggtgatgtttctgatcagatgatgcaagacgcaacggacccggcggtgc | |
| | gggcggcgctgcagagccagccgtccggccttaactccacggacgactggcgccaggtc | |
| | atggaccgcatcatgtcgctgactgcgcgcaatcctgacgcgttccggcagagccgcag | |
| | gccaaccggctctccgcaattctggaagcggtggtcccggcgcgcgcaaaccccacgca | |
| | cgagaaggtgctggcgatcgtaaacgcgctggccgaaaacagggccatccggcccgacg | |
| | aggccggcctggtctacgacgcgctgcttcagcgcgtggctcgttacaacagcggcaac | |
| | gtgcagaccaacctggaccggctggtgggggatgtgcgcgaggccgtggcgcagcgtga | |
| | gcgcgcgcagcagcagggcaacctgggctccatggttgcactaaacgccttcctgagta | |
| | cacagcccgccaacgtgccgcggggacaggaggactacaccaactttgtgagcgcactg | |
| | cggctaatggtgactgagacaccgcaaagtgaggtgtaccagtctgggccagactattt | |
| | tttccagaccagtagacaaggcctgcagaccgtaaacctgagccaggctttcaaaaact | |
| | tgcaggggctgtggggggtgcgggctcccacaggcgaccgcgcgaccgtgtctagcttg | |
| | ctgacgcccaactcgcgcctgttgctgctgctaatagcgcccttcacggacagtggcag | |
| | cgtgtcccgggacacatacctaggtcacttgctgacactgtaccgcgaggccataggtc | |
| | aggcgcatgtggacgagcatactttccaggagattacaagtgtcagccgcgcgctgggg | |
| | caggaggacacgggcagcctggaggcaaccctaaactacctgctgaccaaccggcggca | |
| | gaagatcccctcgttgcacagtttgcaccctttggcgcatcccattctccagtaacttt | |
| | atgtccatgggcgcactcacagacctgggccaaaaccttctctacgccaactccgccca | |
| | cgcgctagacatgacttttgaggtggatcccatggacgagcccacccttctttatgttt | |
| | tgtttgaagtctttgacgtggtccgtgtgcaccagccgcaccgcggcgtcatcgaaacc | |
| | gtgtacctgcgcacgcccttctcggccggcaacgccacaacataaagaagcaagcaaca | |
| | tcaacaacagctgccgccatgggctccagtgagcaggaactgaaagccattgtcaaaga | |
| | tcttggttgtgggccatattttttgggcacctatgacaagcgctttccaggctttgttt | |
| | ctccacacaagctcgcctgcgccatagtcaatacggccggtcgcgagactgggggcgta | |
| | cactggatggcctttgcctggaacccgcactcaaaaacatgctacctctttgagccctt | |
| | tggcttttctgaccagcgactcaagcaggtttaccagtttgagtacgagtcactcctgc | |
| | gccgtagcgccattgcttcttcccccgaccgctgtataacgctggaaaagtccacccaa | |
| | agcgtacaggggcccaactcggccgcctgtggactattctgctgcatgtttctccacgc | |
| | ctttgccaactggccccaaactcccatggatcacaaccccaccatgaaccttattaccg | |
| | gggtacccaactccatgctcaacagtccccaggtacagcccaccctgcgtcgcaaccag | |
| | gaacagctctacagcttcctggagcgccactcgccctacttccgcagccacagtgcgca | |
| | gattaggagcgccacttctttttgtcacttgaaaaacatgtaaaaataatgtactagag | |
| | acactttcaataaaggcaaatgcttttatttgtacactctcgggtgattatttaccccc | |
| | acccttgccgtctgcgccgtttaaaaatcaaaggggttctgccgcgcatcgctatgcgc | |
| | cactggcagggacacgttgcgatactggtgtttagtgctccacttaaactcaggcacaa | |
| | ccatccgcggcagctcggtgaagttttcactccacaggctgcgcaccatcaccaacgcg | |
| | tttagcaggtcgggcgccgatatcttgaagtcgcagttggggcctccgccctgcgcgcg | |
| | cgagttgcgatacacagggttgcagcactggaacactatcagcgccgggtggtgcacgc | |
| | tggccagcacgctcttgtcggagatcagatccgcgtccaggtcctccgcgttgctcagg | |
| | gcgaacggagtcaactttggtagctgccttcccaaaaagggcgcgtgcccaggctttga | |
| | gttgcactcgcaccgtagtggcatcaaaaggtgaccgtgcccggtctgggcgttaggat | |
| | acagcgcctgcataaaagccttgatctgcttaaaagccacctgagcctttgcgccttca | |
| | gagaagaacatgccgcaagacttgccggaaaactgattggccggacaggccgcgtcgtg | |
| | cacgcagcaccttgcgtcggtgttggagatctgcaccacatttcggccccaccggttct | |
| | tcacgatcttggccttgctagactgctccttcagcgcgcgctgcccgttttcgctcgtc | |
| | acatccatttcaatcacgtgctccttatttatcataatgcttccgtgtagacacttaag | |
| | ctcgccttcgatctcagcgcagcggtgcagccacaacgcgcagcccgtgggctcgtgat | |
| | gcttgtaggtcacctctgcaaacgactgcaggtacgcctgcaggaatcgccccatcatc | |
| | gtcacaaaggtcttgttgctggtgaaggtcagctgcaacccgcggtgctcctcgttcag | |
| | ccaggtcttgcatacggccgccagagcttccacttggtcaggcagtagtttgaagttcg | |
| | cctttagatcgttatccacgtggtacttgtccatcagcgcgcgcgcagcctccatgccc | |
| | ttctcccacgcagacacgatcggcacactcagcgggttcatcaccgtaatttcactttc | |
| | cgcttcgctgggctcttcctcttcctcttgcgtccgcataccacgcgccactgggtcgt | |
| | cttcattcagccgccgcactgtgcgcttacctcctttgccatgcttgattagcaccggt | |
| | gggttgctgaaacccaccatttgtagcgccacatcttctctttcttcctcgctgtccac | |
| | gattacctctggtgatgggggcgctcgggcttgggagaagggcgcttctttttcttctt | |
| | gggcgcaatggccaaatccgccgccgaggtcgatggccgcgggctgggtgtgcgcggca | |
| | ccagcgcgtcttgtgatgagtcttcctcgtcctcggactcgatacgccgcctcatccgc | |
| | ttttttgggggcgcccggggaggcggggcgacggggacggggacgacacgtcctccatg | |
| | gttgggggacgtcgcgccgcaccgcgtccgcgctcgggggtggtttcgcgctgctcctc | |
| | ttcccgactggccatttccttctcctataggcagaaaaagatcatggagtcagtcgaga | |
| | agaaggacagcctaaccgccccctctgagttcgccaccaccgcctccaccgatgccgcc | |
| | aacgcgcctaccaccttccccgtcgaggcacccccgcttgaggaggaggaagtgattat | |
| | cgagcaggacccaggttttgtaagcgaagacgacgaggaccgctcagtaccaacagagg | |
| | ataaaaagcaagaccaggacaacgcagaggcaaacgaggaacaagtcgggcggggggac | |
| | gaaaggcatggcgactacctagatgtgggagacgacgtgctgttgaagcatctgcagcg | |
| | ccagtgcgccattatctgcgacgcgttgcaagagcgcagcgatgtgcccctcgccatag | |
| | cggatgtcagccttgcctacgaacgccacctattctcaccgcgcgtaccccccaaacgc | |
| | caagaaaacggcacatgcgagcccaacccgcgcctcaacttctaccccgtatttgccgt | |
| | gccagaggtgcttgccacctatcacatctttttccaaaactgcaagatacccctatcct | |
| | gccgtgccaaccgcagccgagcggacaagcagctggccttgcggcagggcgctgtcata | |
| | cctgatatcgcctcgctcaacgaagtgccaaaaatctttgagggtcttggacgcgacga | |
| | gaagcgcgcggcaaacgctctgcaacaggaaaacagcgaaaatgaaagtcactctggag | |
| | tgttggtggaactcgagggtgacaacgcgcgcctagccgtactaaaacgcagcatcgag | |
| | gtcacccactttgcctacccggcacttaacctaccccccaaggtcatgagcacagtcat | |
| | gagtgagctgatcgtgcgccgtgcgcagcccctggagagggatgcaaatttgcaagaac | |
| | aaacagaggagggcctacccgcagttggcgacgagcagctagcgcgctggcttcaaacg | |
| | cgcgagcctgccgacttggaggagcgacgcaaactaatgatggccgcagtgctcgttac | |
| | cgtggagcttgagtgcatgcagcggttctttgctgacccggagatgcagcgcaagctag | |
| | aggaaacattgcactacacctttcgacagggctacgtacgccaggcctgcaagatctcc | |
| | aacgtggagctctgcaacctggtctcctaccttggaattttgcacgaaaaccgccttgg | |
| | gcaaaacgtgcttcattccacgctcaagggcgaggcgcgccgcgactacgtccgcgact | |
| | gcgtttacttatttctatgctacacctggcagacggccatgggcgtttggcagcagtgc | |
| | ttggaggagtgcaacctcaaggagctgcagaaactgctaaagcaaaacttgaaggacct | |
| | atggacggccttcaacgagcgctccgtggccgcgcacctggcggacatcattttccccg | |
| | aacgcctgcttaaaaccctgcaacagggtctgccagacttcaccagtcaaagcatgttg | |
| | cagaactttaggaactttatcctagagcgctcaggaatcttgcccgccacctgctgtgc | |
| | acttcctagcgactttgtgcccattaagtaccgcgaatgccctccgccgctttggggcc | |
| | actgctaccttctgcagctagccaactaccttgcctaccactctgacataatggaagac | |
| | gtgagcggtgacggtctactggagtgtcactgtcgctgcaacctatgcaccccgcaccg | |
| | ctccctggtttgcaattcgcagctgcttaacgaaagtcaaattatcggtacctttgagc | |
| | tgcagggtccctcgcctgacgaaaagtccgcggctccggggttgaaactcactccgggg | |
| | ctgtggacgtcggcttaccttcgcaaatttgtacctgaggactaccacgcccacgagat | |
| | taggttctacgaagaccaatcccgcccgcctaatgcggagcttaccgcctgcgtcatta | |
| | cccagggccacattcttggccaattgcaagccatcaacaaagcccgccaagagtttctg | |
| | ctacgaaagggacggggggtttacttggacccccagtccggcgaggagctcaacccaat | |
| | ccccccgccgccgcagccctatcagcagcagccgcgggcccttgcttcccaggatggca | |
| | cccaaaaagaagctgcagctgccgccgccacccacggacgaggaggaatactgggacag | |
| | tcaggcagaggaggttttggacgaggaggaggaggacatgatggaagactgggagagcc | |
| | tagacgaggaagcttccgaggtcgaagaggtgtcagacgaaacaccgtcaccctcggtc | |
| | gcattcccctcgccggcgccccagaaatcggcaaccggttccagcatggctacaacctc | |
| | cgctcctcaggcgccgccggcactgcccgttcgccgacccaaccgtagatgggacacca | |
| | ctggaaccagggccggtaagtccaagcagccgccgccgttagcccaagagcaacaacag | |
| | cgccaaggctaccgctcatggcgcgggcacaagaacgccatagttgcttgcttgcaaga | |
| | ctgtgggggcaacatctccttcgcccgccgctttcttctctaccatcacggcgtggcct | |
| | tcccccgtaacatcctgcattactaccgtcatctctacagcccatactgcaccggcggc | |
| | agcggcagcaacagcagcggccacacagaagcaaaggcgaccggatagcaagactctga | |
| | caaagcccaagaaatccacagcggcggcagcagcaggaggaggagcgctgcgtctggcg | |
| | cccaacgaacccgtatcgacccgcgagcttagaaacaggatttttcccactctgtatgc | |
| | tatatttcaacagagcaggggccaagaacaagagctgaaaataaaaaacaggtctctgc | |
| | gatccctcacccgcagctgcctgtatcacaaaagcgaagatcagcttcggcgcacgctg | |
| | gaagacgcggaggctctcttcagtaaatactgcgcgctgactcttaaggactagtttcg | |
| | cgccctttctcaaatttaagcgcgaaaactacgtcatctccagcggccacacccggcgc | |
| | cagcacctgttgtcagcgccattatgagcaaggaaattcccacgccctacatgtggagt | |
| | taccagccacaaatgggacttgcggctggagctgcccaagactactcaacccgaataaa | |
| | ctacatgagcgcgggaccccacatgatatcccgggtcaacggaatacgcgcccaccgaa | |
| | accgaattctcctggaacaggcggctattaccaccacacctcgtaataaccttaatccc | |
| | cgtagttggcccgctgccctggtgtaccaggaaagtcccgctcccaccactgtggtact | |
| | tcccagagacgcccaggccgaagttcagatgactaactcaggggcgcagcttgcggggg | |
| | ctttcgtcacagggtgcggtcgcccgggcagggtataactcacctgacaatcagagggc | |
| | gaggtattcagctcaacgacgagtcggtgagctcctcgcttggtctccgtccggacggg | |
| | acatttcagatcggcggcgccggccgctcttcattcacgcctcgtcaggcaatcctaac | |
| | tctgcagacctcgtcctctgagccgcgctctggaggcattggaactctgcaatttattg | |
| | aggagtttgtgccatcggtctactttaaccccttctcgggacctcccggccactatccg | |
| | gatcaatttattcctaactttgacgcggtaaaggactcggcggacggctacgactgaat | |
| | gttaagtggagaggcagagcaactgcgcctgaaacacctggtccactgtcgccgccaca | |
| | agtgctttgcccgcgactccggtgagttttgctactttgaattgcccgaggatcatatc | |
| | gagggcccggcgcacggcgtccggcttaccgcccagggagagcttgcccgtagcctgat | |
| | tcgggagtttacccagcgccccctgctagttgagcgggacaggggaccctgtgttctca | |
| | ctgtgatttgcaactgtcctaaccctggattacatcaagatcctctagttaattaacta | |
| | gagtacccggggatcttattccctttaactaataaaaaaaaataataaagcatcactta | |
| | cttaaaatcagttagcaaatttctgtccagtttattcagcagcacctccttgccctcct | |
| | cccagctctggtattgcagcttcctcctggctgcaaactttctccacaatctaaatgga | |
| | atgtcagtttcctcctgttcctgtccatccgcacccactatcttcatgttgttgcagat | |
| | gaagcgcgcaagaccgtctgaagataccttcaaccccgtgtatccatatgacacggaaa | |
| | ccggtcctccaactgtgccttttcttactcctccctttgtatcccccaatgggtttcaa | |
| | gagagtccccctggggtactctctttgcgcctatccgaacctctagttacctccaatgg | |
| | catgcttgcgctcaaaatgggcaacggcctctctctggacgaggccggcaaccttacct | |
| | cccaaaatgtaaccactgtgagcccacctctcaaaaaaaccaagtcaaacataaacctg | |
| | gaaatatctgcacccctcacagttacctcagaagccctaactgtggctgccgccgcacc | |
| | tctaatggtcgcgggcaacacactcaccatgcaatcacaggccccgctaaccgtgcacg | |
| | actccaaacttagcattgccacccaaggacccctcacagtgtcagaaggaaagctagcc | |
| | ctgcaaacatcaggccccctcaccaccaccgatagcagtacccttactatcactgcctc | |
| | accccctctaactactgccactggtagcttgggcattgacttgaaagagcccatttata | |
| | cacaaaatggaaaactaggactaaagtacggggctcctttgcatgtaacagacgaccta | |
| | aacactttgaccgtagcaactggtccaggtgtgactattaataatacttccttgcaaac | |
| | taaagttactggagccttgggttttgattcacaaggcaatatgcaacttaatgtagcag | |
| | gaggactaaggattgattctcaaaacagacgccttatacttgatgttagttatccgttt | |
| | gatgctcaaaaccaactaaatctaagactaggacagggccctctttttataaactcagc | |
| | ccacaacttggatattaactacaacaaaggcctttacttgtttacagcttcaaacaatt | |
| | ccaaaaagcttgaggttaacctaagcactgccaaggggttgatgtttgacgctacagcc | |
| | atagccattaatgcaggagatgggcttgaatttggttcacctaatgcaccaaacacaaa | |
| | tcccctcaaaacaaaaattggccatggcctagaatttgattcaaacaaggctatggttc | |
| | ctaaactaggaactggccttagttttgacagcacaggtgccattacagtaggaaacaaa | |
| | aataatgataagctaactttgtggaccacaccagctccatctcctaactgtagactaaa | |
| | tgcagagaaagatgctaaactcactttggtcttaacaaaatgtggcagtcaaatacttg | |
| | ctacagtttcagttttggctgttaaaggcagtttggctccaatatctggaacagttcaa | |
| | agtgctcatcttattataagatttgacgaaaatggagtgctactaaacaattccttcct | |
| | ggacccagaatattggaactttagaaatggagatcttactgaaggcacagcctatacaa | |
| | acgctgttggatttatgcctaacctatcagcttatccaaaatctcacggtaaaactgcc | |
| | aaaagtaacattgtcagtcaagtttacttaaacggagacaaaactaaacctgtaacact | |
| | aaccattacactaaacggtacacaggaaacaggagacacaactccaagtgcatactcta | |
| | tgtcattttcatgggactggtctggccacaactacattaatgaaatatttgccacatcc | |
| | tcttacactttttcatacattgcccaagaataaagaatcgtttgtgttatgtttcaacg | |
| | tgtttatttttcaattgcagaaaatttcaagtcatttttcattcagtagtatagcccca | |
| | ccaccacatagcttatacagatcaccgtaccttaatcaaactcacagaaccctagtatt | |
| | caacctgccacctccctcccaacacacagagtacacagtcctttctccccggctggcct | |
| | taaaaagcatcatatcatgggtaacagacatattcttaggtgttatattccacacggtt | |
| | tcctgtcgagccaaacgctcatcagtgatattaataaactccccgggcagctcacttaa | |
| | gttcatgtcgctgtccagctgctgagccacaggctgctgtccaacttgcggttgcttaa | |
| | cgggcggcgaaggagaagtccacgcctacatgggggtagagtcataatcgtgcatcagg | |
| | atagggcggtggtgctgcagcagcgcgcgaataaactgctgccgccgccgctccgtcct | |
| | gcaggaatacaacatggcagtggtctcctcagcgatgattcgcaccgcccgcagcataa | |
| | ggcgccttgtcctccgggcacagcagcgcaccctgatctcacttaaatcagcacagtaa | |
| | ctgcagcacagcaccacaatattgttcaaaatcccacagtgcaaggcgctgtatccaaa | |
| | gctcatggcggggaccacagaacccacgtggccatcataccacaagcgcaggtagatta | |
| | agtggcgacccctcataaacacgctggacataaacattacctcttttggcatgttgtaa | |
| | ttcaccacctcccggtaccatataaacctctgattaaacatggcgccatccaccaccat | |
| | cctaaaccagctggccaaaacctgcccgccggctatacactgcagggaaccgggactgg | |
| | aacaatgacagtggagagcccaggactcgtaaccatggatcatcatgctcgtcatgata | |
| | tcaatgttggcacaacacaggcacacgtgcatacacttcctcaggattacaagctcctc | |
| | ccgcgttagaaccatatcccagggaacaacccattcctgaatcagcgtaaatcccacac | |
| | tgcagggaagacctcgcacgtaactcacgttgtgcattgtcaaagtgttacattcgggc | |
| | agcagcggatgatcctccagtatggtagcgcgggtttctgtctcaaaaggaggtagacg | |
| | atccctactgtacggagtgcgccgagacaaccgagatcgtgttggtcgtagtgtcatgc | |
| | caaatggaacgccggacgtagtcatatttcctgaagcaaaaccaggtgcgggcgtgaca | |
| | aacagatctgcgtctccggtctcgccgcttagatcgctctgtgtagtagttgtagtata | |
| | tccactctctcaaagcatccaggcgccccctggcttcgggttctatgtaaactccttca | |
| | tgcgccgctgccctgataacatccaccaccgcagaataagccacacccagccaacctac | |
| | acattcgttctgcgagtcacacacgggaggagcgggaagagctggaagaaccatgtttt | |
| | tttttttattccaaaagattatccaaaacctcaaaatgaagatctattaagtgaacgcg | |
| | ctcccctccggtggcgtggtcaaactctacagccaaagaacagataatggcatttgtaa | |
| | gatgttgcacaatggcttccaaaaggcaaacggccctcacgtccaagtggacgtaaagg | |
| | ctaaacccttcagggtgaatctcctctataaacattccagcaccttcaaccatgcccaa | |
| | ataattctcatctcgccaccttctcaatatatctctaagcaaatcccgaatattaagtc | |
| | cggccattgtaaaaatctgctccagagcgccctccaccttcagcctcaagcagcgaatc | |
| | atgattgcaaaaattcaggttcctcacagacctgtataagattcaaaagcggaacatta | |
| | acaaaaataccgcgatcccgtaggtcccttcgcagggccagctgaacataatcgtgcag | |
| | gtctgcacggaccagcgcggccacttccccgccaggaaccatgacaaaagaacccacac | |
| | tgattatgacacgcatactcggagctatgctaaccagcgtagccccgatgtaagcttgt | |
| | tgcatgggggcgatataaaatgcaaggtgctgctcaaaaaatcaggcaaagcctcgcgc | |
| | aaaaaagaaagcacatcgtagtcatgctcatgcagataaaggcaggtaagctccggaac | |
| | caccacagaaaaagacaccatttttctctcaaacatgtctgcgggtttctgcataaaca | |
| | caaaataaaataacaaaaaaacatttaaacattagaagcctgtcttacaacaggaaaaa | |
| | caacccttataagcataagacggactacggccatgccggcgtgaccgtaaaaaaactgg | |
| | tcaccgtgattaaaaagcaccaccgacagctcctcggtcatgtccggagtcataatgta | |
| | agactcggtaaacacatcaggttgattcacatcggtcagtgctaaaaagcgaccgaaat | |
| | agcccgggggaatacatacccgcaggcgtagagacaacattacagcccccataggaggt | |
| | ataacaaaattaataggagagaaaaacacataaacacctgaaaaaccctcctgcctagg | |
| | caaaatagcaccctcccgctccagaacaacatacagcgcttccacagcggcagccataa | |
| | cagtcagccttaccagtaaaaaagaaaacctattaaaaaaacaccactcgacacggcac | |
| | cagctcaatcagtcacagtgtaaaaaagggccaagtgcagagcgagtatatataggact | |
| | aaaaaatgacgtaacggttaaagtccacaaaaaacacccagaaaaccgcacgcgaacct | |
| | acgcccagaaacgaaagccaaaaaacccacaacttcctcaaatcgtcacttccgttttc | |
| | ccacgttacgtcacttcccattttaagaaaactacaattcccaacacatacaagttact | |
| | ccgccctaaaacctacgtcacccgccccgttcccacgccccgcgccacgtcacaaactc | |
| | caccccctcattatcatattggcttcaatccaaaataatcatcaataatataccttatt | |
| | ttggattgaagccaatatgataatgagggggtggagtttgtgacgtggcgcggggcgtg | |
| | ggaacggggcgggtgacgtagtagtgtggcggaagtgtgatgttgcaagtgtggcggaa | |
| | cacatgtaagcgacggatgtggcaaaagtgacgtttttggtgtgcgccggatccacagg | |
| | acgggtgtggtcgccatgatcgcgtagtcgatagtggctccaagtagcgaagcgagcag | |
| | gactgggcggcggccaaagcggtcggacagtgctccgagaacgggtgcgcatagaaatt | |
| | gcatcaacgcatatagcgctagcagcacgccatagtgactggcgatgctgtcggaatgg | |
| | acgatatcccgcaagaggcccggcagtaccggcataaccaagcctatgcctacagcatc | |
| | cagggtgacggtgccgaggatgacgatgagcgcattgttagatttcatacacggtgcct | |
| | gactgcgttagcaatttaactgtgataaactaccgcattaaagcttatcgaattcgtaa | |
| | tcatgtcatagctgtttcctgtgtgaaattgttatccgctcacaattccacacaacata | |
| | cgagccggaagcataaagtgtaaagcctggggtgcctaatgagtgagctaactcacatt | |
| | aattgcgttgcgctcactgcccgctttccagtcgggaaacctgtcgtgccagctgcatt | |
| | aatgaatcggccaacgcgcggggagaggcggtttgcgtattgggcgc | |
| 48 | ccatatgcggtgtgaaataccgcacagatgcgtaaggagaaaataccgcatcaggcgct | Helper + |
| | cttccgcttcctcgctcactgactcgctgcgctcggtcgttcggctgcggcgagcggta | Rep/Cap |
| | tcagctcactcaaaggcggtaatacggttatccacagaatcaggggataacgcaggaaa | plasmid - |
| | gaacatgtgagcaaaaggccagcaaaaggccaggaaccgtaaaaaggccgcgttgctgg | pDG-KanR |
| | cgtttttccataggctccgcccccctgacgagcatcacaaaaatccctgccgcttaccg | |
| | gatacctgtccgcctttctcccttcgggaagcgtggcgctttctcatagctcacgctgt | |
| | aggtatctcagttcggtgtaggtcggacgctcaagtcagaggtggcgaaacccgacagg | |
| | actataaagataccaggcgtttccccctggaagctccctcgtgcgctctcctgttccga | |
| | cttcgctccaagctgggctgtgtgcacgaaccccccgttcagcccgaccgctgcgcctt | |
| | atccggtaactatcgtcttgagtccaacccggtaagacacgacttatcgccactggcag | |
| | cagccactggtaacaggattagcagagcgaggtatgtaggcggtgctacagagttcttg | |
| | aagtggtggcctaactacggctacactagaaggacagtatttggtatctgcgctctgct | |
| | gaagccagttaccttcggaaaaagagttggtagctcttgatccggcaaacaaaccaccg | |
| | ctggtagcggtggtttttttgtttgcaagcagcagattacgcgcagaaaaaaaggatct | |
| | caagaagatcctttgatcttttctacggggtctgacgctcagtggaacgaaaactcacg | |
| | ttaagggattttggtcatgagattatcaaaaaggatcttcacctagatccttttaaatt | |
| | aaaaatgaagttttaaatcaatctaaagtatatatgagtaaacttggtctgacagttac | |
| | caatgcttaatcagtgaggcacctatctcagcgatctgtctatttcgttcatccatagt | |
| | tgcctgactccccgtcgtgtagataactacgatacgggagggcttaccatctggcccca | |
| | gtgctgcaatgataccgcgagacccacgctcaccggctccagatttatcagcaataaac | |
| | cagccagccggaagggccgagcgcagaagtggtcctgcaactttatccgcctccatcca | |
| | gtctattaattgttgccgggaagctagagtaagtagttcgccagttaatagtttgcgca | |
| | acgttgttgccattgctgcagccatgagattatcaaaaaggatcttcacctagatcctt | |
| | ttcacgtagaaagccagtccgcagaaacggtgctgaccccggatgaatgtcagctactg | |
| | ggctatctggacaagggaaaacgcaagcgcaaagagaaagcaggtagcttgcagtgggc | |
| | ttacatggcgatagctagactgggcggttttatggacagcaagcgaaccggaattgcca | |
| | gctggggcgccctctggtaaggttgggaagccctgcaaagtaaactggatggctttctt | |
| | gccgccaaggatctgatggcgcaggggatcaagctctgatcaagagacaggatgaggat | |
| | cgtttcgcatgattgaacaagatggattgcacgcaggttctccggccgcttgggtggag | |
| | aggctattcggctatgactgggcacaacagacaatcggctgctctgatgccgccgtgtt | |
| | ccggctgtcagcgcaggggcgcccggttctttttgtcaagaccgacctgtccggtgccc | |
| | tgaatgaactgcaagacgaggcagcgcggctatcgtggctggccacgacgggcgttcct | |
| | tgcgcagctgtgctcgacgttgtcactgaagcgggaagggactggctgctattgggcga | |
| | agtgccggggcaggatctcctgtcatctcaccttgctcctgccgagaaagtatccatca | |
| | tggctgatgcaatgcggcggctgcatacgcttgatccggctacctgcccattcgaccac | |
| | caagcgaaacatcgcatcgagcgagcacgtactcggatggaagccggtcttgtcgatca | |
| | ggatgatctggacgaagagcatcaggggctcgcgccagccgaactgttcgccaggctca | |
| | aggcgagcatgcccgacggcgaggatctcgtcgtgacccatggcgatgcctgcttgccg | |
| | aatatcatggtggaaaatggccgcttttctggattcatcgactgtggccggctgggtgt | |
| | ggcggaccgctatcaggacatagcgttggctacccgtgatattgctgaagagcttggcg | |
| | gcgaatgggctgaccgcttcctcgtgctttacggtatcgccgctcccgattcgcagcgc | |
| | atcgccttctatcgccttcttgacgagttcttctgaattttgttaaaatttttgttaaa | |
| | tcagctcattttttaaccaataggccgaaatcggcaaaatcccttataaatcaaaagaa | |
| | tagaccgagatagggttgagtgttgttccagtttggaacaagagtccactattaaagaa | |
| | cgtggactccaacgtcaaagggcgaaaaaccgtctatcagggcgatggcccactacgtg | |
| | aaccatcaccctaatcaagttttttggggtcgaggtgccgtaaagcactaaatcggaac | |
| | cctaaagggagcccccgatttagagcttgacggggaaagccggcgaacgtggcgagaaa | |
| | ggaagggaagaaagcgaaaggagcgggcgctagggcgctggcaagtgtagcggtcacgc | |
| | tgcgcgtaaccaccacacccgccgcgcttaatgcgccgctacagggcgcgtccattcgc | |
| | cattcaggatcgaattaattcttaattaacatcatcaataatataccttattttggatt | |
| | gaagccaatatgataatgagggggtggagtttgtgacgtggcgcggggcgtgggaacgg | |
| | ggcgggtgacgtagtagtgtggcggaagtgtgatgttgcaagtgtggcggaacacatgt | |
| | aagcgacggatgtggcaaaagtgacgtttttggtgtgcgccggtgtacacaggaagtga | |
| | caattttcgcgcggttttaggcggatgttgtagtaaatttgggcgtaaccgagtaagat | |
| | ttggccattttcgcgggaaaactgaataagaggaagtgaaatctgaataattttgtgtt | |
| | actcatagcgcgtaatatttgtctagggccgcggggactttgaccgtttacgtggagac | |
| | tcgcccaggtgtttttctcaggtgttttccgcgttccgggtcaaagttggcgttttatt | |
| | attatagtcagggggatcctctagaactagtggatccgtccctagaagtaaaaaaggga | |
| | aaaaagagtgtgtttgtcaaaataggagacaggtggtggcaaccagggacttatagggg | |
| | accttacatctacagaccaacagatgcccccttaccatatacaggaagatatgacttaa | |
| | attgggataggtgggtcacaatcaacggctataaagtgttatacagatccctccccttt | |
| | cgtgaaagactcgccagagctagacctccttggtgtatgctaactgagaaagagaaaga | |
| | cgacatgaaacaacaggtacatgattatatttatctaggaacaggaatgcacttttggg | |
| | gaaaggttttccataccaaggaaggggcagtggctggactgatagaacattattctgca | |
| | aaaacttatggtatgagttattatgattagcctttatttgcccaaccttgcggttccca | |
| | gggtttaaataagtttatggttacaaactgttcttaaaacaaggatgtgagacaagtgg | |
| | tttcctgagttggtttggtatcaaatgttctgatctgagctcttagtgttctattttcc | |
| | tatgttcttttggaatctatccaagtcttatgtaaatgcttatgtaaaccataatataa | |
| | aagagtgctgattttttgagtaaacttgcaacagtcctaacattcttctctcgtgtgtt | |
| | tgtgtctgttcgccatcccgtctccgctcgtcacttatccttcacttttcagagggtcc | |
| | ccccgcagatcccggtcaccctcaggtcgggacctgcagaagacgcccgagtgagcacg | |
| | cagggtctccattttgaagcgggaggtttgaacgcgcagccgccatgccggggttttac | |
| | gagattgtgattaaggtccccagcgaccttgacgagcatctgcccggcatttctgacag | |
| | ctttgtgaactgggtggccgagaaggaatgggagttgccgccagattctgacatggatc | |
| | tgaatctgattgagcaggcacccctgaccgtggccgagaagctgcagcgcgactttctg | |
| | acggaatggcgccgtgtgagtaaggccccggaggcccttttctttgtgcaatttgagaa | |
| | gggagagagctacttccacatgcacgtgctcgtggaaaccaccggggtgaaatccatgg | |
| | ttttgggacgtttcctgagtcagattcgcgaaaaactgattcagagaatttaccgcggg | |
| | atcgagccgactttgccaaactggttcgcggtcacaaagaccagaaatggcgccggagg | |
| | cgggaacaaggtggtggatgagtgctacatccccaattacttgctccccaaaacccagc | |
| | ctgagctccagtgggcgtggactaatatggaacagtatttaagcgcctgtttgaatctc | |
| | acggagcgtaaacggttggtggcgcagcatctgacgcacgtgtcgcagacgcaggagca | |
| | gaacaaagagaatcagaatcccaattctgatgcgccggtgatcagatcaaaaacttcag | |
| | ccaggtacatggagctggtcgggtggctcgtggacaaggggattacctcggagaagcag | |
| | tggatccaggaggaccaggcctcatacatctccttcaatgcggcctccaactcgcggtc | |
| | ccaaatcaaggctgccttggacaatgcgggaaagattatgagcctgactaaaaccgccc | |
| | ccgactacctggtgggccagcagcccgtggaggacatttccagcaatcggatttataaa | |
| | attttggaactaaacgggtacgatccccaatatgcggcttccgtctttctgggatgggc | |
| | cacgaaaaagttcggcaagaggaacaccatctggctgtttgggcctgcaactaccggga | |
| | agaccaacatcgcggaggccatagcccacactgtgcccttctacgggtgcgtaaactgg | |
| | accaatgagaactttcccttcaacgactgtgtcgacaagatggtgatctggtgggagga | |
| | ggggaagatgaccgccaaggtcgtggagtcggccaaagccattctcggaggaagcaagg | |
| | tgcgcgtggaccagaaatgcaagtcctcggcccagatagacccgactcccgtgatcgtc | |
| | acctccaacaccaacatgtgcgccgtgattgacgggaactcaacgaccttcgaacacca | |
| | gcagccgttgcaagaccggatgttcaaatttgaactcacccgccgtctggatcatgact | |
| | ttgggaaggtcaccaagcaggaagtcaaagactttttccggtgggcaaaggatcacgtg | |
| | gttgaggtggagcatgaattctacgtcaaaaagggtggagccaagaaaagacccgcccc | |
| | cagtgacgcagatataagtgagcccaaacgggtgcgcgagtcagttgcgcagccatcga | |
| | cgtcagacgcggaagcttcgatcaactacgcagacaggtaccaaaacaaatgttctcgt | |
| | cacgtgggcatgaatctgatgctgtttccctgcagacaatgcgagagaatgaatcagaa | |
| | ttcaaatatctgcttcactcacggacagaaagactgtttagagtgctttcccgtgtcag | |
| | aatctcaacccgtttctgtcgtcaaaaaggcgtatcagaaactgtgctacattcatcat | |
| | atcatgggaaaggtgccagacgcttgcactgcctgcgatctggtcaatgtggatttgga | |
| | tgactgcatctttgaacaataaatgatttaaatcaggtatggctgccgatggttatctt | |
| | ccagattggctcgaggacactctctctgaaggaataagacagtggtggaagctcaaacc | |
| | tggcccaccaccaccaaagcccgcagagcggcataaggacgacagcaggggtcttgtgc | |
| | ttcctgggtacaagtacctcggacccttcaacggactcgacaagggagagccggtcaac | |
| | gaggcagacgccgcggccctcgagcacgacaaagcctacgaccggcagctcgacagcgg | |
| | agacaacccgtacctcaagtacaaccacgccgacgcggagtttcaggagcgccttaaag | |
| | aagatacgtcttttgggggcaacctcggacgagcagtcttccaggcgaaaaagagggtt | |
| | cttgaacctctgggcctggttgaggaacctgttaagacggctccgggaaaaaagaggcc | |
| | ggtagagcactctcctgtggagccagactcctcctcgggaaccggaaaggcgggccagc | |
| | agcctgcaagaaaaagattgaattttggtcagactggagacgcagactcagtacctgac | |
| | ccccagcctctcggacagccaccagcagccccctctggtctgggaactaatacgatggc | |
| | tacaggcagtggcgcaccaatggcagacaataacgagggcgccgacggagtgggtaatt | |
| | cctcgggaaattggcattgcgattccacatggatgggcgacagagtcatcaccaccagc | |
| | acccgaacctgggccctgcccacctacaacaaccacctctacaaacaaatttccagcca | |
| | atcaggagcctcgaacgacaatcactactttggctacagcaccccttgggggtattttg | |
| | acttcaacagattccactgccacttttcaccacgtgactggcaaagactcatcaacaac | |
| | aactggggattccgacccaagagactcaacttcaagctctttaacattcaagtcaaaga | |
| | ggtcacgcagaatgacggtacgacgacgattgccaataaccttaccagcacggttcagg | |
| | tgtttactgactcggagtaccagctcccgtacgtcctcggctcggcgcatcaaggatgc | |
| | ctcccgccgttcccagcagacgtcttcatggtgccacagtatggatacctcaccctgaa | |
| | caacgggagtcaggcagtaggacgctcttcattttactgcctggagtactttccttctc | |
| | agatgctgcgtaccggaaacaactttaccttcagctacacttttgaggacgttcctttc | |
| | cacagcagctacgctcacagccagagtctggaccgtctcatgaatcctctcatcgacca | |
| | gtacctgtattacttgagcagaacaaacactccaagtggaaccaccacgcagtcaaggc | |
| | ttcagttttctcaggccggagcgagtgacattcgggaccagtctaggaactggcttcct | |
| | ggaccctgttaccgccagcagcgagtatcaaagacatctgcggataacaacaacagtga | |
| | atactcgtggactggagctaccaagtaccacctcaatggcagagactctctggtgaatc | |
| | cgggcccggccatggcaagccacaaggacgatgaagaaaagttttttcctcagagcggg | |
| | gttctcatctttgggaagcaaggctcagagaaaacaaatgtggacattgaaaaggtcat | |
| | gattacagacgaagaggaaatcaggacaaccaatcccgtggctacggagcagtatggtt | |
| | ctgtatctaccaacctccagagaggcaacagacaagcagctaccgcagatgtcaacaca | |
| | caaggcgttcttccaggcatggtctggcaggacagagatgtgtaccttcaggggcccat | |
| | ctgggcaaagattccacacacggacggacattttcacccctctcccctcatgggtggat | |
| | tcggacttaaacaccctcctccacagattctcatcaagaacaccccggtacctgcgaat | |
| | ccttcgaccaccttcagtgcggcaaagtttgcttccttcatcacacagtactccacggg | |
| | acaggtcagcgtggagatcgagtgggagctgcagaaggaaaacagcaaacgctggaatc | |
| | ccgaaattcagtacacttccaactacaacaagtctgttaatgtggactttactgtggac | |
| | actaatggcgtgtattcagagcctcgccccattggcaccagatacctgactcgtaatct | |
| | gtaattgcttgttaatcaataaaccgtttaattcgtttcagttgaactttggtctctgc | |
| | gtatttctttcttatctagtttccatggctacatcagcttatcgatgcatgtccttggg | |
| | tccggcctgctgaatgcgcaggcggtcggccatgccccaggcttcgttttgacatcggc | |
| | gcaggtctttgtagtagtcttgcatgagcctttctaccggcacttcttcttctccttcc | |
| | tcttgtcctgcatctcttgcatctatcgctgcggcggcggcggagtttggccgtaggtg | |
| | gcgccctcttcctcccatcgtgtgaccccgaagcccctcatcggctgaagcagggctag | |
| | gtcggcgacaacgcgctcggctaatatggcctgctgcacctgcgtgagggtagactgga | |
| | agtcatccatgtccacaaagcggtggtatgcgcccgtgttgatggtgtaagtgcagttg | |
| | gccataacggaccagttaacggtctggtgacccggctgcgagagctcggtgtacctgag | |
| | acgcgagtaagccctcgagtcaaatacgtagtcgttacaagtccgcaccaggtactggt | |
| | atcccaccaaaaagtgcggcggcggctggcggtagaggggccagcgtagggtggccggg | |
| | gctccgggggcgagatcttccaacataaggcgatgatatccgtagatgtacctggacat | |
| | ccaggtgatgccggcggcggtggtggaggcgcgcggaaagtcgcggacgcggttccaga | |
| | tgttgcgcagcggcaaaaagtgctccatggtcgggacgctctggccggtcaggcgcgcg | |
| | caatcgttgacgctctagaccgtgcaaaaggagagcctgtaagcgggcactcttccgtg | |
| | gtctggtggataaattcgcaagggtatcatggcggacgaccggggttcgagccccgtat | |
| | ccggccgtccgccgtgatccatgcggttaccgcccgcgtgtcgaacccaggtgtgcgac | |
| | gtcagacaacgggggagtgctccttttggcttccttccaggcgcggcggctgctgcgct | |
| | agcttttttggccactggccgcgcgcagcgtaagcggttaggctggaaagcgaaagcat | |
| | taagtggctcgctccctgtagccggagggttattttccaagggttgagtcgcgggaccc | |
| | ccggttcgagtctcggaccggccggactgcggcgaacgggggtttgtctccccgtcatg | |
| | caagaccccgcttgcaaattcctccggaaacagggacgagccccttttttgcttttccc | |
| | agatgcatccggtgctgcggcagatgcgcccccctcctcagcagcggcaagagcaagag | |
| | cagcggcagacatgcagggcaccctcccctcctcctaccgcgtcaggaggggcgacatc | |
| | cgcggttgacgcggcagcagatggtgattacgaacccccgcggcgccgggcccggcact | |
| | acctggacttggaggagggcgagggcctggcgcggctaggagcgccctctcctgagcgg | |
| | cacccaagggtgcagctgaagcgtgatacgcgtgaggcgtacgtgccgcggcagaacct | |
| | gtttcgcgaccgcgagggagaggagcccgaggagatgcgggatcgaaagttccacgcag | |
| | ggcgcgagctgcggcatggcctgaatcgcgagcggttgctgcgcgaggaggactttgag | |
| | cccgacgcgcgaaccgggattagtcccgcgcgcgcacacgtggcggccgccgacctggt | |
| | aaccgcatacgagcagacggtgaaccagggcgatcgcaccctttggcgcatcccattct | |
| | ccagtaactttatgtccatgggcgcactcacagacctgggccaaaaccttctctacgcc | |
| | aactccgcccacgcgctagacatgacttttgaggtggatcccatggacgagcccaccct | |
| | tctttatgttttgtttgaagtctttgacgtggtccgtgtgcaccagccgcaccgcggcg | |
| | tcatcgaaaccgtgtacctgcgcacgcccttctcggccggcaacgccacaacataaaga | |
| | agcaagcaacatcaacaacagctgccgccatgggctccagtgagcaggaactgaaagcc | |
| | attgtcaaagatcttggttgtgggccatattttttgggcacctatgacaagcgctttcc | |
| | aggctttgtttctccacacaagctcgcctgcgccatagtcaatacggccggtcgcgaga | |
| | ctgggggcgtacactggatggcctttgcctggaacccgcactcaaaaacatgctacctc | |
| | tttgagccctttggcttttctgaccagcgactcaagcaggtttaccagtttgagtacga | |
| | gtcactcctgcgccgtagcgccattgcttcttcccccgaccgctgtataacgctggaaa | |
| | agtccacccaaagcgtacaggggcccaactcggccgcctgtggactattctgctgcatg | |
| | tttctccacgcctttgccaactggccccaaactcccatggatcacaaccccaccatgaa | |
| | ccttattaccggggtacccaactccatgctcaacagtccccaggtacagcccaccctgc | |
| | gtcgcaaccaggaacagctctacagcttcctggagcgccactcgccctacttccgcagc | |
| | cacagtgcgcagattaggagcgccacttctttttgtcacttgaaaaacatgtaaaaata | |
| | atgtactagagacactttcaataaaggcaaatgcttttatttgtacactctcgggtgat | |
| | tatttacccccacccttgccgtctgcgccgtttaaaaatcaaaggggttctgccgcgca | |
| | tcgctatgcgccactggcagggacacgttgcgatactggtgtttagtgctccacttaaa | |
| | ctcaggcacaaccatccgcggcagctcggtgaagttttcactccacaggctgcgcacca | |
| | tcaccaacgcgtttagcaggtcgggcgccgatatcttgaagtcgcagttggggcctccg | |
| | ccctgcgcgcgcgagttgcgatacacagggttgcagcactggaacactatcagcgccgg | |
| | gtggtgcacgctggccagcacgctcttgtcggagatcagatccgcgtccaggtcctccg | |
| | cgttgctcagggcgaacggagtcaactttggtagctgccttcccaaaaagggcgcgtgc | |
| | ccaggctttgagttgcactcgcaccgtagtggcatcaaaaggtgaccgtgcccggtctg | |
| | ggcgttaggatacagcgcctgcataaaagccttgatctgcttaaaagccacctgagcct | |
| | ttgcgccttcagagaagaacatgccgcaagacttgccggaaaactgattggccggacag | |
| | gccgcgtcgtgcacgcagcaccttgcgtcggtgttggagatctgcaccacatttcggcc | |
| | ccaccggttcttcacgatcttggccttgctagactgctccttcagcgcgcgctgcccgt | |
| | tttcgctcgtcacatccatttcaatcacgtgctccttatttatcataatgcttccgtgt | |
| | agacacttaagctcgccttcgatctcagcgcagcggtgcagccacaacgcgcagcccgt | |
| | gggctcgtgatgcttgtaggtcacctctgcaaacgactgcaggtacgcctgcaggaatc | |
| | gccccatcatcgtcacaaaggtcttgttgctggtgaaggtcagctgcaacccgcggtgc | |
| | tcctcgttcagccaggtcttgcatacggccgccagagcttccacttggtcaggcagtag | |
| | tttgaagttcgcctttagatcgttatccacgtggtacttgtccatcagcgcgcgcgcag | |
| | cctccatgcccttctcccacgcagacacgatcggcacactcagcgggttcatcaccgta | |
| | atttcactttccgcttcgctgggctcttcctcttcctcttgcgtccgcataccacgcgc | |
| | cactgggtcgtcttcattcagccgccgcactgtgcgcttacctcctttgccatgcttga | |
| | ttagcaccggtgggttgctgaaacccaccatttgtagcgccacatcttctctttcttcc | |
| | tcgctgtccacgattacctctggtgatggcgggcgctcgggcttgggagaagggcgctt | |
| | ctttttcttcttgggcgcaatggccaaatccgccgccgaggtcgatggccgcgggctgg | |
| | gtgtgcgcggcaccagcgcgtcttgtgatgagtcttcctcgtcctcggactcgatacgc | |
| | cgcctcatccgcttttttgggggcgcccggggaggcggcggcgacggggacggggacga | |
| | cacgtcctccatggttgggggacgtcgcgccgcaccgcgtccgcgctcgggggtggttt | |
| | cgcgctgctcctcttcccgactggccatttccttctcctataggcagaaaaagatcatg | |
| | gagtcagtcgagaagaaggacagcctaaccgccccctctgagttcgccaccaccgcctc | |
| | caccgatgccgccaacgcgcctaccaccttccccgtcgaggcacccccgcttgaggagg | |
| | aggaagtgattatcgagcaggacccaggttttgtaagcgaagacgacgaggaccgctca | |
| | gtaccaacagaggataaaaagcaagaccaggacaacgcagaggcaaacgaggaacaagt | |
| | cgggggggggacgaaaggcatggcgactacctagatgtgggagacgacgtgctgttgaa | |
| | gcatctgcagcgccagtgcgccattatctgcgacgcgttgcaagagcgcagcgatgtgc | |
| | ccctcgccatagcggatgtcagccttgcctacgaacgccacctattctcaccgcgcgta | |
| | ccccccaaacgccaagaaaacggcacatgcgagcccaacccgcgcctcaacttctaccc | |
| | cgtatttgccgtgccagaggtgcttgccacctatcacatctttttccaaaactgcaaga | |
| | tacccctatcctgccgtgccaaccgcagccgagcggacaagcagctggccttgcggcag | |
| | ggcgctgtcatacctgatatcgcctcgctcaacgaagtgccaaaaatctttgagggtct | |
| | tggacgcgacgagaagcgcgcggcaaacgctctgcaacaggaaaacagcgaaaatgaaa | |
| | gtcactctggagtgttggtggaactcgagggtgacaacgcgcgcctagccgtactaaaa | |
| | cgcagcatcgaggtcacccactttgcctacccggcacttaacctaccccccaaggtcat | |
| | gagcacagtcatgagtgagctgatcgtgcgccgtgcgcagcccctggagagggatgcaa | |
| | atttgcaagaacaaacagaggagggcctacccgcagttggcgacgagcagctagcgcgc | |
| | tggcttcaaacgcgcgagcctgccgacttggaggagcgacgcaaactaatgatggccgc | |
| | agtgctcgttaccgtggagcttgagtgcatgcagcggttctttgctgacccggagatgc | |
| | agcgcaagctagaggaaacattgcactacacctttcgacagggctacgtacgccaggcc | |
| | tgcaagatctccaacgtggagctctgcaacctggtctcctaccttggaattttgcacga | |
| | aaaccgccttgggcaaaacgtgcttcattccacgctcaagggcgaggcgcgccgcgact | |
| | acgtccgcgactgcgtttacttatttctatgctacacctggcagacggccatgggcgtt | |
| | tggcagcagtgcttggaggagtgcaacctcaaggagctgcagaaactgctaaagcaaaa | |
| | cttgaaggacctatggacggccttcaacgagcgctccgtggccgcgcacctggcggaca | |
| | tcattttccccgaacgcctgcttaaaaccctgcaacagggtctgccagacttcaccagt | |
| | caaagcatgttgcagaactttaggaactttatcctagagcgctcaggaatcttgcccgc | |
| | cacctgctgtgcacttcctagcgactttgtgcccattaagtaccgcgaatgccctccgc | |
| | cgctttggggccactgctaccttctgcagctagccaactaccttgcctaccactctgac | |
| | ataatggaagacgtgagcggtgacggtctactggagtgtcactgtcgctgcaacctatg | |
| | caccccgcaccgctccctggtttgcaattcgcagctgcttaacgaaagtcaaattatcg | |
| | gtacctttgagctgcagggtccctcgcctgacgaaaagtccgcggctccggggttgaaa | |
| | ctcactccggggctgtggacgtcggcttaccttcgcaaatttgtacctgaggactacca | |
| | cgcccacgagattaggttctacgaagaccaatcccgcccgcctaatgcggagcttaccg | |
| | cctgcgtcattacccagggccacattcttggccaattgcaagccatcaacaaagcccgc | |
| | caagagtttctgctacgaaagggacggggggtttacttggacccccagtccggcgagga | |
| | gctcaacccaatccccccgccgccgcagccctatcagcagcagccgcgggcccttgctt | |
| | cccaggatggcacccaaaaagaagctgcagctgccgccgccacccacggacgaggagga | |
| | atactgggacagtcaggcagaggaggttttggacgaggaggaggaggacatgatggaag | |
| | actgggagagcctagacgaggaagcttccgaggtcgaagaggtgtcagacgaaacaccg | |
| | tcaccctcggtcgcattcccctcgccggcgccccagaaatcggcaaccggttccagcat | |
| | ggctacaacctccgctcctcaggcgccgccggcactgcccgttcgccgacccaaccgta | |
| | gatgggacaccactggaaccagggccggtaagtccaagcagccgccgccgttagcccaa | |
| | gagcaacaacagcgccaaggctaccgctcatggcgcgggcacaagaacgccatagttgc | |
| | ttgcttgcaagactgtgggggcaacatctccttcgcccgccgctttcttctctaccatc | |
| | acggcgtggccttcccccgtaacatcctgcattactaccgtcatctctacagcccatac | |
| | tgcaccggcggcagcggcagcaacagcagcggccacacagaagcaaaggcgaccggata | |
| | gcaagactctgacaaagcccaagaaatccacagcggcggcagcagcaggaggaggagcg | |
| | ctgcgtctggcgcccaacgaacccgtatcgacccgcgagcttagaaacaggatttttcc | |
| | cactctgtatgctatatttcaacagagcaggggccaagaacaagagctgaaaaaaaaaa | |
| | caggtctctgcgatccctcacccgcagctgcctgtatcacaaaagcgaagatcagcttc | |
| | ggcgcacgctggaagacgcggaggctctcttcagtaaatactgcgcgctgactcttaag | |
| | gactagtttcgcgccctttctcaaatttaagcgcgaaaactacgtcatctccagcggcc | |
| | acacccggcgccagcacctgttgtcagcgccattatgagcaaggaaattcccacgccct | |
| | acatgtggagttaccagccacaaatgggacttgcggctggagctgcccaagactactca | |
| | acccgaataaactacatgagcggggaccccacatgatatcccgggtcaacggaatacgc | |
| | gcccaccgaaaccgaattctcctggaacaggcggctattaccaccacacctcgtaataa | |
| | ccttaatccccgtagttggcccgctgccctggtgtaccaggaaagtcctgctcccacca | |
| | ctgtggtacttcccagagacgcccaggccgaagttcagatgactaactcaggggcgcag | |
| | cttgcgggggctttcgtcacagggtgcggtcgcccgggcagggtataactcacctgaca | |
| | atcagagggcgaggtattcagctcaacgacgagtcggtgagctcctcgcttggtctccg | |
| | tccggacgggacatttcagatcggggcgccggccgctcttcattcacgcctcgtcaggc | |
| | aatcctaactctgcagacctcgtcctctgagccgcgctctggaggcattggaactctgc | |
| | aatttattgaggagtttgtgccatcggtctactttaaccccttctcgggacctcccggc | |
| | cactatccggatcaatttattcctaactttgacgcggtaaaggactcggcggacggcta | |
| | cgactgaatgttaagtggagaggcagagcaactgcgcctgaaacacctggtccactgtc | |
| | gccgccacaagtgctttgcccgcgactccggtgagttttgctactttgaatcgcccgag | |
| | gatcatatcgagggcccggcgcacggcgtccggcttaccgcccagggagagcttgcccg | |
| | tagcctgattcgggagtttacccagcgccccctgctagttgagcgggacaggggaccct | |
| | gtgttctcactgtgatttgcaactgtcctaaccctggattacatcaagatctttgttgc | |
| | catctctgtgctgagtataataaatacagaaattaaaatatactggggctcctatcgcc | |
| | atcctgtaaacgccaccgtcttcacccgcccaagcaaaccaaggcgaaccttacctggt | |
| | acttttaacatctctccctctgtgatttacaacagtttcaacccagacggagtgagtct | |
| | acgagagaacctctccgagctcagctactccatcagaaaaaacaccaccctccttacct | |
| | gccgggaacgtacgagtgcgtcaccggccgctgcaccacacctaccgcctgaccgtaaa | |
| | ccagactttttccggacagacctcaataactctgtttaccagaacaggaggtgagctta | |
| | gaaaacccttagggtattaggccaaaggcgcagctactgtggggtttatgaacaattca | |
| | agcaactctacgggctattctaattcaggtttctctagaaatggacggaattattacag | |
| | agcagcgcctgctagaaagacgcagggcagcggccgagcaacagcgcatgaatcaagag | |
| | ctccaagacatggttaacttgcaccagtgcaaaaggggtatcttttgtctggtaaagca | |
| | ggccaaagtcacctacgacagtaataccaccggacaccgccttagctacaagttgccaa | |
| | ccaagcgtcagaaattggtggtcatggtgggagaaaagcccattaccataactcagcac | |
| | tcggtagaaaccgaaggctgcattcactcaccttgtcaaggacctgaggatctctgcac | |
| | ccttattaagaccctgtgcggtctcaaagatcttattccctttaactaataaaaaaaaa | |
| | taataaagcatcacttacttaaaatcagttagcaaatttctgtccagtttattcagcag | |
| | cacctccttgccctcctcccagctctggtattgcagcttcctcctggctgcaaactttc | |
| | tccacaatctaaatggaatgtcagtttcctcctgttcctgtccatccgcacccactatc | |
| | ttcatgttgttgcagatgaagcgcgcaagaccgtctgaagataccttcaaccccgtgta | |
| | tccatatgacacggaaaccggtcctccaactgtgccttttcttactcctccctttgtat | |
| | cccccaatgggtttcaagagagtccccctggggtactctctttgcgcctatccgaacct | |
| | ctagttacctccaatggcatgcttgcgctcaaaatgggcaacggcctctctctggacga | |
| | ggccggcaaccttacctcccaaaatgtaaccactgtgagcccacctctcaaaaaaacca | |
| | agtcaaacataaacctggaaatatctgcacccctcacagttacctcagaagccctaact | |
| | gtggctgccgccgcacctctaatggtcgcgggcaacacactcaccatgcaatcacaggc | |
| | cccgctaaccgtgcacgactccaaacttagcattgccacccaaggacccctcacagtgt | |
| | cagaaggaaagctagccctgcaaacatcaggccccctcaccaccaccgatagcagtacc | |
| | cttactatcactgcctcaccccctctaactactgccactggtagcttgggcattgactt | |
| | gaaagagcccatttatacacaaaatggaaaactaggactaaagtacggggctcctttgc | |
| | atgtaacagacgacctaaacactttgaccgtagcaactggtccaggtgtgactattaat | |
| | aatacttccttgcaaactaaagttactggagccttgggttttgattcacaaggcaatat | |
| | gcaacttaatgtagcaggaggactaaggattgattctcaaaacagacgccttatacttg | |
| | atgttagttatccgtttgatgctcaaaaccaactaaatctaagactaggacagggccct | |
| | ctttttataaactcagcccacaacttggatattaactacaacaaaggcctttacttgtt | |
| | tacagcttcaaacaattccaaaaagcttgaggttaacctaagcactgccaaggggttga | |
| | tgtttgacgctacagccatagccattaatgcaggagatgggcttgaatttggttcacct | |
| | aatgcaccaaacacaaatcccctcaaaacaaaaattggccatggcctagaatttgattc | |
| | aaacaaggctatggttcctaaactaggaactggccttagttttgacagcacaggtgcca | |
| | ttacagtaggaaacaaaaataatgataagctaactttgtggaccacaccagctccatct | |
| | cctaactgtagactaaatgcagagaaagatgctaaactcactttggtcttaacaaaatg | |
| | tggcagtcaaatacttgctacagtttcagttttggctgttaaaggcagtttggctccaa | |
| | tatctggaacagttcaaagtgctcatcttattataagatttgacgaaaatggagtgcta | |
| | ctaaacaattccttcctggacccagaatattggaactttagaaatggagatcttactga | |
| | aggcacagcctatacaaacgctgttggatttatgcctaacctatcagcttatccaaaat | |
| | ctcacggtaaaactgccaaaagtaacattgtcagtcaagtttacttaaacggagacaaa | |
| | actaaacctgtaacactaaccattacactaaacggtacacaggaaacaggagacacaac | |
| | tccaagtgcatactctatgtcattttcatgggactggtctggccacaactacattaatg | |
| | aaatatttgccacatcctcttacactttttcatacattgcccaagaataaagaatcgtt | |
| | tgtgttatgtttcaacgtgtttatttttcaattgcagaaaatttcaagtcatttttcat | |
| | tcagtagtatagccccaccaccacatagcttatacagatcaccgtaccttaatcaaact | |
| | cacagaaccctagtattcaacctgccacctccctcccaacacacagagtacacagtcct | |
| | ttctccccggctggccttaaaaagcatcatatcatgggtaacagacatattcttaggtg | |
| | ttatattccacacggtttcctgtcgagccaaacgctcatcagtgatattaataaactcc | |
| | ccgggcagctcacttaagttcatgtcgctgtccagctgctgagccacaggctgctgtcc | |
| | aacttgcggttgcttaacgggcggcgaaggagaagtccacgcctacatgggggtagagt | |
| | cataatcgtgcatcaggatagggggtggtgctgcagcagcgcgcgaataaactgctgcc | |
| | gccgccgctccgtcctgcaggaatacaacatggcagtggtctcctcagcgatgattcgc | |
| | accgcccgcagcataaggcgccttgtcctccgggcacagcagcgcaccctgatctcact | |
| | taaatcagcacagtaactgcagcacagcaccacaatattgttcaaaatcccacagtgca | |
| | aggcgctgtatccaaagctcatggggggaccacagaacccacgtggccatcataccaca | |
| | agcgcaggtagattaagtggcgacccctcataaacactaaaccagctggccaaaacctg | |
| | cccgccggctatacactgcagggaaccgggactggaacaatgacagtggagagcccagg | |
| | actcgtaaccatggatcatcatgctcgtcatgatatcaatgttggcacaacacaggcac | |
| | acgtgcatacacttcctcaggattacaagctcctcccgcgttagaaccatatcccaggg | |
| | aacaacccattcctgaatcagcgtaaatcccacactgcagggaagacctcgcacgtaac | |
| | tcacgttgtgcattgtcaaagtgttacattcgggcagcagcggatgatcctccagtatg | |
| | gtagcgcgggtttctgtctcaaaaggaggtagacgatccctactgtacggagtgcgccg | |
| | agacaaccgagatcgtgttggtcgtagtgtcatgccaaatggaacgccggacgtagtca | |
| | tatttcctgaagcaaaaccaggtgcgggcgtgacaaacagatctgcgtctccggtctgc | |
| | cgcttagatcgctctgtgtagtagttgtagtatatccactctctcaaagcatccaggcg | |
| | ccccctggcttcgggttctatgtaaactccttcatgcgccgctgccctgataacatcca | |
| | ccaccgcagaataagccacacccagccaacctacacattcgttctgcgagtcacacacg | |
| | ggaggagcgggaagagctggaagaaccatgtttttttttttattccaaaagattatcca | |
| | aaacctcaaaatgaagatctattaagtgaacgcgctcccctccggtggcgtggtcaaac | |
| | tctacagccaaagaacagataatggcatttgtaagatgttgcacaatggcttccaaaag | |
| | gcaaacggccctcacgtccaagtggacgtaaaggctaaacccttcagggtgaatctcct | |
| | ctataaacattccagcaccttcaaccatgcccaaataattctcatctcgccaccttctc | |
| | aatatatctctaagcaaatcccgaatattaagtccggccattgtaaaaatctgctccag | |
| | agcgccctccaccttcagcctcaagcagcgaatcatgattgcaaaaattcaggttcctc | |
| | acagacctgtataagattcaaaagcggaacattaacaaaaataccgcgatcccgtaggt | |
| | cccttcgcagggccagctgaacataatcgtgcaggtctgcacggaccagcgcggccact | |
| | tccccgccaggaaccatgacaaaagaacccacactgattatgacacgcatactcggagc | |
| | tatgctaaccaggtagccccgatgtaagcttgttgcatgggcggcgatataaaatgcaa | |
| | ggtgctgctcaaaaaatcaggcaaagcctcgcgcaaaaaagaaagcacatcgtagtcat | |
| | gctcatgcagataaaggcaggtaagctccggaaccaccacagaaaaagacaccattttt | |
| | ctctcaaacatgtctgcgggtttctgcataaacacaaaataaaataacaaaaaaacatt | |
| | taaacattagaagcctgtcttacaacaggaaaaacaacccttataagcataagacggac | |
| | tacggccatgccggcgtgaccgtaaaaaaactggtcaccgtgattaaaaagcaccaccg | |
| | acagctcctcggtcatgtccggagtcataatgtaagactcggtaaacacatcaggttga | |
| | ttcacatcggtcagtgctaaaaagcgaccgaaatagcccgggggaatacatacccgcag | |
| | gcgtagagacaacattacagcccccataggaggtataacaaaattaataggagagaaaa | |
| | acacataaacacctgaaaaaccctcctgcctaggcaaaatagcaccctcccgctccaga | |
| | acaacatacagcgcttccacagcggcagccataacagtcagccttaccagtaaaaaaga | |
| | aaacctattaaaaaaacaccactcgacacggcaccagctcaatcagtcacagtgtaaaa | |
| | aagggccaagtgcagagcgagtatatataggactaaaaaatgacgtaacggttaaagtc | |
| | cacaaaaaacacccagaaaaccgcacgcgaacctacgcccagaaacgaaagccaaaaaa | |
| | cccacaacttcctcaaatcgtcacttccgttttcccacgttacgtcacttcccatttta | |
| | agaaaactacaattcccaacacatacaagttactccgccctaaaacctacgtcacccgc | |
| | cccgttcccacgccccgcgccacgtcacaaactccaccccctcattatcatattggctt | |
| | caatccaaaataaggtatattattgatgatgttaattaacatgcatggat | |
| 49 | acggcggggttttacgagattgtgattaaggtccccagcgaccttgacgggcatctgcc | Rep2 |
| | cggcatttctgacagctttgtgaactgggtggccgagaaggaatgggagttgccgccag | |
| | attctgacatggatctgaatctgattgagcaggcacccctgaccgtggccgagaagctg | |
| | cagcgcgactttctgacggaatggcgccgtgtgagtaaggccccggaggcccttttctt | |
| | tgtgcaatttgagaagggagagagctacttccacatgcacgtgctcgtggaaaccaccg | |
| | gggtgaaatccatggttttgggacgtttcctgagtcagattcgcgaaaaactgattcag | |
| | agaatttaccgcgggatcgagccgactttgccaaactggttcgcggtcacaaagaccag | |
| | aaatggcgccggaggcgggaacaaggtggtggatgagtgctacatccccaattacttgc | |
| | tccccaaaacccagcctgagctccagtgggcgtggactaatatggaacagtatttaagc | |
| | gcctgtttgaatctcacggagcgtaaacggttggtggcgcagcatctgacgcacgtgtc | |
| | gcagacgcaggagcagaacaaagagaatcagaatcccaattctgatgcgccggtgatca | |
| | gatcaaaaacttcagccaggtacatggagctggtcgggtggctcgtggacaaggggatt | |
| | acctcggagaagcagtggatccaggaggaccaggcctcatacatctccttcaatgcggc | |
| | ctccaactcgcggtcccaaatcaaggctgccttggacaatgcgggaaagattatgagcc | |
| | tgactaaaaccgcccccgactacctggtgggccagcagcccgtggaggacatttccagc | |
| | aatcggatttataaaattttggaactaaacgggtacgatccccaatatgcggcttccgt | |
| | ctttctgggatgggccacgaaaaagttcggcaagaggaacaccatctggctgtttgggc | |
| | ctgcaactaccgggaagaccaacatcgcggaggccatagcccacactgtgcccttctac | |
| | gggtgcgtaaactggaccaatgagaactttcccttcaacgactgtgtcgacaagatggt | |
| | gatctggtgggaggaggggaagatgaccgccaaggtcgtggagtcggccaaagccattc | |
| | tcggaggaagcaaggtgcgcgtggaccagaaatgcaagtcctcggcccagatagacccg | |
| | actcccgtgatcgtcacctccaacaccaacatgtgcgccgtgattgacgggaactcaac | |
| | gaccttcgaacaccagcagccgttgcaagaccggatgttcaaatttgaactcacccgcc | |
| | gtctggatcatgactttgggaaggtcaccaagcaggaagtcaaagactttttccggtgg | |
| | gcaaaggatcacgtggttgaggtggagcatgaattctacgtcaaaaagggtggagccaa | |
| | gaaaagacccgcccccagtgacgcagatataagtgagcccaaacgggtgcgcgagtcag | |
| | ttgcgcagccatcgacgtcagacgcggaagcttcgatcaactacgcagacaggtaccaa | |
| | aacaaatgttctcgtcacgtgggcatgaatctgatgctgtttccctgcagacaatgcga | |
| | gagaatgaatcagaattcaaatatctgcttcactcacggacagaaagactgtttagagt | |
| | gctttcccgtgtcagaatctcaacccgtttctgtcgtcaaaaaggcgtatcagaaactg | |
| | tgctacattcatcatatcatgggaaaggtgccagacgcttgcactgcctgcgatctggt | |
| | caatgtggatttggatgactgcatctttgaacaataa | |
| 50 | atggctgccgatggttatcttccagattggctcgaggacactctctctgaaggaataag | AAV2 cap |
| | acagtggtggaagctcaaacctggcccaccaccaccaaagcccgcagagcggcataagg | |
| | acgacagcaggggtcttgtgcttcctgggtacaagtacctcggacccttcaacggactc | |
| | gacaagggagagccggtcaacgaggcagacgccgcggccctcgagcacgacaaagccta | |
| | cgaccggcagctcgacagcggagacaacccgtacctcaagtacaaccacgccgacgcgg | |
| | agtttcaggagcgccttaaagaagatacgtcttttgggggcaacctcggacgagcagtc | |
| | ttccaggcgaaaaagagggttcttgaacctctgggcctggttgaggaacctgttaagac | |
| | ggctccgggaaaaaagaggccggtagagcactctcctgtggagccagactcctcctcgg | |
| | gaaccggaaaggcgggccagcagcctgcaagaaaaagattgaattttggtcagactgga | |
| | gacgcagactcagtacctgacccccagcctctcggacagccaccagcagccccctctgg | |
| | tctgggaactaatacgatggctacaggcagtggcgcaccaatggcagacaataacgagg | |
| | gcgccgacggagtgggtaattcctcgggaaattggcattgcgattccacatggatgggc | |
| | gacagagtcatcaccaccagcacccgaacctgggccctgcccacctacaacaaccacct | |
| | ctacaaacaaatttccagccaatcaggagcctcgaacgacaatcactactttggctaca | |
| | gcaccccttgggggtattttgacttcaacagattccactgccacttttcaccacgtgac | |
| | tggcaaagactcatcaacaacaactggggattccgacccaagagactcaacttcaagct | |
| | ctttaacattcaagtcaaagaggtcacgcagaatgacggtacgacgacgattgccaata | |
| | accttaccagcacggttcaggtgtttactgactcggagtaccagctcccgtacgtcctc | |
| | ggctcggcgcatcaaggatgcctcccgccgttcccagcagacgtcttcatggtgccaca | |
| | gtatggatacctcaccctgaacaacgggagtcaggcagtaggacgctcttcattttact | |
| | gcctggagtactttccttctcagatgctgcgtaccggaaacaactttaccttcagctac | |
| | acttttgaggacgttcctttccacagcagctacgctcacagccagagtctggaccgtct | |
| | catgaatcctctcatcgaccagtacctgtattacttgagcagaacaaacactccaagtg | |
| | gaaccaccacgcagtcaaggcttcagttttctcaggccggagcgagtgacattcgggac | |
| | cagtctaggaactggcttcctggaccctgttaccgccagcagcgagtatcaaagacatc | |
| | tgcggataacaacaacagtgaatactcgtggactggagctaccaagtaccacctcaatg | |
| | gcagagactctctggtgaatccgggcccggccatggcaagccacaaggacgatgaagaa | |
| | aagttttttcctcagagcggggttctcatctttgggaagcaaggctcagagaaaacaaa | |
| | tgtggacattgaaaaggtcatgattacagacgaagaggaaatcaggacaaccaatcccg | |
| | tggctacggagcagtatggttctgtatctaccaacctccagagaggcaacagacaagca | |
| | gctaccgcagatgtcaacacacaaggcgttcttccaggcatggtctggcaggacagaga | |
| | tgtgtaccttcaggggcccatctgggcaaagattccacacacggacggacattttcacc | |
| | cctctcccctcatgggggattcggacttaaacaccctcctccacagattctcatcaaga | |
| | acaccccggtacctgcgaatccttcgaccaccttcagtgcggcaaagtttgcttccttc | |
| | atcacacagtactccacgggacaggtcagcgtggagatcgagtgggagctgcagaagga | |
| | aaacagcaaacgctggaatcccgaaattcagtacacttccaactacaacaagtctgtta | |
| | atgtggactttactgtggacactaatggcgtgtattcagagcctcgccccattggcacc | |
| | agatacctgactcg | |
| 51 | aggaacccctagtgatggagttggccactccctctctgcgcgctcgctcgctcactgag | ITR |
| | gccgggcgaccaaaggtcgcccgacgcccgggctttgcccgggcggcctcagtgagcga | |
| | gcgagcgcgcagagagggagtggccaa | |
| 52 | SAAGADXAXDS | |

1. A nucleic acid stuffer sequence comprising one or more of the following features: no CpG island and no more than four contiguous nucleobases of the same identity; wherein the nucleic acid stuffer sequences does not include an open reading frame greater than 20 amino acids in length.

2. The nucleic acid stuffer sequence of claim 1, having no CpG island.

3. The nucleic acid stuffer sequence of claim 1 or claim 2, having no more than four contiguous nucleobases of the same identity.

4. The nucleic acid stuffer sequence of any one of claims 1-3, comprising between about 40% and about 50% GC content.

5. The nucleic acid stuffer sequence of any one of claims 1-4, wherein the nucleic acid stuffer sequence does not comprise a restriction enzyme cleavage site.

6. The nucleic acid stuffer sequence of any one of claims 1-5, wherein the nucleic acid stuffer sequence has a length of about 100 nucleobases to about 5000 nucleobases.

7. The nucleic acid stuffer sequence of claim 6, wherein the nucleic acid stuffer sequence has a length of about 2200 nucleobases to about 2300 nucleobases.

8. The nucleic acid stuffer sequence of claim 6, wherein the nucleic acid stuffer sequence has a length of about 3000 nucleobases to about 3100 nucleobases.

9. The nucleic acid stuffer sequence of claim 6, wherein the nucleic acid stuffer sequence has a length of about 300 nucleobases to about 400 nucleobases.

10. The nucleic acid stuffer sequence of any one of claims 1-6, wherein the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 7, 8, or 11.

11. A nucleic acid stuffer sequence comprising a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 7.

12. A nucleic acid stuffer sequence comprising a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 8.

13. A nucleic acid stuffer sequence comprising a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 11.

14. The nucleic acid stuffer sequence of any one of claims 11-13, wherein the nucleic acid stuffer sequence comprises no CpG island.

15. The nucleic acid sequence of any one of claims 11-14, wherein the nucleic acid stuffer sequence comprises no more than four contiguous nucleobases of the same identity.

16. The nucleic acid stuffer sequence of any one of claims 11-15, wherein the nucleic acid stuffer sequence comprises between about 40% and about 50% GC content.

17. The nucleic acid stuffer sequence of any one of claims 11-16, wherein the nucleic acid stuffer sequence does not comprise a restriction enzyme cleavage site.

18. The nucleic acid stuffer sequence of any one of claims 11-17, wherein the nucleic acid stuffer sequence has a length of about 100 to about 5000 nucleobases.

19. An adeno-associated virus (AAV) plasmid comprising the nucleic acid stuffer sequence of any one of claims 1-18.

20. The AAV plasmid of claim 19, wherein the AAV plasmid comprises an expression cassette comprising a heterologous sequence positioned between two inverted terminal repeat (ITR) sequences.

21. The AAV plasmid of claim 20, wherein the AAV plasmid comprises a backbone having a length of about 2000 to about 8000 nucleobases.

22. The AAV plasmid of claim 21, wherein the AAV plasmid comprises a backbone having a length of about 5500 to about 6000 nucleobases.

23. The AAV plasmid of claim 20-22, wherein the expression cassette has a length of about 3000 to about 6000 nucleobases.

24. The AAV plasmid of claim 23, wherein the expression cassette has a length of about 4000 nucleobases to about 5000 nucleobases.

25. The AAV plasmid of any one of claims 20-24, wherein the heterologous sequence encodes for a therapeutic peptide.

26. The AAV plasmid of claim 25, wherein the therapeutic peptide is selected from the group consisting of GUCY2D, MYO7A, RS1, CNBG3, ADAMTS10, ABCA4, and frataxin.

27. The AAV plasmid of any one of claims 19-26, comprising an antibiotic resistance gene.

28. The AAV plasmid of claim 26, wherein the antibiotic resistance gene comprises a kanamycin resistance gene.

29. The AAV plasmid of any one of claims 19-26, wherein the AAV plasmid does not comprise an antibiotic resistance gene.

30. The AAV plasmid of any one of claims 19-29, wherein the AAV plasmid does not comprise an ampicillin antibiotic resistance gene.

31. The AAV plasmid of any one of claims 19-30, wherein the ITR is derived from AAV serotype 1, AAV serotype 2, AAV serotype 3, AAV serotype 4, AAV serotype 5, AAV serotype 6, AAV serotype 7, AAV serotype 8, AAV serotype 9, AAV serotype 10, or AAV449.5(E531D).

32. The AAV plasmid of any one of claims 19-31, further comprising a promoter.

33. The AAV plasmid of any one of claims 19-32, further comprising a splice donor/splice acceptor sequence.

34. The AAV plasmid of any one of claims 19-33, further comprising a WPRE sequence.

35. The AAV plasmid of any one of claims 20-34, wherein the nucleic acid stuffer sequence is positioned outside of the expression cassette.

36. The AAV plasmid of any one of claims 20-35, comprising an origin of replication.

37. The AAV plasmid of claim 36, wherein the nucleic acid stuffer sequence is positioned 3′ to the origin of replication.

38. The AAV plasmid of claim 36 or claim 37, wherein the nucleic acid stuffer sequence is positioned between the origin of replication and an ITR.

39. The AAV plasmid of claim 38, wherein the nucleic acid stuffer sequence is located such that the ITR is about 1000 to about 4000 nucleobases away from the origin of replication.

40. The AAV plasmid of any one of claims 35-39, wherein the nucleic acid stuffer sequence comprises a first stuffer sequence and a second stuffer sequence.

41. The AAV plasmid of claim 40, wherein the first stuffer sequence has a length of about 3000 to about 3500 nucleobases.

42. The AAV plasmid of claim 40, wherein the first stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 8.

43. The AAV plasmid of claim 40, wherein the second stuffer sequence has a length of about 100 to about 500 nucleobases.

44. The AAV plasmid of claim 40, wherein the second stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 11.

45. The AAV plasmid of any one of claims 20-44, wherein the nucleic acid stuffer sequence is positioned within the expression cassette.

46. The AAV plasmid of claim 45, wherein the nucleic acid stuffer sequence has a length of about 2000 to about 3000 nucleobases.

47. The AAV plasmid of claim 44 or 45, wherein the nucleic acid stuffer sequence comprises a sequence about or at least about 80%, 81%, 82%, 83%, 84%, 85%, 86%, 87%, 88%, 89%, 90%, 91%, 92%, 93%, 94%, 95%, 96%, 97%, 98%, or 99% identical to at least 100 contiguous bases of SEQ ID NO: 7.

48. The AAV plasmid of any one of claims 19-47, wherein presence of the nucleic acid stuffer sequence reduces mutation of one or both of ITRs as compared to an AAV plasmid that does not comprise the nucleic acid stuffer sequence.

49. The AAV plasmid of any one of claims 19-48, wherein the AAV plasmid has no more than one origin of replication.

50. The AAV plasmid of any one of claims 19-49, wherein the AAV plasmid does not have a M13 origin of replication.

51. The AAV plasmid of any one of claims 19-50, wherein the AAV plasmid does not comprise a polyG/C sequence.

52. The AAV plasmid of any one of claims 19-51, wherein the AAV plasmid does not comprise a sequence having more than 2, 3, 4, 5, 6, 7, 8, 9, or 10 contiguous guanine bases.

53. A composition comprising the AAV plasmid of any one of claims 19-52, and a packaging plasmid comprising a viral replication (rep) gene and/or a viral capsid (cap) gene.

54. The composition of claim 53, wherein the packaging plasmid comprises the rep gene.

55. The composition of claim 54, wherein the rep gene encodes for Rep78, Rep68, Rep52, and Rep40.

56. The composition of any one of claims 53-55, wherein the packaging plasmid comprises the cap gene.

57. The composition of any one of claims 53-56, wherein the cap gene encodes for VP1, VP2 and VP3.

58. The composition of claim 57, wherein the cap gene comprises at least about 70%, 75%, 80%, 85%, 90%, 95%, 96%, 97%, 98%, 99% or 100% sequence identity with SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, or SEQ ID NO: 31.

59. The composition of any one of claims 53-58 and a helper plasmid.

60. A composition comprising the AAV plasmid of any one of claims 19-59, and a helper plasmid.

61. The composition of claim 59 or claim 60, wherein the helper plasmid comprises a E1a gene, a E1b gene, a E4 gene, a E2a, a e3 gene, a E5 gene, a Fiber gene, or a VA gene or a combination thereof.

62. The composition of claim 61, wherein the helper plasmid comprises a mutated Fiber gene.

63. The composition of claim 61, wherein the helper plasmid does not comprise a Fiber gene.

64. A cell comprising the AAV plasmid of any one of claims 19-52 or the composition of any one of claims 53-63.

65. An AAV particle comprising a nucleic acid and a capsid, wherein the AAV particle is produced by the AAV plasmid of any one of claims 19-52, the composition of any one of claims 53-63, or the cell of claim 64.

66. A pharmaceutical composition comprising the AAV particle of claim 65 and a pharmaceutically acceptable, carrier, buffer, diluent, or excipient, or any combination thereof.

67. A method for transducing a cell, the method comprising administering to the cell the AAV vector of any one of claims 19-52, the composition of any one of claims 53-63, the AAV particle of claim 65, or the pharmaceutical composition of claim 66.

68. The method of claim 64 or claim 67, wherein the cell is a photoreceptor cell.

69. The method of claim 68, wherein the cell is a retinal pigment epithelial (RPE) cell.

70. The method of claim 68, wherein the cell is a retinal ganglion cell.

71. A method for treating a disease or condition of an eye in a mammal, the method comprising administering to the mammal the AAV particle of claim 65, or the pharmaceutical composition of claim 66.

72. The method of claim 71, wherein the disease or condition comprises Retinitis pigmentosa, Leber Congenital Amaurosis (e.g., LCA10), Age Related Macular Degeneration (AMD), wet AMD, dry AMD, uveitis, Best disease, Stargardt disease, Usher Syndrome, Geographic Atrophy, Diabetic Retinopathy, Retinoschisis, Achromatopsia, Choroideremia, Bardet Biedl Syndrome, or glycogen storage diseases (ocular manifestation).

73. The method of claim 71 or 72, wherein the administration is to one or both eyes of the mammal.

74. The method of claim 73, wherein the AAV particle is administered intravitreally or subretinally.

75. The nucleic acid stuffer sequence of any one of claims 1-18, the AAV plasmid of any one of claims 19-52, the composition of any one of claims 53-63, the cell of claim 64, the AAV particle of claim 65, or the pharmaceutical composition of claim 66 for use in the treatment of a disease or condition of the eye

76. Use of the nucleic acid stuffer sequence of any one of claims 1-18, the AAV plasmid of any one of claims 19-52, the composition of any one of claims 53-63, the cell of claim 64, the AAV particle of claim 65, or the pharmaceutical composition of claim 66 in the manufacture of a medicament for use in the treatment of a disease or condition of the eye.


SEQUENCE LISTING (52 sequences)

SEQ ID NO: 1 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 292 bp, synthetic construct
  source 1..292: mol_type = other DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggc

SEQ ID NO: 3 - DNA, 164 bp, synthetic construct
  source 1..164: mol_type = other DNA
tctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttac

SEQ ID NO: 4 - DNA, 676 bp, synthetic construct
  source 1..676: mol_type = other DNA
atgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgag

SEQ ID NO: 5 - DNA, 588 bp, synthetic construct
  source 1..588: mol_type = other DNA
tcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcg

SEQ ID NO: 6 - DNA, 229 bp, synthetic construct
  source 1..229: mol_type = other DNA
tcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatc

SEQ ID NO: 7 - DNA, 2235 bp, synthetic construct
  source 1..2235: mol_type = other DNA
cagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcaggcgcgccttgcatgc

SEQ ID NO: 8 - DNA, 3048 bp, synthetic construct
  source 1..3048: mol_type = other DNA
catgaccgagtaacgactcagtcgtcccacgaggcataagccagctgtaataagcgtacaagcgcacacacttgagtgggacaccttagggattcccaataattcggcaatctactgtgatacgacacttcagaaacgatgacctgaccgacaagctgaactctacttcacctactactcaacactacaaccagtcctctgttgacagtataacttgttggtactattatgactgaagggtaggtttgattggatttgtttgcttcgtctggtcagcaggaggatctctcctttatacctgcagataaccgacgggtgcctattgtatcgaagtcattactacatccacctggtccacttgataaatcaatccaaccgatacctacctgactaaggtcccttcacgaggacctttgtcacccagtaatcagatttagaagtcgtattcttacacaacctcacaactgattctgaacgcaccgtcgaagtcaacaccacagtatctgaggaagtaagaacagtttgtactttgagcggcgcccactgtgaatacgagagactttaggtcactttcaactatttagtgggtcaccttcggaactcagacgaggacaccaccacagtagaggtggatagtccaaacacagttgttgtacagacccagtggattgaataggatcttgatcaggtgtgtccagacgctccagactatagttaaataggagtaagaccgctaagcgcagcaggagttcccaatcgttacaactagatcctgtggtgatatatctagtgtccgacttacatgcggcctctgtattagttcatattcattggtctgcacgcctcaggagagatattcgacctgaggataccaggacactttggatcagtatgggtcggaccagttgaacactgataaagtactgaccgagtgggttgtgacggcccagagattcagtcctactttctcaactggagaggaaccacctgttctgagtaacccacaatcgtgtccagagactggacaacactgaatgcaggttcctttctgttcacccagataagggagaccacactaagtggtgtattcgcacgacgatactggtgtcatttatttccctggccagtgtgtacctgcgtgactgcttccgaaggagtgttacaaggagaaaggagagtataactacaaggaatgtagacactgcctgcagcccatgaattgcaatataatcggacttgctcggagtccctctactacttaggaagaggattgatcaccacaggtcctctggtcagtttaggatgcataatgatccatgccgagacttggaattgatatatactggtatcaccgtataggttgctggttgtagtgtctttagcccacccagtgtaatagcatgtatagttaatctgcgtaacgatccctacgaacgacctagaggtcagtcacaccacggcgcgaagaagactcttacactaatagtaagctattgtatctagttaagtgaccaacccatttggttcacatcgtagttgactttccgggtccttggacccgaaattctatcacatcagtccctaatgcgatgagtgcgtctttctggttcacgtggtctgtatcatcaattcctaaatcttagtgactaattattatttcgagacattgtacacctggatggtactgtttctgtcccacgccctacaacggacaaccaacaacgcaccttgttgaacaacaatgatccactacacaagctccgaggtcgttctcgtcttgatttgaaacatgggtgcaatgtgtattagaggtttcaatccatatatcctaatacgttgatattgttctttagtaggtggctcctaaagctcaaagttgcaagcaagctaacaataccatttcacgacaaggaggataaccacgtgtgttattctgactccatgtaatctgctaatcccaccttcttcagtcgagctttattatattcgcgtaccggagacccaggcagaggtgaggacgaatatgttgcaggagtaaagaagctattgaaggatttcttaggacgcgctgctggtgttctaaagtagtatagaagctaagtcaagccggagcatactccgatatttggagcctgataccagttactgggtgtttagcagaagcgttacgtgacttacacgatataatgactatacttgatctgaaggcatcaagtatcaacgaaagtcctttctgattgacaaacagactctcactttacagggataggattggaaccaccaatctgaggtattcaccagtatccagccactgctctctcaagtccaacgtaatccaaggagactccggcaattccttaattggcgttagctactgtgttgcgagcatttattactatctggcaaggagtgacaaattagcaggattgattgaggtttgtgatccaccagaaggaaggactcaccgttaggtatggtgaagatacccagcttgtgctgactgaggatagcaagacagatcacttatcactcgaacagatcggacccaccgcacacctcaatatctctttaggaccaattcaagaacaacttagacgctgtacacttaaacagcacagctgtgggtaagagaagtaatagagcgcgatatcagcagcagcctcttgtagatcggcttcaagatatctatgtgtggaaataggtcactagagtgagcggtactttcgctcttacaggtttgaaaggtcttagcagcgctatttagcgagtcctggagaccacgacaacgagcgctgtggtcctcacaaataggaccaacaataacaatacaggatttatcaagagatagaagtcggtataccgattctgctgtttcgtagtcggctacagatcaatagattattgtgttcagattagttgaggaagttgaatgggtcaaggaccttacctaataccaggtcaccggacttggtaattttccctcttggggttggtgagtagtactgtctaccaactggtgactggtgatcccttacttgtttggaccct

SEQ ID NO: 9 - DNA, 671 bp, synthetic construct
  source 1..671: mol_type = other DNA
tgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggg

SEQ ID NO: 10 - DNA, 810 bp, synthetic construct
  source 1..810: mol_type = other DNA
atgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 11 - DNA, 381 bp, synthetic construct
  source 1..381: mol_type = other DNA
ccctgttggttcaagtctccctaatagtttgaggggaaggagttggtgtcaagggtctgaccaccatattacgggactagagacaggtggaggtctggacaccctattggtggatcattcattatggatacggtcctggacaagtggactgatcccaattaggagactggtccagtgtcccaccttgagaggtactgatactcacttacctcaaacactaggttcttaagagggtgatggagagagattaggaccaaaccagagactttggtcctaactccagaggtagtgagtacaaaacaccactaggtgaggttgggaccagtctattttctccaaactgagaaggtgtcagtggaggcacacttaccctctcactgg

SEQ ID NO: 12 - DNA, 1803 bp, synthetic construct
  source 1..1803: mol_type = other DNA
atgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 13 - DNA, 1809 bp, synthetic construct
  source 1..1809: mol_type = other DNA
agctcagtttcctcctgttcctgtccatccgcacccactatcttcatgttgttgcagtaaaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcattcttgcgctcaaaataggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatcgtcgcgggcaacacactcaccatacaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatctgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatatttgacgctacagccatagccattaatgcaggagatgggcttgaatttggttcacctaatgcaccaaacacaaatcccctcaaaacaaaaattggccatggcctagaatttgattcaaacaaggctattgttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggattttaacctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccgctagcacaactacattaatgaaatatttgccacatcctcttacactttttcatacattgcccaagaataa

SEQ ID NO: 14-20 - skipped (7 entries)

SEQ ID NO: 21 - protein, 728 aa, unidentified
AADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGTTMASGGGAPMADNNEGADGVGNASGNWHCDSTWLGDRVITTSTRTWALPTYNNHLYKQISSASTGASNDNHYFGYSTPWGYFDFNRFHCHFSPRDWQLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTNNNSNFTWTGASKYNLNGRESIINPGTAMASHKDDEDKFFPMSGVMIFGKESAGASNTALDNVMITDEEEIKATNPVATERFGTVAVNFQSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWEQKENSKRWNPEVQYTSNYAKSANVDFTVDNNGLYTEPRPIGTRYLTRPL

SEQ ID NO: 22 - protein, 727 aa, unidentified
AADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSANNNSEYSWTGATKYHLNGRDSLVNPGPAMASHKDDEEKFFPQSGVLIFGKQGSEKTNVDIEKVMITDEEEIRTTNPVATEQYGSVSTNLQRNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWEQKENSKRWNPEIQYTSNYNKSVNVDFTVDTNGVYSEPRPIGTRYLTRNL

SEQ ID NO: 23 - protein, 728 aa, unidentified
AADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANNNNSNFPWTAASKYHLNGRDSLVNPGPAMASHKDDEEKFFPMHGNLIFGKEGTTASNAELDNVMITDEEEIRTTNPVATEQYGTVANNLQSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWEQKENSKRWNPEIQYTSNYNKSVNVDFTVDTNGVYSEPRPIGTRYLTRNL

SEQ ID NO: 24 - protein, 727 aa, unidentified
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSMRAAAGGAAVEGGQGADGVGNASGDWHCDSTWSEGHVTTTSTRTWVLPTYNNHLYKRLGESLQSNTYNGFSTPWGYFDFNRFHCHFSPRDWQLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANNYKIPATGSDSLIKYETHSTLDGRWSALTPGPPMATAGPADSKFSNSQLIFAGPKQNGNTATVPGTLIFTSEEELAATNATDTDMWGNLPGGDQSSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEQKERSKRWNPEVQFTSNYGQQNSLLWAPDAAGKYTEPRAIGTRYLTHHL

SEQ ID NO: 25 - protein, 716 aa, unidentified
SFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFEKLADDTSFGGNLGKAVFQAKKRVLEPFGLVEEGAKTAPTGKRIDDHFPKRKKARTEEDSKPSTSSDAEAGPSGSQQLQIPAQPASSLGDTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGNRASVSAFATTNRMELEGASYQVPPQPNGMTNNLQGSNTYALENTMIFNSQPANPGTTATYLEGNMLITSESETQPVNRVAYNVGGQMATNNQSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWEKKENSKRWNPEIQYTNNYNDPQFVDFAPDSTGEYRTTRPIGTRYLTRPL

SEQ ID NO: 26 - protein, 728 aa, unidentified
AADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGTTMASGGGAPMADNNEGADGVGNASGNWHCDSTWLGDRVITTSTRTWALPTYNNHLYKQISSASTGASNDNHYFGYSTPWGYFDFNRFHCHFSPRDWQLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTNNNSNFTWTGASKYNLNGRESIINPGTAMASHKDDKDKFFPMSGVMIFGKESAGASNTALDNVMITDEEEIKATNPVATERFGTVAVNLQSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWEQKENSKRWNPEVQYTSNYAKSANVDFTVDNNGLYTEPRPIGTRYLTRPL

SEQ ID NO: 27 - protein, 729 aa, unidentified
AADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDNNNSNFAWTGATKYHLNGRNSLVNPGVAMATHKDDEDRFFPSSGVLIFGKTGATNKTTLENVLMTNEEEIRPTNPVATEEYGIVSSNLQANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWEQKENSKRWNPEIQYTSNFEKQTGVDFAVDSQGVYSEPRPIGTRYLTRNL

SEQ ID NO: 28 - protein, 730 aa, unidentified
AADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGNNNSNFAWTAGTKYHLNGRNSLANPGIAMATHKDDEERFFPSNGILIFGKQNAARDNADYSDVMLTSEEEIKTTNPVATEEYGIVADNLQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWEQKENSKRWNPEIQYTSNYYKSTSVDFAVNTEGVYSEPRPIGTRYLTRNL

SEQ ID NO: 29 - protein, 728 aa, unidentified
AADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTNNNSEFAWPGASSWALNGRNSLMNPGPAMASHKEGEDRFFPLSGSLIFGKQGTGRDNVDADKVMITNEEEIKTTNPVATESYGQVATNHQSQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWEQKENSKRWNPEIQYTSNYYKSNNVEFAVNTEGVYSEPRPIGTRYLTRNL

SEQ ID NO: 30 - protein, 738 aa, unidentified
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 31 - protein, 736 aa, unidentified
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMASQARNWVPGPSYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 32 - DNA, 10067 bp, synthetic construct
  source 1..10067: mol_type = other DNA
ggcctcttcgcttaattaacgccaggctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatccagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcaggcgcgccttgcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctgcagcctgcttaattaatcatatgcatgaccgagtaacgactcagtcgtcccacgaggcataagccagctgtaataagcgtacaagcgcacacacttgagtgggacaccttagggattcccaataattcggcaatctactgtgatacgacacttcagaaacgatgacctgaccgacaagctgaactctacttcacctactactcaacactacaaccagtcctctgttgacagtataacttgttggtactattatgactgaagggtaggtttgattggatttgtttgcttcgtctggtcagcaggaggatctctcctttatacctgcagataaccgacgggtgcctattgtatcgaagtcattactacatccacctggtccacttgataaatcaatccaaccgatacctacctgactaaggtcccttcacgaggacctttgtcacccagtaatcagatttagaagtcgtattcttacacaacctcacaactgattctgaacgcaccgtcgaagtcaacaccacagtatctgaggaagtaagaacagtttgtactttgagcggcgcccactgtgaatacgagagactttaggtcactttcaactatttagtgggtcaccttcggaactcagacgaggacaccaccacagtagaggtggatagtccaaacacagttgttgtacagacccagtggattgaataggatcttgatcaggtgtgtccagacgctccagactatagttaaataggagtaagaccgctaagcgcagcaggagttcccaatcgttacaactagatcctgtggtgatatatctagtgtccgacttacatgcggcctctgtattagttcatattcattggtctgcacgcctcaggagagatattcgacctgaggataccaggacactttggatcagtatgggtcggaccagttgaacactgataaagtactgaccgagtgggttgtgacggcccagagattcagtcctactttctcaactggagaggaaccacctgttctgagtaacccacaatcgtgtccagagactggacaacactgaatgcaggttcctttctgttcacccagataagggagaccacactaagtggtgtattcgcacgacgatactggtgtcatttatttccctggccagtgtgtacctgcgtgactgcttccgaaggagtgttacaaggagaaaggagagtataactacaaggaatgtagacactgcctgcagcccatgaattgcaatataatcggacttgctcggagtccctctactacttaggaagaggattgatcaccacaggtcctctggtcagtttaggatgcataatgatccatgccgagacttggaattgatatatactggtatcaccgtataggttgctggttgtagtgtctttagcccacccagtgtaatagcatgtatagttaatctgcgtaacgatccctacgaacgacctagaggtcagtcacaccacggcgcgaagaagactcttacactaatagtaagctattgtatctagttaagtgaccaacccatttggttcacatcgtagttgactttccgggtccttggacccgaaattctatcacatcagtccctaatgcgatgagtgcgtctttctggttcacgtggtctgtatcatcaattcctaaatcttagtgactaattattatttcgagacattgtacacctggatggtactgtttctgtcccacgccctacaacggacaaccaacaacgcaccttgttgaacaacaatgatccactacacaagctccgaggtcgttctcgtcttgatttgaaacatgggtgcaatgtgtattagaggtttcaatccatatatcctaatacgttgatattgttctttagtaggtggctcctaaagctcaaagttgcaagcaagctaacaataccatttcacgacaaggaggataaccacgtgtgttattctgactccatgtaatctgctaatcccaccttcttcagtcgagctttattatattcgcgtaccggagacccaggcagaggtgaggacgaatatgttgcaggagtaaagaagctattgaaggatttcttaggacgcgctgctggtgttctaaagtagtatagaagctaagtcaagccggagcatactccgatatttggagcctgataccagttactgggtgtttagcagaagcgttacgtgacttacacgatataatgactatacttgatctgaaggcatcaagtatcaacgaaagtcctttctgattgacaaacagactctcactttacagggataggattggaaccaccaatctgaggtattcaccagtatccagccactgctctctcaagtccaacgtaatccaaggagactccggcaattccttaattggcgttagctactgtgttgcgagcatttattactatctggcaaggagtgacaaattagcaggattgattgaggtttgtgatccaccagaaggaaggactcaccgttaggtatggtgaagatacccagcttgtgctgactgaggatagcaagacagatcacttatcactcgaacagatcggacccaccgcacacctcaatatctctttaggaccaattcaagaacaacttagacgctgtacacttaaacagcacagctgtgggtaagagaagtaatagagcgcgatatcagcagcagcctcttgtagatcggcttcaagatatctatgtgtggaaataggtcactagagtgagcggtactttcgctcttacaggtttgaaaggtcttagcagcgctatttagcgagtcctggagaccacgacaacgagcgctgtggtcctcacaaataggaccaacaataacaatacaggatttatcaagagatagaagtcggtataccgattctgctgtttcgtagtcggctacagatcaatagattattgtgttcagattagttgaggaagttgaatgggtcaaggaccttacctaataccaggtcaccggacttggtaattttccctcttggggttggtgagtagtactgtctaccaactggtgactggtgatcccttacttgtttggaccctggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcacgtagaaacccagtccgcagaaacggtgctgacccctgatgaatgtcacctactgggttatctggacaagggaaaacccaagcgcaaagagaaagcaggtagcttggagtggggttacatggtgataggtagactgggaggttttatggacaggaaccgaacctcaattcccaggtggggtgtcctctggtaaggttgggaagtcctggaaagtaaactggatggctttctcgccgctgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaattttgttaaaatttttgttaaatcagctcattttttaaccaataggccctgttggttcaagtctccctaatagtttgaggggaaggagttggtgtcaagggtctgaccaccatattacgggactagagacaggtggaggtctggacaccctattggtggatcattcattatggatacggtcctggacaagtggactgatcccaattaggagactggtccagtgtcccaccttgagaggtactgatactcacttacctcaaacactaggttcttaagagggtgatggagagagattaggaccaaaccagagactttggtcctaactccagaggtagtgagtacaaaacaccactaggtgaggttgggaccagtctattttctccaaactgagaaggtgtcagtggaggcacacttaccctctcactggctattcgccattcaggctgcgcaactgttgggaagggcgatcggtgcg

SEQ ID NO: 33 - DNA, 10052 bp, synthetic construct
  source 1..10052: mol_type = other DNA
cctcttcgcttaattaacgccaggctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggtctcccaggggcttcccagtggtccccaggaaccctcgacagggcccggtctctctcgtccagcaagggcagggacgggccacaggccaagggctctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgtcacgcaagatagaaggctttttgttattacttctctttggctatgaagccacattgggattatcgtctaccgaggatgaaggcgaggacccatggtatcaaaaagcctgcaagtgcgattgccaaggaggacccaatgctctgtggtctgcaggtgccacctccttggactgtataccagaatgcccatatcacaagcctctgggtttcgagtcaggggaggtcacaccggaccagatcacctgctctaacccggagcagtatgtgggctggtattcttcgtggactgcaaacaaggcccggctcaacagtcaaggctttgggtgtgcctggctctccaagttccaggacagtagccagtggttacagatagatctgaaggagatcaaagtgatttcaggcatcctcacccaggggcgctgtgacatcgatgagtggatgaccaagtacagcgtgcagtacaggaccgatgagcgcctgaactggatttactacaaggaccagactggaaacaaccgggtcttctatggcaactcggaccgcacctccacggttcagaacctgctgcggccccccatcatctcccgcttcatccgcctcatcccgctgggctggcacgtccgcattgccatccggatggagctgctggagtgcgtcagcaagtgtgcctgagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatccgattcgagtgagcagaccacatgatgattggtattctccctgaagaaagaatgtcagcaaggagtatctgaagtccgaccaagcctagaatccgcccggttcttatttgctgaagtaatactgttgtcaagtcagctacagggagtggtccctgcctggagtttgaagggctgtcaaagacattgtggtcacttactcaagtggtaaattccttacagactggactgcagttaagggtgataacgctactactaggacaagggtatccttgatcctgtggatctgtagtgacctgagactgtcaaggtatggtgagtatgacccttgaccctttccaacggttgtaaccgtgggcgctgtcaaatctgattaaccactactaggatagaatttggcagagctactgaaatggttacctggtcaagagactggcccacagcataagggcgagcaagacgatgctcagattaactaagtgaccgaagtatcctagatggtccactaccagcaaatgtgtcagacctccctcagttggtagtacaatgtatccttctagtcgcgacttattatatcgacgagcttatgtgtaagagaccagattctttgtaacctgacacttgagttcaaggtgtgtagtaggagagtgcaaaccctgaagcaacagtagccgaaccgggtcgaggacctagcgttgattgactttgtctggtgtatcctgtaaatttctccaatcgagatcgaggtaaagtggacctcacaggactttatcaaacaacagagttaatacctcaaatctctcgctgatatatgtgacaaattcattgcgtcacttatcgcagctatgttcggtcagcatattctggcgaacacccacgctgcagttcgacaaggatagtatctggcgctctcgcccaattcaagcacgcgaattactctattaacttgttgtctcttgtgtatatcataatcaattcctcctagttagtgattgaagacgaccgtgcacttggctgccttgatgcagaccacttcttgtggccggcacaccctataatagagatgtgataaataacttagctggactgtagttatttcgtcctagggattctgacgggtgcaaatcactaagtgtctttggtattgggtctgtaacaatgttgagaactgtggtgtatgtacgaacgatgtgatctgtagactacagcacagggtattgctgtttcgcaccagtgtgacacttgtgtggctgtcctctgtgctatagcgccgtatatcttatgcaggttgtatagcccaacgactgtttaggtacgtccgtataagaataggagtgctacactctacattgaatctatccagagagagttctgtagaattaggtgacgaacaaatctacgcgataggaaggagagacctggttgtcccaagggccgtgggtatgatgtttagtacaactaggaggctctaggtgtattggttgttggtcacgtagtgtccctacttggtcaggactaaagtttgtcagtgaactctctactattattcatcgaacctgtggtcgtcggcggtctgatatatgctcggtagattgcccgacaggacgtgaaggagcctaactattaaagcactctaactagcctatcgtaacaacttatattgggtatttgggtacgacggtgtactggaaatctatctaattcttctgatcgtatatcgggtcgtatattgatttatgacggaccgaccactcacgcggtccgatacgcggtagcgctatccaggaccctcttatgaattggtatcaatagattgtggaacttgttgcgtacactacagaatgtctttgcaaagaatgccaaattacaagctatagtcctcccactatactgcttcgtgactccaactagggtagttatagttgttcaagactggtactctgttaccaattagtagcgatattgtgtgggatactggtgtctgtcatacgagaactgggagtagcgctgtaaattctaaatagtcacactgttccagctggacgaccgctgtgtactctttgtagaaactcactgcggcttccacttatgagatttggttataggtcacaaagtggcagtccagttgcaggtctcttgttgtaatatgactggattgaattgactaggaagacgtagtctgttcttgttaggtccgacgtggtcccaatttgcttatccaatatgaggtcctagttagcagtcgctccgaggtcgaaatagatacccttgtggtttcgtagattgtctgattcgcaacaatatcctgctaatcaatctacggaaacaccgcttcagaggaagattgcgacggacagaagagtcagtttcggctgcttgtggtttggcctaatataaagattacttacggcttaggtatactgcacatcctccggccttgtttggtgcttgagattggtgcgcatatatgtaaggtgctatcgtatccgcctccagtacttgatcttgtcctgcggctttagggctgcttatccctccagcggtccaacctctgaagggtcaggccatacttcacctcctttgtctaggattctgttccgaatttccggtctgggtcatgtactctcaccatcgagtgtagggagacgattctcgtaacagccgtagccgaatcactttcacttgagtctatgggttgtgcctgagttctagcttagtatttaacaacggcgtcggtccaaagacggtacacacttaaccatagtgaacgaatgaaacagtaatcgctccaggttggcgcgccttgcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctgcagcctgcttaattaatcatatgcatgaccgagtaacgactcagtcgtcccacgaggcataagccagctgtaataagcgtacaagcgcacacacttgagtgggacaccttagggattcccaataattcggcaatctactgtgatacgacacttcagaaacgatgacctgaccgacaagctgaactctacttcacctactactcaacactacaaccagtcctctgttgacagtataacttgttggtactattatgactgaagggtaggtttgattggatttgtttgcttcgtctggtcagcaggaggatctctcctttatacctgcagataaccgacgggtgcctattgtatcgaagtcattactacatccacctggtccacttgataaatcaatccaaccgatacctacctgactaaggtcccttcacgaggacctttgtcacccagtaatcagatttagaagtcgtattcttacacaacctcacaactgattctgaacgcaccgtcgaagtcaacaccacagtatctgaggaagtaagaacagtttgtactttgagcggcgcccactgtgaatacgagagactttaggtcactttcaactatttagtgggtcaccttcggaactcagacgaggacaccaccacagtagaggtggatagtccaaacacagttgttgtacagacccagtggattgaataggatcttgatcaggtgtgtccagacgctccagactatagttaaataggagtaagaccgctaagcgcagcaggagttcccaatcgttacaactagatcctgtggtgatatatctagtgtccgacttacatgcggcctctgtattagttcatattcattggtctgcacgcctcaggagagatattcgacctgaggataccaggacactttggatcagtatgggtcggaccagttgaacactgataaagtactgaccgagtgggttgtgacggcccagagattcagtcctactttctcaactggagaggaaccacctgttctgagtaacccacaatcgtgtccagagactggacaacactgaatgcaggttcctttctgttcacccagataagggagaccacactaagtggtgtattcgcacgacgatactggtgtcatttatttccctggccagtgtgtacctgcgtgactgcttccgaaggagtgttacaaggagaaaggagagtataactacaaggaatgtagacactgcctgcagcccatgaattgcaatataatcggacttgctcggagtccctctactacttaggaagaggattgatcaccacaggtcctctggtcagtttaggatgcataatgatccatgccgagacttggaattgatatatactggtatcaccgtataggttgctggttgtagtgtctttagcccacccagtgtaatagcatgtatagttaatctgcgtaacgatccctacgaacgacctagaggtcagtcacaccacggcgcgaagaagactcttacactaatagtaagctattgtatctagttaagtgaccaacccatttggttcacatcgtagttgactttccgggtccttggacccgaaattctatcacatcagtccctaatgcgatgagtgcgtctttctggttcacgtggtctgtatcatcaattcctaaatcttagtgactaattattatttcgagacattgtacacctggatggtactgtttctgtcccacgccctacaacggacaaccaacaacgcaccttgttgaacaacaatgatccactacacaagctccgaggtcgttctcgtcttgatttgaaacatgggtgcaatgtgtattagaggtttcaatccatatatcctaatacgttgatattgttctttagtaggtggctcctaaagctcaaagttgcaagcaagctaacaataccatttcacgacaaggaggataaccacgtgtgttattctgactccatgtaatctgctaatcccaccttcttcagtcgagctttattatattcgcgtaccggagacccaggcagaggtgaggacgaatatgttgcaggagtaaagaagctattgaaggatttcttaggacgcgctgctggtgttctaaagtagtatagaagctaagtcaagccggagcatactccgatatttggagcctgataccagttactgggtgtttagcagaagcgttacgtgacttacacgatataatgactatacttgatctgaaggcatcaagtatcaacgaaagtcctttctgattgacaaacagactctcactttacagggataggattggaaccaccaatctgaggtattcaccagtatccagccactgctctctcaagtccaacgtaatccaaggagactccggcaattccttaattggcgttagctactgtgttgcgagcatttattactatctggcaaggagtgacaaattagcaggattgattgaggtttgtgatccaccagaaggaaggactcaccgttaggtatggtgaagatacccagcttgtgctgactgaggatagcaagacagatcacttatcactcgaacagatcggacccaccgcacacctcaatatctctttaggaccaattcaagaacaacttagacgctgtacacttaaacagcacagctgtgggtaagagaagtaatagagcgcgatatcagcagcagcctcttgtagatcggcttcaagatatctatgtgtggaaataggtcactagagtgagcggtactttcgctcttacaggtttgaaaggtcttagcagcgctatttagcgagtcctggagaccacgacaacgagcgctgtggtcctcacaaataggaccaacaataacaatacaggatttatcaagagatagaagtcggtataccgattctgctgtttcgtagtcggctacagatcaatagattattgtgttcagattagttgaggaagttgaatgggtcaaggaccttacctaataccaggtcaccggacttggtaattttccctcttggggttggtgagtagtactgtctaccaactggtgactggtgatcccttacttgtttggaccctggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcacgtagaaacccagtccgcagaaacggtgctgacccctgatgaatgtcacctactgggttatctggacaagggaaaacccaagcgcaaagagaaagcaggtagcttggagtggggttacatggtgataggtagactgggaggttttatggacaggaaccgaacctcaattcccaggtggggtgtcctctggtaaggttgggaagtcctggaaagtaaactggatggctttctcgccgctgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaattttgttaaaatttttgttaaatcagctcattttttaaccaataggccctgttggttcaagtctccctaatagtttgaggggaaggagttggtgtcaagggtctgaccaccatattacgggactagagacaggtggaggtctggacaccctattggtggatcattcattatggatacggtcctggacaagtggactgatcccaattaggagactggtccagtgtcccaccttgagaggtactgatactcacttacctcaaacactaggttcttaagagggtgatggagagagattaggaccaaaccagagactttggtcctaactccagaggtagtgagtacaaaacaccactaggtgaggttgggaccagtctattttctccaaactgagaaggtgtcagtggaggcacacttaccctctcactggctattcgccattcaggctgcgcaactgttgggaagggcgatcggtgcggg

SEQ ID NO: 34 - protein, 2177 aa, Homo sapiens [sequence omitted]

SEQ ID NO: 35 - protein, 2215 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 36 - protein, 3144 aa, Homo sapiens
MTDKSIVILSLMVFHSSFINGKTCRRQLVEEWHPQPSSYVVNWTLTENICLDFYRDCWFLGVNTKIDTSGNQAVPQICPLQIQLGDILVISSEPSLQFPEINLMNVSETSFVGCVQNTTTEDQLLFGCRLKGMHTVNSKWLSVGTHYFITVMASGPSPCPLGLRLNVTVKQQFCQESLSSEFCSGHGKCLSEAWSKTYSCHCQPPFSGKYCQELDACSFKPCKNNGSCINKRENWDEQAYECVCHPPFTGKNCSEIIGQCQPHVCFHGNCSNITSNSFICECDEQFSGPFCEVSAKPCVSLLFWKRGICPNSSSAYTYECPKGSSSQNGETDVSEFSLVPCQNGTDCIKISNDVMCICSPIFTDLLCKSIQTSCESFPLRNNATCKKCEKDYPCSCISGFTEKNCEKAIDHCKLLSINCLNEEWCFNIIGRFKYVCIPGCTKNPCWFLKNVYLIHQHLCYCGVTFHGICQDKGPAQFEYVWQLGFAGSEGEKCQGVIDAYFFLAANCTEDATYVNDPEDNNSSCWFPHEGTKEICANGCSCLSEEDSQEYRYLCFLRWAGNMYLENTTDDQENECQHEAVCKDEINRPRCSCSLSYIGRLCVVNVDYCLGNHSISVHGLCLALSHNCNCSGLQRYERNICEIDTEDCKSASCKNGTTSTHLRGYFFRKCVPGFKGTQCEIDIDECASHPCKNGATCIDQPGNYFCQCVPPFKVVDGFSCLCNPGYVGIRCEQDIDDCILNACEHNSTCKDLHLSYQCVCLSDWEGNFCEQESNECKMNPCKNNSTCTDLYKSYRCECTSGWTGQNCSEEINECDSDPCMNGGLCHESTIPGQFVCLCPPLYTGQFCHQRYNLCDLLHNPCRNNSTCLALVDANQHCICREEFEGKNCEIDVKDCLFLSCQDYGDCEDMVNNFRCICRPGFSGSLCEIEINECSSEPCKNNGTCVDLTNRFFCNCEPEYHGPFCELDVNKCKISPCLDEENCVYRTDGYNCLCAPGYTGINCEINLDECLSEPCLHDGVCIDGINHYTCDCKSGFFGTHCETNANDCLSNPCLHGRCTELINEYPCSCDADGTSTQCKIKINDCTSIPCMNEGFCQKSAHGFTCICPRGYTGAYCEKSIDNCAEPELNSVICLNGGICVDGPGHTFDCRCLPGFSGQFCEININECSSSPCLHGADCEDHINGYVCKCQPGWSGHHCENELECIPNSCVHELCMENEPGSTCLCTPGFMTCSIGLLCGDEIRRITCLTPIFQRTDPISTQTYTIPPSETLVSSFPSIKATRIPAIMDTYPVDQGPKQTGIVKHDILPTTGLATLRISTPLESYLLQELIVTRELSAKHSLLSSADVSSSRFLNFGIRDPAQIVQDKTSVSHMPIRTSAATLGFFFPDRRARTPFIMSSLMSDFIFPTQSLLFENCQTVALSATPTTSVIRSIPGADIELNRQSLLSRGFLLIAASISATPVVSRGAQEDIEEYSADSLISRREHWRLLSPSMSPIFPAKVIISKQVTILNSSALHRFSTKAFNPSEYQAITEASSNQRLTNIKSQAADSLRELSQTCATCSMTEIKSSREFSDQVLHSKQSHFYETFWMNSAILASWYALMGAQTITSGHSFSSATEITPSVAFTEVPSLFPSKKSAKRTILSSSLEESITLSSNLDVNLCLDKTCLSIVPSQTISSDLMNSDLTSKMTTDELSVSENILKLLKIRQYGITMGPTEVLNQESLLDMEKSKGSHTLFKLHPSDSSLDFELNLQIYPDVTLKTYSEITHANDFKNNLPPLTGSVPDFSEVTTNVAFYTVSATPALSIQTSSSMSVIRPDWPYFTDYMTSLKKEVKTSSEWSKWELQPSVQYQEFPTASRHLPFTRSLTLSSLESILAPQRLMISDFSCVRYYGDSYLEFQNVALNPQNNISLEFQTFSSYGLLLYVKQDSNLVDGFFIQLFIENGTLKYHFYCPGEAKFKSINTTVRVDNGQKYTLLIRQELDPCNAELTILGRNTQICESINHVLGKPLPKSGSVFIGGFPDLHGKIQMPVPVKNFTGCIEVIEINNWRSFIPSKAVKNYHINNCRSQGFMLSPTASFVDASDVTQGVDTMWTSVSPSVAAPSVCQQDVCHNGGTCHAIFLSSGIVSFQCDCPLHFTGRFCEKDAGLFFPSFNGNSYLELPFLKFVLEKEHNRTVTIYLTIKTNSLNGTILYSNGNNCGKQFLHLFLVEGRPSVKYGCGNSQNILTVSANYSINTNAFTPITIRYTTPVGSPGVVCMIEMTADGKPPVQKKDTEISHASQAYFESMFLGHIPANVQIHKKAGPVYGFRGCILDLQVNNKEFFIIDEARHGKNIENCHVPWCAHHLCRNNGTCISDNENLFCECPRLYSGKLCQFASCENNPCGNGATCVPKSGTDIVCLCPYGRSGPLCTDAINITQPRFSGTDAFGYTSFLAYSRISDISFHYEFHLKFQLANNHSALQNNLIFFTGQKGHGLNGDDFLAVGLLNGSVVYSYNLGSGIASIRSEPLNLSLGVHTVHLGKFFQEGWLKVDDHKNKSIIAPGRLVGLNVFSQFYVGGYSEYTPDLLPNGADFKNGFQGCIFTLQVRTEKDGHFRGLGNPEGHPNAGRSVGQCHASPCSLMKCGNGGTCIESGTSVYCNCTTGWKGSFCTETVSTCDPEHDPPHHCSRGATCISLPHGYTCFCPLGTTGIYCEQALSISDPSFRSNELSWMSFASFHVRKKTHIQLQFQPLAADGILFYAAQHLKAQSGDFLCISLVNSSVQLRYNLGDRTIILETLQKVTINGSTWHIIKAGRVGAEGYLDLDGINVTEKASTKMSSLDTNTDFYIGGVSSLNLVNPMAIENEPVGFQGCIRQVIINNQELQLTEFGAKGGSNVGDCDGTACGYNTCRNGGECTVNGTTFSCRCLPDWAGNTCNQSVSCLNNLCLHQSLCIPDQSFSYSCLCTLGWVGRYCENKTSFSTAKFMGNSYIKYIDPNYRMRNLQFTTISLNFSTTKTEGLIVWMGIAQNEENDFLAIGLHNQTLKIAVNLGERISVPMSYNNGTFCCNKWHHVVVIQNQTLIKAYINNSLILSEDIDPHKNFVALNYDGICYLGGFEYGRKVNIVTQEIFKTNFVGKIKDVVFFQEPKNIELIKLEGYNVYDGDEQNEVT

SEQ ID NO: 37 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 38 - protein, 224 aa, Homo sapiens
MSRKIEGFLLLLLFGYEATLGLSSTEDEGEDPWYQKACKCDCQGGPNALWSAGATSLDCIPECPYHKPLGFESGEVTPDQITCSNPEQYVGWYSSWTANKARLNSQGFGCAWLSKFQDSSQWLQIDLKEIKVISGILTQGRCDIDEWMTKYSVQYRTDERLNWIYYKDQTGNNRVFYGNSDRTSTVQNLLRPPIISRFIRLIPLGWHVRIAIRMELLECVSKCA

SEQ ID NO: 39 - protein, 809 aa, Homo sapiens
MFKSLTKVNKVKPIGENNENEQSSRRNEEGSHPSNQSQQTTAQEENKGEEKSLKTKSTPVTSEEPHTNIQDKLSKKNSSGDLTTNPDPQNAAEPTGTVPEQKEMDPGKEGPNSPQNKPPAAPVINEYADAQLHNLVKRMRQRTALYKKKLVEGDLSSPEASPQTAKPTAVPPVKESDDKPTEHYYRLLWFKVKKMPLTEYLKRIKLPNSIDSYTDRLYLLWLLLVTLAYNWNCCFIPLRLVFPYQTADNIHYWLIADIICDIIYLYDMLFIQPRLQFVRGGDIIVDSNELRKHYRTSTKFQLDVASIIPFDICYLFFGFNPMFRANRMLKYTSFFEFNHHLESIMDKAYIYRVIRTTGYLLFILHINACVYYWASNYEGIGTTRWVYDGEGNEYLRCYYWAVRTLITIGGLPEPQTLFEIVFQLLNFFSGVFVFSSLIGQMRDVIGAATANQNYFRACMDDTIAYMNNYSIPKLVQKRVRTWYEYTWDSQRMLDESDLLKTLPTTVQLALAIDVNFSIISKVDLFKGCDTQMIYDMLLRLKSVLYLPGDFVCKKGEIGKEMYIIKHGEVQVLGGPDGTKVLVTLKAGSVFGEISLLAAGGGNRRTANVVAHGFANLLTLDKKTLQEILVHYPDSERILMKKARVLLKQKAKTAEATPPRKDLALLFPPKEETPKLFKTLLGGTGKASLARLLKLKREQAAQKKENSEGGEEEGKENEDKQKENEDKQKENEDKGKENEDKDKGREPEEKPLDRPECTASPIAVEEEPHSVRRTVLPRGTSRQSLIISMAPSAEGGEEVLTIEVKEKAKQ

SEQ ID NO: 40 - protein, 694 aa, Homo sapiens
MAKINTQYSHPSRTHLKVKTSDRDLNRAENGLSRAHSSSEETSSVLQPGIAMETRGLADSGQGSFTGQGIARLSRLIFLLRRWAARHVHHQDQGPDSFPDRFRGAELKEVSSQESNAQANVGSQEPADRGRSAWPLAKCNTNTSNNTEEEKKTKKKDAIVVDPSSNLYYRWLTAIALPVFYNWYLLICRACFDELQSEYLMLWLVLDYSADVLYVLDVLVRARTGFLEQGLMVSDTNRLWQHYKTTTQFKLDVLSLVPTDLAYLKVGTNYPEVRFNRLLKFSRLFEFFDRTETRTNYPNMFRIGNLVLYILIIIHWNACIYFAISKFIGFGTDSWVYPNISIPEHGRLSRKYIYSLYWSTLTLTTIGETPPPVKDEEYLFVVVDFLVGVLIFATIVGNVGSMISNMNASRAEFQAKIDSIKQYMQFRKVTKDLETRVIRWFDYLWANKKTVDEKEVLKSLPDKLKAEIAINVHLDTLKKVRIFQDCEAGLLVELVLKLRPTVFSPGDYICKKGDIGKEMYIINEGKLAVVADDGVTQFVVLSDGSYFGEISILNIKGSKSGNRRTANIRSIGYSDLFCLSKDDLMEALTEYPEAKKALEEKGRQILMKDNLIDEELARAGADPKDLEEKVEQLGSSLDTLQTRFARLLAEYNATQMKMKQRLSQLESQVKGGGDKPLADGEVPGDATKTEDKQQ

SEQ ID NO: 41 - protein, 364 aa, Homo sapiens
MAQQWSLQRLAGRHPQDSYEDSTQSSIFTYTNSNSTRGPFEGPNYHIAPRWVYHLTSVWMIFVVIASVFTNGLVLAATMKFKKLRHPLNWILVNLAVADLAETVIASTISVVNQVYGYFVLGHPMCVLEGYTVSLCGITGLWSLAIISWERWMVVCKPFGNVRFDAKLAIVGIAFSWIWAAVWTAPPIFGWSRYWPHGLKTSCGPDVFSGSSYPGVQSYMIVLMVTCCITPLSIIVLCYLQVWLAIRAVAKQQKESESTQKAEKEVTRMVVVMVLAFCFCWGPYAFFACFAAANPGYPFHPLMAALPAFFAKSATIYNPVIYVFMNRQFRNCILQLFGKKVDDGSELSSASKTEVSSVSSVSPA

SEQ ID NO: 42 - protein, 364 aa, Homo sapiens
MAQQWSLQRLAGRHPQDSYEDSTQSSIFTYTNSNSTRGPFEGPNYHIAPRWVYHLTSVWMIFVVTASVFTNGLVLAATMKFKKLRHPLNWILVNLAVADLAETVIASTISIVNQVSGYFVLGHPMCVLEGYTVSLCGITGLWSLAIISWERWMVVCKPFGNVRFDAKLAIVGIAFSWIWAAVWTAPPIFGWSRYWPHGLKTSCGPDVFSGSSYPGVQSYMIVLMVTCCIIPLAIIMLCYLQVWLAIRAVAKQQKESESTQKAEKEVTRMVVVMIFAYCVCWGPYTFFACFAAANPGYAFHPLMAALPAYFAKSATIYNPVIYVFMNRQFRNCILQLFGKKVDDGSELSSASKTEVSSVSSVSPA

SEQ ID NO: 43 - protein, 815 aa, Homo sapiens
MREPEELMPDSGAVFTFGKSKFAENNPGKFWFKNDVPVHLSCGDEHSAVVTGNNKLYMFGSNNWGQLGLGSKSAISKPTCVKALKPEKVKLAACGRNHTLVSTEGGNVYATGGNNEGQLGLGDTEERNTFHVISFFTSEHKIKQLSAGSNTSAALTEDGRLFMWGDNSEGQIGLKNVSNVCVPQQVTIGKPVSWISCGYYHSAFVTTDGELYVFGEPENGKLGLPNQLLGNHRTPQLVSEIPEKVIQVACGGEHTVVLTENAVYTFGLGQFGQLGLGTFLFETSEPKVIENIRDQTISYISCGENHTALITDIGLMYTFGDGRHGKLGLGLENFTNHFIPTLCSNFLRFIVKLVACGGCHMVVFAAPHRGVAKEIEFDEINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLNSNEKSLKLSPVQKQKKQQTIGELTQDTALTENDDSDEYEEMSEMKEGKACKQHVSQGIFMTQPATTIEAFSDEEVEIPEEKEGAEDSKGNGIEEQEVEANEENVKVHGGRKEKTEILSDDLTDKAEDHEFSKTEELKLEDVDEEINAENVESKKKTVGDDESVPTGYHSKTEGAERTNDDSSAETIEKKEKANLEERAICEYNENPKGYMLDDADSSSLEILENSETTPSKDMKKTKKIFLFKRVPSINQKIVKNNNEPLPEIKSIGDQIILKSDNKDADQNHMSQNHQNIPPTNTERRSKSCTIL

SEQ ID NO: 44 - protein, 1152 aa, Homo sapiens
MREPEELMPDSGAVFTFGKSKFAENNPGKFWFKNDVPVHLSCGDEHSAVVTGNNKLYMFGSNNWGQLGLGSKSAISKPTCVKALKPEKVKLAACGRNHTLVSTEGGNVYATGGNNEGQLGLGDTEERNTFHVISFFTSEHKIKQLSAGSNTSAALTEDGRLFMWGDNSEGQIGLKNVSNVCVPQQVTIGKPVSWISCGYYHSAFVTTDGELYVFGEPENGKLGLPNQLLGNHRTPQLVSEIPEKVIQVACGGEHTVVLTENAVYTFGLGQFGQLGLGTFLFETSEPKVIENIRDQTISYISCGENHTALITDIGLMYTFGDGRHGKLGLGLENFTNHFIPTLCSNFLRFIVKLVACGGCHMVVFAAPHRGVAKEIEFDEINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLNSNEKSLKLSPVQKQKKQQTIGELTQDTALTENDDSDEYEEMSEMKEGKACKQHVSQGIFMTQPATTIEAFSDEEVEIPEEKEGAEDSKGNGIEEQEVEANEENVKVHGGRKEKTEILSDDLTDKAEVSEGKAKSVGEAEDGPEGRGDGTCEEGSSGAEHWQDEEREKGEKDKGRGEMERPGEGEKELAEKEEWKKRDGEEQEQKEREQGHQKERNQEMEEGGEEEHGEGEEEEGDREEEEEKEGEGKEEGEGEEVEGEREKEEGERKKEERAGKEEKGEEEGDQGEGEEEETEGRGEEKEEGGEVEGGEVEEGKGEREEEEEEGEGEEEEGEGEEEEGEGEEEEGEGKGEEEGEEGEGEEEGEEGEGEGEEEEGEGEGEEEGEGEGEEEEGEGEGEEEGEGEGEEEEGEGKGEEEGEEGEGEGEEEEGEGEGEDGEGEGEEEEGEWEGEEEEGEGEGEEEGEGEGEEGEGEGEEEEGEGEGEEEEGEEEGEEEGEGEEEGEGEGEEEEEGEVEGEVEGEEGEGEGEEEEGEEEGEEREKEGEGEENRRNREEEEEEEGKYQETGEEENERQDGEEYKKVSKIKGSVKYGKHKTYQKKSVTNTQGNGKEQRSKMPVQSKRLLKNGPSGSKKFWNNVLPHYLELK

SEQ ID NO: 45 - DNA, 7199 bp, synthetic construct
  source 1..7199: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcctgcaggcggccgccgccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctgacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtgagcggccgcgcggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgacctcgagcagtgtggttttgcaagaggaagcaaaaagcctctccacccaggcctggaatgtttccacccaagtcgaaggcagtgtggttttgcaagaggaagcaaaaagcctctccacccaggcctggaatgtttccacccaatgtcgagcaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 46 - DNA, 8176 bp, synthetic construct
  source 1..8176: mol_type = other DNA
aattcccatcatcaataatataccttattttggattgaagccaatatgataatgagggggtggagtttgtgacgtggcgcggggcgtgggaacggggcgggtgacgtagtagtctctagagtcctgtattagaggtcacgtgagtgttttgcgacattttgcgacaccatgtggtcacgctgggtatttaagcccgagtgagcacgcagggtctccattttgaagcgggaggtttgaacgcgcagccaccacggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatgctctagactactacgtcacccgccccgttcccacgccccgcgccacgtcacaaactccaccccctcattatcatattggcttcaatccaaaataaggtatattattgatgatgcatcgctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 47 - DNA, 18876 bp, synthetic construct
  source 1..18876: mol_type = other DNA
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaaggtgcacggcccacgtggccactagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 48 - DNA, 22754 bp, synthetic construct
  source 1..22754: mol_type = other DNA
ccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagccatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggatcgaattaattcttaattaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagggggatcctctagaactagtggatccgtccctagaagtaaaaaagggaaaaaagagtgtgtttgtcaaaataggagacaggtggtggcaaccagggacttataggggaccttacatctacagaccaacagatgcccccttaccatatacaggaagatatgacttaaattgggataggtgggtcacaatcaacggctataaagtgttatacagatccctcccctttcgtgaaagactcgccagagctagacctccttggtgtatgctaactgagaaagagaaagacgacatgaaacaacaggtacatgattatatttatctaggaacaggaatgcacttttggggaaaggttttccataccaaggaaggggcagtggctggactgatagaacattattctgcaaaaacttatggtatgagttattatgattagcctttatttgcccaaccttgcggttcccagggtttaaataagtttatggttacaaactgttcttaaaacaaggatgtgagacaagtggtttcctgagttggtttggtatcaaatgttctgatctgagctcttagtgttctattttcctatgttcttttggaatctatccaagtcttatgtaaatgcttatgtaaaccataatataaaagagtgctgattttttgagtaaacttgcaacagtcctaacattcttctctcgtgtgtttgtgtctgttcgccatcccgtctccgctcgtcacttatccttcacttttcagagggtccccccgcagatcccggtcaccctcaggtcgggacctgcagaagacgcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacatcagcttatcgatgcatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttacaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgtctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccagggcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcctgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaatcgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggat

SEQ ID NO: 49 - DNA, 1866 bp, synthetic construct
  source 1..1866: mol_type = other DNA
acggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 50 - DNA, 2198 bp, synthetic construct
  source 1..2198: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcg

SEQ ID NO: 51 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 52 - protein, 11 aa, synthetic construct
  VARIANT 7: note = Any amino acid
  VARIANT 9: note = Any amino acid
SAAGADXAXDS